(19) United States
(12) Patent Application Publication
(10) Pub. No.: US 2008/0207614 A1
(43) Pub. Date: Aug. 28, 2008

(54) QUINAZOLINE DERIVATIVES FOR THE TREATMENT AND PREVENTION OF DIABETES AND OBESITY

(75) Inventors: Nam Kyu Lee, Suwon-si (KR); Jun Won Lee, Gunpo-si (KR); Sukho Lee, Suwon-si (KR); Guang-Jin Im, Ansan-si (KR); Hye Young Han, Seoul (KR); Tae Kon Kim, Suwon-si (KR); Yong Hyuk Kim, Suwon-si (KR); Wie-Jong Kwak, Seoul (KR); Sang Woong Kim, Daejeon (KR); Joohun Ha, Seoul (KR); Eon Kyum Kim, Daejeon (KR); Jung Kyu Lee, Daejeon (KR); Choong Yeul Yoo, Daejeon (KR); Dae Yeon Lee, Daegu (KR)

Correspondence Address: JHK LAW, P.O. BOX 1078, LA CANADA, CA, 91012-1078, US

(73) Assignees: SK CHEMICALS CO., LTD., Suwon-si (KR); LEADGENEX INC., Daejeon (KR); INDUSTRY ACADEMIC COOPERATION FOUNDATION OF KYUNGHEE UNIVERSITY, Yongsin-si (KR)

(21) Appl. No.: 11/772,098

(22) Filed: Jun. 29, 2007

(30) Foreign Application Priority Data
Dec. 31, 2004 (KR) 10-2004-0117706

(51) Int. Cl. A61K 31/517; A61P 3/10; C07D 239/72; C07D 413/02; C07D 401/02; C07D 239/86; A61P 3/04

(52) U.S. Cl. 514/234.5; 544/283; 514/266.1; 544/287; 544/293; 544/116; 544/284; 514/266.2

(57) ABSTRACT

The present invention relates to novel quinazoline derivatives effective in lowering blood glucose level and body weight, and a medicine for treatment and/or prevention of diabetes and/or obesity, which comprises the compound as an active ingredient.

### TECHNICAL FIELD

The present invention relates to novel quinazoline derivatives effective in lowering blood glucose level and reducing body weight, and a preparation for treatment and/or prevention of diabetes and/or obesity, which comprises the compound as an active ingredient.

### BACKGROUND ART

Diabetes, one of the most common modern diseases, is on the increase in Korea, too. Because of its severe symptoms and sequela, the disease is becoming a social problem. Improvement of living standards and environments resulting form economic development has led to lack of exercises, while calorie intake has increased significantly. As a result, obesity and type 2 diabetes are also on the increase. If diabetes persists, blood lipid level increases, not to mention the blood glucose level, leading to cardiovascular diseases such as arteriosclerosis and coronary heart disease and complications such as diabetic kidney and retinal diseases. Therefore, development of a treatment for the above disease is an important issue.

Diabetes is classified roughly into the insulin-dependent diabetes mellitus (hereunder referred to as “IDDM”) and the non-insulin-dependent diabetes mellitus (hereunder referred to as “NIDDM”). The insulin-dependent diabetes mellitus (IDDM), or the type 1 diabetes, refers to a condition in which the beta cells of the pancreas produce little or no insulin due to immunological cause. In contrast, the non-insulin-dependent diabetes mellitus (NIDDM), or the type 2 diabetes, is caused by insulin secretory defect and insulin resistance and accounts for over 95% of diabetes. Insulin secretory defect refers to the condition in which the beta cells of the pancreas cannot secrete an adequate amount of insulin depending on the blood glucose level and includes both quantitative and functional secretory defects. Insulin resistance refers to the condition in which target cells fail to respond to ordinary levels of circulating insulin. The cause of insulin resistance is generally thought of as hindered signal transfer after binding to the cell membrane receptor, resulting from genetic factors, obesity, reduced physical activity, hyperglycemia, abnormal blood lipid level, etc. What is important is that the two factors are related with each other. If there is insulin resistance, more insulin has to be secreted to overcome the resistance. And, hyperglycemia caused by insufficient secretion of insulin may aggravate the insulin resistance. Accordingly, extensive researches have been conducted to study insulin signal transfer system, glucose transfer and metabolism processes to accurately identify the cause of NIDDM. However, the exact pathological mechanism has yet to be found because the cause of NIDDM is very complicated and diverse and improvement of adequate treatment is being retarded.

It is well known that the type 2 diabetes (NIDDM) is related with obesity, but the exact mechanism of the interaction or interrelationship still needs to be elucidated. On the assumption that obesity and fatty acid metabolism disturbance resulting therefrom may be inseparably related with metabolic diseases, the focus of the research on the cause of diabetes has recently shifted from “glucose metabolism” to “fatty acid metabolism.” In type 2 diabetes patients, such lipid metabolism disturbances as increase in blood fatty acid level, increase in neutral fats, decrease in high-density lipoprotein cholesterol, accumulation of fats in muscles, increase in abdominal fats and fatty liver are frequently found, as well as hyperglycemia. Of course, it is not certain as yet whether the trouble with lipid homeostasis results from hyperglycemia due to diabetes or it causes diabetes. Insulin resistance is preceded by excessive accumulation of neutral fats in target organs of insulin, including skeletal muscles, cardiac muscles and liver tissues, or fatty acid metabolism disturbance including increase of free fatty acids in fat tissues. When such a state persists, insulin resistance is aggravated and hyperglycemia occurs after meals or during fasting, which, in turn, aggravates the insulin resistance and fatty acid metabolism disturbance. Other metabolic diseases are induced as the cycle is repeated. This pathological phenomenon is corroborated with relative consistency in in vitro experiments, animal tests and human tests. However, it is still uncertain what causes the lipid metabolism disturbance or by what mechanism the insulin resistance is brought about, interfering with physiological homeostasis.

At present, insulin resistance is thought to be the most important pathological cause of NIDDM. Insulin resistance is the condition in which glucose is not fully utilized even when there is a sufficient amount of insulin. Currently, various hypoglycemic drugs are used to treat insulin resistance. The hypoglycemic drugs are classified as follows depending on the treatment mechanism and the target site:

1) Sulfonylurea based drugs promote the movement of insulin-containing granules in the beta cells of the pancreas, thereby promoting the secretion of insulin at second hand. There was a report of severe side effects, including inducement of hypoglycemia, but recently, a drug with minimized side effects was developed.

2) Biguanide based drugs transfer glucose to muscle cells and inhibit gluconeogenesis in the liver. They are advantageous in that they do not cause hypoglycemia, but care should be taken for aged people and patients with cardiovascular diseases.

3) α-Glucosidase inhibitors inhibit the enzymatic action of glucose production in the small intestine, preventing abrupt increase of blood glucose level after meals. With few side effects, the drugs are used to treat mild cases of diabetes.

4) Recently developed thiazolidione (TZD) based drugs are known to activate PPAR-γ, which participates in the differentiation of fat cells.

However, because these oral hypoglycemic drugs are not so effective in reducing blood glucose level and cause side effects, development of safer diabetic treatments is imminent. And, since the type 2 diabetes is caused by various reasons in insulin signal transfer system and carbohydrate and lipid metabolism processes, search for new target proteins capable of regulating the signal transfer system and metabolism processes is essential for development of such treatment drugs.

Recently, as the role of 5′AMP-activated protein kinase (hereunder referred to as “AMPK”) in carbohydrate and lipid metabolism and glucose absorption control is elucidated, the possibility of utilizing the activators of the enzyme as treatment for type 2 diabetes is proposed. The basis is as follows.

1) In the muscle cells of exercised mice, increase of AMP resulting from ATP consumption, which increases activity of AMPK, was found. As a result, increase in glucose absorption was observed.

2) The increase of glucose absorption caused by AMPK is independent of the insulin signal transfer system, which implies that it can be a new target material for most of type 2 diabetics showing abnormality in the insulin signal transfer system.

3) In fact, a lot of type 2 diabetics could reduce their blood glucose level through exercise. Also in the diabetic model animal test, it was found that exercise promotes activation of AMPK and decrease in blood glucose level.

4) AMPK plays a variety of other roles in the body. Besides controlling insulin production in pancreatic cells, it inhibits activation of acetyl CoA carboxylase (ACC) and HMG-CoA reductase in fat cells, thereby inhibiting fat production and cholesterol synthesis. Thus, it is important in controlling obesity.

To conclude, AMPK plays a key role in energy homeostasis control and carbohydrate and lipid metabolism control, as activated by AMP's accumulated by muscle contraction, exercise or energy depletion in cells. Also, it is viewed as new target material for treating metabolic diseases, such as diabetes and obesity, caused by energy imbalance. The importance of AMPK in glucose absorption during exercise is widely known. The fact that the action of AMPK is independent of the insulin signal transfer system means that it can become a new target material for treatment of type 2 diabetics, most of whom show troubles with the insulin signal transfer system. Actually, a lot of type 2 diabetics could reduce their blood glucose level through exercise and the diabetic model animal test confirmed that exercise promotes activation of AMPK and decrease in blood glucose level. There is a report that metformin or thiazolidinedione (TZD) based drugs, which are widely used as diabetic treatments at present, reduce blood sugar level partly by an AMPK-involved mechanism. Thus, search of the drugs that can activate AMPK will lead to the development of a new-concept treatment that imitates the therapeutic effect of exercise on a variety of metabolic diseases.

However, there are a few reports about AMPK activators which are selectively effective against type 2 diabetes and obesity and significantly reduce blood glucose level in diabetic animal tests.

The present inventors synthesized a novel material that has a remarkable effect in increase of AMPK activity and prevention and/or treatment of obesity and diabetes and confirmed the effect.

To solve the above-mentioned problems, the present inventors synthesized several compounds with quinazoline backbones and confirmed that they are superior in reducing blood glucose level and body weight.

Therefore, in an embodiment of the present invention there is provided a novel quinazoline compound effective in reducing blood sugar level and body weight, which is essential in treating diabetes and obesity.

In an embodiment of the present invention there is provided a treatment for diabetes and obesity with no toxicity or side effects, which comprises the novel quinazoline compound, a pharmaceutically available salt thereof or a pharmaceutically available prodrug thereof as an active ingredient.

### DISCLOSURE OF THE INVENTION

The present invention relates to a quinazoline derivative, a pharmaceutically available salt thereof or a pharmaceutically available prodrug thereof effective in treating diabetes and obesity, which is represented by the formula 1 below:

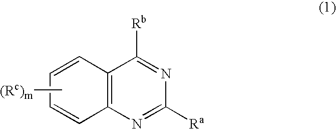

wherein

R^{a} is a substituent selected from hydrogen, (C₁-C₁₈)alkyl, (C₂-C₈)alkenyl, (C₂-C₈)alkynyl, halogeno-(C₁-C₆)alkyl, hydroxy-(C₁-C₆)alkyl, (C₁-C₆)alkoxy-(C₁-C₆)alkyl, cyano-(C₁-C₆)alkyl, amino-(C₁-C₆)alkyl, (C₁-C₆)alkylamino-(C₁-C₆)alkyl, di-[(C₁-C₆)alkyl]amino-(C₁-C₆)alkyl, (C₁-C₆)alkylthio-(C₁-C₆)alkyl, (C₁-C₆)alkylsulfinyl-(C₁-C₆)alkyl and (C₁-C₆)alkylsulfonyl-(C₁-C₆)alkyl or —X¹-Q¹ [wherein X¹ is a direct bonding or selected from O, S, SO, SO₂, N(R⁴), CO, CO₂, CH(OR⁴), CON(R⁴), N(R⁴)CO, SO₂N(R⁴), N(R⁴)SO₂, C(R⁴)₂₋₀, C(R⁴)₂S, N(R⁴)C(R⁴)₂, N(R⁴)CON(R⁴) and N(R⁴)CSN(R⁴), wherein R⁴ is a group selected from hydrogen, (C₁-C₆)alkyl, aryl, aryl-(C₁-C₆)alkyl, (C₃-C₇)cycloalkyl, (C₃-C₇)cycloalkyl-(C₁-C₆)alkyl, (C₃-C₇)cycloalkenyl, (C₃-C₇)cycloalkenyl-(C₁-C₆)alkyl, heteroaryl, heteroaryl-(C₁-C₆)alkyl, heterocyclyl and heterocyclyl-(C₁-C₆)alkyl, and Q¹ and R⁴ may be identical or different],

wherein the neighboring carbon atoms in (C₂-C₆)alkylene of R^{a} may be separated by a group selected from the group consisting of O, S, SO, SO₂, N(R⁵), CO, CH(OR⁵), CON(R⁵), N(R⁵)CO, SO₂N(R⁵), N(R⁵)SO₂, C(R⁵)═C(R⁵) and C∫C, wherein R⁵ is a hydrogen atom or (C₁-C₆)alkyl,

any CH₂═CH— or HC≡C— group of R^{a} may have a substituent, at the terminal or at CH₂═ or HC≡, selected from halogeno, carboxyl, carbamoyl, (C₁-C₆)alkoxycarbonyl, N—(C₁-C₆)alkylcarbamoyl, N,N-di-[(C₁-C₆)alkyl]carbamoyl, amino-(C₁-C₆)alkyl, (C₁-C₆)alkylamino-(C₁-C₆)alkyl and di-[(C₁-C₆)alkyl]amino-(C₁-C₆)alkyl or -Q²-X² [wherein X² is a direct bonding or selected from CO and N(R⁶)CO, wherein R⁶ is a hydrogen atom or (C₁-C₆)alkyl, and Q² is aryl, aryl-(C₁-C₆)alkyl, heteroaryl, heteroaryl-(C₁-C₆)alkyl, heterocyclyl or heterocyclyl-(C₁-C₆)alkyl],

any CH₂ or CH₃ of R^{a} may have a substituent selected from halogeno, (C₁-C₆)alkyl, hydroxy, cyano, amino, carboxyl, carbamoyl, (C₁-C₆)alkoxy, (C₁-C₆)alkylthio, (C₁-C₆)alkylsulfinyl, (C₁-C₆)alkylsulfonyl, (C₁-C₆)alkylamino, di-[(C₁-C₆)alkyl]amino, (C₁-C₆)alkoxycarbonyl, N—(C₁-C₆)alkylcarbamoyl, N,N-di-[(C₁-C₆)alkyl]carbamoyl, (C₂-C₆)alkanoyl, (C₂-C₆)alkanoyloxy, (C₂-C₆)alkanoylamino, N—(C₁-C₆)alkyl-(C₂-C₆)alkanoylamino, N—(C₁-C₆)alkylsulfamoyl, N,N-di-[(C₁-C₆)alkyl]sulfamoyl, (C₁-C₆)alkanesulfonylamino and N—(C₁-C₆)alkyl-(C₁-C₆)alkanesulfonylamino or —X³-Q³ [wherein X³ is a direct bonding or selected from O, S, SO, SO₂, N(R⁷), CO, CH(OR⁷), CON(R⁷), N(R⁷)CO, SO₂N(R⁷), N(R⁷)SO₂, C(R⁷)₂O, C(R⁷)₂S, N(R⁷)C(R⁷)₂, N(R⁷)CON(R⁷) and N(R⁷)CSN(R⁷), wherein R⁷ is a hydrogen atom, (C₁-C₆)alkyl, aryl, aryl-(C₁-C₆)alkyl, (C₃-C₇)cycloalkyl, (C₃-C₇)cycloalkyl-(C₁-C₆)alkyl, (C₃-C₇)cycloalkenyl, (C₃-C₇)cycloalkenyl-(C₁-C₆)alkyl, heteroaryl, heteroaryl-(C₁-C₆)alkyl, heterocyclyl or heterocyclyl-(C₁-C₆)alkyl, and Q³ may be identical with or different from R⁷],

any aryl, heteroaryl or heterocyclyl of R^{a} may have 0 to 3 substituents, which may be identical or different and are selected from halogeno, trifluoromethyl, cyano, nitro, hydroxy, amino, carboxyl, carbamoyl, (C₁-C₆)alkyl, (C₂-C₈)alkenyl, (C₂-C₈)alkynyl, (C₁-C₆)alkoxy, (C₂-C₆)alkenyloxy, (C₂-C₆)alkynyloxy, (C₁-C₆)alkylthio, (C₁-C₆)alkylsulfinyl, (C₁-C₆)alkylsulfonyl, (C₁-C₆)alkylamino, di-[(C₁-C₆)alkyl]amino, (C₁-C₆)alkoxycarbonyl, N—(C₁-C₆)alkylcarbamoyl, N,N-di-[(C₁-C₆)alkyl]carbamoyl, (C₂-C₆)alkanoyl, (C₂-C₆)alkanoyloxy, (C₂-C₆)alkanoylamino, N—(C₁-C₆)alkyl-(C₂-C₆)alkanoylamino, N—(C₁-C₆)alkylsulfamoyl, N,N-di-[(C₁-C₆)alkyl]sulfamoyl, (C₁-C₆)alkanesulfonylamino and N—(C₁-C₆)alkyl-(C₁-C₆)alkanesulfonylamino or —X⁴—R⁸ [wherein X⁴ is a direct bonding or selected from O and N(R⁹), wherein R⁹ is a hydrogen atom or (C₁-C₆)alkyl, and R⁸ is selected from halogeno-(C₁-C₆)alkyl, hydroxy-(C₁-C₆)alkyl, (C₁-C₆)alkoxy-(C₁-C₆)alkyl, cyano-(C₁-C₆)alkyl, amino-(C₁-C₆)alkyl, (C₁-C₆)alkylamino-(C₁-C₆)alkyl, di-[(C₁-C₆)alkyl]amino-(C₁-C₆)alkyl, (C₂-C₆)alkanoylamino-(C₁-C₆)alkyl or (C₁-C₆)alkoxycarbonylamino-(C₁-C₆)alkyl] or —X⁵-Q⁴ [wherein X⁵ is a direct bonding or selected from O, CO and N(R¹⁰), wherein R¹⁰ is a hydrogen atom or (C₁-C₆)alkyl, and Q⁴ is aryl, aryl-(C₁-C₆)alkyl, heteroaryl, heteroaryl-(C₁-C₆)alkyl, heterocyclyl or heterocyclyl-(C₁-C₆)alkyl, which may have 1 or 2 identical or different substituents selected from halogeno, (C₁-C₆)alkyl and (C₁-C₆)alkoxy] and

heterocyclyl of R^{a} may have 1 or 2 oxo or thioxo substituents;

R^{b} is a substituent selected from hydrogen, (C₁-C₁₈)alkyl, (C₂-C₈)alkenyl, (C₂-C₈)alkynyl, halogeno-(C₁-C₆)alkyl, hydroxy-(C₁-C₆)alkyl, (C₁-C₆)alkoxy-(C₁-C₆)alkyl, cyano-(C₁-C₆)alkyl, amino-(C₁-C₆)alkyl, (C₁-C₆)alkylamino-(C₁-C₆)alkyl, di-[(C₁-C₆)alkyl]amino-(C₁-C₆)alkyl, (C₁-C₆)alkylthio-(C₁-C₆)alkyl, (C₁-C₆)alkylsulfinyl-(C₁-C₆)alkyl or (C₁-C₆)alkylsulfonyl-(C₁-C₆)alkyl or —X¹-Q¹ [wherein X¹ is a direct bonding or selected from O, S, SO, SO₂, N(R⁴), CO, CO₂(R⁴), CH(OR⁴), CON(R⁴), N(R⁴)CO, SO₂N(R⁴), N(R⁴)SO₂, C(R⁴)₂O, C(R⁴)₂S, N(R⁴)C(R⁴)₂, N(R⁴)CON(R⁴) and N(R⁴)CSN(R⁴), wherein R⁴ is a hydrogen atom, (C₁-C₆)alkyl, aryl, aryl-(C₁-C₆)alkyl, (C₃-C₇)cycloalkyl, (C₃-C₇)cycloalkyl-(C₁-C₆)alkyl, (C₃-C₇)cycloalkenyl, (C₃-C₇)cycloalkenyl-(C₁-C₆)alkyl, heteroaryl, heteroaryl-(C₁-C₆)alkyl, heterocyclyl or heterocyclyl-(C₁-C₆)alkyl, and Q¹ and R⁴ may be identical or different,

wherein the neighboring carbon atoms in (C₂-C₆)alkylene of R^{b} may be separated by a group selected from O, S, SO, SO₂, N(R⁵), CO, CH(OR⁵), CON(R⁵), N(R⁵)CO, SO₂N(R⁵), N(R⁵)SO₂, CH═CH and C≡C, wherein R⁵ is a hydrogen atom or (C₁-C₆)alkyl,

any CH₂═CH— or HC≡C— of R^{b} may have a substituent, at the terminal or at CH₂═ or HC≡, selected from halogeno, carboxyl, carbamoyl, (C₁-C₆)alkoxycarbonyl, N—(C₁-C₆)alkylcarbamoyl, N,N-di-[(C₁-C₆)alkyl]carbamoyl, amino-(C₁-C₆)alkyl, (C₁-C₆)alkylamino-(C₁-C₆)alkyl and di-[(C₁-C₆)alkyl]amino-(C₁-C₆)alkyl or -Q²-X² [wherein X² is a direct bonding or selected from CO and N(R⁶)CO, wherein R⁶ is a hydrogen atom or (C₁-C₆)alkyl, and Q² is aryl, aryl-(C₁-C₆)alkyl, heteroaryl, heteroaryl-(C₁-C₆)alkyl, heterocyclyl or heterocyclyl-(C₁-C₆)alkyl],

any CH₂ or CH₃ of R^{b} may have at least one substituent selected from halogeno, (C₁-C₆)alkyl, hydroxy, cyano, amino, carboxyl, carbamoyl, (C₁-C₆)alkoxy, (C₁-C₆)alkylthio, (C₁-C₆)alkylsulfinyl, (C₁-C₆)alkylsulfonyl, (C₁-C₆)alkylamino, di-[(C₁-C₆)alkyl]amino, (C₁-C₆)alkoxycarbonyl, N—(C₁-C₆)alkylcarbamoyl, N,N-di-[(C₁-C₆)alkyl]carbamoyl, (C₂-C₆)alkanoyl, (C₂-C₆)alkanoyloxy, (C₂-C₆)alkanoylamino, N—(C₁-C₆)alkyl-(C₂-C₆)alkanoylamino, N—(C₁-C₆)alkylsulfamoyl, N,N-di-[(C₁-C₆)alkyl]sulfamoyl, (C₁-C₆)alkanesulfonylamino and N—(C₁-C₆)alkyl-(C₁-C₆)alkanesulfonylamino or —X³-Q³ [wherein X³ is a direct bonding or selected from O, S, SO, SO₂, N(R⁷), CO, CH(OR⁷), CON(R⁷), N(R⁷)CO, SO₂N(R⁷), N(R⁷)SO₂, C(R⁷)₂O, C(R⁷)₂S, N(R⁷)C(R⁷)₂, N(R⁷)CON(R⁷) and N(R⁷)CSN(R⁷), wherein R⁷ is a hydrogen atom, (C₁-C₆)alkyl, aryl, aryl-(C₁-C₆)alkyl, (C₃-C₇)cycloalkyl, (C₃-C₇)cycloalkyl-(C₁-C₆)alkyl, (C₃-C₇)cycloalkenyl, (C₃-C₇)cycloalkenyl-(C₁-C₆)alkyl, heteroaryl, heteroaryl-(C₁-C₆)alkyl, heterocyclyl or heterocyclyl-(C₁-C₆)alkyl, and Q³ and R⁷ may be identical or different],

any aryl, heteroaryl or heterocyclyl of R^{b} may have 1, 2 or 3 substituents, which may be identical or different and are selected from hydrogen, halogeno, trifluoromethyl, cyano, nitro, hydroxy, amino, carboxyl, carbamoyl, (C₁-C₆)alkyl, (C₂-C₈)alkenyl, (C₂-C₈)alkynyl, (C₁-C₆)alkoxy, (C₂-C₆)alkenyloxy, (C₂-C₆)alkynyloxy, (C₁-C₆)alkylthio, (C₁-C₆)alkylsulfinyl, (C₁-C₆)alkylsulfonyl, (C₁-C₆)alkylamino, di-[(C₁-C₆)alkyl]amino, (C₁-C₆)alkoxycarbonyl, N—(C₁-C₆)alkylcarbamoyl, N,N-di-[(C₁-C₆)alkyl]carbamoyl, (C₂-C₆)alkanoyl, (C₂-C₆)alkanoyloxy, (C₂-C₆)alkanoylamino, N—(C₁-C₆)alkyl-(C₂-C₆)alkanoylamino, (C₃-C₆)alkenoylamino, N—(C₁-C₆)alkyl-(C₃-C₆)alkenoylamino, (C₃-C₆)alkynoylamino, N—(C₁-C₆)alkyl-(C₃-C₆)alkynoylamino, N—(C₁-C₆)alkylsulfamoyl, N,N-di-[(C₁-C₆)alkyl]sulfamoyl, (C₁-C₆)alkanesulfonylamino and N—(C₁-C₆)alkyl-(C₁-C₆)alkanesulfonylamino or —X⁴—R⁸ [wherein X⁴ is a direct bonding or selected from O and N(R⁹), wherein R⁹ is a hydrogen atom or (C₁-C₆)alkyl, and R⁸ is selected from halogeno-(C₁-C₆)alkyl, hydroxy-(C₁-C₆)alkyl, (C₁-C₆)alkoxy-(C₁-C₆)alkyl, cyano-(C₁-C₆)alkyl, amino-(C₁-C₆)alkyl, (C₁-C₆)alkylamino-(C₁-C₆)alkyl and di-[(C₁-C₆)alkyl]amino-(C₁-C₆)alkyl] or —X⁵-Q⁴ [wherein X⁵ is a direct bonding or selected from O, CO and N(R¹⁰), wherein R¹⁰ is a hydrogen atom or (C₁-C₆)alkyl, and Q⁴ is aryl, aryl-(C₁-C₆)alkyl, heteroaryl, heteroaryl-(C₁-C₆)alkyl, heterocyclyl or heterocyclyl-(C₁-C₆)alkyl, which may have 1 or 2 identical or different substituents selected from halogeno, (C₁-C₆)alkyl and (C₁-C₆)alkoxy]],

heterocyclyl of R^{b} may have 1 or 2 oxo or thioxo substituents and

any identical or different substituents of aryl, heteroaryl or heterocyclyl of R^{b} may bond with each other to form —CH═CH—CH═CH—, —N═CH—CH═CH—, —CH═N—CH═CH—, —CH═CH—N═CH—, CH═CH—CH═N—, —N═CH—N═CH—, —CH═N—CH═N—, —N═CH—CH═N—, —N═N—CH═CH—, —CH═CH—N═N—, —CH═CH—O—, —O—CH═CH—, —CH═CH—S—, —S—CH═CH—, —C(R¹¹)₂—C(R¹¹)₂—O—, —O—C(R¹¹)₂—C(R¹¹)₂—, —C(R¹¹)₂—C(R¹¹)₂—S—, —S—C(R¹¹)₂—C(R¹¹)₂—, —O—C(R¹¹)₂—O—, —O—C(R¹¹)₂—C(R¹¹)₂—O—, —S—C(R¹¹)₂—S—, —S—C(R¹¹)₂—C(R¹¹)₂—S—, —CH═CH—NH—, —NH—CH═CH—, —C(R¹¹)₂—C(R¹¹)₂—NH—, —NH—C(R¹¹)₂—C(R¹¹)₂—, —N═CH—NH—, —NH—CH═N—, —NH—C(R¹¹)₂—NH—, —O—CH═N—, —N═CH—O—, —S—CH═N—, —N═CH—S—, —O—C(R¹¹)₂—NH—, —NH—C(R¹¹)₂—O—, —S—C(R¹¹)₂—NH—, —NH—C(R¹¹)₂—S—, —O—N═CH—, —CH═N—O—, —S—N═CH—, —CH═N—S—, —O—NH—C(R¹¹)₂—, —C(R¹¹)₂—NH—O—, —S—NH—C(R¹¹)₂—, —C(R¹¹)₂—NH—S—, —NH—N═CH—, —CH═N—NH—, —NH—NH—C(R¹¹)₂—, —C(R¹¹)₂—NH—NH, —N═N—NH— or —NH—N═N— [wherein R¹¹ is a hydrogen atom or (C₁-C₆)alkyl];

m is 0, 1, 2, 3 or 4; and

each R^{c} may be identical or different and is selected from hydrogen, halogeno, trifluoromethyl, cyano, isocyano, nitro, hydroxy, mercapto, amino, formyl, carboxyl, carbamoyl, (C₁-C₆)alkyl, (C₂-C₈)alkenyl, (C₂-C₈)alkynyl, (C₁-C₆)alkoxy, (C₂-C₆)alkenyloxy, (C₂-C₆)alkynyloxy, (C₁-C₆)alkylthio, (C₁-C₆)alkylsulfinyl, (C₁-C₆)alkylsulfonyl, (C₁-C₆)alkylamino, di-[(C₁-C₆)alkyl]amino, (C₁-C₆)alkoxycarbonyl, N—(C₁-C₆)alkylcarbamoyl, N,N-di-[(C₁-C₆)alkyl]carbamoyl, (C₂-C₆)alkanoyl, (C₂-C₆)alkanoyloxy, (C₂-C₆)alkanoylamino, N—(C₁-C₆)alkyl-(C₂-C₆)alkanoylamino, (C₃-C₆)alkenoylamino, N—(C₁-C₆)alkyl-(C₃-C₆)alkenoylamino, (C₃-C₆)alkynoylamino, N—(C₁-C₆)alkyl-(C₃-C₆)alkynoylamino, N—(C₁-C₆)alkylsulfamoyl, N,N-di-[(C₁-C₆)alkyl]sulfamoyl, (C₁-C₆)alkanesulfonylamino, N—(C₁-C₆)alkyl-(C₁-C₆)alkanesulfonylamino, aryl, aryl-(C₁-C₆)alkyl, (C₃-C₇)cycloalkyl, (C₃-C₇)cycloalkyl-(C₁-C₆)alkyl, (C₃-C₇)cycloalkenyl, (C₃-C₇)cycloalkenyl-(C₁-C₆)alkyl, heteroaryl, heteroaryl-(C₁-C₆)alkyl, heterocyclyl and heterocyclyl-(C₁-C₆)alkyl;

wherein the neighboring carbon atoms of (C₂-C₆)alkylene of R^{c} may be separated by a group selected from O, S, SO, SO₂, N(R¹²), CO, CH(OR¹²), CON(R¹²), N(R¹²)CO, SO₂N(R¹²), N(R¹²)SO₂, CH═CH and C≡C, wherein R¹² is a hydrogen atom or (C₁-C₆)alkyl,

any CH₂═CH— or HC≡C— of R^{c} may have a substituent, at the terminal or at CH₂═ or HC≡, selected from halogeno, carboxyl, carbamoyl, (C₁-C₆)alkoxycarbonyl, N—(C₁-C₆)alkylcarbamoyl, N,N-di-[(C₁-C₆)alkyl]carbamoyl, amino-(C₁-C₆)alkyl, (C₁-C₆)alkylamino-(C₁-C₆)alkyl and di-[(C₁-C₆)alkyl]amino-(C₁-C₆)alkyl or -Q⁵-X⁶ [wherein X⁶ is a direct bonding or selected from CO and N(R¹³)CO, wherein R¹³ is a hydrogen atom or (C₁-C₆)alkyl, and Q⁵ is aryl, aryl-(C₁-C₆)alkyl, heteroaryl, heteroaryl-(C₁-C₆)alkyl, heterocyclyl or heterocyclyl-(C₁-C₆)alkyl],

any CH₂ or CH₃ of R^{c} may have at least one substituent selected from halogeno, (C₁-C₆)alkyl, hydroxy, cyano, amino, carboxyl, carbamoyl, (C₁-C₆)alkoxy, (C₁-C₆)alkylthio, (C₁-C₆)alkylsulfinyl, (C₁-C₆)alkylsulfonyl, (C₁-C₆)alkylamino, di-[(C₁-C₆)alkyl]amino, (C₁-C₆)alkoxycarbonyl, N—(C₁-C₆)alkylcarbamoyl, N,N-di-[(C₁-C₆)alkyl]carbamoyl, (C₂-C₆)alkanoyl, (C₂-C₆)alkanoyloxy, (C₂-C₆)alkanoylamino, N—(C₁-C₆)alkyl-(C₂-C₆)alkanoylamino, N—(C₁-C₆)alkylsulfamoyl, N,N-di-[(C₁-C₆)alkyl]sulfamoyl, (C₁-C₆)alkanesulfonylamino and N—(C₁-C₆)alkyl-(C₁-C₆)alkanesulfonylamino or —X⁷-Q⁶ [wherein X⁷ is a direct bonding or selected from O, S, SO, SO₂, N(R¹⁴), CO, CH(OR¹⁴), CON(R¹⁴), N(R¹⁴)CO, SO₂N(R¹⁴), N(R¹⁴)SO₂, C(R¹⁴)₂O, C(R¹⁴)₂S and N(R¹⁴)C(R¹⁴)₂, wherein R¹⁴ is a hydrogen atom or (C₁-C₆)alkyl, and Q⁶ is aryl, aryl-(C₁-C₆)alkyl, (C₃-C₇)cycloalkyl, (C₃-C₇)cycloalkyl-(C₁-C₆)alkyl, (C₃-C₇)cycloalkenyl, (C₃-C₇)cycloalkenyl-(C₁-C₆)alkyl, heteroaryl, heteroaryl-(C₁-C₆)alkyl, heterocyclyl or heterocyclyl-(C₁-C₆)alkyl],

any aryl, heteroaryl or heterocyclyl of R^{c} may have 1, 2 or 3 identical or different substituents selected from hydrogen, halogeno, trifluoromethyl, cyano, nitro, hydroxy, amino, carboxyl, carbamoyl, (C₁-C₆)alkyl, (C₂-C₈)alkenyl, (C₂-C₈)alkynyl, (C₁-C₆)alkoxy, (C₂-C₆)alkenyloxy, (C₂-C₆)alkynyloxy, (C₁-C₆)alkylthio, (C₁-C₆)alkylsulfinyl, (C₁-C₆)alkylsulfonyl, (C₁-C₆)alkylamino, di-[(C₁-C₆)alkyl]amino, (C₁-C₆)alkoxycarbonyl, N—(C₁-C₆)alkylcarbamoyl, N,N-di-[(C₁-C₆)alkyl]carbamoyl, (C₂-C₆)alkanoyl, (C₂-C₆)alkanoyloxy, (C₂-C₆)alkanoylamino, N—(C₁-C₆)alkyl-(C₂-C₆)alkanoylamino, (C₃-C₆)alkenoylamino, N—(C₁-C₆)alkyl-(C₃-C₆)alkenoylamino, (C₃-C₆)alkynoylamino, N—(C₁-C₆)alkyl-(C₃-C₆)alkynoylamino, N—(C₁-C₆)alkylsulfamoyl, N,N-di-[(C₁-C₆)alkyl]sulfamoyl, (C₁-C₆)alkanesulfonylamino and N—(C₁-C₆)alkyl-(C₁-C₆)alkanesulfonylamino or —X⁸—R¹⁵ [wherein X⁸ is a direct bonding or selected from O and N(R¹⁶), wherein R¹⁶ is a hydrogen atom or (C₁-C₆)alkyl, and R¹⁵ is selected from halogeno-(C₁-C₆)alkyl, hydroxy-(C₁-C₆)alkyl, (C₁-C₆)alkoxy-(C₁-C₆)alkyl, cyano-(C₁-C₆)alkyl, amino-(C₁-C₆)alkyl, (C₁-C₆)alkylamino-(C₁-C₆)alkyl and di-[(C₁-C₆)alkyl]amino-(C₁-C₆)alkyl or —X⁹-Q⁷ [wherein X⁹ is a direct bonding or selected from O, CO and N(R¹⁷), wherein R¹⁷ is a hydrogen atom or (C₁-C₆)alkyl, and Q⁷ is aryl, aryl-(C₁-C₆)alkyl, heteroaryl, heteroaryl-(C₁-C₆)alkyl, heterocyclyl or heterocyclyl-(C₁-C₆)alkyl, which may have 1 or 2 identical or different substituents selected from halogeno, (C₁-C₆)alkyl and (C₁-C₆)alkoxy, wherein any heterocyclyl of Q⁷ may have 1 or 2 oxo or thioxo substituents]],

heterocyclyl of R^{c} may have 1 or 2 oxo or thioxo substituents and

any identical or different substituents of aryl, heteroaryl or heterocyclyl of R^{c} may bond with each other to form —CH═CH—CH═CH—, —N═CH—CH═CH—, —CH═N—CH═CH—, —CH═CH—N═CH—, CH═CH—CH═N—, —N═CH—N═CH—, —CH═N—CH═N—, —N═CH—CH═N—, —N═N—CH═CH—, —CH═CH—N═N—, —CH═CH—O—, —O—CH═CH—, —CH═CH—S—, —S—CH═CH—, —C(R¹⁸)₂—C(R¹⁸)₂—O—, —O—C(R¹⁸)₂—C(R¹⁸)₂—, —C(R¹⁸)₂—C(R¹⁸)₂—S—, —S—C(R¹⁸)₂—C(R¹⁸)₂—, —O—C(R¹⁸)₂—O—, —O—C(R¹⁸)₂—C(R¹⁸)₂—O—, —S—C(R¹⁸)₂—S—, —S—C(R¹⁸)₂—C(R¹⁸)₂—S—, —CH═CH—NH—, —NH—CH═CH—, —C(R¹⁸)₂—C(R¹⁸)₂—NH—, —NH—C(R¹⁸)₂—C(R¹⁸)₂—, —N═CH—NH—, —NH—CH═N—, —NH—C(R¹⁸)₂—NH—, —O—CH═N—, —N═CH—O—, —S—CH═N—, —N═CH—S—, —O—C(R¹⁸)₂—NH—, —NH—C(R¹⁸)₂—O—, —S—C(R¹⁸)₂—NH—, —NH—C(R¹⁸)₂—S—, —O—N═CH—, —CH═N—O—, —S—N═CH—, —CH═N—S—, —O—NH—C(R¹⁸)₂—, —C(R¹⁸)₂—NH—O—, —S—NH—C(R¹⁸)₂—, —C(R¹⁸)₂—NH—S—, —NH—N═CH—, —CH═N—NH—, —NH—NH—C(R¹⁸)₂—, —C(R¹⁸)₂—NH—NH, —N═N—NH— or —NH—N═N— [wherein R¹⁸ is a hydrogen atom or (C₁-C₆)alkyl].

Of the quinazoline compounds represented by the formula 1, the compound represented by the formula 1-1 below is preferred:

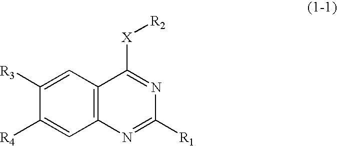

wherein

X is a direct bonding or selected from O, S, SO, SO₂ and NR⁵, wherein R⁵ is a hydrogen atom, (C₁-C₆)alkyl, aryl, aryl-(C₁-C₆)alkyl, (C₃-C₇)cycloalkyl, (C₃-C₇)cycloalkyl-(C₁-C₆)alkyl, heteroaryl, heteroaryl-(C₁-C₆)alkyl, heterocyclyl or heterocyclyl-(C₁-C₆)alkyl;

R¹ is a substituent selected from hydrogen, (C₁-C₈)alkyl, halogeno-(C₁-C₆)alkyl, hydroxy-(C₁-C₆)alkyl, (C₁-C₆)alkoxy-(C₁-C₆)alkyl, amino-(C₁-C₆)alkyl, (C₁-C₆)alkylamino-(C₁-C₆)alkyl, di-[(C₁-C₆)alkyl]amino-(C₁-C₆)alkyl and (C₁-C₆)alkylsulfinyl or —X¹-Q¹ [wherein X¹ is a direct bonding or selected from O, S and NR⁵ and Q¹ and R⁵ may be identical or different],

the neighboring carbon atoms of (C₂-C₆)alkylene of R₁ may be separated by a group selected from O, S and N(R⁵),

any CH₂ or CH₃ of R₁ may have at least one substituent selected from halogeno, (C₁-C₆)alkyl substituent, hydroxy, amino, (C₁-C₆)alkoxy (C₁-C₆)alkylthio, (C₁-C₆)alkylsulfinyl, (C₁-C₆)alkylamino and di-[(C₁-C₆)alkyl]amino,

any aryl, heteroaryl or heterocyclyl of R₁ may have 0 to 3 identical or different substituents selected from halogeno, hydroxy, amino, (C₁-C₆)alkyl, aryl, aryl-(C₁-C₆)alkyl, heteroaryl, heteroaryl-(C₁-C₆)alkyl, heterocyclyl, heterocyclyl-(C₁-C₆)alkyl, (C₁-C₆)alkoxy, (C₁-C₆)alkylthio and (C₁-C₆)alkylsulfinyl and

heterocyclyl of R₁ may have 1 or 2 oxo or thioxo substituents;

R₂ is a substituent selected from hydrogen, (C₁-C₁₈)alkyl, (C₂-C₈)alkenyl, halogeno-(C₁-C₈)alkyl, hydroxy-(C₁-C₈)alkyl, (C₁-C₈)alkoxy-(C₁-C₈)alkyl, amino-(C₁-C₈)alkyl, (C₁-C₈)alkylamino-(C₁-C₈)alkyl, di-[(C₁-C₈)alkyl]amino-(C₁-C₈)alkyl, (C₁-C₈)alkylsulfinyl, aryl, aryl-(C₁-C₈)alkyl, (C₃-C₇)cycloalkyl, (C₃-C₇)cycloalkyl-(C₁-C₈)alkyl, heteroaryl, heteroaryl-(C₁-C₈)alkyl, heterocyclyl and heterocyclyl-(C₁-C₈)alkyl,

wherein the neighboring carbon atoms of (C₂-C₆)alkylene of R₂ may be separated by a group selected from O, S, N(R⁵), CO, CH(OR⁵), CON(R⁵), N(R⁵)CO, SO₂N(R⁵), N(R⁵)SO₂ and CH═CH,

any CH₂ or CH₃ of R₂ may have at least one substituent selected from halogeno, (C₁-C₆)alkyl, hydroxy, cyano, amino, carboxyl, carbamoyl, (C₁-C₆)alkoxy (C₁-C₆)alkylthio, (C₁-C₆)alkylsulfinyl, (C₁-C₆)alkylamino, di-[(C₁-C₆)alkyl]amino, (C₁-C₆)alkoxycarbonyl, N—(C₁-C₆)alkylcarbamoyl, N,N-di-[(C₁-C₆)alkyl]carbamoyl, aryl, aryl-(C₁-C₆)alkyl, (C₃-C₇)cycloalkyl, (C₃-C₇)cycloalkyl-(C₁-C₆)alkyl, heteroaryl, heteroaryl-(C₁-C₆)alkyl, heterocyclyl and heterocyclyl-(C₁-C₆)alkyl,

any aryl, heteroaryl or heterocyclyl of R₂ may have 0 to 3 identical or different substituents selected from halogeno, trifluoromethyl, cyano, nitro, hydroxy, amino, carboxyl, carbamoyl, (C₁-C₆)alkyl, aryl, aryl-(C₁-C₆)alkyl, heteroaryl, heteroaryl-(C₁-C₆)alkyl, heterocyclyl, heterocyclyl-(C₁-C₆)alkyl, (C₂-C₈)alkenyl, (C₂-C₈)alkynyl, (C₁-C₆)alkoxy, (C₁-C₆)alkylthio, (C₁-C₆)alkylsulfinyl, (C₁-C₆)alkylamino, di-[(C₁-C₆)alkyl]amino, (C₁-C₆)alkoxycarbonyl, N—(C₁-C₆)alkylcarbamoyl and N,N-di-[(C₁-C₆)alkyl]carbamoyl,

heterocyclyl of R₂ may have 1 or 2 oxo or thioxo substituents and

any identical or different substituents of aryl, heteroaryl or heterocyclyl of R₂ may bond with each other to form —CH═CH—CH═CH—, —N═CH—CH═CH—, —CH═N—CH═CH—, —CH═CH—N═CH—, CH═CH—CH═N—, —N═CH—N═CH—, —CH═N—CH═N—, —N═CH—CH═N—, —CH═CH—O—, —O—CH═CH—, —CH═CH—S—, —S—CH═CH—, —C(R⁶)₂—C(R⁶)₂—O—, —O—C(R⁶)₂—C(R⁶)₂—, —C(R⁶)₂—C(R⁶)₂—S—, —S—C(R⁶)₂—C(R⁶)₂—, —O—C(R⁶)₂—O—, —O—C(R⁶)₂—C(R⁶)₂—O—, —S—C(R⁶)₂—S—, —S—C(R⁶)₂—C(R⁶)₂—S—, —CH═CH—NH—, —NH—CH═CH—, —C(R⁶)₂—C(R⁶)₂—NH—, —NH—C(R⁶)₂—C(R⁶)₂—, —N═CH—NH—, —NH—CH═N—, —NH—C(R⁶)₂—NH—, —O—CH═N—, —N═CH—O—, —S—CH═N—, —N═CH—S—, —O—C(R⁶)₂—NH—, —NH—C(R⁶)₂—O—, —S—C(R⁶)₂—NH—, —NH—C(R⁶)₂—S—, —N═N—NH— or —NH—N═N— [wherein R⁶ is a hydrogen atom or (C₁-C₃)alkyl]; and

each of R₃ and R₄ is, identically or differently, selected from hydrogen, halogeno, trifluoromethyl, cyano, nitro, hydroxy, mercapto, amino, formyl, carboxyl, carbamoyl,(C₁-C₆)alkyl, (C₂-C₈)alkenyl, (C₂-C₆)alkoxy, (C₁-C₆)alkylthio, (C₁-C₆)alkylsulfinyl, (C₁-C₆)alkylamino, di-[(C₁-C₆)alkyl]amino, (C₁-C₆)alkoxycarbonyl, N—(C₁-C₆)alkylcarbamoyl, N,N-di-[(C₁-C₆)alkyl]carbamoyl, aryl, aryl-(C₁-C₆)alkyl, (C₃-C₇)cycloalkyl, (C₃-C₇)cycloalkyl-(C₁-C₆)alkyl, heteroaryl, heteroaryl-(C₁-C₆)alkyl, heterocyclyl and heterocyclyl-(C₁-C₆)alkyl,

wherein any CH₂ or CH₃ of R₃ and R₄ may have at least one substituent selected from halogeno, hydroxy, amino, carboxyl, carbamoyl, (C₁-C₆)alkoxy, (C₁-C₆)alkylthio, (C₁-C₆)alkylsulfinyl, (C₁-C₆)alkylamino, di-[(C₁-C₆)alkyl]amino, (C₁-C₆)alkoxycarbonyl, N—(C₁-C₆)alkylcarbamoyl, N,N-di-[(C₁-C₆)alkyl]carbamoyl, aryl, aryl-(C₁-C₆)alkyl, (C₃-C₇)cycloalkyl, (C₃-C₇)cycloalkyl-(C₁-C₆)alkyl, heteroaryl, heteroaryl-(C₁-C₆)alkyl, heterocyclyl and heterocyclyl-(C₁-C₆)alkyl,

any aryl, heteroaryl or heterocyclyl of R₃ and R₄ may have 1, 2 or 3 identical or different substituents selected from halogeno, trifluoromethyl, nitro, hydroxy, amino, (C₁-C₆)alkoxy, (C₁-C₆)alkylthio, (C₁-C₆)alkylamino, di-[(C₁-C₆)alkyl]amino, (C₁-C₆)alkoxycarbonyl, N—(C₁-C₆)alkylcarbamoyl and N,N-di-[(C₁-C₆)alkyl]carbamoyl and

heterocyclyl of R₃ and R₄ may have 1 or 2 oxo or thioxo substituents.

More preferably, in the quinazoline compound represented by the formula 1-1:

X is O, S or NR₅;

R₁ is a hydrogen atom, C₁-C₆ alkyl, C₁-C₄ haloalkyl, C₁-C₆ alkylhydroxy, C₁-C₆ alkylsulfanyl, phenyl, phenyl C₁-C₈ alkyl, pyridino, furano, thiopheno, C₃-C₇ cycloalkyl, pyrrolidino, piperidino, morpholino or N—(C₁-C₆)alkylpiperazino; R₂ is C₁-C₁₈ alkyl, C₂-C₈ haloalkyl, C₃-C₇ cycloalkyl, (C₃-C₇)cycloalkyl C₁-C₈ alkyl, C₂-C₈ alkenyl, (C₃-C₇)cycloalkenyl C₁-C₈ alkyl, (C₁-C₈)alkyl C₁-C₈ alkoxyalkyl, C₁-C₈ hydroxyalkyl, hydroxy-(C₁-C₈)alkyl-(C₁-C₈)alkoxy alkyl, phenyl, phenyl C₁-C₈ alkyl, di-phenyl C₁-C₈ alkyl, phenyl C₁-C₈ alkylhydroxy, phenyl C₁-C₈ alkenyl, piperidine C₁-C₈ alkyl, N-[phenyl-(C₁-C₆)alkyl]piperidino, N—(C₁-C₆)alkylpiperidine C₁-C₈ alkyl, pyrrolidinone N—(C₁-C₈)alkyl, naphthalene C₁-C₈ alkyl, morpholine C₁-C₈ alkyl, piperazine C₁-C₈ alkyl, N—(C₁-C₆)alkylpiperazine C₁-C₈ alkyl, pyridine C₁-C₈ alkyl, pyrrolidine C₁-C₈ alkyl, N—(C₁-C₆)alkylpyrrolidine C₁-C₈ alkyl, thiopene C₁-C₈ alkyl or benzodioxol C₁-C₈ alkyl; each of R₃ and R₄ is a hydrogen atom, nitro or C₁-C₈ alkoxy; R₅ is a hydrogen atom, C₁-C₄ alkyl or C₁-C₈ hydroxyalkyl; and the substituent selected from C₃-C₇ cycloalkyl, phenyl, piperidine, naphthalene, morpholine, pyridine, pyrrolidine, pyrrolidinone, piperazine, thiopene and benzodioxol mentioned above has 1 to 3 substituents selected from C₁-C₈ alkyl, halogen, hydroxy, C₁-C₈ alkoxy, nitro and trifluoromethyl.

Specific examples of the compounds represented by the formula 1-1 are given in Table 1 below:

TABLE 1

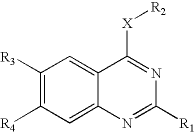

| Comp. | | | | | |
|---|---|---|---|---|---|
| No. | X | R₁ | R₂ | R₃ | R₄ |
| 1 | NH | phenyl | 2-(2-hydroxy-ethoxy)-ethyl | H | H |
| 2 | NH | trichloromethyl | isopropyl | H | H |
| 3 | NH | trichloromethyl | cyclopropyl | H | H |
| 4 | NH | trichloromethyl | 2-piperidino-1-ylethyl | H | H |
| 5 | NH | trichloromethyl | 1-phenylethyl | H | H |
| 6 | NH | trichloromethyl | 4-phenylbutyl | H | H |
| 7 | NH | trichloromethyl | n-propyl | H | H |
| 8 | NH | ethylsulfanyl | isopropyl | H | H |
| 9 | NH | ethylsulfanyl | 3-fluorobenzyl | H | H |
| 10 | NH | ethylsulfanyl | 2-(3-fluorophenyl)-ethyl | H | H |
| 11 | NH | ethylsulfanyl | 2-morpholino-4-ylethyl | H | H |
| 12 | NH | ethylsulfanyl | 2-(3-trifluoromethyl-phenyl)-ethyl | H | H |
| 13 | NH | H | phenyl | nitro | H |
| 14 | NH | H | naphthaleno-1-ylmethyl | ethoxy | ethoxy |
| 15 | NH | H | 3-morpholino-4-ylpropyl | ethoxy | ethoxy |
| 16 | NH | H | allyl | ethoxy | ethoxy |
| 17 | NH | H | 2-pyridino-2-ylethyl | ethoxy | ethoxy |
| 18 | NH | H | 4-chlorobenzyl | ethoxy | ethoxy |
| 19 | NH | ethylsulfanyl | n-butyl | H | H |
| 20 | NH | ethylsulfanyl | sec-butyl | H | H |
| 21 | NH | ethylsulfanyl | isobutyl | H | H |
| 22 | NH | ethylsulfanyl | benzyl | H | H |
| 23 | NH | ethylsulfanyl | 1-phenylethyl | H | H |
| 24 | NH | ethylsulfanyl | 3-methoxybenzyl | H | H |
| 25 | NH | ethylsulfanyl | 4-methoxybenzyl | H | H |
| 26 | NH | ethylsulfanyl | 3-trifluoromethyl | H | H |
| 27 | NH | ethylsulfanyl | cyclohexyl | H | H |
| 28 | NH | ethylsulfanyl | cyclopentyl | H | H |
| 29 | NH | ethylsulfanyl | phenethyl | H | H |
| 30 | NH | ethylsulfanyl | 4-phenylbutyl | H | H |
| 31 | NH | ethylsulfanyl | n-propyl | H | H |
| 32 | NH | ethylsulfanyl | 3-phenylpropyl | H | H |
| 33 | NH | ethylsulfanyl | 3-isopropoxy-propyl | H | H |
| 34 | NH | ethylsulfanyl | 2-methoxy-ethyl | H | H |
| 35 | NH | ethylsulfanyl | naphthaleno-1-ylmethyl | H | H |
| 36 | NH | ethylsulfanyl | 3-morpholino-4-ylpropyl | H | H |
| 37 | NH | ethylsulfanyl | benzo[1,3]dioxol-5-ylmethyl | H | H |
| 38 | NH | ethylsulfanyl | 3,5-dimethoxybenzyl | H | H |
| 39 | NH | ethylsulfanyl | 2-fluorobenzyl | H | H |
| 40 | NH | ethylsulfanyl | 1-methoxymethylpropyl | H | H |
| 41 | NH | ethylsulfanyl | 1-benzyl-piperidino-4-yl | H | H |
| 42 | NH | ethylsulfanyl | allyl | H | H |
| 43 | NH | ethylsulfanyl | n-hexadecyl | H | H |
| 44 | NH | ethylsulfanyl | 2-piperidino-1-ylethyl | H | H |
| 45 | NH | ethylsulfanyl | 2-pyrrolidino-1-ylethyl | H | H |
| 46 | NH | ethylsulfanyl | 2-pyridino-2-ylethyl | H | H |
| 47 | NH | ethylsulfanyl | 2-ethanol | H | H |
| 48 | NH | ethylsulfanyl | 3-(2-methyl-piperidino-1-yl)-propy | H | H |
| 49 | NH | ethylsulfanyl | 2-chloro-6-fluorobenzyl | H | H |
| 50 | NH | ethylsulfanyl | 2-(2-hydroxy-ethoxy)-ethyl | H | H |
| 51 | NH | phenyl | cyclohexyl | H | H |
| 52 | NH | phenyl | 4-phenylbutyl | H | H |
| 53 | NH | phenyl | 5-phenyl-pentyl | H | H |
| 54 | NH | phenyl | 4-methylbenzyl | H | H |
| 55 | NH | phenyl | isobutyl | H | H |
| 56 | NH | phenyl | n-butyl | H | H |
| 57 | NH | phenyl | naphthaleno-1-ylmethyl | H | H |
| 58 | NH | phenyl | pyridino-2-ylmethyl | H | H |
| 59 | NH | phenyl | thiopenyl-2-ylmethyl | H | H |
| 60 | NH | phenyl | isopropyl | H | H |
| 61 | NH | phenyl | n-propyl | H | H |
| 62 | NH | phenyl | 3-isopropoxy-propyl | H | H |
| 63 | NH | phenyl | 3-fluorobenzyl | H | H |
| 64 | NH | phenyl | 2-(3-trifluoromethyl-phenyl)-ethyl | H | H |
| 65 | NH | phenyl | benzo[1,3]dioxol-5-ylmethyl | H | H |
| 66 | NH | phenyl | 3-trifluoromethylbenzyl | H | H |
| 67 | NH | phenyl | 2-methoxy-ethyl | H | H |
| 68 | NH | phenyl | phenethyl | H | H |
| 69 | NH | phenyl | phenyl | H | H |
| 70 | NH | phenyl | 4-methoxyphenyl | H | H |
| 71 | NH | phenyl | o-tollyl | H | H |
| 72 | NH | phenyl | 4-isopropyl-phenyl | H | H |
| 73 | NH | phenyl | 2,4-dimethyl-phenyl | H | H |
| 74 | NH | phenyl | 4-n-undecyl-phenyl | H | H |
| 75 | NH | phenyl | 4-ethyl-phenyl | H | H |
| 76 | NH | phenyl | 4-fluorophenyl | H | H |
| 77 | NH | phenyl | 2-methoxyphenyl | H | H |
| 78 | NH | phenyl | 2,4,6-trimethyl-phenyl | H | H |
| 79 | NH | phenyl | 4-n-butyl-phenyl | H | H |
| 80 | NH | phenyl | 2-phenoxy-phenyl | H | H |
| 81 | NH | phenyl | 3-phenoxy-phenyl | H | H |
| 82 | NH | phenyl | 4-phenoxy-phenyl | H | H |
| 83 | NH | phenyl | 3-bromophenyl | H | H |
| 84 | NH | phenyl | 2-methoxy-5-phenol | H | H |
| 85 | NH | phenyl | 5-hydroxy-pentane-1-yl | H | H |
| 86 | NH | phenyl | benzyl | H | H |
| 87 | NH | phenyl | 1-phenylethyl | H | H |
| 88 | NH | phenyl | 4-chlorobenzyl | H | H |
| 89 | NH | phenyl | 3,4-dichlorobenzyl | H | H |
| 90 | NH | phenyl | 3-methoxybenzyl | H | H |
| 91 | NH | phenyl | 2-methoxybenzyl | H | H |
| 92 | NH | phenyl | 3,5-dimethoxybenzyl | H | H |
| 93 | NH | phenyl | 2,3-dimethoxybenzyl | H | H |
| 94 | NH | phenyl | 2-fluorobenzyl | H | H |
| 95 | NH | phenyl | cyclopentyl | H | H |
| 96 | NH | phenyl | sec-butyl | H | H |
| 97 | NH | phenyl | allyl | H | H |
| 98 | NH | phenyl | 2-trifluoromethylbenzyl | H | H |
| 99 | NH | phenyl | n-hexadecyl | H | H |
| 100 | NH | phenyl | 2-pyrrolidino-1-ylethyl | H | H |
| 101 | NH | phenyl | 2-piperidino-1-ylethyl | H | H |
| 102 | NH | phenyl | 2-pyridino-2-ylethyl | H | H |
| 103 | NH | phenyl | 2-(3-fluorophenyl)-ethyl | H | H |
| 104 | NH | phenyl | 1-methoxymethylpropyl | H | H |
| 105 | NH | phenyl | 1-benzyl-piperidino-4-yl | H | H |
| 106 | NH | phenyl | 2-morpholino-4-ylethyl | H | H |
| 107 | NH | phenyl | 2-(3,4-dimethoxyphenyl)-ethyl | H | H |
| 108 | NH | phenyl | 2,4-dichlorobenzyl | H | H |
| 109 | NH | phenyl | 2-chlorobenzyl | H | H |
| 110 | NH | phenyl | cyclopropyl | H | H |
| 111 | NH | phenyl | pyridino-3-ylmethyl | H | H |
| 112 | NH | phenyl | 1-hydroxy-propane-2-yl | H | H |
| 113 | NH | phenyl | (4-hydroxy-3-methoxyphenyl)-met | H | H |
| 114 | NH | phenyl | 2-fluoro-ethyl | H | H |
| 115 | NH | phenyl | 2,3-dichlorobenzyl | H | H |
| 116 | NH | phenyl | 3-nitrobenzyl | H | H |
| 117 | NH | phenyl | 1-ethyl-pyrrolidino-2-ylmethyl | H | H |
| 118 | NH | phenyl | 3,4-dimethoxybenzyl | H | H |
| 119 | NH | phenyl | 2-p-tollylethyl | H | H |
| 120 | NH | phenyl | 2-(3-methoxyphenyl)-ethyl | H | H |
| 121 | NH | phenyl | 1-ethylpropyl | H | H |
| 122 | NH | phenyl | 2-(2,5-dimethoxyphenyl)-ethyl | H | H |
| 123 | NH | phenyl | 2-(4-chlorophenyl)-ethyl | H | H |
| 124 | NH | phenyl | 2-(4-methoxyphenyl)-ethyl | H | H |
| 125 | NH | phenyl | 1,3-dimethylbutyl | H | H |
| 126 | NH | phenyl | 2-(3-chlorophenyl)-ethyl | H | H |
| 127 | NH | phenyl | 2-(2,4-dichlorophenyl)-ethyl | H | H |
| 128 | NH | phenyl | 2-(3,4-difluorophenyl)-ethyl | H | H |
| 129 | NH | phenyl | 3,4,5-trimethoxybenzyl | H | H |
| 130 | NH | phenyl | 3-methylbutyl | H | H |
| 131 | NH | phenyl | 2-cyclohex-1-enylethyl | H | H |
| 132 | NH | phenyl | cyclohexylmethyl | H | H |
| 133 | NH | phenyl | 2,2-diphenylethyl | H | H |
| 134 | NH | phenyl | 2,4-difluorobenzyl | H | H |
| 135 | NH | phenyl | 2,5-difluorobenzyl | H | H |
| 136 | NH | phenyl | 3,3-diphenylpropyl | H | H |
| 137 | NH | phenyl | 2-(4-bromophenyl)-ethyl | H | H |
| 138 | NMe | phenyl | n-butyl | H | H |
| 139 | NH | phenyl | 4-trifluoromethylbenzyl | H | H |
| 140 | NH | phenyl | 2-(2-fluorophenyl)-ethyl | H | H |
| 141 | NH | phenyl | pyridino-4-ylmethyl | H | H |
| 142 | NH | phenyl | pyridino-2-ylmethyl | H | H |
| 143 | NH | phenyl | 2,3-dimethylcyclohexyl | H | H |
| 144 | NH | phenyl | 2,4-dimethoxybenzyl | H | H |
| 145 | NH | phenyl | 3-chlorobenzyl | H | H |
| 146 | NH | phenyl | 2-hydroxy-2-phenylethyl | H | H |
| 147 | NH | phenyl | 2,5-dimethoxybenzyl | H | H |
| 148 | NH | phenyl | 4-bromobenzyl | H | H |
| 149 | NH | phenyl | 2-(3,4-dichlorophenyl)-ethyl | H | H |
| 150 | NH | phenyl | 3-(2-oxo-pyrrolidino-1-yl)-propane | H | H |
| 151 | NH | phenyl | 2-(2-chlorophenyl)-ethyl | H | H |
| 152 | NH | phenyl | 2-(2-hydroxy-phenyl)-ethyl | H | H |
| 153 | NH | phenyl | 3-bromobenzyl | H | H |
| 154 | NH | phenyl | 2,6-dimethoxybenzyl | H | H |
| 155 | NH | phenyl | 2-bromobenzyl | H | H |
| 156 | NH | phenyl | 3-morpholino-4-ylpropyl | H | H |
| 157 | N-n-Bu | phenyl | 2-hydroxy-ethyl | H | H |
| 158 | NH | phenyl | 2-(4-methylpiperazino-1-yl)-ethyl | H | H |
| 159 | NMe | phenyl | cyclopentyl | H | H |
| 160 | NH | phenyl | 2-piperazino-1-ylethyl | H | H |
| 161 | NH | phenyl | O-(4-nitrobenzyl)-hydroxyl | H | H |
| 162 | NH | phenyl | 4-chlorophenyl | H | H |
| 163 | NH | methyl | benzyl | H | H |
| 164 | NH | methyl | 3-methoxybenzyl | H | H |
| 165 | NH | methyl | 4-methoxybenzyl | H | H |
| 166 | NH | methyl | 3-trifluoromethylbenzyl | H | H |
| 167 | NH | methyl | phenethyl | H | H |
| 168 | NH | methyl | 4-phenylbutyl | H | H |
| 169 | NH | methyl | naphthaleno-1-ylmethyl | H | H |
| 170 | NH | methyl | benzo[1,3]dioxol-5-ylmethyl | H | H |
| 171 | NH | methyl | 3-fluorobenzyl | H | H |
| 172 | NH | methyl | 2-(3-fluorophenyl)-ethyl | H | H |
| 173 | NH | methyl | 2-(2-hydroxy-ethoxy)-ethyl | H | H |
| 174 | NH | methyl | 2-hydroxy-ethyl | H | H |
| 175 | NH | isopropyl | benzyl | H | H |
| 176 | NH | isopropyl | 3-methoxybenzyl | H | H |
| 177 | NH | isopropyl | 4-methoxybenzyl | H | H |
| 178 | NH | isopropyl | 3-trifluoromethylbenzyl | H | H |
| 179 | NH | isopropyl | phenethyl | H | H |
| 180 | NH | isopropyl | 4-phenylbutyl | H | H |
| 181 | NH | isopropyl | naphthaleno-1-ylmethyl | H | H |
| 182 | NH | isopropyl | benzo[1,3]dioxol-5-ylmethyl | H | H |
| 183 | NH | isopropyl | 3-fluorobenzyl | H | H |
| 184 | NH | isopropyl | 2-(3-fluorophenyl)-ethyl | H | H |
| 185 | NH | isopropyl | 2-(2-hydroxy-ethoxy)-ethyl | H | H |
| 186 | NH | isopropyl | 2-hydroxy-ethyl | H | H |
| 187 | NH | tert-butyl | benzyl | H | H |
| 188 | NH | tert-butyl | 3-methoxybenzyl | H | H |
| 189 | NH | tert-butyl | 4-methoxybenzyl | H | H |
| 190 | NH | tert-butyl | 3-trifluoromethylbenzyl | H | H |
| 191 | NH | tert-butyl | phenethyl | H | H |
| 192 | NH | tert-butyl | 4-phenylbutyl | H | H |
| 193 | NH | tert-butyl | naphthaleno-1-ylmethyl | H | H |
| 194 | NH | tert-butyl | benzo[1,3]dioxol-5-ylmethyl | H | H |
| 195 | NH | tert-butyl | 3-fluorobenzyl | H | H |
| 196 | NH | tert-butyl | 2-(3-fluorophenyl)-ethyl | H | H |
| 197 | NH | tert-butyl | 2-(2-hydroxy-ethoxy)-ethyl | H | H |
| 198 | NH | tert-butyl | 2-hydroxy-ethyl | H | H |
| 199 | NH | cyclohexyl | benzyl | H | H |
| 200 | NH | cyclohexyl | 3-methoxybenzyl | H | H |
| 201 | NH | cyclohexyl | 4-methoxybenzyl | H | H |
| 202 | NH | cyclohexyl | 3-trifluoromethylbenzyl | H | H |
| 203 | NH | cyclohexyl | phenethyl | H | H |
| 204 | NH | cyclohexyl | 4-phenylbutyl | H | H |
| 205 | NH | cyclohexyl | naphthaleno-1-ylmethyl | H | H |
| 206 | NH | cyclohexyl | benzo[1,3]dioxol-5-ylmethyl | H | H |
| 207 | NH | cyclohexyl | 3-fluorobenzyl | H | H |
| 208 | NH | cyclohexyl | 2-(3-fluorophenyl)-ethyl | H | H |
| 209 | NH | cyclohexyl | 2-(2-hydroxy-ethoxy)-ethyl | H | H |
| 210 | NH | cyclohexyl | 2-hydroxy-ethyl | H | H |
| 211 | NH | benzyl | benzyl | H | H |
| 212 | NH | benzyl | 3-methoxybenzyl | H | H |
| 213 | NH | benzyl | 4-methoxybenzyl | H | H |
| 214 | NH | benzyl | 3-trifluoromethylbenzyl | H | H |
| 215 | NH | benzyl | phenethyl | H | H |
| 216 | NH | benzyl | 4-phenylbutyl | H | H |
| 217 | NH | benzyl | naphthaleno-1-ylmethyl | H | H |
| 218 | NH | benzyl | benzo[1,3]dioxol-5-ylmethyl | H | H |
| 219 | NH | benzyl | 3-fluorobenzyl | H | H |
| 220 | NH | benzyl | 2-(3-fluorophenyl)-ethyl | H | H |
| 221 | NH | benzyl | 2-(2-hydroxy-ethoxy)-ethyl | H | H |
| 222 | NH | benzyl | 2-hydroxy-ethyl | H | H |
| 223 | NH | pyridino-2-yl | benzyl | H | H |
| 224 | NH | pyridino-2-yl | 3-methoxybenzyl | H | H |
| 225 | NH | pyridino-2-yl | 4-methoxybenzyl | H | H |
| 226 | NH | pyridino-2-yl | 3-trifluoromethylbenzyl | H | H |
| 227 | NH | pyridino-2-yl | phenethyl | H | H |
| 228 | NH | pyridino-2-yl | 4-phenylbutyl | H | H |
| 229 | NH | pyridino-2-yl | naphthaleno-1-ylmethyl | H | H |
| 230 | NH | pyridino-2-yl | benzo[1,3]dioxol-5-ylmethyl | H | H |
| 231 | NH | pyridino-2-yl | 3-fluorobenzyl | H | H |
| 232 | NH | pyridino-2-yl | 2-(3-fluorophenyl)-ethyl | H | H |
| 233 | NH | pyridino-2-yl | 2-(2-hydroxy-ethoxy)-ethyl | H | H |
| 234 | NH | pyridino-2-yl | 2-hydroxy-ethyl | H | H |
| 235 | NH | pyridino-3-yl | benzyl | H | H |
| 236 | NH | pyridino-3-yl | 3-methoxybenzyl | H | H |
| 237 | NH | pyridino-3-yl | 4-methoxybenzyl | H | H |
| 238 | NH | pyridino-3-yl | 3-trifluoromethylbenzyl | H | H |
| 239 | NH | pyridino-3-yl | phenethyl | H | H |
| 240 | NH | pyridino-3-yl | 4-phenylbutyl | H | H |
| 241 | NH | pyridino-3-yl | naphthaleno-1-ylmethyl | H | H |
| 242 | NH | pyridino-3-yl | benzo[1,3]dioxol-5-ylmethyl | H | H |
| 243 | NH | pyridino-3-yl | 3-fluorobenzyl | H | H |
| 244 | NH | pyridino-3-yl | 2-(3-fluorophenyl)-ethyl | H | H |
| 245 | NH | pyridino-3-yl | 2-(2-hydroxy-ethoxy)-ethyl | H | H |
| 246 | NH | pyridino-3-yl | 2-hydroxy-ethyl | H | H |
| 247 | NH | pyridino-4-yl | benzyl | H | H |
| 248 | NH | pyridino-4-yl | 3-methoxybenzyl | H | H |
| 249 | NH | pyridino-4-yl | 4-methoxybenzyl | H | H |
| 250 | NH | pyridino-4-yl | 3-trifluoromethylbenzyl | H | H |
| 251 | NH | pyridino-4-yl | phenethyl | H | H |
| 252 | NH | pyridino-4-yl | 4-phenylbutyl | H | H |
| 253 | NH | pyridino-4-yl | naphthaleno-1-ylmethyl | H | H |
| 254 | NH | pyridino-4-yl | benzo[1,3]dioxol-5-ylmethyl | H | H |
| 255 | NH | pyridino-4-yl | 3-fluorobenzyl | H | H |
| 256 | NH | pyridino-4-yl | 2-(3-fluorophenyl)-ethyl | H | H |
| 257 | NH | pyridino-4-yl | 2-(2-hydroxy-ethoxy)-ethyl | H | H |
| 258 | NH | pyridino-4-yl | 2-hydroxy-ethyl | H | H |
| 259 | NH | 4-chlorophenyl | benzyl | H | H |
| 260 | NH | 4-chlorophenyl | 3-methoxybenzyl | H | H |
| 261 | NH | 4-chlorophenyl | 4-methoxybenzyl | H | H |
| 262 | NH | 4-chlorophenyl | 3-trifluoromethylbenzyl | H | H |
| 263 | NH | 4-chlorophenyl | phenethyl | H | H |
| 264 | NH | 4-chlorophenyl | 4-phenylbutyl | H | H |
| 265 | NH | 4-chlorophenyl | naphthaleno-1-ylmethyl | H | H |
| 266 | NH | 4-chlorophenyl | benzo[1,3]dioxol-5-ylmethyl | H | H |
| 267 | NH | 4-chlorophenyl | 3-fluorobenzyl | H | H |
| 268 | NH | 4-chlorophenyl | 2-(3-fluorophenyl)-ethyl | H | H |
| 269 | NH | 4-chlorophenyl | 2-(2-hydroxy-ethoxy)-ethyl | H | H |
| 270 | NH | 4-chlorophenyl | 2-hydroxy-ethyl | H | H |
| 271 | NH | isopropyl | 2,4-dimethoxybenzyl | H | H |
| 272 | NH | benzyl | 2,4-dimethoxybenzyl | H | H |
| 273 | O | phenyl | 2,4-dimethoxybenzyl | H | H |
| 274 | S | phenyl | 2,4-dimethoxybenzyl | H | H |
| 275 | NH | pyrrolidino-1-yl | 2-methoxybenzyl | H | H |
| 276 | NH | piperidino-1-yl | 2-methoxybenzyl | H | H |
| 277 | NH | morpholino-4-yl | 2-methoxybenzyl | H | H |
| 278 | NH | 4-methylpiperazi | 2-methoxybenzyl | H | H |
| 279 | NH | pyrrolidino-1-yl | 3-methoxybenzyl | H | H |
| 280 | NH | piperidino-1-yl | 3-methoxybenzyl | H | H |
| 281 | NH | morpholino-4-yl | 3-methoxybenzyl | H | H |
| 282 | NH | 4-methylpiperazi | 3-methoxybenzyl | H | H |
| 283 | NH | pyrrolidino-1-yl | 4-methoxybenzyl | H | H |
| 284 | NH | piperidino-1-yl | 4-methoxybenzyl | H | H |
| 285 | NH | morpholino-4-yl | 4-methoxybenzyl | H | H |
| 286 | NH | 4-methylpiperazi | 4-methoxybenzyl | H | H |
| 287 | NH | pyrrolidino-1-yl | 2,3-dimethoxybenzyl | H | H |
| 288 | NH | piperidino-1-yl | 2,3-dimethoxybenzyl | H | H |
| 289 | NH | morpholino-4-yl | 2,3-dimethoxybenzyl | H | H |
| 290 | NH | 4-methylpiperazi | 2,3-dimethoxybenzyl | H | H |
| 291 | NH | pyrrolidino-1-yl | 2,4-dimethoxybenzyl | H | H |
| 292 | NH | piperidino-1-yl | 2,4-dimethoxybenzyl | H | H |
| 293 | NH | morpholino-4-yl | 2,4-dimethoxybenzyl | H | H |
| 294 | NH | 4-methylpiperazi | 2,4-dimethoxybenzyl | H | H |
| 295 | NH | pyrrolidino-1-yl | 2,5-dimethoxybenzyl | H | H |
| 296 | NH | piperidino-1-yl | 2,5-dimethoxybenzyl | H | H |
| 297 | NH | morpholino-4-yl | 2,5-dimethoxybenzyl | H | H |
| 298 | NH | 4-methylpiperazi | 2,5-dimethoxybenzyl | H | H |
| 299 | NH | pyrrolidino-1-yl | 3,4-dimethoxybenzyl | H | H |
| 300 | NH | piperidino-1-yl | 3,4-dimethoxybenzyl | H | H |
| 301 | NH | morpholino-4-yl | 3,4-dimethoxybenzyl | H | H |
| 302 | NH | 4-methylpiperazi | 3,4-dimethoxybenzyl | H | H |
| 303 | NH | pyrrolidino-1-yl | 3,5-dimethoxybenzyl | H | H |
| 304 | NH | piperidino-1-yl | 3,5-dimethoxybenzyl | H | H |
| 305 | NH | morpholino-4-yl | 3,5-dimethoxybenzyl | H | H |
| 306 | NH | 4-methylpiperazi | 3,5-dimethoxybenzyl | H | H |
| 307 | NH | pyrrolidino-1-yl | benzo[1,3]dioxol-5-ylmethyl | H | H |
| 308 | NH | piperidino-1-yl | benzo[1,3]dioxol-5-ylmethyl | H | H |
| 309 | NH | morpholino-4-yl | benzo[1,3]dioxol-5-ylmethyl | H | H |
| 310 | NH | 4-methylpiperazi | benzo[1,3]dioxol-5-ylmethyl | H | H |
| 311 | NH | pyrrolidino-1-yl | 3,4,5-trimethoxybenzyl | H | H |
| 312 | NH | piperidino-1-yl | 3,4,5-trimethoxybenzyl | H | H |
| 313 | NH | morpholino-4-yl | 3,4,5-trimethoxybenzyl | H | H |
| 314 | NH | 4-methylpiperazi | 3,4,5-trimethoxybenzyl | H | H |
| 315 | NH | pyrrolidino-1-yl | 2,4,6-trimethoxybenzyl | H | H |
| 316 | NH | piperidino-1-yl | 2,4,6-trimethoxybenzyl | H | H |
| 317 | NH | morpholino-4-yl | 2,4,6-trimethoxybenzyl | H | H |
| 318 | NH | 4-methylpiperazi | 2,4,6-trimethoxybenzyl | H | H |
| 319 | NH | ethoxy | 2,4-dimethoxybenzyl | H | H |
| 320 | NH | ethoxy | benzo[1,3]dioxol-5-ylmethyl | H | H |
| 321 | NH | isopropyl | 2-methoxybenzyl | H | H |
| 322 | NH | isopropyl | 2,3-dimethoxybenzyl | H | H |
| 323 | NH | isopropyl | 3,5-dimethoxybenzyl | H | H |
| 324 | NH | isopropyl | 3,4-dimethoxybenzyl | H | H |
| 325 | NH | isopropyl | 3,4,5-trimethoxybenzyl | H | H |
| 326 | NH | isopropyl | 2,5-dimethoxybenzyl | H | H |
| 327 | NH | isopropyl | 2,4,6-trimethoxybenzyl | H | H |
| 328 | NH | benzyl | 2-methoxybenzyl | H | H |
| 329 | NH | benzyl | 2,3-dimethoxybenzyl | H | H |
| 330 | NH | benzyl | 3,5-dimethoxybenzyl | H | H |
| 331 | NH | benzyl | 3,4-dimethoxybenzyl | H | H |
| 332 | NH | benzyl | 3,4,5-trimethoxybenzyl | H | H |
| 333 | NH | benzyl | 2,4-dimethoxybenzyl | H | H |
| 334 | NH | benzyl | 2,5-dimethoxybenzyl | H | H |
| 335 | NH | benzyl | 2,4,6-trimethoxybenzyl | H | H |
| 336 | NH | phenyl | 3,4,5-trimethoxybenzyl | H | H |
| 337 | NH | phenyl | 2,4,6-trimethoxybenzyl | H | H |
| 338 | NH | furan-2-yl | 2-methoxybenzyl | H | H |
| 339 | NH | furan-2-yl | 3-methoxybenzyl | H | H |
| 340 | NH | furan-2-yl | 4-methoxybenzyl | H | H |
| 341 | NH | furan-2-yl | benzo[1,3]dioxol-5-ylmethyl | H | H |
| 342 | NH | furan-2-yl | 2,3-dimethoxybenzyl | H | H |
| 343 | NH | furan-2-yl | 3,5-dimethoxybenzyl | H | H |
| 344 | NH | furan-2-yl | 3,4-dimethoxybenzyl | H | H |
| 345 | NH | furan-2-yl | 3,4,5-trimethoxybenzyl | H | H |
| 346 | NH | furan-2-yl | 2,4-dimethoxybenzyl | H | H |
| 347 | NH | furan-2-yl | 2,5-dimethoxybenzyl | H | H |
| 348 | NH | furan-2-yl | 2,4,6-trimethoxybenzyl | H | H |
| 349 | NH | thiopenyl-2-yl | 2-methoxybenzyl | H | H |
| 350 | NH | thiopenyl-2-yl | 3-methoxybenzyl | H | H |
| 351 | NH | thiopenyl-2-yl | 4-methoxybenzyl | H | H |
| 352 | NH | thiopenyl-2-yl | benzo[1,3]dioxol-5-ylmethyl | H | H |
| 353 | NH | thiopenyl-2-yl | 2,3-dimethoxybenzyl | H | H |
| 354 | NH | thiopenyl-2-yl | 3,5-dimethoxybenzyl | H | H |
| 355 | NH | thiopenyl-2-yl | 3,4-dimethoxybenzyl | H | H |
| 356 | NH | thiopenyl-2-yl | 3,4,5-trimethoxybenzyl | H | H |
| 357 | NH | thiopenyl-2-yl | 2,4-dimethoxybenzyl | H | H |
| 358 | NH | thiopenyl-2-yl | 2,5-dimethoxybenzyl | H | H |
| 359 | NH | thiopenyl-2-yl | 2,4,6-trimethoxybenzyl | H | H |
| 360 | NH | methoxyphenyl | 2-methoxybenzyl | H | H |
| 361 | NH | methoxyphenyl | 3-methoxybenzyl | H | H |
| 362 | NH | methoxyphenyl | 4-methoxybenzyl | H | H |
| 363 | NH | methoxyphenyl | benzo[1,3]dioxol-5-ylmethyl | H | H |
| 364 | NH | methoxyphenyl | 2,3-dimethoxybenzyl | H | H |
| 365 | NH | methoxyphenyl | 3,5-dimethoxybenzyl | H | H |
| 366 | NH | methoxyphenyl | 3,4-dimethoxybenzyl | H | H |
| 367 | NH | methoxyphenyl | 3,4,5-trimethoxybenzyl | H | H |
| 368 | NH | methoxyphenyl | 2,4-dimethoxybenzyl | H | H |
| 369 | NH | methoxyphenyl | 2,5-dimethoxybenzyl | H | H |
| 370 | NH | methoxyphenyl | 2,4,6-trimethoxybenzyl | H | H |
| 371 | NH | 1-phenylethyl | 2-methoxybenzyl | H | H |
| 372 | NH | 1-phenylethyl | 3-methoxybenzyl | H | H |
| 373 | NH | 1-phenylethyl | 4-methoxybenzyl | H | H |
| 374 | NH | 1-phenylethyl | benzo[1,3]dioxol-5-ylmethyl | H | H |
| 375 | NH | 1-phenylethyl | 2,3-dimethoxybenzyl | H | H |
| 376 | NH | 1-phenylethyl | 3,5-dimethoxybenzyl | H | H |
| 377 | NH | 1-phenylethyl | 3,4-dimethoxybenzyl | H | H |
| 378 | NH | 1-phenylethyl | 3,4,5-trimethoxybenzyl | H | H |
| 379 | NH | 1-phenylethyl | 2,4-dimethoxybenzyl | H | H |
| 380 | NH | 1-phenylethyl | 2,5-dimethoxybenzyl | H | H |
| 381 | NH | 1-phenylethyl | 2,4,6-trimethoxybenzyl | H | H |
| 382 | NH | cyclopropyl | 2-methoxybenzyl | H | H |
| 383 | NH | cyclopropyl | 3-methoxybenzyl | H | H |
| 384 | NH | cyclopropyl | 4-methoxybenzyl | H | H |
| 385 | NH | cyclopropyl | benzo[1,3]dioxol-5-ylmethyl | H | H |
| 386 | NH | cyclopropyl | 2,3-dimethoxybenzyl | H | H |
| 387 | NH | cyclopropyl | 3,5-dimethoxybenzyl | H | H |
| 388 | NH | cyclopropyl | 3,4-dimethoxybenzyl | H | H |
| 389 | NH | cyclopropyl | 3,4,5-trimethoxybenzyl | H | H |
| 390 | NH | cyclopropyl | 2,4-dimethoxybenzyl | H | H |
| 391 | NH | cyclopropyl | 2,5-dimethoxybenzyl | H | H |
| 392 | NH | cyclopropyl | 2,4,6-trimethoxybenzyl | H | H |
| 393 | NH | n-propyl | 2-methoxybenzyl | H | H |
| 394 | NH | n-propyl | 3-methoxybenzyl | H | H |
| 395 | NH | n-propyl | 4-methoxybenzyl | H | H |
| 396 | NH | n-propyl | benzo[1,3]dioxol-5-ylmethyl | H | H |
| 397 | NH | n-propyl | 2,3-dimethoxybenzyl | H | H |
| 398 | NH | n-propyl | 3,5-dimethoxybenzyl | H | H |
| 399 | NH | n-propyl | 3,4-dimethoxybenzyl | H | H |
| 400 | NH | n-propyl | 3,4,5-trimethoxybenzyl | H | H |
| 401 | NH | n-propyl | 2,4-dimethoxybenzyl | H | H |
| 402 | NH | n-propyl | 2,5-dimethoxybenzyl | H | H |
| 403 | NH | n-propyl | 2,4,6-trimethoxybenzyl | H | H |
| 404 | NH | H | 2-methoxybenzyl | H | H |
| 405 | NH | H | 3-methoxybenzyl | H | H |
| 406 | NH | H | 4-methoxybenzyl | H | H |
| 407 | NH | H | benzo[1,3]dioxol-5-ylmethyl | H | H |
| 408 | NH | H | 2,3-dimethoxybenzyl | H | H |
| 409 | NH | H | 3,5-dimethoxybenzyl | H | H |
| 410 | NH | H | 3,4-dimethoxybenzyl | H | H |
| 411 | NH | H | 3,4,5-trimethoxybenzyl | H | H |
| 412 | NH | H | 2,4-dimethoxybenzyl | H | H |
| 413 | NH | H | 2,5-dimethoxybenzyl | H | H |
| 414 | NH | H | 2,4,6-trimethoxybenzyl | H | H |
| 415 | O | isopropyl | benzyl | H | H |
| 416 | S | isopropyl | benzyl | H | H |

The compound represented by the formula 1 may form a pharmaceutically available salt, including an inorganic acid salt such as hydrochloride, sulfate, phosphate, bisphosphate, hydrobromate and nitrate or an organic acid salt such as malate, maleate, fumarate, tartrate, succinate, citrate, acetate, lactate, methanesulfonate, p-toluenesulfonate, palmitate, salicylate and stearate.

Some of the compounds of the present invention may be crystallized or recrystallized using an aqueous or organic solvent. In that case, a solvate may be formed. In addition to various water-containing compounds with different water contents that can be prepared by lyophilization, etc., hydrates and other stoichiometric solvates belong to the scope of the present invention.

The compound represented by the formula 1 may be an enantiomer, a stereoisomer or a tautomer. The isomers may be separated or decomposed by common methods or may be obtained by common stereospecific or asymmetrical synthesis methods.

Radioactive derivatives of the compound represented by the formula 1, which are useful for biological studies, also belong to the scope of the present invention.

The present invention also provides a preparation method of the quinazoline derivatives represented by the formula 1 and a pharmaceutically available salt thereof.

The present invention also provides a preparation method of the compound represented by the formula 1-1. As shown in the scheme 1 below, it may be synthesized from the substitution reaction of a 4-chloro-2-substituted-quinazoline derivative represented by the formula 2 below and a compound represented by the formula 3 below:

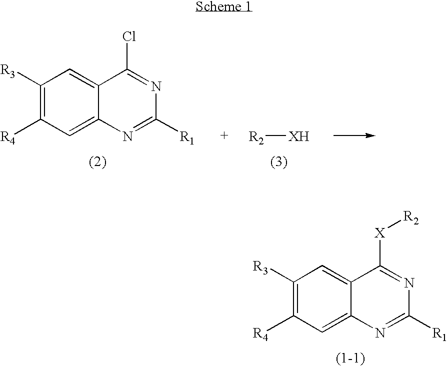

wherein R₁, R₂, R₃, R₄ and X are the same as defined in the formula 1.

The substitution reaction of the scheme 1 is carried out in the presence of a base and an organic polar solvent. For the base, an organic base such as pyridine or an alkylamine or an inorganic base such as an alkali metal salt or an alkaline earth metal salt may be used.

Alternatively, the compound represented by the formula 1-1 may be prepared from the substitution reaction of a 2-chloro-4-substituted-quinazoline derivative represented by the formula 4 below and a compound represented by the formula 5 below, as shown in the scheme 2 below:

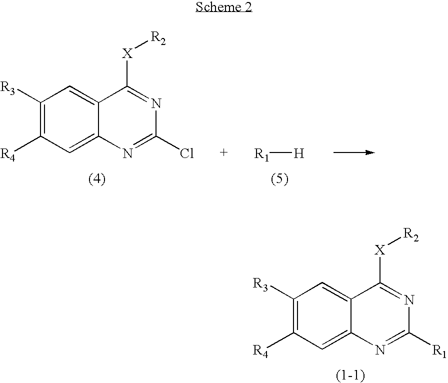

wherein R₁, R₂, R₃, R₄ and X are the same as defined in the formula 1.

The substitution reaction of the scheme 2 is carried out in the presence of a base and an organic polar solvent. For the base, an organic base such as pyridine or an alkylamine or an inorganic base such as an alkali metal salt or an alkaline earth metal salt may be used.

Alternatively, the compound represented by the formula 1-1 may be prepared from the substitution reaction of a 2-methyl-3H-quinazolino-4-one derivative represented by the formula 6 below, which is substituted by X at the number 4 position, and a compound represented by the formula 7 below, as shown in the scheme 3 below:

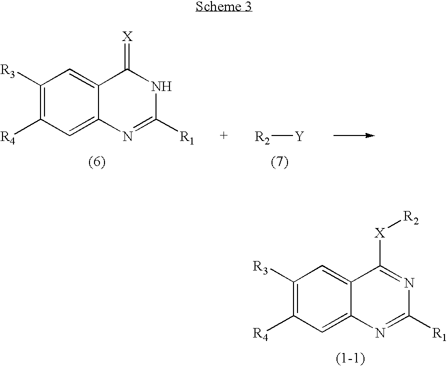

wherein R₁, R₂, R₃, R₄ and X are the same as defined in the formula 1 and Y is a leaving group selected from Cl, Br, I, OMs and OTs.

The substitution reaction of the scheme 3 is carried out in the presence of a base and an organic polar solvent. As a base, an organic base such as pyridine or an alkylamine or an inorganic base such as an alkali metal salt or an alkaline earth metal salt may be used.

The compounds represented by the formulas 2 and 3 in the scheme 1, which are used as starting materials of the substitution reaction, may be prepared by the methods well known in the field of organic synthesis.

Of the quinazoline derivatives represented by the formula 2, one in which R₁ is ethylsulfanyl may be prepared by the scheme 4 below:

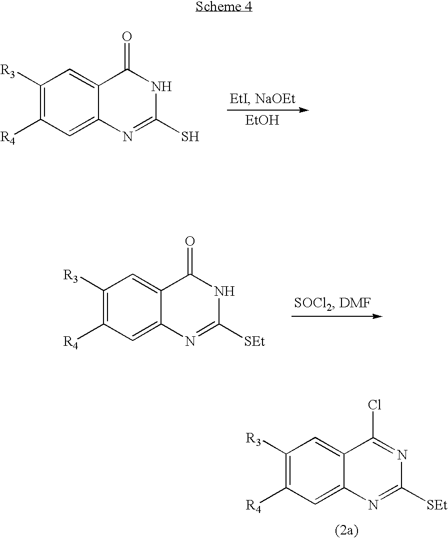

wherein R₃ and R₄ are the same as defined in the formula 1.

First, 2-mercapto-3H-quinazolino-4-one is reacted with sodium ethoxide (NaOEt) and ethyl iodide (EtI) in the temperature range from room temperature to the reflux temperature of the solvent in order to introduce the ethylsulfanyl group at the C-2 position. Subsequently, to the resultant 2-ethylsulfanyl-3H-quinazolino-4-one is added thionyl chloride (SOCl₂) at −20 to 10° C. and reflux reaction is performed in the presence of an N,N-dimethylformamide catalyst in order to transform the ketone group at the C-4 position into chlorine (Cl).

Of the quinazoline derivatives represented by the formula 2, one in which R₁ is a hydrogen atom, alkyl or aryl may be prepared by the scheme 5 below:

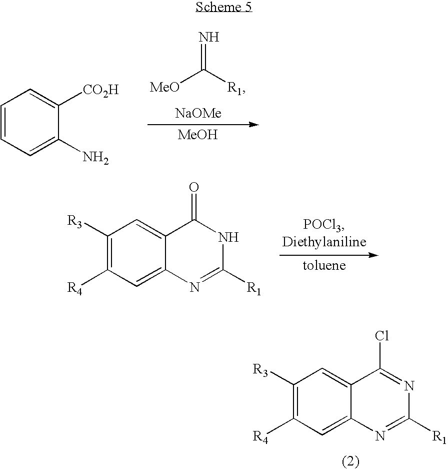

wherein R₃ and R₄ are the same as defined in the formula 1.

First, an imidate compound is reacted with anthranilic acid and sodium methoxide (NaOMe) in the temperature range from room temperature to the reflux temperature of the solvent while stirring in order to prepare 3H-quinazolino-4-one with the substituent R₁. Subsequently, phosphorus oxychloride and diethylaniline are added and reaction is carried out in the temperature range from room temperature to the reflux temperature of the solvent while stirring to transform the ketone group at the C-4 position into chlorine (Cl).

Of the quinazoline derivatives represented by the formula 2, one in which R₁ is a hydrogen atom, alkyl or aryl may also be prepared by the scheme 6 below:

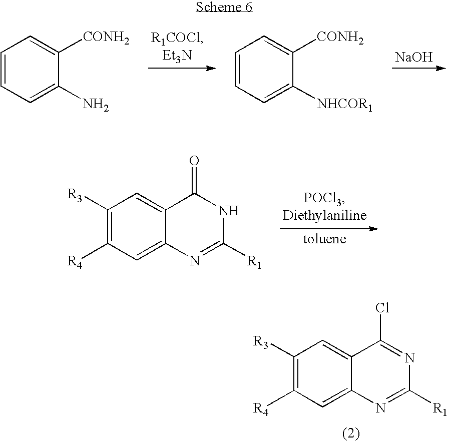

wherein R₁, R₃ and R₄ are the same as defined in the formula 1.

First, an acyl halide is reacted with anthranyl amide and reflux reaction is carried out in the presence of NaOH to prepare 3H-quinazolino-4-one with the substituent R₁. Subsequently, phosphorus oxychloride and diethylaniline are added and reaction is performed in the temperature range from room temperature to the reflux temperature of the solvent while stirring in order to transform the ketone group at the C-4 position into chlorine (Cl).

The compounds represented by the formulas 4 and 5 in the scheme 2, which are used as starting materials of the substitution reaction, may be prepared by the methods well known in the field of organic synthesis. For example, the compound represented by the formula 4 may be prepared by the scheme 7 below:

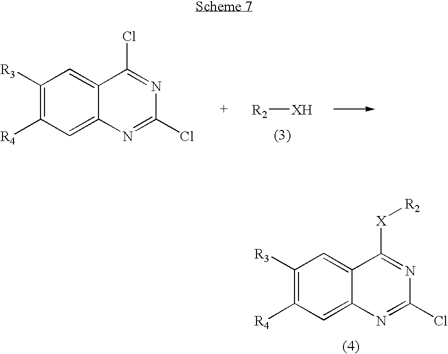

wherein R₂, R₃, R₄ and X are the same as defined in the formula 1.

In the substitution reaction of the scheme 7, the compound represented by the formula 3 may be used in excess as both organic solvent and reactant or another solvent may be used and the compound may be used stoichiometrically. And the reaction may be carried out in the presence of a base, if deemed necessary. As a base, an organic base such as pyridine and an alkylamine or an inorganic base such as an alkali metal salt and an alkaline earth metal salt may be used.

The prepared intermediate compound and the target compound may be separated and purified by such common methods as chromatography and recrystallization.

The compounds of the present invention may be administered by an adequate administration method, for example, orally, intraorally, sublingually, rectally, vaginally, intranasally, locationally or non-orally (including intravenously, intracavernously, intramuscularly, subcutaneously and intraluminally).

For oral, intraoral or sublingual administration, the general dosage of the compound represented by the formula 1 for treatment or prevention of diabetes and obesity is 0.1 to 400 mg/day for an adult weighing 70 kg. Thus, the compound can be prepared into a tablet or capsule, using a pharmaceutically available excipient or vehicle, with the content of the compound of 0.05 to 200 mg, and administered once or several times a day.

For non-oral administration, the compound is administered in an amount of 0.01 to 100 mg. The adequate dosage is determined by the doctor, considering the patient's age, body weight and other characteristics. The aforementioned dosages are for general cases. The dosage may be higher or lower from a patient to another.

When administered to humans, the compound represented by the formula 1 may be administered alone or along with a pharmaceutical vehicle that is selected in consideration of the administration and other standard pharmaceutical practices. For example, the compound may be administered orally, intraorally or sublingually in the form of a tablet containing starch or lactose, ovules by itself or along with excipients or elixir or emulsion containing chemicals that offer specific flavors or colors.

The liquid preparation may be prepared by adding a pharmaceutically available additive such as a suspension (e.g., a mixture of semisynthetic glyceride like methylcellulose and witepsol or apricot kernel oil with PEG-6 ester or a glyceride mixture of PEG-8 and caprylic/capric glyceride). It may be administered non-orally, for example, intravenously, intracavernously, intramuscularly, subcutaneously or intraluminally. For non-oral administration, it is most preferable to use the compound in the form of sterile aqueous solution. The solution may comprise other substances (for example, salts or monosaccharides such as mannitol and glucose) for isotonicity with blood.

Thus, the present invention provides a pharmaceutical composition comprising the compound represented by the formula 1 or a pharmaceutically available salt thereof, as an active ingredient, and a pharmaceutically available diluent or excipient.

The present invention also provides a pharmaceutical composition for medical use comprising the compound represented by the formula 1, a pharmaceutically available salt thereof or a pharmaceutically available prodrug thereof as an active ingredient.

### BRIEF DESCRIPTION OF THE DRAWINGS

FIG. 1 shows the result of Western blot analysis showing the degree of phosphorylation of the serine-79 for the compound of the present invention.

FIGS. 2a to 2g show the results of Western blot analysis showing the degree of phosphorylation of the serine-79 and threonine-172 for the compound of the present invention.

FIGS. 3a and 3b are graphs showing the change of blood glucose level when the compound of the present invention was abdominally administered to db/db mice.

FIG. 4a and FIG. 4b are graphs showing the change of body weight when the compound of the present invention was abdominally administered to db/db mice.

FIGS. 5a, 5b and 5c are graphs showing the change of blood glucose level when the compound of the present invention was orally administered to db/db mice.

FIGS. 6a, 6b and 6c are graphs showing the change of body weight when the compound of the present invention was orally administered to db/db mice.

FIGS. 7a and 7b are graphs showing the change of body weight when the compound of the present invention was abdominally administered to ob/ob mice.

FIGS. 8a and 8b are graphs showing the change of blood glucose level when the compound of the present invention was abdominally administered to ob/ob mice.

FIG. 9 is a graph showing the change of body weight when the compound of the present invention was orally administered to ob/ob mice.

### BEST MODE FOR CARRYING OUT THE INVENTION

Hereinafter, the present invention is described in further detail through examples. However, the following examples are only for the understanding of the present invention and they should not be construed as limiting the scope of the present invention.

Examples 1 to 3 are for the case wherein X═NH and R₁=ethylsulfanyl.

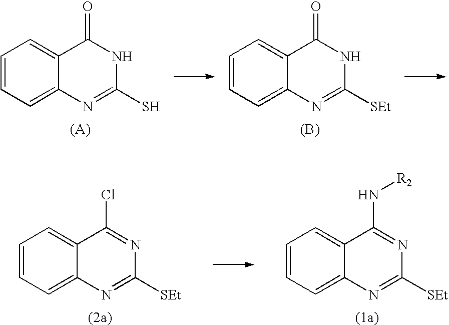

### Example 1

### Preparation of 2-ethylsulfanyl-3H-quinazolino-4-one (formula B)

2-Mercapto-3H-quinazolino-4-one (formula A; 3 g, 16.83 mmol) was dissolved in 100 mL of ethanol and NaOEt (1.5 eq, 9.4 mL, 25.25 mmol) and EtI (1.5 eq, 2.02 mL, 25.25 mmol) were added at room temperature. After 3 hours of reflux, ethanol was removed by distillation under reduced pressure from the mixture when the starting materials disappeared. Ethyl acetate was added and the mixture was washed with water and then dried with anhydrous magnesium sulfate. The organic solvent was removed by distillation under reduced pressure and the target compound was obtained with a yield of 80% (2.78 g) by column chromatography (silica gel; 230-400 mesh).

¹H NMR (CDCl₃, 300 MHz) δ 8.27 (d, J=7.8 Hz, 1H), 7.73 (t, J=7.5 Hz, 1H), 7.71 (d, J=7.8 Hz, 1H), 7.41 (t, J=7.5, 1H), 3.34 (q, J=14.4 Hz, 2H), 1.49 (t, J=7.35 Hz, 3H).

### Example 2

### Preparation of 4-chloro-2-ethylsulfanylquinazoline (formula 2a)

To 2-ethylsulfanyl-3H-quinazolino-4-one (formula B; 1.5 g, 7.27 mmol) was slowly added 50 mL of thionyl chloride at 0° C. After adding 2-3 drops of N,N-dimethylformamide, reflux was performed for 3-4 hours. When the starting materials disappeared, thionyl chloride was removed by distillation under reduced pressure. Residual thionyl chloride was washed with a saturated sodium carbonate solution. The mixture was extracted with ethyl acetate and the organic layer was washed with brine and dried with anhydrous magnesium sulfate. The organic solvent was removed by distillation under reduced pressure and the target compound was obtained with a yield of 79% (1.29 g) by column chromatography.

¹H NMR (CDCl₃, 300 MHz) δ 8.02 (d, J=7.8 Hz, 1H), 7.85 (m, 2H), 7.56 (t, J=7.5 Hz, 1H), 3.29 (q, J=14.4 Hz, 2H), 1.49 (t, J=7.35 Hz, 3H).

### Example 3

### Preparation of 4-substituted amine-2-ethylsulfanylquinazoline (formula 1a)

4-Chloro-2-ethylsulfanylquinazoline (1.0 g, 4.45 mmol) was dissolved in 40 mL of THF. Triethylamine (TEA; 3 eq, 1.86 mL, 13.35 mmol) and amine (1.5 eq) were added and the mixture was stirred at room temperature overnight. When the starting materials disappeared, THF was removed by distillation under reduced pressure and the target compound was obtained with a yield of 90% by column chromatography (silica gel; 230-400 mesh).

Example 4 shows a case wherein X═NH and R₁=phenyl.

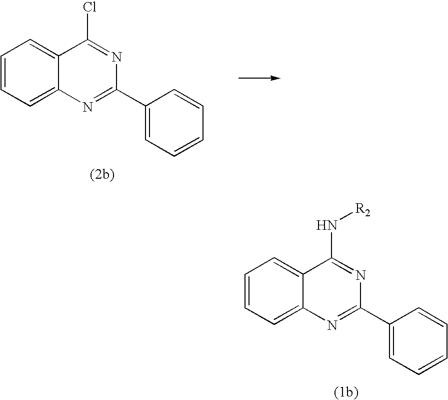

### Example 4

### Preparation of 4-substituted amine-2-phenylquinazoline (formula 1b)

4-Chloro-2-phenylquinazoline (formula 2b; 0.5 g, 2.08 mmol) was dissolved in 30 mL of THF. TEA (5 eq, 1.45 mL, 10.38 mmol) and amine (1.5 eq) were added and the mixture was stirred at room temperature overnight. When the starting materials disappeared, THF was removed by distillation under reduced pressure and the target compound was obtained with a yield of 90% by column chromatography (silica gel; 230-400 mesh).

Examples 5 to 7 are cases wherein X═NH and R₁=hydrogen, alkyl or aryl.

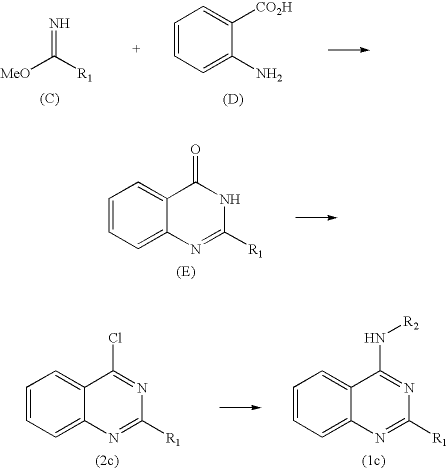

### Example 5

### Preparation of 2-substituted-3H-quinazolino-4-one (formula E)

A hydrochloride of the imidate compound having the R₁ substitute (formula C; 50 mmol) was dissolved in 100 mL of methanol and NaOMe (1.2 eq, 60 mmol) and anthranilic acid (formula D, 1.0 eq, 50 mmol) were slowly added at 0° C. Reflux was performed overnight after slowly heating the mixture to the reflux temperature. After confirming with TLC that all the starting materials had disappeared, the mixture was cooled to 0° C. Water (100 mL) was added to the mixture and the formed crystal was filtered and dried to obtain the target compound with a yield of 70% (35 mmol).

### Example 6

### Preparation of 4-chloro-2-substituted-quinazoline (formula 2c)

2-Substituted-3H-quinazolino-4-one (formula E; 35 mmol) was dissolved in 100 mL of toluene and N,N-diethylaniline (1.5 eq, 52.5 mmol) and phosphorus oxychloride (1.0 eq, 35 mmol) were added. The mixture was stirred at 80° C. for 3 hours and cooled to 0° C. when the starting materials disappeared. The mixture was washed with 1N-NaOH and 1N—HCl and dried with anhydrous sodium sulfate. The organic solvent was removed by distillation under reduced pressure and the target compound was obtained with a yield of 98% (34 mmol) by column chromatography (silica gel; 230-400 mesh).

### Example 7

### Preparation of R₂-(2-(R₁-substituted)-quinazolino-4-yl)-amine (formula 1c)

4-Chloro-2-substituted-quinazoline (formula 2c; 10 mmol) was dissolved in 30 mL of THF. Triethylamine (2 eq, 20 mmol) and amine (1.5 eq, 15 mmol) were added and the mixture was stirred at room temperature overnight. When the starting materials disappeared, THF was removed by distillation under reduced pressure. After adding chloroform, the mixture was washed with a saturated sodium carbonate solution and brine and dried with anhydrous sodium sulfate. The organic solvent was removed by distillation under reduced pressure and the target compound was obtained with a yield of 95% (9.5 mmol) by column chromatography (silica gel; 230-400 mesh).

Examples 8 and 9 are cases wherein R₁═H, alkyl or aryl.

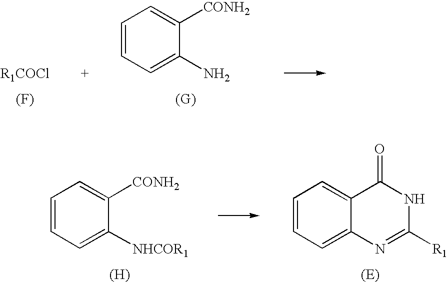

### Example 8

### Preparation of 2-(substituted-carbonyl-amino)-benzamide (formula H)

An acyl chloride with the substituent R₁ (formula F; 50 mmol), anthranilamide (1.0 eq, formula G, 50 mmol) and triethylamine (1.5 eq, 75 mmol) were dissolved in THF and the mixture was stirred at room temperature overnight. When the starting materials disappeared, THF was removed by distillation under reduced pressure. After adding chloroform, the mixture was washed with 1N-NaOH and brine and dried with sodium sulfate. The organic solvent was removed by distillation under reduced pressure and the remainder was crystallized with diethyl ether to obtain the target compound with a yield of 95% (48 mmol).

### Example 9

### Preparation of 2-substituted-3H-quinazolino-4-one (formula E)

2-(Substituted-carbonyl-amino)-benzamide (formula H; 45 mmol) was dissolved in 60 mL of 2.5N-NaOH and the mixture was refluxed for 1 hour. When the starting materials disappeared, the mixture was cooled to 0° C. and neutralized with 3N—HCl. The formed crystal was filtered and dried to obtain the target compound with a yield of 95% (43 mmol). The prepared target compound (formula E) can be utilized in the preparation of the compound with X═NH and R₁═H, alkyl or aryl by the preparation processes of Examples 6 and 7.

Examples 10 and 11 are for the case wherein X═NH and R₁=amine.

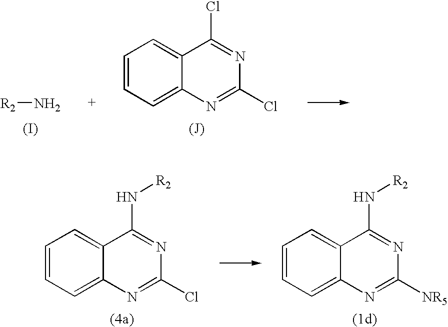

### Example 10

### Preparation of (2-chloro-quinazolino-4-yl)-substituted-amine (formula 4a)

2,4-Dichloro-quinazoline (formula J; 10 mmol) was dissolved in 30 mL of THF. Triethylamine (1.2 eq, 12 mmol) and an amine compound with the R₂ substituent (formula I, 1.05 eq, 10.5 mmol) were added and the mixture was stirred at room temperature for 3 hours. When the starting materials disappeared, the organic solvent was removed by distillation under reduced pressure. After adding ethyl acetate, the mixture was washed with a saturated sodium carbonate solution and brine and dried with anhydrous sodium sulfate. The organic solvent was removed by distillation under reduced pressure and the mixture was crystallized with diethyl ether to obtain the target compound with a yield of 100% (10 mmol).

### Example 11

### Preparation of (2-(R₅-substituted-amine)-quinazolino-4-yl)-R₁-substituted-amine (1d)

(2-Chloro-quinazolino-4-yl)-substituted-amine (formula 4a; 1.5 mmol) and an amine with the R₅ substituent (3 eq, 4.5 mmol) were dissolved in 15 mL of ethanol. The mixture was refluxed overnight while stirring. After confirming with TLC that all the starting materials had disappeared, the organic solvent was removed by distillation under reduced pressure. Ethyl acetate was added and the mixture was washed with a saturated sodium carbonate solution and brine and dried with anhydrous sodium sulfate. The organic solvent was removed by distillation under reduced pressure and the target compound was obtained with a yield of 95% (1.4 mmol) by column chromatography (silica gel; 230-400 mesh).

Example 12 is a case wherein X═NH and R₁=alkoxy.

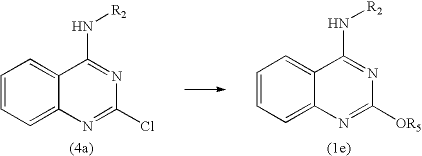

### Example 12

### Preparation of (2-(R₅-substituted-alkoxy)-quinazolino-4-yl)-R₁-substituted-amine (1d)

(2-Chloro-quinazolino-4-yl)-substituted-amine (formula 4a; 1.5 mmol) was dissolved in 15 mL of ethanol and NaOEt (2 eq, 3.0 mmol) was added at 0° C. The mixture was stirred at room temperature for 3 hours and ethanol was removed by distillation under reduced pressure when the starting materials disappeared. Ethyl acetate was added and the mixture was washed with water and dried with sodium sulfate. The organic solvent was removed by distillation under reduced pressure and the target compound was obtained with a yield of 85% (1.3 mmol) by column chromatography (silica gel; 230-400 mesh).

Examples 13 and 14 are cases wherein X═S.

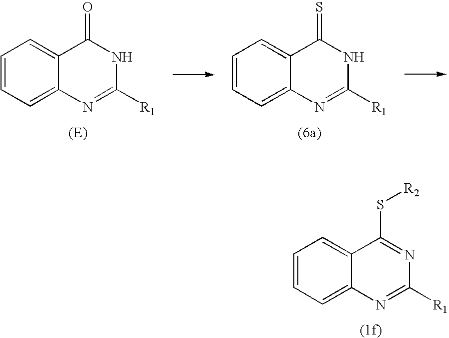

### Example 13

### Preparation of 2-substituted-3H-quinazolino-4-thione (formula 6a)

2-Substituted-3H-quinazolino-4-one (formula E; 3 mmol) and a Lawesson's reagent (0.5 eq, 1.5 mmol) were dissolved in 9 mL of toluene and the mixture was refluxed for 1 hour while stirring. When the starting materials disappeared, the organic solvent was removed by distillation under reduced pressure and the target compound was obtained with a yield of 95% (2.9 mmol) by column chromatography (silica gel; 230-400 mesh).

### Example 14

### Preparation of 2-(R₁-substituted)-4-(R₂-Sulfanyl)-quinazoline (formula 1f)

2-Substituted-3H-quinazolino-4-thione (formula 6a, 2 mmol) and potassium carbonate (1.5 eq, 3 mmol) were dissolved in 8 mL of N,N-dimethylformamide and an R₂-substituted bromine compound (1.2 eq, 2.4 mmol) was added at room temperature. When the starting materials disappeared after 1 hour of stirring, the mixture was diluted with ethyl acetate, washed with water and brine and dried with anhydrous sodium sulfate. The organic solvent was removed by distillation under reduced pressure and the target compound was obtained with a yield of 95% (1.9 mmol) by column chromatography (silica gel; 230-400 mesh).

Example 15 is a case wherein X═O.

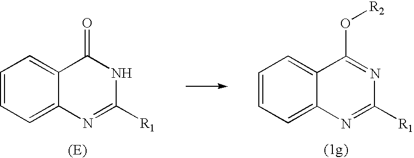

### Example 15

### Preparation of 2-(R₁-substituted)-4-(R₂-substituted-oxy)-quinazoline (formula 1g)

2-Substituted-3H-quinazolino-4-one (formula E; 3 mmol) and potassium carbonate (1.0 eq, 3 mmol) were dissolved in 9 mL of N,N-dimethylformamide and an R₂-substituted bromine compound (1.5 eq, 4.5 mmol) was added at room temperature. When the starting materials disappeared after stirring overnight at room temperature, the mixture was diluted with ethyl acetate, washed with water and brine and dried with anhydrous sodium sulfate. The organic solvent was removed by distillation under reduced pressure and the target compound was obtained with a yield of 45% (1.4 mmol) by column chromatography (silica gel; 230-400 mesh).

Example 16 is a case wherein X═O.

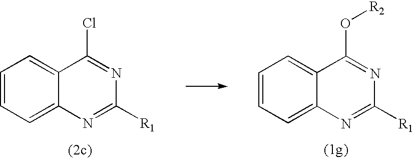

### Example 16

### Preparation of 2-(R₁-substituted)-4-(R₂-substituted-oxy)-quinazoline (formula 1g)

An R₂-substituted alcohol compound (1.2 eq, 24 mmol) and sodium hydride (1.2 eq, 24 mmol) were dissolved in 60 mL of N,N-dimethylformamide at 0° C. and 4-chloro-2-substituted-quinazoline (formula 2c; 20 mmol) was added. After stirring overnight at room temperature, the mixture was diluted with ethyl acetate, washed with water and brine and dried with anhydrous sodium sulfate. The organic solvent was removed by distillation under reduced pressure and the target compound was obtained with a yield of 46% (9.2 mmol) by column chromatography (silica gel; 230-400 mesh).

Those skilled in the art can easily prepare the compound represented by the formula 1 according to the preparation processes of Examples 1 to 4. Some examples of thus prepared compounds are as follows.
Compound No. 1: 2-[2-(2-Phenylquinazolino-4-ylamino)-ethoxy]-ethanol

¹H NMR (CDCl₃, 300 MHz) δ 8.56 (m, 2H), 7.91 (d, J=8.1 Hz, 1H), 7.70 (m, 2H), 7.50 (m, 3H), 7.37 (t, J=7.5 Hz, 1H), 6.40 (m, 1H), 4.00 (m, 2H), 3.82 (m, 4H), 3.64 (m, 2H), 2.83 (br s, 1H)
Compound No. 2: Isopropyl-(2-trichloromethylquinazolino-4-yl)-amine
Compound No. 3: Cyclopropyl-(2-trichloromethylquinazolino-4-yl)-amine
Compound No. 4: (2-Piperidino-1-ylethyl)-(2-trichloromethylquinazolino-4-yl)-amine
Compound No. 5: (1-Phenylethyl)-(2-trichloromethylquinazolino-4-yl)-amine
Compound No. 6: (4-Phenylbutyl)-(2-trichloromethylquinazolino-4-yl)-amine
Compound No. 7: Propyl-(2-trichloromethylquinazolino-4-yl)-amine
Compound No. 8: (2-Ethylsulfanylquinazolino-4-yl)-isopropylamine
Compound No. 9: (2-Ethylsulfanylquinazolino-4-yl)-(3-fluorobenzyl)-amine

¹H NMR (CDCl₃, 300 MHz) δ 7.68 (m, 3H), 7.30 (m, 2H), 7.15 (d, J=7.5 Hz, 1H), 7.05 (d, J=7.5 Hz, 1H), 6.95 (t, J=7.5 Hz, 1H), 6.24 (s, 1H), 4.85 (d, J=5.4 Hz, 2H), 3.15 (q, J=14.4 Hz, 2H), 1.37 (t, J=7.35 Hz, 3H)
Compound No. 10: (2-Ethylsulfanylquinazolino-4-yl)-[2-(3-fluorophenyl)-ethyl]-amine
Compound No. 11: (2-Ethylsulfanylquinazolino-4-yl)-(2-morpholino-4-ylethyl)-amine
Compound No. 12: (2-Ethylsulfanylquinazolino-4-yl)-[2-(3-trifluoromethyl-phenyl)ethyl]-amine
Compound No. 13: (6-Nitro-quinazolino-4-yl)-phenylamine
Compound No. 14: (6,7-Diethoxyquinazolino-4-yl)-naphthaleno-1-ylmethylamine
Compound No. 15: (6,7-Diethoxyquinazolino-4-yl)-(3-morpholino-4-ylpropyl)-amine
Compound No. 16: Allyl-(6,7-diethoxyquinazolino-4-yl)-amine
Compound No. 17: (6,7-Diethoxyquinazolino-4-yl)-(2-pyridino-2-ylethyl)-amine
Compound No. 18: (4-Chlorobenzyl)-(6,7-diethoxyquinazolino-4-yl)-amine
Compound No. 19: Butyl-(2-ethylsulfanylquinazolino-4-yl)-amine
Compound No. 20: sec-Butyl-(2-ethylsulfanylquinazolino-4-yl)-amine

¹H NMR (CDCl₃, 300 MHz) δ 7.86 (d, J=7.5 Hz, 1H), 7.61 (m, 2H), 7.30 (d, J=7.5 Hz, 1H), 6.57 (s, 1H), 4.42 (m, 1H), 3.23 (q, J=14.4 Hz, 2H), 1.74 (m, 2H), 1.47-1.22 (m, 6H), 1.00 (t, J=7.35 Hz, 3H)
Compound No. 21: (2-Ethylsulfanylquinazolino-4-yl)-isobutylamine
Compound No. 22: Benzyl-(2-Ethylsulfanylquinazolino-4-yl)-amine

¹H NMR (CDCl₃, 300 MHz) δ 7.65 (m, 3H), 7.34 (m, 6H), 6.01 (s, 1H), 4.86 (d, J=5.4 Hz, 2H), 3.65 (q, J=14.4 Hz, 2H), 1.40 (t, J=7.35 Hz, 3H)
Compound No. 23: (2-Ethylsulfanylquinazolino-4-yl)-(1-phenylethyl)-amine
Compound No. 24: (2-Ethylsulfanylquinazolino-4-yl)-(3-methoxybenzyl)-amine

¹H NMR (CDCl₃, 300 MHz) δ 7.66 (m, 3H), 7.29 (m, 2H), 6.95 (m, 2H), 6.88 (m, 1H), 6.02 (s, 1H), 4.82 (d, J=5.5 Hz, 2H), 3.80 (s, 3H), 3.20 (q, J=14.4 Hz, 2H), 1.40 (t, J=7.35 Hz, 3H)
Compound No. 25: (2-Ethylsulfanylquinazolino-4-yl)-(4-methoxybenzyl)-amine

¹H NMR (CDCl₃, 300 MHz) δ 7.62 (m, 3H), 7.33 (m, 3H), 6.90 (d, J=8.4 Hz, 2H), 4.78 (d, J=5.7 Hz, 2H), 3.81 (s, 3H), 3.23 (q, J=14.4 Hz, 2H), 1.43 (t, J=7.36 Hz, 3H)
Compound No. 26: (2-Ethylsulfanylquinazolino-4-yl)-(3-trifluoromethylbenzyl)-amine

¹H NMR (CDCl₃, 300 MHz) δ 7.92 (d, J=8.3 Hz, 1H), 7.63 (m, 4H), 7.55 (m, 2H), 7.46 (m, 2H), 7.33 (m, 1H), 4.94 (d, J=5.4 Hz, 2H), 3.12 (q, J=14.4 Hz, 2H), 1.30 (t, J=7.35 Hz, 3H)
Compound No. 27: Cyclohexyl-(2-ethylsulfanylquinazolino-4-yl)-amine
Compound No. 28: Cyclopentyl-(2-ethylsulfanylquinazolino-4-yl)-amine
Compound No. 29: (2-Ethylsulfanylquinazolino-4-yl)-phenethylamine

¹H NMR (CDCl₃, 300 MHz) δ 7.66 (m, 2H), 7.45 (d, J=8.2 Hz, 1H), 7.33 (m, 6H), 5.84 (br s, 1H), 3.91 (q, J=12.6 Hz, 2H), 3.26 (q, J=14.4 Hz, 2H), 3.03 (t, J=6.9 Hz, 2H), 1.47 (t, J=7.36 Hz, 3H)
Compound No. 30: (2-Ethylsulfanylquinazolino-4-yl)-(4-phenylbutyl)-amine

¹H NMR (CDCl₃, 300 MHz) δ 7.61 (m, 3H), 7.30 (m, 3H), 7.20 (m, 3H), 5.74 (br s, 1H), 3.68 (m, 2H), 3.23 (q, J=14.4 Hz, 2H), 2.69 (m, 2H), 1.76 (m, 4H), 1.44 (t, J=7.36 Hz, 3H)
Compound No. 31: (2-Ethylsulfanylquinazolino-4-yl)-propylamine
Compound No. 32: (2-Ethylsulfanylquinazoline-4-yl)-(3-phenylpropyl)-amine
Compound No. 33: (2-Ethylsulfanylquinazolino-4-yl)-(3-isopropoxypropyl)-amine
Compound No. 34: (2-Ethylsulfanylquinazolino-4-yl)-(2-methoxy-ethyl)-amine
Compound No. 35: (2-Ethylsulfanylquinazolino-4-yl)-naphthaleno-1-ylmethyl amine

¹H NMR (CDCl₃, 300 MHz) δ 8.11 (m, 1H), 7.90 (m, 2H), 7.65 (m, 2H), 7.53 (m, 5H), 7.24 (m, 1H), 5.92 (br s, 1H), 5.27 (d, J=4.5 Hz, 2H), 3.26 (q, J=14.4 Hz, 2H), 1.22 (t, J=7.4 Hz, 3H)
Compound No. 36: (2-Ethylsulfanylquinazolino-4-yl)-(3-morpholino-4-ylpropyl)-amine

¹H NMR (CDCl₃, 300 MHz) δ 7.93 (m, 1H), 7.70 (m, 3H), 7.31 (m, 1H), 3.78 (m, 6H), 3.22 (q, J=14.4 Hz, 2H), 2.59 (m, 6H), 1.87 (m, 2H), 1.43 (t, J=7.41 Hz, 3H)
Compound No. 37: Benzo[1,3]dioxol-5-ylmethyl-(2-ethylsulfanylquinazolino-4-yl)-amine

¹H NMR (CDCl₃, 300 MHz) δ 7.66 (m, 3H), 7.31 (m, 1H), 6.80 (m, 3H), 5.97 (s, 2H), 5.90 (br s, 1H), 4.76 (d, J=5.1 Hz, 2H), 3.23 (q, J=14.4 Hz, 2H), 1.44 (t, J=7.36 Hz, 3H)
Compound No. 38: (3,5-Dimethoxybenzyl)-(2-ethylsulfanylquinazolino-4-yl)-amine
Compound No. 39: (2-Ethylsulfanylquinazolino-4-yl)-(2-fluorobenzyl)-amine
Compound No. 40: (2-Ethylsulfanylquinazolino-4-yl)-(1-methoxymethylpropyl)-amine

¹H NMR (CDCl₃, 300 MHz) δ 7.65 (m, 3H), 7.32 (m, 1H), 5.96 (d, J=7.5 Hz, 1H), 4.48 (d, J=3.6 Hz, 1H), 3.60 (m, 2H), 3.41 (s, 3H), 3.20 (q, J=14.4 Hz, 2H), 1.75 (m, 2H), 1.44 (t, J=7.4 Hz, 3H), 1.01 (t, J=7.35 Hz, 3H)
Compound No. 41: (1-Benzyl-piperidino-4-yl)-(2-ethylsulfanylquinazolino-4-yl)-amine

¹H NMR (CDCl₃, 300 MHz) δ 7.60 (m, 3H), 7.34 (m, 6H), 5.54 (d, J=6.9 Hz, 1H), 4.26 (m, 1H), 3.50 (m, 2H), 3.22 (q, J=14.4 Hz, 2H), 2.90 (m, 2H), 2.17 (m, 4H), 1.65 (m, 2H), 1.22 (t, J=7.35 Hz, 3H)
Compound No. 42: Allyl-(2-ethylsulfanylquinazolino-4-yl)-amine

¹H NMR (CDCl₃, 300 MHz) δ 7.66 (m, 3H), 7.33 (m, 1H), 6.03 (m, 1H), 5.79 (br s, 1H), 5.29 (m, 2H), 4.31 (t, J=5.55 Hz, 2H), 3.23 (q, J=14.5 Hz, 2H), 1.44 (t, J=7.35 Hz, 3H)
Compound No. 43: (2-Ethylsulfanylquinazolino-4-yl)-hexadecylamine
Compound No. 44: (2-Ethylsulfanylquinazolino-4-yl)-(2-piperidino-1-ylethyl)-amine
Compound No. 45: (2-Ethylsulfanylquinazolino-4-yl)-(2-pyrrolidino-1-ylethyl)-amine
Compound No. 46: (2-Ethylsulfanylquinazolino-4-yl)-(2-pyridino-2-ylethyl)-amine
Compound No. 47: 2-(2-Ethylsulfanylquinazolino-4-ylamino)-ethanol

¹H NMR (DMSO-d₆, 300 MHz) δ 8.33 (m, 1H), 8.14 (d, J=8.2 Hz, 1H), 7.67 (t, J=7.5 Hz, 1H), 7.48 (d, J=8.1 Hz, 1H), 7.36 (t, J=7.6 Hz, 1H), 4.81 (br s, 1H), 3.60 (m, 4H), 3.13 (q, J=14.4 Hz, 2H), 1.32 (t, J=7.35 Hz, 3H)
Compound No. 48: (2-Ethylsulfanylquinazolino-4-yl)-[3-(2-methyl-piperidino-1-yl)-propyl]-amine
Compound No. 49: (2-Chloro-6-fluorobenzyl)-(2-ethylsulfanylquinazolino-4-yl)-amine

¹H NMR (CDCl₃, 300 MHz) δ 7.64 (m, 3H), 7.25 (m, 3H), 7.05 (m, 1H), 6.00 (br s, 1H), 5.06 (d, J=5.6 Hz, 2H), 3.28 (q, J=14.4 Hz, 2H), 1.46 (t, J=7.35 Hz, 3H)
Compound No. 50: 2-[2-(2-Ethylsulfanylquinazolino-4-ylamino)-ethoxy]-ethanol

¹H NMR (CDCl₃, 300 MHz) δ 7.60 (m, 3H), 7.27 (m, 1H), 6.50 (br s, 1H), 3.85 (m, 4H), 3.78 (m, 2H), 3.65 (m, 2H), 3.20 (q, J=14.4 Hz, 2H), 1.42 (t, J=7.35 Hz, 3H)
Compound No. 51: Cyclohexyl-(2-phenylquinazolino-4-yl)-amine

¹H NMR (CDCl₃, 300 MHz) δ 8.58 (m, 4H), 7.93 (m, 1H), 7.72 (m, 2H), 7.50 (m, 4H), 5.58 (d, J=7.2 Hz, 1H), 4.43 (m, 1H), 2.27 (m, 2H), 1.85 (m, 3H), 1.54 (m, 2H), 1.34 (m, 3H)
Compound No. 52: (4-Phenylbutyl)-(2-phenylquinazolino-4-yl)-amine
Compound No. 53: (5-Phenyl-pentyl)-(2-phenylquinazolino-4-yl)-amine
Compound No. 54: (4-Methylbenzyl)-(2-phenylquinazolino-4-yl)-amine
Compound No. 55: Isobutyl-(2-phenylquinazolino-4-yl)-amine
Compound No. 56: Butyl-(2-phenylquinazolino-4-yl)-amine
Compound No. 57: Naphthaleno-1-ylmethyl-(2-phenylquinazolino-4-yl)-amine
Compound No. 58: (2-Phenylquinazolino-4-yl)-pyridino-2-ylmethylamine

¹H NMR (CDCl₃, 300 MHz) δ 8.61 (m, 3H), 7.91 (t, J=8.2 Hz, 1H), 7.72 (t, J=8.2 Hz, 1H), 7.52 (m, 6H), 7.28 (m, 1H), 5.07 (d, J=4.5 Hz, 2H)
Compound No. 59: (1-Phenylquinazolino-4-yl)-thiopenyl-2-ylmethylamine

¹H NMR (CDCl₃, 300 MHz) δ 8.66 (m, 2H), 7.96 (d, J=8.1 Hz, 1H), 7.69 (m, 2H), 7.53 (m, 3H), 7.41 (m, 1H), 7.24 (t, J=7.4 Hz, 1H), 7.13 (t, J=7.4 Hz, 1H), 7.00 (t, J=7.36 Hz, 1H), 6.09 (d, J=5.1 Hz, 1H), 5.17 (d, J=5.4 Hz, 2H),
Compound No. 60: Isopropyl-(2-phenylquinazolino-4-yl)-amine
Compound No. 61: (2-Phenylquinazolino-4-yl)-propylamine

¹H NMR (CDCl₃, 300 MHz) δ 8.60 (m, 1H), 7.94 (d, J=8.2 Hz, 1H), 7.70 (m, 2H), 7.41 (m, 4H), 5.78 (t, J=5.4 Hz, 1H), 3.76 (q, J=13.2 Hz, 2H), 1.80 (q, J=14.4 Hz, 2H), 1.08 (t, J=8.4 Hz, 3H)
Compound No. 62: (3-Isopropoxypropyl)-(2-phenylquinazolino-4-yl)-amine
Compound No. 63: (3-Fluorobenzyl)-(2-phenylquinazolino-4-yl)-amine
Compound No. 64: (2-Phenylquinazolino-4-yl)-[2-(3-trifluoromethyl-phenyl)-ethyl]-amine

¹H NMR (CDCl₃, 300 MHz) δ 8.64 (m, 2H), 7.95 (d, J=8.3 Hz, 1H), 7.74 (t, J=7.4 Hz, 1H), 7.50 (m, 4H), 7.40 (t, J=6.6 Hz, 1H), 7.30 (m, 1H), 7.03 (m, 3H), 5.85 (t, J=5.4 Hz, 1H), 4.03 (q, J=13.2 Hz, 2H), 3.10 (t, J=7.9 Hz, 2H)
Compound No. 65: Benzo[1,3]dioxol-5-ylmethyl-(2-phenylquinazolino-4-yl)-amine
Compound No. 66: (2-Phenylquinazolino-4-yl)-(3-trifluoromethylbenzyl)-amine
Compound No. 67: (2-Methoxy-ethyl)-(2-phenylquinazolino-4-yl)-amine
Compound No. 68: Phenethyl-(2-phenylquinazolino-4-yl)-amine
Compound No. 69: Phenyl-(2-phenylquinazolino-4-yl)-amine

¹H NMR (DMSO, 300 MHz) δ 11.56 (br s, 1H), 8.92 (d, J=8.2 Hz, 1H), 8.35 (m, 3H), 8.09 (t, J=7.4 Hz, 1H), 7.83 (m, 3H), 7.60 (m, 5H), 7.38 (d, J=8.4 Hz, 1H)
Compound No. 70: (4-Methoxyphenyl)-(2-phenylquinazolino-4-yl)-amine
Compound No. 71: (2-Phenylquinazolino-4-yl)-o-tollylamine
Compound No. 72: (4-Isopropyl-phenyl)-(2-phenylquinazolino-4-yl)-amine

¹H NMR (DMSO, 300 MHz) δ 11.45 (br s, 1H), 8.90 (d, J=8.2 Hz, 1H), 8.35 (m, 3H), 8.08 (t, J=7.4 Hz, 1H), 7.80 (m, 3H), 7.68 (m, 3H), 7.41 (d, J=8.4 Hz, 1H)
Compound No. 73: (2,4-Dimethyl-phenyl)-(2-phenylquinazolino-4-yl)-amine

¹H NMR (DMSO, 300 MHz) δ 11.41 (br s, 1H), 8.82 (d, J=8.2 Hz, 1H), 8.33 (d, J=8.2 Hz, 1H), 8.18 (m, 3H), 7.83 (t, J=7.4 Hz, 1H), 7.65 (m, 3H), 7.38 (d, J=8.1 Hz, 1H), 7.19 (m, 3H), 2.37 (s, 3H), 2.27 (s, 3H)
Compound No. 74: (2-Phenylquinazolino-4-yl)-(4-undecyl-phenyl)-amine
Compound No. 75: (4-Ethyl-phenyl)-(2-phenylquinazolino-4-yl)-amine
Compound No. 76: (4-Fluorophenyl)-(2-phenylquinazolino-4-yl)-amine

¹H NMR (DMSO, 300 MHz) δ 11.42 (br s, 1H), 8.90 (d, J=8.2 Hz, 1H), 8.34 (m, 3H), 8.09 (t, J=7.4 Hz, 1H), 7.86 (m, 3H), 7.68 (m, 3H), 7.40 (m, 2H)
Compound No. 77: (2-Methoxyphenyl)-(2-phenylquinazolino-4-yl)-amine
Compound No. 78: (2-Phenylquinazolino-4-yl)-(2,4,6-trimethyl-phenyl)-amine

¹H NMR (DMSO, 300 MHz) δ 11.39 (br s, 1H), 8.83 (d, J=8.2 Hz, 1H), 8.27 (t, J=7.4 Hz, 1H), 8.11 (m, 3H), 7.85 (t, J=7.5 Hz, 1H), 7.67 (t, J=7.5 Hz, 1H), 7.60 (m, 2H), 7.07 (s, 2H), 2.33 (s, 3H), 2.18 (s, 6H)
Compound No. 79: (4-Butyl-phenyl)-(2-phenylquinazolino-4-yl)-amine
Compound No. 80: (2-Phenoxy-phenyl)-(2-phenylquinazolino-4-yl)-amine

¹H NMR (DMSO, 300 MHz) δ 11.62 (br s, 1H), 8.69 (d, J=8.2 Hz, 1H), 8.28 (m, 3H), 8.09 (t, J=7.5 Hz, 1H), 7.79 (t, J=7.5 Hz, 1H), 7.68 (m, 3H), 7.34 (d, J=7.8 Hz, 1H), 7.16 (d, J=7.8 Hz, 1H), 7.12 (m, 3H), 6.90 (m, 3H)
Compound No. 81: (3-Phenoxy-phenyl)-(2-phenylquinazolino-4-yl)-amine
Compound No. 82: (4-Phenoxy-phenyl)-(2-phenylquinazolino-4-yl)-amine
Compound No. 83: (3-Bromophenyl)-(2-phenylquinazolino-4-yl)-amine
Compound No. 84: 2-Methoxy-5-(2-phenylquinazolino-4-ylamino)phenol

¹H NMR (DMSO, 300 MHz) δ 11.29 (br s, 1H), 9.45 (br s, 1H), 8.82 (d, J=8.2 Hz, 1H), 8.36 (m, 2H), 8.25 (d, J=8.1 Hz, 1H), 8.06 (t, J=7.4 Hz, 1H), 7.79 (t, J=7.4 Hz, 1H), 7.64 (m, 3H), 7.33 (s, 1H), 7.25 (d, J=8.7 Hz, 1H), 7.05 (d, J=8.7 Hz, 1H), 3.83 (s, 3H)
Compound No. 85: 5-(2-Phenylquinazolino-4-ylamino)-pentane-1-ol
Compound No. 86: Benzyl-(2-phenylquinazolino-4-yl)-amine
Compound No. 87: (1-Phenylethyl)-(2-phenylquinazolino-4-yl)-amine
Compound No. 88: (4-Chlorobenzyl)-(2-phenylquinazolino-4-yl)-amine
Compound No. 89: (3,4-Dichlorobenzyl)-(2-phenylquinazolino-4-yl)-amine
Compound No. 90: (3-Methoxybenzyl)-(2-phenylquinazolino-4-yl)-amine
Compound No. 91: (2-Methoxybenzyl)-(2-phenylquinazolino-4-yl)-amine
Compound No. 92: (3,5-Dimethoxybenzyl)-(2-phenylquinazolino-4-yl)-amine
Compound No. 93: (2,3-Dimethoxybenzyl)-(2-phenylquinazolino-4-yl)-amine
Compound No. 94: (2-Fluorobenzyl)-(2-phenylquinazolino-4-yl)-amine
Compound No. 95: Cyclopentyl-(2-phenylquinazolino-4-yl)-amine

¹H NMR (CDCl₃, 300 MHz) δ 8.61 (m, 2H), 7.93 (d, J=8.1 Hz, 1H), 7.72 (m, 2H), 7.50 (m, 4H), 5.67 (d, J=6.3 Hz, 1H), 4.80 (q, J=13.5 Hz, 1H), 2.31 (m, 2H), 1.80 (m, 4H), 1.65 (m, 2H)
Compound No. 96: sec-Butyl-(2-phenylquinazolino-4-yl)-amine
Compound No. 97: Allyl-(2-phenylquinazolino-4-yl)-amine
Compound No. 98: (2-Phenylquinazolino-4-yl)-(2-trifluoromethylbenzyl)-amine
Compound No. 99: Hexadecyl-(2-phenylquinazolino-4-yl)-amine
Compound No. 100: (2-Phenylquinazolino-4-yl)-(2-pyrrolidino-1-ylethyl)-amine
Compound No. 101: (2-Phenylquinazolino-4-yl)-(2-piperidino-1-ylethyl)-amine
Compound No. 102: (2-Phenylquinazolino-4-yl)-(2-pyridino-2-ylethyl)-amine
Compound No. 103: [2-(3-Fluorophenyl)-ethyl]-(2-phenylquinazolino-4-yl)-amine

¹H NMR (CDCl₃, 300 MHz) δ 8.59 (dd, J₁=7.8 Hz, J₂=1.8 Hz, 2H), 7.93 (d, J=8.1 Hz, 1H), 7.72 (t, J=7.5 Hz, 1H), 7.47 (m, 7H), 6.99 (m, 3H), 5.76 (br s, 1H), 4.06 (m, 2H), 3.12 (t, J=7.2 Hz, 2H)
Compound No. 104: (1-Methoxymethylpropyl)-(2-phenylquinazolino-4-yl)-amine

¹H NMR (CDCl₃, 300 MHz) δ8.56 (t, J=3.8 Hz, 2H), 7.96 (d J=8.3 Hz, 1H), 7.72 (m, 2H), 7.49-7.39 (m, 5H), 6.06 (d J=7.2 Hz, 1H), 4.72 (t J=3.8 Hz, 1H), 3.65 (m, 2H), 3.43 (s, 1H), 1.84 (m, 2H), 1.06 (t, J=7.4 Hz, 3H)
Compound No. 105: (1-Benzyl-piperidino-4-yl)-(2-phenylquinazolino-4-yl)-amine

¹H NMR (CDCl₃, 300 MHz) δ 8.54 (t J=6.7 Hz, 2H), 7.92 (d, J=8.3 Hz, 1H), 7.69 (m, 2H), 7.50-2.27 (m, 8H), 5.59 (d J=7.1 Hz, 1H), 4.46 (m, 1H), 3.61 (s, 2H), 2.95 (m, 2H), 2.68 (s, 1H), 2.30 (m, 4H), 1.70 (m, 2H)
Compound No. 106: (2-Morpholino-4-ylethyl)-(2-phenylquinazolino-4-yl)-amine

¹H NMR (CDCl₃, 300 MHz) δ 8.56 (t J=6.2 Hz, 2H), 7.92 (d, J=8.5 Hz, 2H), 7.71 (m, 2H), 7.49-7.26 (m, 4H), 6.56 (s, 1H), 3.86-3.74 (m. 6H), 2.73 (t, J=5.9 Hz 2H), 3.76 (m, 4H)
Compound No. 107: [2-(3,4-Dimethoxyphenyl)-ethyl]-(2-phenylquinazolino-4-yl)-amine

¹H NMR (CDCl₃, 300 MHz) δ 8.60 (t J=5.2 Hz, 2H), 7.93 (d J=8.3 Hz, 1H), 7.69 (t J=7.5 Hz, 1H), 7.57 (t J=8.1 Hz, 1H), 7.50 (m, 3H), 7.36 (t J=3.6 Hz, 1H), 6.84 (m, 3H), 5.96 (s, 1H), 4.12 (m, 2H), 3.87 (s, 3H), 3.81 (s, 3H), 3.03 (t, J=6.7 Hz, 2H)
Compound No. 108: (2,4-Dichlorobenzyl)-(2-phenylquinazolino-4-yl)-amine

¹H NMR (CDCl₃, 300 MHz) δ8.53 (m, 2H), 7.93 (d, J=8.1 Hz, 1H), 7.74 (t, J=7.7 Hz, 2H), 7.47 (m, 6H), 7.17 (dd, J₁=8.4 Hz, J₂=2.0 Hz, 1H), 6.17 (br, 1H), 5.06 (d, J=6.0 Hz, 2H)
Compound No. 109: (2-Chlorobenzyl)-(2-phenylquinazolino-4-yl)-amine

1H NMR (CDCl₃, 300 MHz) δ 8.56 (t, J=3.3 Hz, 2H), 7.95 (d J=8.6 Hz, 1H), 7.71 (m, 2H), 7.55-7.37 (m, 6H), 7.21 (t, J=3.6 Hz, 2H), 6.31 (s, 1H), 5.09 (d, J=5.8 Hz, 2H)
Compound No. 110: Cyclopropyl-(2-phenylquinazolino-4-yl)-amine

¹H NMR (CDCl₃, 300 MHz) δ 8.63 (d, J=7.3 Hz, 2H), 7.93 (d, J=8.3 Hz, 2H), 7.70 (m, 2H), 7.49 (m, 3H), 7.37 (t, J=7.5 Hz, 1H), 6.03 (s, 1H), 3.17 (m, 1H), 0.97 (m, 2H), 0.73 (m, 2H)
Compound No. 111: (2-Phenylquinazolino-4-yl)-pyridino-3-ylmethylamine

¹H NMR (CDCl₃, 300 MHz) δ 8.73 (m, 1H), 8.51 (m, 3H), 8.04 (d J=8.2 Hz, 1H,), 7.94 (d, J=8.2 Hz, 1H), 7.85 (d, J=7.1 Hz, 1H), 7.67 (t, J=7.7 Hz, 1H), 7.47 (m, 3H), 7.37 (m, 1H), 7.26 (m, 2H), 5.03 (d, J=5.1 Hz, 2H)
Compound No. 112: 2-(2-Phenylquinazolino-4-ylamino)-propane-1-ol

¹H NMR (CDCl₃, 300 MHz) δ 8.49 (m, 2H), 7.93 (t, J=7.8 Hz, 2H), 7.58 (m, 5H), 7.34 (t, J=7.6 Hz, 1H), 7.71 (t, J=7.7 Hz, 1H), 3.91 (m, 2H), 1.45 (m, 4H)
Compound No. 113: 2-Methoxy-4-[(2-phenylquinazolino-4-ylamino)-methyl]-phenol

¹H NMR (MeOD, 300 MHz) δ 8.35 (d, J=2.2 Hz, 2H), 8.02 (m, 1H), 7.80 (m, 2H), 7.45 (m, 4H), 7.07 (s, 1H), 6.89 (t, J=8.0 Hz, 1H), 6.75 (t, J=8.0 Hz, 1H), 4.85 (s, 2H), 3.72 (s, 3H)
Compound No. 114: (2-Fluoro-ethyl)-(2-phenylquinazolino-4-yl)-amine

¹H NMR (CDCl₃, 300 MHz) δ 8.53 (m, 2H), 7.93 (d, J=8.4 Hz, 1H), 7.74 (t, J=7.5 Hz, 2H), 7.50 (m, 4H), 6.03 (br s, 1H), 4.86 (t, J=4.8 Hz, 1H), 4.70 (t, J=5.1 Hz, 1H), 4.20 (q, J=5.1 Hz, 1H), 4.10 (q, J=5.1 Hz, 1H)
Compound No. 115: (2,3-Dichlorobenzyl)-(2-phenylquinazolino-4-yl)-amine

¹H NMR (CDCl₃, 300 MHz) δ 8.52 (t, J=3.7 Hz, 2H), 7.92 (d, J=8.5 Hz, 1H), 7.72 (t, J=6.6 Hz, 2H), 7.42 (m, 6H), 7.08 (t, J=7.8 Hz, 1H), 6.24 (s, 1H), 5.09 (d, J=5.9 Hz, 2H)
Compound No. 116: (3-Nitrobenzyl)-(2-phenylquinazolino-4-yl)-amine

¹H NMR (CDCl₃, 300 MHz) δ 8.46 (m, 2H), 8.19 (m, 1H), 8.03 (d, J=7.5 Hz, 1H), 7.91 (d, J=7.9 Hz, 1H), 7.82 (d, J=7.7 Hz, 1H), 7.70 (d, J=6.0 Hz, 1H), 7.42 (m, 5H), 6.54 (s, 1H), 4.96 (d, J=4.4 Hz, 2H)
Compound No. 117: (1-Ethyl-pyrrolidino-2-ylmethyl)-(2-phenylquinazolino-4-yl)-amine

¹H NMR (CDCl₃, 300 MHz) δ 8.47 (d, J=5.6 Hz, 2H), 8.10 (d, J=8.1 Hz, 1H), 7.94 (s, 1H), 7.84 (d, J=8.3 Hz, 1H), 7.68 (t, J=7.6 Hz, 1H), 7.42 (m, 4H), 5.97 (s, 1H), 4.08 (m, 2H), 3.58 (m, 2H), 3.01 (t, J=4.7 Hz, 1H), 2.67 (m, 2H), 2.09-1.91 (m, 4H), 1.19 (m, 3H)
Compound No. 118: (3,4-Dimethoxybenzyl)-(2-phenylquinazolino-4-yl)-amine

¹H NMR (CDCl₃, 300 MHz) δ 8.59 (t, J=3.8 Hz, 2H), 7.93 (d, J=8.1 Hz, 1H), 7.71 (m, 2H), 7.45 (m, 3H), 7.36 (t, J=7.6 Hz, 1H), 7.04 (s, 1H), 6.97 (d, J=8.4 Hz, 1H), 6.80 (t, J=4.1 Hz, 1H), 6.21 (s, 1H), 4.90 (d, J=5.2 Hz, 2H), 3.87 (s, 3H), 3.78 (s, 3H)
Compound No. 119: (2-Phenylquinazolino-4-yl)-(2-p-tollylethyl)-amine

¹H NMR (CDCl₃, 300 MHz) δ 8.59 (t, J=3.3 Hz, 2H), 7.93 (d, J=8.4 Hz, 1H), 7.69 (t, J=7.6 Hz, 1H), 7.55 (m, 4H), 7.36 (t, J=7.5 Hz, 1H), 7.16 (m, 4H), 5.91 (s, 1H), 4.01 (m, 2H), 3.05 (t, J=6.9 Hz, 2H), 2.43 (s, 3H)
Compound No. 120: [2-(3-Methoxyphenyl)-ethyl]-(2-phenylquinazolino-4-yl)-amine

¹H NMR (CDCl₃, 300 MHz) δ 8.61 (t, J=3.8 Hz, 2H), 7.93 (d, J=8.3H, 1Hz), 7.70 (m, 1H), 7.49 (m, 4H), 7.30 (m, 2H), 6.85 (m, 3H), 5.90 (s, 1H), 4.02 (m, 2H), 3.76 (s, 3H), 3.05 (t, J=6.9 Hz, 2H)
Compound No. 121: (1-Ethylpropyl)-(2-phenylquinazolino-4-yl)-amine

¹H NMR (CDCl₃, 300 MHz) δ 8.55 (t, J=3.5 Hz, 2H), 7.95 (d, J=8.2 Hz, 1H), 7.69 (m, 2H), 7.46 (m, 4H), 5.59 (s, 1H), 4.53 (d, J=6.7 Hz, 1H), 1.85-1.62 (m, 4H), 1.00 (m, 6H)
Compound No. 122: [2-(2,5-Dimethoxyphenyl)-ethyl]-(2-phenylquinazolino-4-yl)-amine

¹H NMR (CDCl₃, 300 MHz) δ 8.59 (t, J=3.2 Hz, 2H), 7.91 (d, J=8.4 Hz, 1H), 7.67 (t, J=7.6 Hz, 1H), 7.59 (d, J=8.1 Hz, 1H), 7.47 (m, 3H), 7.36 (t, J=7.5 Hz, 1H), 6.80 (m, 3H), 6.39 (s, 1H), 3.98 (m, 2H), 3.86 (s, 3H), 3.71 (s, 3H), 3.08 (t, J=6.5 Hz, 2H)
Compound No. 123: [2-(4-chlorophenyl)-ethyl]-(2-phenylquinazolino-4-yl)-amine

¹H NMR (CDCl₃, 300 MHz) δ 8.57 (t, J=3.6 Hz, 2H), 7.91 (m, 1H), 7.69 (m, 1H), 7.53 (m, 4H), 7.38 (d, J=6.9 Hz, 1H), 7.25 (m, 2H), 7.18 (m, 2H), 5.76 (s, 1H), 4.00 (m, 2H), 3.05 (m, 2H)
Compound No. 124: [2-(4-Methoxyphenyl)-ethyl]-(2-phenylquinazolino-4-yl)-amine

¹H NMR (CDCl₃, 300 MHz) δ 8.60 (t, J=3.2 Hz, 2H), 7.93 (d, J=8.3 Hz, 1H), 7.69 (t, J=7.6 Hz, 1H), 7.50 (m, 4H), 7.35 (t, J=7.5 Hz, 1H), 7.20 (d, J=8.3 Hz, 2H), 6.87 (d, J=8.3 Hz, 2H), 5.85 (s, 1H), 4.00 (m, 2H), 3.79 (s, 3H), 3.02 (t, J=6.9 Hz, 2H)
Compound No. 125: (1,3-Dimethylbutyl)-(2-phenylquinazolino-4-yl)-amine

¹H NMR (CDCl₃, 300 MHz) δ 8.58 (t, J=3.8 Hz, 2H), 7.93 (d, J=8.5 Hz, 1H), 7.69 (m, 2H), 7.48-7.24 (m, 4H), 5.52 (d, J=6.9 Hz, 1H), 4.80 (t, J=6.86 Hz, 1H), 1.78-1.63 (m, 2H), 1.46 (t, J=6.7 Hz, 1H), 1.39 (m, 3H), 0.97 (m, 6H)
Compound No. 126: [2-(3-Chlorophenyl)-ethyl]-(2-phenylquinazolino-4-yl)-amine

¹H NMR (CDCl₃, 300 MHz) δ 8.59 (t, J=3.3 Hz, 2H), 7.93 (d, J=8.3 Hz, 1H), 7.69 (m, 1H), 7.58 (m, 1H), 7.49 (m, 3H), 7.38 (m, 1H), 7.24 (m, 1H), 7.12 (m, 2H), 7.11 (m, 1H), 5.92 (s, 1H), 3.95 (m, 2H), 3.02 (t, J=7.1 Hz, 2H)
Compound No. 127: [2-(2,4-Dichlorophenyl)-ethyl]-(2-phenylquinazolino-4-yl)-amine

¹H NMR (CDCl₃, 300 MHz) δ 8.57 (t, J=3.6 Hz, 2H), 7.92 (d, J=8.2 Hz, 1H), 7.70 (m, 1H), 7.59 (d, J=8.1 Hz, 1H), 7.47 (m, 2H), 7.37 (m, 2H), 7.18 (m, 2H), 5.81 (s, 1H), 4.04 (m, 2H), 3.23 (, J=6.8 Hz, 2H)
Compound No. 128: [2-(3,4-Difluorophenyl)-ethyl]-(2-phenylquinazolino-4-yl)-amine

¹H NMR (CDCl₃, 300 MHz) δ 8.50 (t, J=4.0 Hz, 2H), 7.93 (d, J=8.6 Hz, 1H), 7.72 (m, 2H), 7.41 (m, 4H), 7.22 (t, J=8.8 Hz, 1H), 7.07 (m, 2H), 6.11 (s, 1H), 4.91 (d, J=5.5 Hz, 2H)
Compound No. 129: (2-Phenylquinazolino-4-yl)-(3,4,5-trimethoxybenzyl)-amine

¹H NMR (CDCl₃, 300 MHz) δ 8.58 (t, J=3.5 Hz, 2H), 7.96 (d, J=8.4 Hz, 2H), 7.78 (d, J=8.3 Hz, 1H), 7.67 (t, J=7.6 Hz, 1H), 7.47 (m, 3H), 7.36 (t, J=7.6 Hz, 1H), 6.73 (s, 2H), 6.43 (s, 1H), 4.91 (d, J=5.2 Hz, 2H), 3.86 (d, J=6.6 Hz, 2H), 3.82 (m, 9H)
Compound No. 130: (3-Methylbutyl)-(2-phenylquinazolino-4-yl)-amine

¹H NMR (CDCl₃, 300 MHz) δ 8.57 (t, J=3.4 Hz, 2H), 7.92 (d, J=8.3 Hz, 1H), 7.68 (m, 2H), 7.46 (m, 3H), 7.37 (t, J=7.5 Hz, 1H), 5.76 (s, 1H), 3.80 (m, 2H), 1.76 (m, 1H), 1.65 (m, 2H), 0.99 (m, 6H)
Compound No. 131: (2-Cyclohex-1-enylethyl)-(2-phenylquinazolino-4-yl)-amine

¹H NMR (CDCl₃, 300 MHz) δ 8.57 (t, J=3.4 Hz, 2H), 7.92 (d, J=8.3 Hz, 1H), 7.69 (t, J=7.7 Hz, 1H), 7.61 (d, J=8.1 Hz, 1H), 7.49 (m, 3H), 7.38 (t, J=7.4 Hz, 1H), 5.82 (s, 1H), 5.61 (1H), 3.83 (m, 2H), 2.39 (t, J=6.6 Hz, 2H), 2.03 (m, 4H), 1.62 (m, 4H)
Compound No. 132: Cyclohexylmethyl-(2-phenylquinazolino-4-yl)-amine

¹H NMR (CDCl₃, 300 MHz) δ 8.56 (t, J=3.1 Hz, 2H), 7.92 (d, J=8.7 Hz, 1H), 7.69 (t, J=6.1 Hz, 2H), 7.49 (m, 4H), 5.85 (s, 1H), 3.64 (t, J=6.1 Hz, 2H), 1.88-1.70 (m, 6H), 1.29-1.05 (m, 5H)
Compound No. 133: (2,2-Diphenylethyl)-(2-phenylquinazolino-4-yl)-amine

¹H NMR (CDCl₃, 300 MHz) δ 8.63 (t, J=3.8 Hz, 2H), 7.93 (d, J=8.4 Hz, 1H), 7.67 (t, J=7.5 Hz, 1H), 7.51 (m, 3H), 7.29 (m, 12H), 5.73 (s, 1H), 4.56 (t, J=7.7 Hz, 1H), 4.42 (t, J=6.5 Hz, 2H)
Compound No. 134: (2,4-Difluorobenzyl)-(2-phenylquinazolino-4-yl)-amine

¹H NMR (CDCl₃, 300 MHz) δ 8.55 (t, J=3.9 Hz, 2H), 7.93 (d, J=8.4 Hz, 1H), 7.71 (m, 2H), 7.49 (m, 5H), 6.80 (m, 2H), 6.24 (s, 1H), 5.00 (d, J=5.6 Hz, 2H)
Compound No. 135: (2,5-Difluorobenzyl)-(2-phenylquinazolino-4-yl)-amine

¹H NMR (CDCl₃, 300 MHz) δ 8.52 (t, J=3.6 Hz, 2H), 7.93 (d, J=8.4 Hz, 1H), 7.72 (t, J=7.9 Hz, 2H), 7.41 (m, 4H), 7.18 (m, 1H), 7.00 (m, 1H), 6.92 (t, J=3.9 Hz, 1H), 6.20 (s, 1H), 5.00 (d, J=5.6 Hz, 2H)
Compound No. 136: (3,3-Diphenylpropyl)-(2-phenylquinazolino-4-yl)-amine

¹H NMR (CDCl₃, 300 MHz) δ 8.51 (m, 2H), 7.92 (d, J=8.4 Hz, 1H), 7.67 (m, 1H), 7.43 (m, 3H), 7.34-7.20 (m, 12H), 5.73 (s, 1H), 4.09 (t, J=7.7 Hz, 1H), 2.53 (m, 2H)
Compound No. 137: [2-(4-Bromophenyl)-ethyl]-(2-phenylquinazolino-4-yl)-amine

¹H NMR (CDCl₃, 300 MHz) δ 8.57 (t, 2.8 Hz, 2H), 7.92 (d, J=8.4 Hz, 1H), 7.71 (m, 1H), 7.50 (m, 7H), 7.20 (m, 2H), 5.75 (s, 1H), 3.98 (m, 2H), 3.04 (t, J=6.7 Hz, 2H)
Compound No. 138: Butylmethyl-(2-phenylquinazolino-4-yl)-amine

¹H NMR (CDCl₃, 300 MHz) δ 8.55 (dd, J₁=7.5 Hz, J₂=1.5 Hz, 2H), 7.99-7.91 (m, 2H), 7.67 (m, 1H), 7.46 (m, 3H), 7.34 (m, 1H), 3.80 (t, J=7.8 Hz, 2H), 3.41 (s, 3H), 1.86 (m, 2H), 1.43 (m, 2H), 1.00 (t, 3H, J=7.5 Hz)
Compound No. 139: (2-Phenylquinazolino-4-yl)-(4-trifluoromethylbenzyl)-amine

¹H NMR (CDCl₃, 300 MHz) δ 8.50 (m, 2H), 7.96 (d, J=8.1 Hz, 1H), 7.75 (m, 2H), 7.59 (m, 4H), 7.46 (m, 4H), 6.06 (br, 1H), 5.07 (d, J=6.0 Hz, 2H)
Compound No. 140: [2-(2-Fluorophenyl)-ethyl]-(2-phenylquinazolino-4-yl)-amine

¹H NMR (CDCl₃, 300 MHz) δ 8.60 (t, J=3.5 Hz, 2H), 7.93 (d, J=8.3 Hz, 1H), 7.69 (, J=7.6 Hzt, 1H), 7.60 (d, J=8.1 Hz, 1H), 7.49 (m, 3H), 7.36 (t, J=7.5 Hz, 1H), 7.24 (t, J=7.2 Hz, 2H), 7.06 (t, J=9.0 Hz, 2H), 4.00 (m, 2H), 3.14 (t, J=6.7 Hz, 2H)
Compound No. 141: (2-Phenylquinazolino-4-yl)-pyridino-4-ylmethylamine

¹H NMR (CDCl₃, 300 MHz) δ 8.51 (m, 2H), 8.42 (t, J=3.4 Hz, 2H), 8.00 (d, J=8.4 Hz, 1H), 7.89 (d, J=8.1 Hz, 1H), 7.71 (t, J=7.6 Hz, 1H), 7.43 (m, 4H), 7.31 (m, 2H), 4.99 (d, J=5.5 Hz, 2H)
Compound No. 142: (2-Phenylquinazolino-4-yl)-pyridino-2-ylmethylamine

¹H NMR (CDCl₃, 300 MHz) δ 8.60 (m, 2H), 7.91 (m, 2H), 7.69 (m, 3H), 7.43 (m, 5H), 7.25 (m, 1H), 5.00 (d, J=4.3 Hz, 2H)
Compound No. 143: (2,3-Dimethylcyclohexyl)-(2-phenylquinazolino-4-yl)-amine

¹H NMR (CDCl₃, 300 MHz) δ 8.53 (t, J=3.5 Hz, 2H), 7.92 (d, J=8.3 Hz, 1H), 7.69 (m, 2H), 7.41 (m, 4H), 5.76 (m, 1H), 4.56 (m, 1H), 1.94-1.46 (m, 7H), 1.00 (m, 5H), 0.86 (d, J=7.1 Hz, 1H)
Compound No. 144: (2,4-Dimethoxybenzyl)-(2-phenylquinazolino-4-yl)-amine

¹H NMR (CDCl₃, 300 MHz) δ 8.60 (m, 2H), 7.90 (d, J=8.4 Hz, 1H), 7.72-7.63 (m, 2H), 7.48 (m, 3H), 7.38 (m, 2H), 6.51 (d, 1H, J=2.1 Hz), 6.45 (dd, J₁=8.1 Hz, J₂=2.4 Hz, 1H), 6.16 (br, 1H), 4.94 (d, J=5.4 Hz, 2H), 3.90 (s, 3H), 3.79 (s, 3H)
Compound No. 145: (3-Chlorobenzyl)-(2-phenylquinazolino-4-yl)-amine

¹H NMR (CDCl₃, 300 MHz) δ 8.51 (m, 2H), 7.92 (d, J=8.8 Hz, 1H), 7.71 (m, 2H), 7.45 (m, 5H), 7.27 (m, 3H), 5.13 (s, 1H), 4.92 (d, J=5.5 Hz, 2H)
Compound No. 146: 1-Phenyl-2-(2-phenylquinazolino-4-ylamino)-ethanol

¹H NMR (DMSO-d₆, 300 MHz) δ 8.49 (m, 2H), 8.25 (dH, J=8.3 Hz, 1), 7.74 (m, 2H), 7.50-7.31 (m, 7H), 7.23 (t, J=7.1 Hz, 1H), 5.62 (d, J=4.3 Hz, 1H), 5.05 (m, 1H), 4.01-3.90 (m, 2H), 3.57 (m, 1H), 3.28 (m, 2H)
Compound No. 147: (2,5-Dimethoxybenzyl)-(2-phenylquinazolino-4-yl)-amine

¹H NMR (CDCl₃, 300 MHz) δ 8.60 (dd, J₁=8.1 Hz, J₂=2.1 Hz, 2H), 7.91 (d, J=8.4 Hz, 1H), 7.67 (m, 2H), 7.52-7.36 (m, 4H), 7.10 (d, J=3.0 Hz, 1H), 6.85 (d, J=9.0 Hz, 1H), 6.78 (dd, J₁=9.0 Hz, J₂=3.0 Hz, 1H), 6.272 (br, 1H), 4.99 (d, J=6.0 Hz, 2H), 3.89 (s, 3H), 3.71 (s, 3H)
Compound No. 148: (4-Bromobenzyl)-(2-phenylquinazolino-4-yl)-amine

¹H NMR (CDCl₃, 300 MHz) δ 8.54 (t, J=3.6 Hz, 2H), 7.92 (d, J=8.3 Hz, 1H), 7.71 (t, J=6.6 Hz, 1H), 7.44 (m, 6H), 7.26 (t, J=7.0 Hz, 2H), 6.09 (s, 1H), 4.91 (d, J=5.5 Hz, 2H)
Compound No. 149: [2-(3,4-Dichlorophenyl)-ethyl]-(2-phenylquinazolino-4-yl)-amine

¹H NMR (CDCl₃, 300 MHz) δ 8.57 (m, 2H), 7.94 (d, J=8.4 Hz, 1H), 7.74 (m, 1H), 7.59 (d, J=7.8 Hz, 1H), 7.55-7.37 (m, 6H), 7.10 (dd, J₁=8.4 Hz, J₂=1.8 Hz, 1H), 5.75 (br, 1H), 4.03 (q, J=7.2 Hz, 2H), 3.08 (t, J=7.2 Hz, 2H)
Compound No. 150: 1-[3-(2-Phenylquinazolino-4-ylamino)-propyl]-pyrrolidino-2-one

¹H NMR (CDCl₃, 300 MHz) δ 8.55 (m, 2H), 8.00 (d, J=8.1 Hz, 1H), 7.89 (d, J=8.6 Hz, 1H), 7.69 (d, J=7.3 Hz, 1H), 7.59 (s, 1H), 7.44 (m, 4H), 3.76 (m, 2H), 3.41 (m, 4H), 2.45 (t, J=8.1 Hz, 2H), 2.06 (m, 2H), 1.87 (m, 2H)
Compound No. 151: [2-(2-Chlorophenyl)-ethyl]-(2-phenylquinazolino-4-yl)-amine

¹H NMR (CDCl₃, 300 MHz) δ 8.59 (m, 2H), 7.92 (d, J=8.4 Hz, 1H), 7.71 (d, J=7.3 Hz, 1H), 7.63 (d, J=8.1 Hz, 1H), 7.48 (m, 3H), 7.37 (t, J=7.2 Hz, 2H), 7.25 (t, J=3.5 Hz, 1H), 7.17 (t, J=4.5 Hz, 2H), 5.99 (s, 1H), 4.05 (m, 2H), 3.24 (t, J=6.9 Hz, 2H)
Compound No. 152: 2-[2-(2-Phenylquinazolino-4-ylamino)-ethyl]-phenol

¹H NMR (DMSO-d₆, 300 MHz) δ 9.18 (s, 1H), 8.46 (m, 3H), 8.19 (d, J=8.1 Hz, 1H), 7.73 (m, 2H), 7.47 (m, 4H), 7.07 (d, J=8.2 Hz, 2H), 6.66 (d, J=8.2 Hz, 2H), 4.12 (s, 1H), 3.77 (m, 2H), 2.91 (t, J=7.4 Hz, 2H)
Compound No. 153: (3-Bromobenzyl)-(2-phenylquinazolino-4-yl)-amine

¹H NMR (CDCl₃, 300 MHz) δ 8.52 (m, 2H), 7.92 (d, J=8.5 Hz, 1H), 7.71 (t, J=3.1 Hz, 2H), 7.56 (s, 1H), 7.45 (m, 6H), 7.14 (m, 1H), 6.13 (s, 1H), 4.91 (d, J=5.3 Hz, 2H)
Compound No. 154: (2,6-Dimethoxybenzyl)-(2-phenylquinazolino-4-yl)-amine

¹H NMR (CDCl₃, 300 MHz) δ 8.70 (m, 2H), 7.92 (d, J=8.4 Hz, 1H), 7.84 (m, 2H), 7.51 (m, 3H), 7.46 (m, 1H), 7.23 (m, 1H), 6.58 (d, J=8.4 Hz, 2H), 6.38 (s, 1H), 5.13 (d, J=5.3 Hz, 2H), 3.88 (s, 6H)
Compound No. 155: (2-Bromobenzyl)-(2-phenylquinazolino-4-yl)-amine

¹H NMR (CDCl₃, 300 MHz) δ 8.56 (t, J=3.8 Hz, 2H), 7.92 (d, J=8.4 Hz, 1H), 7.72 (m, 2H), 7.50 (m, 5H), 7.46 (m, 1H), 7.24 (m, 1H), 6.34 (s, 1H), 5.06 (d, J=5.8 Hz, 2H)
Compound No. 156: (3-Morpholino-4-ylpropyl)-(2-phenylquinazolino-4-yl)-amine

¹H NMR (CDCl₃, 300 MHz) δ 8.55 (m, 2H), 7.89 (d, J=8.3 Hz, 1H), 7.78 (d, J=8.1 Hz, 1H), 7.68 (m, 3H), 7.45 (m, 3H), 3.81 (m, 6H), 2.57 (m, 7H), 1.89 (m, 2H)
Compound No. 157: 2-[Butyl-(2-phenylquinazolino-4-yl)-amino]-ethanol

¹H NMR (CDCl₃, 300 MHz) δ 8.53 (m, 2H), 8.18-7.91 (m, 2H), 7.69 (m, 1H), 7.45 (m, 4H), 4.85 (t, J=5.2 Hz, 1H), 4.01 (s, 2H), 3.67 (t, J=8.0 Hz, 1H), 3.21 (t, J=5.2 Hz, 1H), 2.74 (t, J=7.3 Hz, 1H), 1.95 (m, 1H), 1.60 (m, 1H), 1.03-0.87 (m, 3H)
Compound No. 158: [2-(4-Methylpiperazino-1-yl)-ethyl]-(2-phenylquinazolino-4-yl)-amine

¹H NMR (CDCl₃, 300 MHz) δ 8.51 (t, J=3.9 Hz, 2H), 8.03 (s, 1H), 7.92 (d, J=8.3 Hz, 1H), 7.84 (d, J=8.8 Hz, 1H), 7.65 (t, J=7.6 Hz, 1H), 7.39 (m, 4H), 5.84 (br s, 1H), 3.83 (m, 2H), 2.98 (m, 4H), 2.66 (m, 10H), 2.33 (s, 3H), 1.05 (m, 2H), 1.29 (m, 6H)
Compound No. 159: Cyclopentylmethyl-(2-phenylquinazolino-4-yl)-amine

¹H NMR (CDCl₃, 300 MHz) δ 8.84 (m, 2H), 7.98 (t, J=8.1 Hz, 2H), 7.68 (t, J=7.7 Hz, 1H), 7.47 (m, 3H), 7.35 (t, J=7.6 Hz, 1H), 4.99 (t, J=7.7 Hz, 1H), 3.26 (s, 3H), 2.08 (m, 2H), 1.71 (m, 6H)
Compound No. 160: (2-Phenylquinazolino-4-yl)-(2-piperazino-1-ylethyl)-amine

¹H NMR (CDCl₃, 300 MHz) δ 8.54 (m, 2H), 7.97 (m, 2H), 7.74 (m, 1H), 7.46 (m, 4H), 4.08 (m, 4H), 3.06 (m, 4H)
Compound No. 161: O-(4-Nitrobenzyl)-N-(2-phenylquinazolino-4-yl)-hydroxylamine

¹H NMR (CDCl₃, 300 MHz) δ 8.21 (m, 3H), 7.97 (m, 2H), 7.88 (d, J=7.9 Hz, 1H), 7.57 (m, 8H), 7.28 (m, 1H)
Compound No. 162: (4-Chlorophenyl)-(2-phenylquinazolino-4-yl)-amine

¹H NMR (DMSO-d₆, 300 MHz) δ 9.92 (s, 1H), 8.53 (d, J=8.3 Hz, 1H), 8.41 (t, J=3.9 Hz, 2H), 7.98 (d, J=8.8 Hz, 2H), 7.85 (d, J=3.8 Hz, 2H), 7.62-7.48 (m, 6H)
Compound No. 163: Benzyl-(2-methylquinazolino-4-yl)-amine

¹H NMR (DMSO-d₆, 300 MHz) δ 9.69 (s, 1H), 8.47 (d, J=8.1 Hz, 1H), 8.20 (t, J=7.2 Hz, 2H), 7.81 (d, J=3.6 Hz, 2H), 7.55 (t, J=4.1 Hz, 1H), 7.46-7.23 (m, 7H), 2.22 (s, 3H)
Compound No. 164: (3-Methoxybenzyl)-(2-methylquinazolino-4-yl)-amine

¹H NMR (CDCl₃, 300 MHz) δ 7.77-7.63 (m, 3H), 7.35 (t, J=7.6 Hz, 1H), 7.24 (t, J=6.3 Hz, 1H), 6.96 (m, 2H), 6.82 (m, 1H), 6.32 (s, 1H), 4.84 (m, 2H), 3.74 (s, 3H), 2.62 (s, 3H)
Compound No. 165: (4-Methoxybenzyl)-(2-methylquinazolino-4-yl)-amine

¹H NMR (CDCl₃, 300 MHz) δ 7.74 (d, J=8.2 Hz, 1H), 7.67 (m, 2H), 7.35 (m, 3H), 6.86 (d, J=8.6 Hz, 2H), 6.12 (s, 1H), 4.77 (d, J=4.0 Hz, 2H), 3.78 (s, 3H), 2.64 (s, 3H)
Compound No. 166: (2-Methylquinazolino-4-yl)-(3-trifluoromethylbenzyl)-amine

¹H NMR (CDCl₃, 300 MHz) δ 7.82 (m, 2H), 7.64 (m, 2H), 7.52 (m, 1H), 7.35 (m, 2H), 6.73 (s, 1H), 4.93 (s, 2H), 2.63 (s, 3H)
Compound No. 167: (2-Methylquinazolino-4-yl)-phenethylamine

¹H NMR (CDCl₃, 300 MHz) δ 7.77 (d, J=8.3 Hz, 1H), 7.60 (m, 2H), 7.33 (m, 3H), 7.24 (m, 3H), 6.12 (br, 1H), 3.94 (m, 2H), 3.01 (t, J=6.9 Hz, 2H), 2.65 (s, 3H)
Compound No. 168: (2-Methylquinazolino-4-yl)-(4-phenylbutyl)-amine

¹H NMR (CDCl₃, 300 MHz) δ 7.70 (m, 3H), 7.36 (t, J=7.5 Hz, 1H), 7.26 (m, 2H), 7.17 (m, 3H), 6.12 (br, 1H), 3.69 (d, J=5.3 Hz, 2H), 2.68 (t, J=6.8 Hz, 2H), 2.62 (s, 3H), 1.75 (t, J=3.4 Hz, 4H)
Compound No. 169: (2-Methylquinazolino-4-yl)-naphthaleno-1-ylmethylamine

¹H NMR (CDCl₃, 300 MHz) δ 8.07 (m, 1H), 7.86 (m, 3H), 7.64 (t, J=7.4 Hz, 2H), 7.55-7.42 (m, 4H), 7.24 (m, 2H), 6.22 (br s, 1H), 5.27 (s, 2H), 2.70 (s, 3H)
Compound No. 170: Benzo[1,3]dioxol-5-ylmethyl-(2-methylquinazolino-4-yl)-amine

¹H NMR (CDCl₃, 300 MHz) δ 7.74 (m, 3H), 7.35 (t, J=7.5 Hz, 1H), 6.84 (t, J=6.5 Hz, 2H), 6.74 (d, J=7.8 Hz, 1H), 6.24 (br, 1H), 5.92 (s, 2H), 4.76 (d, J=4.1 Hz, 2H), 2.65 (s, 3H)
Compound No. 171: (3-Fluorobenzyl)-(2-methylquinazolino-4-yl)-amine

¹H NMR (CDCl₃, 300 MHz) δ 7.79 (m, 2H), 7.66 (t, J=7.7 Hz, 1H), 7.37 (t, J=7.5 Hz, 1H), 7.26 (t, J=3.9 Hz, 1H), 7.14 (m, 2H), 6.96 (m, 1H), 6.51 (s, 1H), 4.87 (d, J=3.3 Hz, 2H), 2.63 (s, 3H)
Compound No. 172: [2-(3-Fluorophenyl)-ethyl]-(2-methylquinazolino-4-yl)-amine

¹H NMR (CDCl₃, 300 MHz) δ 7.78 (d, J=8.5 Hz, 1H), 7.65 (t, J=7.3 Hz, 2H), 7.35 (t, J=7.8 Hz, 1H), 7.27 (m, 1H), 6.94 (m, 3H), 6.24 (br, 1H), 3.93 (m, 2H), 3.02 (t, J=6.99 Hz, 2H), 2.65 (s, 3H)
Compound No. 173: 2-[2-(2-Methylquinazolino-4-ylamino)-ethoxy]-ethanol

¹H NMR (CDCl₃, 300 MHz) δ 7.83 (d, J=8.1 Hz, 1H), 7.72 (d, J=10.7 Hz, 1H), 7.61 (t, J=7.1 Hz, 1H), 7.30 (t, J=6.5 Hz, 1H), 6.96 (br, 1H), 3.82 (m, 6H), 3.68 (m, 2H), 2.60 (s, 3H)
Compound No. 174: 2-(2-Methylquinazolino-4-ylamino)-ethanol

¹H NMR (CDCl₃, 300 MHz) δ 8.27 (d, J=8.2 Hz, 1H), 7.88 (t, J=7.2 Hz, 1H), 7.79 (t, J=7.5 Hz, 1H), 7.51 (m, 1H), 3.93 (m, 4H), 2.70 (s, 3H)
Compound No. 175: Benzyl-(2-isopropylquinazolino-4-yl)-amine

¹H NMR (CDCl₃, 300 MHz) δ 7.82 (d, J=7.8 Hz, 1H), 7.71-7.63 (m, 2H), 7.45-7.26 (m, 6H), 5.88 (br, 1H), 4.90 (d, J=5.4 Hz, 2H), 3.12 (m, 1H), 1.37 (d, J=6.6 Hz, 6H)
Compound No. 176: (2-Isopropylquinazolino-4-yl)-(3-methoxybenzyl)-amine

¹H NMR (CDCl₃, 300 MHz) δ 7.82 (d, J=8.4 Hz, 1H), 7.75 (m, 1H), 7.66 (m, 1H), 7.38 (t, J=7.9 Hz, 1H), 7.25 (t, J=7.7 Hz, 1H), 7.00 (m, 2H), 6.80 (m, 1H), 4.88 (d, J=5.0 Hz, 2H), 3.77 (s, 3H), 3.13 (m, 1H), 1.37 (d, J=6.8 Hz, 6H)
Compound No. 177: (2-Isopropylquinazolino-4-yl)-(4-methoxybenzyl)-amine

¹H NMR (CDCl₃, 300 MHz) δ 7.82 (d, J=8.1 Hz, 1H), 7.70-7.61 (m, 2H), 7.35 (m, 3H), 6.89 (d, J=8.7 Hz, 2H), 5.86 (br, 1H), 4.83 (d, J=5.4 Hz, 2H), 3.81 (s, 3H), 3.14 (m, 1H), 1.38 (d, J=6.6 Hz, 6H)
Compound No. 178: (2-Isopropylquinazolino-4-yl)-(3-trifluoromethylbenzyl)-amine

¹H NMR (CDCl₃, 300 MHz) δ 7.87 (m, 2H), 7.66 (m, 3H), 7.51 (d, J=7.8 Hz, 1H), 7.39 (m, 2H), 4.96 (d, J=5.0 Hz, 2H), 3.17 (m, 1H), 1.33 (d, J=6.8 Hz, 6H)
Compound No. 179: (2-Isopropylquinazolino-4-yl)-phenethylamine

¹H NMR (CDCl₃, 300 MHz) δ 7.82 (d, J=8.1 Hz, 1H), 7.67 (m, 1H), 7.55 (d, J=7.8 Hz, 1H), 7.38-7.21 (m, 6H), 5.84 (br, 1H), 3.95 (q, J=6.9 Hz, 2H), 3.16 (m, 1H), 3.05 (t, J=6.9 Hz, 2H), 1.40 (d, J=6.9 Hz, 6H)
Compound No. 180: (2-Isopropylquinazolino-4-yl)-(4-phenylbutyl)-amine

¹H NMR (CDCl₃, 300 MHz) δ 7.83 (d, J=7.9 Hz, 1H), 7.75 (d, J=7.7 Hz, 1H), 7.65 (t, J=7.6 Hz, 1H), 7.35 (t, J=7.6 Hz, 1H), 7.25 (m, 6H), 3.73 (d, J=5.5 Hz, 2H), 3.14 (m, 1H), 2.68 (d, J=6.5 Hz, 2H), 1.77 (m, 4H), 1.36 (d, J=6.8 Hz, 6H)
Compound No. 181: (2-Isopropylquinazolino-4-yl)-naphthaleno-1-ylmethylamine

¹H NMR (CDCl₃, 300 MHz) δ 8.18 (m, 1H), 7.93-7.81 (m, 3H), 7.68-7.43 (m, 6H), 7.30 (m, 1H), 5.85 (br, 1H), 5.35 (d, J=5.1 Hz, 2H), 3.20 (m, 1H), 1.44 (d, J=6.6 Hz, 6H)
Compound No. 182: Benzo[1,3]dioxol-5-ylmethyl-(2-isopropylquinazolino-4-yl)-amine

¹H NMR (CDCl₃, 300 MHz) δ 7.81 (d, J=7.8 Hz, 1H), 7.70-7.63 (m, 2H), 7.66 (m, 1H), 6.94-6.87 (m, 2H), 6.78 (d, 1H, J=7.8 Hz), 5.94 (s, 2H), 5.90 (br, 1H), 4.80 (d, J=5.4 Hz, 2H), 3.13 (m, 1H), 1.38 (d, J=6.6 Hz, 6H)
Compound No. 183: (3-Fluorobenzyl)-(2-isopropylquinazolino-4-yl)-amine

¹H NMR (CDCl₃, 300 MHz) δ 7.82 (t, J=5.6 Hz, 1H), 7.67 (t, J=7.7 Hz, 1H), 7.37 (t, J=7.5 Hz, 1H), 7.28-7.12 (m, 3H), 6.95 (t, J=7.3 Hz, 1H), 6.34 (br, 1H), 4.89 (d, J=3.5 Hz, 2H), 3.14 (m, 1H), 1.33 (d, J=6.8 Hz, 6H)
Compound No. 184: [2-(3-Fluorophenyl)-ethyl]-(2-isopropylquinazolino-4-yl)-amine

¹H NMR (CDCl₃, 300 MHz) δ 7.83 (d, J=8.7 Hz, 1H), 7.66 (t, J=7.2 Hz, 2H), 7.38-7.23 (m, 2H), 6.89 (m, 3H), 6.32 (br, 1H), 3.94 (m, 1H), 3.17 (t, J=6.8 Hz, 1H), 3.06 (m, 1H), 1.39 (d, J=6.8 Hz, 6H)
Compound No. 185: 2-[2-(2-Isopropylquinazolino-4-ylamino)-ethoxy]-ethanol

¹H NMR (MeOD, 300 MHz) δ 7.78 (t, J=8.1 Hz, 2H), 7.61 (t, J=7.6 Hz, 1H), 7.29 (m, 1H), 6.78 (br, 1H), 3.89 (d, J=4.4 Hz, 2H), 3.80 (m, 4H), 3.66 (t, J=4.4 Hz, 2H), 3.11 (m, 1H), 1.33 (d, J=6.8 Hz, 6H)
Compound No. 186: 2-(2-Isopropylquinazolino-4-ylamino)-ethanol

¹H NMR (CDCl₃+MeOD, 300 MHz) δ 7.85 (d, J=8.2 Hz, 1H), 7.64 (m, 2H), 7.31 (m, 1H), 3.78 (m, 4H), 3.02 (m, 1H), 1.27 (d, J=6.8 Hz, 6H)
Compound No. 187: Benzyl-(2-tert-butylquinazolino-4-yl)-amine

¹H NMR (CDCl₃, 300 MHz) δ 7.90 (d, J=8.2 Hz, 1H), 7.12 (d, J=5.3 Hz, 1H), 7.53 (t, J=7.6 Hz, 1H), 7.36-7.25 (m, 6H), 6.22 (br, 1H), 4.90 (d, J=5.3 Hz, 2H), 1.44 (s, 9H)
Compound No. 188: (2-tert-Butylquinazolino-4-yl)-(3-methoxybenzyl)-amine

¹H NMR (CDCl₃, 300 MHz) δ 7.88 (d, J=8.1 Hz, 1H), 7.66 (m, 2H), 7.44-7.23 (m, 3H, 6.99 (d, J=8.0 Hz, 2H), 6.84 (t, J=4.2 Hz, 1H), 6.16 (br, 1H), 4.87 (d, J=5.3 Hz, 2H), 3.78 (s, 3H), 1.44 (s, 9H)
Compound No. 189: (2-tert-Butylquinazolino-4-yl)-(4-methoxybenzyl)-amine

¹H NMR (CDCl₃, 300 MHz) δ 7.86 (m, 1H), 7.64 (m, 2H), 7.32 (m, 3H), 6.99 (d, J=8.7 Hz, 1H), 6.86 (d, J=5.6 Hz, 2H), 6.19 (br, 1H), 4.82 (d, J=5.4 Hz, 2H), 3.80 (s, 3H), 1.44 (s, 9H)
Compound No. 190: (2-tert-Butylquinazolino-4-yl)-(3-trifluoromethylbenzyl)-amine

¹H NMR (CDCl₃, 300 MHz) δ 7.91 (d, J=8.3 Hz, 1H), 7.79-7.61 (m, 4H), 7.53 (d, J=7.9 Hz, 1H), 7.46-7.34 (m, 2H), 6.21 (br, 1H), 4.95 (d, J=5.3 Hz, 2H), 1.45 (s, 9H)
Compound No. 191: (2-tert-Butylquinazolino-4-yl)-phenethylamine

¹H NMR (CDCl₃, 300 MHz) δ 7.85 (d, J=8.4 Hz, 1H), 7.66 (t, J=7.5 Hz, 1H), 7.53 (d, J=7.8 Hz, 1H), 7.31 (m, 6H), 5.72 (br s, 1H), 3.93 (q, J=6.9 Hz, 2H), 3.05 (t, J=7.2 Hz, 2H), 1.48 (s, 9H)
Compound No. 192: (2-tert-Butylquinazolino-4-yl)-(4-phenylbutyl)-amine

¹H NMR (CDCl₃, 300 MHz) δ 7.87 (d, J=8.3 Hz, 1H), 7.65 (m, 2H), 7.37-7.25 (m, 3H), 7.19 (m, 3H), 5.98 (br, 1H), 3.71 (d, J=5.66 Hz, 2H), 2.68 (d, J=6.5 Hz, 2H), 1.44 (m, 4H), 1.44 (s, 9H)
Compound No. 193: (2-tert-Butylquinazolino-4-yl)-naphthaleno-1-ylmethylamine

¹H NMR (CDCl₃, 300 MHz) δ 8.20 (d, J=9.4 Hz, 1H), 7.89 (m, 3H), 7.61 (m, 3H), 7.50 (m, 3H), 6.10 (br, 1H), 5.35 (d, J=5.1 Hz, 2H), 1.52 (s, 9H)
Compound No. 194: Benzo[1,3]dioxol-5-ylmethyl-(2-tert-butylquinazolino-4-yl)-amine

¹H NMR (CDCl₃, 300 MHz) δ 7.89 (d, J=8.3 Hz, 1H), 7.66 (m, 2H), 7.34 (t, J=7.5 Hz, 1H), 6.91 (m, 2H), 6.76 (d, J=7.9 Hz, 1H), 6.21 (br, 1H), 5.93 (s, 2H), 4.80 (d, J=6.2 Hz, 2H), 1.44 (s, 9H)
Compound No. 195: (2-tert-Butylquinazolino-4-yl)-(3-fluorobenzyl)-amine

¹H NMR (CDCl₃, 300 MHz) δ 7.89 (d, J=8.3 Hz, 1H), 7.69 (m, 2H), 7.35 (m, 1H), 7.28 (m, 1H), 7.14 (m, 2H), 6.94 (d, J=5.4 Hz, 1H), 6.24 (br, 1H), 4.99 (d, J=6.2 Hz, 2H), 1.45 (s, 9H)
Compound No. 196: (2-tert-Butylquinazolino-4-yl)-[2-(3-fluorophenyl)-ethyl]-amine

¹H NMR (CDCl₃, 300 MHz) δ 7.89 (d, J=8.3 Hz, 1H), 7.68 (m, 2H), 7.33 (m, 1H), 7.25 (m, 1H), 6.98 (m, 2H), 6.01 (br, 1H), 3.93 (m, 2H), 3.06 (t, J=2.6 Hz, 2H), 1.45 (s, 9H)
Compound No. 197: 2-[2-(2-tert-Butylquinazolino-4-ylamino)-ethoxy]-ethanol

¹H NMR (CDCl₃, 300 MHz) δ 7.85 (d, J=8.3 Hz, 1H), 7.71 (d, J=8.2 Hz, 1H), 7.63 (t, J=7.8 Hz, 1H), 7.33 (t, J=7.5 Hz, 1H), 6.33 (br, 1H), 3.90 (d, J=4.1 Hz, 2H), 3.80 (t, J=4.6 Hz, 4H), 3.65 (t, J=4.6 Hz, 2H), 1.44 (s, 9H)
Compound No. 198: 2-(2-tert-Butylquinazolino-4-ylamino)-ethanol

¹H NMR (CDCl₃, 300 MHz) δ 7.83 (d, J=8.0 Hz, 1H), 7.69 (t, J=7.6 Hz, 2H), 7.38 (t, J=7.6 Hz, 1H), 6.14 (br, 1H), 4.59 (s, 1H), 3.93-3.83 (m, 4H), 1.44 (s, 9H)
Compound No. 199: Benzyl-(2-cyclohexylquinazolino-4-yl)-amine

¹H NMR (CDCl₃, 300 MHz) δ 7.83 (d, J=8.0 Hz, 1H), 7.68 (m, 2H), 7.43 (d, J=7.5 Hz, 2H), 7.31 (m, 4H), 6.26 (br, 1H), 4.90 ((d, J=5.2 Hz, 2H), 2.82 (t, J=11.5 Hz, 1H), 1.97 (m, 2H), 1.73 (m, 5H), 1.33 (m, 3H)
Compound No. 200: (2-Cyclohexylquinazolino-4-yl)-(3-methoxybenzyl)-amine

¹H NMR (CDCl₃, 300 MHz) δ 7.84 (d, J=8.3 Hz, 1H), 7.73 (d, J=8.2 Hz, 1H), 7.65 (t, J=7.3 Hz, 1H), 7.34 (t, J=7.6 Hz, 1H), 7.26 (t, J=7.8 Hz, 1H), 7.01 (d, J=7.3 Hz, 2H), 6.84 (t, J=4.5 Hz, 1H), 6.43 (br, 1H), 4.88 (d, J=6.8 Hz, 2H), 3.78 (s, 3H), 2.84 (m, 1H), 2.02 (m, 2H), 1.85-1.66 (m, 5H), 1.48-1.28 (m, 3H)
Compound No. 201: (2-Cyclohexylquinazolino-4-yl)-(4-methoxybenzyl)-amine

¹H NMR (CDCl₃, 300 MHz) δ 7.84 (d, J=8.3 Hz, 1H), 7.73 (d, J=8.2 Hz, 1H), 7.64 (t, J=7.4 Hz, 1H), 7.36 (m, 3H), 6.87 (d, J=8.5 Hz, 2H), 6.48n (br, 1H), 4.83 (d, J=6.8 Hz, 2H), 3.80 (s, 3H), 2.85 (m, 1H), 2.02 (m, 2H), 2.04-1.69 (m, 5H), 1.49-1.25 (m, 3H)
Compound No. 202: (2-Cyclohexylquinazolino-4-yl)-(3-trifluoromethylbenzyl)-amine

¹H NMR (CDCl₃, 300 MHz) δ 7.83 (m, 2H), 7.78 (m, 1H), 7.64 (m, 2H), 7.53 (m, 1H), 7.40 (m, 2H), 6.67 (br, 1H), 4.93 (d, J=5.0 Hz, 2H), 2.80 (m, 1H), 1.90 (m, 2H), 1.84-1.60 (m, 5H), 1.45-1.25 (m, 3H)
Compound No. 203: (2-Cyclohexylquinazolino-4-yl)-phenethylamine

¹H NMR (CDCl₃, 300 MHz) δ 7.83 (d, J=8.3 Hz, 1H), 7.65 (m, 2H), 7.28 (m, 6H), 6.06 (br, 1H), 3.95 (m, 2H), 3.05 (t, J=7.0 Hz, 2H), 2.83 (m, 1H), 2.04 (m, 2H), 1.88-1.71 (m, 5H), 1.50-1.35 (m, 3H)
Compound No. 204: (2-Cyclohexylquinazolino-4-yl)-(4-phenylbutyl)-amine

¹H NMR (CDCl₃, 300 MHz) δ 7.83 (d, J=8.3 Hz, 1H), 7.69 (m, 2H), 7.37-7.17 (m, 7H), 6.12 (br, 1H), 3.72 (d, J=5.6 Hz, 2H), 2.78 (t, J=11.1 Hz, 1H), 2.70 (t, J=6.7 Hz, 2H), 2.00 (m, 2H), 1.86-1.68 (m, 9H), 1.44-1.28 (m, 3H)
Compound No. 205: (2-Cyclohexylquinazolino-4-yl)-naphthaleno-1-ylmethylamine

¹H NMR (CDCl₃, 300 MHz) δ 8.20 (d, J=9.4 Hz, 1H), 7.87 (m, 3H), 7.62 (m, 3H), 7.50 (m, 3H), 7.30 (m, 1H), 6.34 (br, 1H), 5.35 (d, J=4.9 Hz, 2H), 2.92 (m, 1H), 2.08 (m, 2H), 1.86-1.73 (m, 5H), 1.47-1.25 (m, 3H)
Compound No. 206: Benzo[1,3]dioxol-5-ylmethyl-(2-cyclohexylquinazolino-4-yl)-amine

¹H NMR (CDCl₃, 300 MHz) δ 7.83 (d, J=8.4 Hz, 1H), 7.73 (d, J=8.2 Hz, 1H), 7.65 (t, J=7.5 Hz, 2H), 6.91 (m, 2H), 6.76 (d, J=7.9 Hz, 1H), 6.34 (br, 1H), 5.93 (s, 2H), 4.80 (d, J=5.2 Hz, 2H), 2.84 (m, 1H), 2.00 (m, 2H), 1.86-1.68 (m, 5H), 1.48-1.25 (m, 3H)
Compound No. 207: (2-Cyclohexylquinazolino-4-yl)-(3-fluorobenzyl)-amine

¹H NMR (CDCl₃, 300 MHz) δ 7.80 (m, 1H), 7.66 (t, J=7.6 Hz, 1H), 7.35 (m, 1H), 7.27 (m, 2H), 7.16 (m, 2H), 6.95 (d, J=8.4 Hz, 1H), 6.47 (br, 1H), 4.90 (d, J=5.2 Hz, 2H), 2.83 (m, 1H), 1.99 (m, 2H), 1.84-1.63 (m, 5H), 1.46-1.25 (m, 3H)
Compound No. 208: (2-Cyclohexylquinazolino-4-yl)-[2-(3-fluorophenyl)-ethyl]-amine

¹H NMR (CDCl₃, 300 MHz) δ 7.83 (d, J=8.3 Hz, 1H), 7.65 (t, J=7.7 Hz, 2H), 7.30 (m, 2H), 6.99 (m, 3H), 6.15 (br, 1H), 3.94 (m, 2H), 3.05 (t, J=7.1 Hz, 2H), 2.83 (m, 1H), 2.00 (m, 2H), 1.88-1.69 (m, 5H), 1.46-1.25 (m, 3H)
Compound No. 209: 2-[2-(2-Cyclohexylquinazolino-4-ylamino)-ethoxy]-ethanol

¹H NMR (CDCl₃, 300 MHz) δ 7.75 (m, 2H), 7.62 (t, J=7.6 Hz, 1H), 7.31 (t, J=7.5 Hz, 1H), 6.48 (br, 1H), 3.90 (m, 2H), 3.80 (m, 4H), 3.36 (m, 2H), 2.78 (m, 1H), 1.90 (m, 2H), 1.85-1.62 (m, 5H), 1.47-1.27 (m, 3H)
Compound No. 210: 2-(2-Cyclohexylquinazolino-4-ylamino)-ethanol

¹H NMR (CDCl₃, 300 MHz) δ 7.80 (d, J=8.4 Hz, 1H), 7.69 (m, 2H), 7.40 (t, J=7.0 Hz, 1H), 6.20 (br, 1H), 5.08 (br, 1H), 3.93 (m, 2H), 3.85 (m, 2H), 2.77 (m, 1H), 1.97 (m, 2H), 1.86-1.66 (m, 5H), 1.47-1.25 (m, 3H)
Compound No. 211: Benzyl-(2-benzylquinazolino-4-yl)-amine

¹H NMR (CDCl₃, 300 MHz) δ 7.82 (d, J=8.4 Hz, 1H), 7.71 (m, 2H), 7.47 (m, 2H), 7.29 (m, 9H), 5.94 (br, 1H), 4.82 (d, J=5.5 Hz, 2H), 4.19 (s, 2H)
Compound No. 212: (2-Benzylquinazolino-4-yl)-(3-methoxybenzyl)-amine

¹H NMR (CDCl₃, 300 MHz) δ 7.82 (d, J=8.1 Hz, 1H), 7.69 (t, J=6.9 Hz, 1H), 7.60 (d, J=8.1 Hz, 1H), 7.45 (d, J=7.2 Hz, 2H), 7.37 (t, J=7.2 Hz, 1H), 7.25 (m, 4H), 6.85 (m, 3H), 5.87 (br, 1H), 4.80 (d, J=5.4 Hz, 2H), 4.19 (s, 2H), 3.76 (s, 3H)
Compound No. 213: (2-Benzylquinazolino-4-yl)-(4-methoxybenzyl)-amine

¹H NMR (CDCl₃, 300 MHz) δ 7.81 (d, J=8.4 Hz, 1H), 7.67 (t, J=7.7 Hz, 1H), 7.59 (d, J=8.1 Hz, 1H), 7.46 (d, J=7.3 Hz, 2H), 7.35 (t, J=7.6 Hz, 1H), 7.23 (m, 5H), 6.83 (d, J=8.5 Hz, 2H), 5.87 (br, 1H), 4.74 (d, J=5.3 Hz, 2H), 4.20 (s, 2H), 3.80 (s, 3H)
Compound No. 214: (2-Benzylquinazolino-4-yl)-(3-trifluoromethylbenzyl)-amine

¹H NMR (CDCl₃, 300 MHz) δ 7.80 (m, 2H), 7.66 (m, 2H), 7.49 (m, 2H), 7.37 (m, 4H), 7.20 (m, 2H), 6.56 (br, 1H), 4.86 (d, J=6.3 Hz, 2H), 4.18 (s, 2H)
Compound No. 215: (2-Benzylquinazolino-4-yl)-phenethylamine

¹H NMR (CDCl₃, 300 MHz) δ 7.82 (d, J=8.3 Hz, 1H), 7.61 (m, 4H), 7.34-7.17 (m, 6H), 6.12 (br, 1H), 4.21 (s, 2H), 3.88 (m, 2H), 2.94 (t, J=7.2 Hz, 2H)
Compound No. 216: (2-Benzylquinazolino-4-yl)-(4-phenylbutyl)-amine

¹H NMR (CDCl₃, 300 MHz) δ 7.81 (d, J=8.3 Hz, 1H), 7.63 (t, J=7.8 Hz, 2H), 7.46 (d, J=7.3 Hz, 2H), 7.33 (m, 5H), 7.24 (m, 4H), 5.98 (br, 1H), 4.18 (s, 2H), 3.62 (d, J=5.7 Hz, 2H), 2.64 (t, J=5.1 Hz, 2H), 1.69 (m, 2H)
Compound No. 217: (2-Benzylquinazolino-4-yl)-naphthaleno-1-ylmethylamine

¹H NMR (CDCl₃, 300 MHz) δ 8.01 (d, J=8.3 Hz, 1H), 7.85 (m, 3H), 7.54 (m, 2H), 7.52-7.39 (m, 6H), 7.25 (m, 4H), 6.32 (br, 1H), 5.28 (d, J=5.0 Hz, 2H), 4.25 (s, 2H)
Compound No. 218: Benzo[1,3]dioxol-5-ylmethyl-(2-benzylquinazolino-4-yl)-amine

¹H NMR (CDCl₃, 300 MHz) δ 7.82 (d, J=8.4 Hz, 1H), 7.68 (m, 1H), 7.61 (d, J=8.1 Hz, 1H), 7.46 (d, J=7.2 Hz, 2H), 7.36 (m, 1H), 7.25 (m, 2H), 7.18 (m, 1H), 6.81-6.71 (m, 3H), 5.94 (s, 2H), 5.89 (br, 1H), 4.71 (d, J=5.7 Hz, 2H), 4.20 (s, 2H)
Compound No. 219: (2-Benzylquinazolino-4-yl)-(3-fluorobenzyl)-amine

¹H NMR (CDCl₃, 300 MHz) δ 7.83 (d, J=8.0 Hz, 1H), 7.69 (t, J=7.3 Hz, 2H), 7.40 (m, 3H), 7.23 (m, 4H), 7.03 (m, 3H), 6.15 (br, 1H), 4.81 (d, J=5.7 Hz, 2H), 4.18 (s, 2H)
Compound No. 220: (2-Benzylquinazolino-4-yl)-[2-(3-fluorophenyl)-ethyl]-amine

¹H NMR (CDCl₃, 300 MHz) δ 7.82 (d, J=8.4 Hz, 1H), 7.68 (d, J=7.5 Hz, 1H), 7.49 (m, 3H), 7.34 (m, 5H), 6.92 (m, 3H), 5.69 (br, 1H), 4.21 (s, 2H), 3.84 (q, J=6.6 Hz, 2H), 2.92 (t, J=7.2 Hz, 2H)
Compound No. 221: 2-[2-(2-Benzylquinazolino-4-ylamino)-ethoxy]-ethanol

¹H NMR (CDCl₃, 300 MHz) δ 7.80 (d, J=8.4 Hz, 1H), 7.66 (d, J=8.4 Hz, 2H), 7.45 (d, J=7.2 Hz, 2H), 7.36 (d, J=7.7 Hz, 1H), 7.28 (m, 3H), 6.12 (br, 1H), 4.17 (s, 2H), 3.78 (m, 6H), 3.59 (t, J=4.5 Hz, 2H), 2.13 (br s, 1H),
Compound No. 222: 2-(2-Benzylquinazolino-4-ylamino)-ethanol

¹H NMR (CDCl₃, 300 MHz) δ 7.80 (d, J=8.3 Hz, 1H), 7.67 (m, 2H), 7.40 (m, 3H), 7.23 (m, 4H), 6.22 (br, 1H), 4.15 (s, 2H), 3.83 (t, J=4.5 Hz, 2H), 3.76 (m, 2H)
Compound No. 223: Benzyl-(2-pyridino-2-ylquinazolino-4-yl)-amine

¹H NMR (CDCl₃, 300 MHz) δ 8.80 (d, J=3.9 Hz, 1H), 8.55 (d, J=7.9 Hz, 1H), 8.08 (d, J=8.4 Hz, 1H), 7.84 (m, 2H), 7.71 (t, J=7.7 Hz, 1H), 7.42 (m, 3H), 7.34 (m, 4H), 6.65 (br, 1H), 4.99 (s, 2H)
Compound No. 224: (3-Methoxybenzyl)-(2-pyridino-2-ylquinazolino-4-yl)-amine

¹H NMR (CDCl₃, 300 MHz) δ 8.81 (d, J=4.2 Hz, 1H), 8.56 (d, J=7.8 Hz, 1H), 8.08 (d, J=8.4 Hz, 1H), 7.92-7.72 (m, 3H), 7.44 (t, J=7.6 Hz, 1H), 7.36 (t, J=6.1 Hz, 1H), 7.24 (m, 2H), 7.02 (m, 1H), 6.83 (m, 1H), 6.80 (br, 1H), 4.97 (s, 2H), 3.75 (s, 3H)
Compound No. 225: (4-Methoxybenzyl)-(2-pyridino-2-ylquinazolino-4-yl)-amine

¹H NMR (CDCl₃, 300 MHz) δ 8.82 (d, J=4.2 Hz, 1H), 8.59 (d, J=7.9 Hz, 1H), 8.08 (d, J=8.3 Hz, 1H), 7.84 (m, 2H), 7.71 (t, J=7.3 Hz, 1H), 7.43 (m, 4H), 6.86 (d, J=8.6 Hz, 2H), 6.61 (br, 1H), 4.93 (s, 2H), 3.78 (s, 3H)
Compound No. 226: (2-Pyridino-2-ylquinazolino-4-yl)-(3-trifluoromethylbenzyl)-amine

¹H NMR (CDCl₃, 300 MHz) δ 8.78 (d, J=4.4 Hz, 1H), 8.44 (d, J=7.9 Hz, 1H), 8.04 (m, 2H), 7.74 (m, 1H), 7.65 (m, 2H), 7.48 (m, 1H), 7.44-7.34 (m, 4H), 6.12 (br, 1H), 5.03 (s, 2H)
Compound No. 227: Phenethyl-(2-pyridino-2-ylquinazolino-4-yl)-amine

¹H NMR (CDCl₃, 300 MHz) δ 8.84 (d, J=4.0 Hz, 1H), 8.62 (d, J=7.9 Hz, 1H), 8.08 (d, J=8.3 Hz, 1H), 7.86 (t, J=3.8 Hz, 1H), 7.73 (m, 2H), 7.45-7.24 (m, 7H), 6.54 (br, 1H), 4.07 (t, J=6.7 Hz, 2H), 3.10 (t, J=7.1 Hz, 2H)
Compound No. 228: (4-Phenylbutyl)-(2-pyridino-2-ylquinazolino-4-yl)-amine

¹H NMR (CDCl₃, 300 MHz) δ 8.83 (d, J=4.2 Hz, 1H), 8.55 (d, J=7.9 Hz, 1H), 8.06 (d, J=8.3H, 1Hz), 7.84 (m, 2H), 7.71 (t, J=7.5 Hz, 1H), 7.44 (t, J=7.5 Hz, 1H), 7.37 (t, J=6.0 Hz, 1H), 7.28-7.16 (m, 5H), 6.44 (br, 1H), 3.83 (m, 2H), 2.70 (t, J=6.3 Hz, 2H), 1.81 (m, 4H)
Compound No. 229: Naphthaleno-1-ylmethyl-(2-pyridino-2-ylquinazolino-4-yl)-amine

¹H NMR (CDCl₃, 300 MHz) δ 8.83 (d, J=4.0 Hz, 1H), 8.60 (d, J=7.9 Hz, 1H), 8.10 (d, J=8.3 Hz, 1H), 8.05 (m, 1H), 7.83 (m, 4H), 7.71 (m, 1H), 7.63 (m, 1H), 7.50-7.36 (m, 5H), 6.78 (br, 1H), 5.43 (s, 2H)
Compound No. 230: Benzo[1,3]dioxol-5-ylmethyl-(2-pyridino-2-ylquinazolino-4-yl)-amine

¹H NMR (CDCl₃, 300 MHz) δ 8.83 (d, J=4.1 Hz, 1H), 8.58 (d, J=8.0 Hz, 1H), 8.08 (d, J=8.3 Hz, 1H), 7.84 (m, 2H), 7.73 (d, J=7.2 Hz, 1H), 7.45 (m, 1H), 7.36 (m, 1H), 6.90 (m, 2H), 6.76 (d, J=7.8 Hz, 1H), 6.61 (br, 1H), 5.92 (s, 2H), 4.90 (s, 2H)
Compound No. 231: (3-Fluorobenzyl)-(2-pyridino-2-ylquinazolino-4-yl)-amine

¹H NMR (CDCl₃, 300 MHz) δ 8.80 (d, J=4.1 Hz, 1H), 8.49 (d, J=8.1 Hz, 1H), 8.05 (m, 2H), 7.83 (m, 1H), 7.73 (t, J=7.7 Hz, 1H), 7.47 (m, 1H), 7.37 (m, 1H), 7.28 (m, 2H), 7.18 (m, 1H), 6.94 (m, 1H), 6.43 (br, 1H), 5.00 (s, 2H)
Compound No. 232: [2-(3-Fluorophenyl)-ethyl]-(2-pyridino-2-ylquinazolino-4-yl)-amine

¹H NMR (CDCl₃, 300 MHz) δ 8.83 (d, J=4.1 Hz, 1H), 8.59 (d, J=8.2 Hz, 1H), 8.07 (d, J=8.3 Hz, 1H), 7.86 (t, J=7.7 Hz, 1H), 7.73 (m, 2H), 7.39 (m, 2H), 7.25 (m, 1H), 7.00 (m, 3H), 6.45 (br, 1H), 4.03 (m, 2H), 3.08 (t, J=6.6 Hz, 2H)
Compound No. 233: 2-[2-(2-Pyridino-2-ylquinazolino-4-ylamino)-ethoxy]-ethanol

¹H NMR (CDCl₃, 300 MHz) δ 8.89 (t, J=6.8 Hz, 2H), 8.46 (d, J=8.0 Hz, 1H), 8.00 (m, 2H), 7.58 (d, J=7.5 Hz, 2H), 7.41 (, J=7.2 Hzt, 1H), 6.63 (br, 1H), 4.08 (m, 4H), 8.83 (m, 4H)
Compound No. 234: 2-(2-Pyridino-2-ylquinazolino-4-ylamino)-ethanol

¹H NMR (CDCl₃, 300 MHz) δ 8.80 (d, J=4.6 Hz, 1H), 8.56 (d, J=8.1 Hz, 1H), 8.01 (d, J=8.3 Hz, 1H), 7.84 (m, 2H), 7.74 (t, J=7.7 Hz, 1H), 7.40 (m, 1H), 7.36 (m, 1H), 6.81 (br, 1H), 3.97 (m, 4H)
Compound No. 235: Benzyl-(2-pyridino-3-ylquinazolino-4-yl)-amine

¹H NMR (CDCl₃, 300 MHz) δ 9.72 (s, 1H), 8.67 (d, J=4.5 Hz, 1H), 8.54 (m, 1H), 7.96 (d, J=8.3 Hz, 1H), 7.79 (m, 2H), 7.46-7.29 (m, 6H), 6.52 (br, 1H), 4.99 (d, J=5.4 Hz, 2H)
Compound No. 236: (3-Methoxybenzyl)-(2-pyridino-3-ylquinazolino-4-yl)-amine

¹H NMR (CDCl₃, 300 MHz) δ 9.72 (s, 1H), 8.86 (d, J=7.9 Hz, 1H), 8.68 (d, J=3.8 Hz, 1H), 8.01 (d, J=8.3 Hz, 1H), 7.85 (d, J=8.1 Hz, 1H), 7.83 (m, 1H), 7.43 (m, 2H), 7.26 (m, 1H), 7.01 (t, J=5.9 Hz, 2H), 6.84 (t, J=4.13 Hz, 1H), 6.68 (br, 1H), 4.97 (d, J=5.4 Hz, 2H), 3.77 (s, 3H)
Compound No. 237: (4-Methoxybenzyl)-(2-pyridino-3-ylquinazolino-4-yl)-amine

¹H NMR (CDCl₃, 300 MHz) δ 9.74 (s, 1H), 8.83 (d, J=7.9 Hz, 1H), 8.66 (d, J=3.6 Hz, 1H), 7.95 (d, J=8.3 Hz, 1H), 7.75 (m, 2H), 7.38 (m, 4H), 6.87 (d, J=8.8 Hz, 2H), 6.38 (br, 1H), 4.90 (d, J=5.4 Hz, 2H), 3.78 (s, 3H)
Compound No. 238: (2-Pyridino-3-ylquinazolino-4-yl)-(3-trifluoromethylbenzyl)-amine

¹H NMR (CDCl₃, 300 MHz) δ 9.89 (s, 1H), 8.74 (m, 2H), 7.96 (d, J=8.3 Hz, 1H), 7.78 (m, 4H), 7.47 (m, 4H), 6.39 (br, 1H), 5.05 (d, J=5.4 Hz, 2H)
Compound No. 239: Phenethyl-(2-pyridino-3-ylquinazolino-4-yl)-amine

¹H NMR (CDCl₃, 300 MHz) δ 9.77 (s, 1H), 8.85 (s, J=8.0 Hz, 1H), 7.95 (d, J=8.5 Hz, 1H), 7.73 (m, 1H), 7.41 (m, 7H), 6.17 (br, 1H), 4.05 (m, 2H), 3.10 (t, J=6.9 Hz, 2H)
Compound No. 240: (4-Phenylbutyl)-(2-pyridino-3-ylquinazolino-4-yl)-amine

¹H NMR (CDCl₃, 300 MHz) δ 9.74 (s, 1H), 8.82 (d, J=8.0 Hz, 1H), 8.68 (d, J=3.9 Hz, 1H), 7.95 (d, J=8.1 Hz, 1H), 7.72 (t, J=7.6 Hz, 2H), 7.41 (m, 2H), 7.24 (m, 5H), 6.12 (br, 1H), 3.80 (t, J=5.9 Hz, 2H), 2.71 (t, J=6.7 Hz, 2H), 1.81 (t, J=3.4 Hz, 4H)
Compound No. 241: Naphthaleno-1-ylmethyl-(2-pyridino-3-ylquinazolino-4-yl)-amine

¹H NMR (CDCl₃, 300 MHz)) δ 9.82 (s, 1H), 8.88 (d, J=7.9 Hz, 1H), 8.69 (m, 1H), 8.12 (m, 1H), 7.92 (m, 3H), 7.80 (m, 1H), 7.61 (m, 2H), 7.50 (m, 5H), 6.05 (br, 1H), 5.43 (d, J=4.8 Hz, 2H)
Compound No. 242: Benzo[1,3]dioxol-5-ylmethyl-(2-pyridino-3-ylquinazolino-4-yl)-amine

¹H NMR (CDCl₃, 300 MHz) δ 9.72 (s, 1H), 8.89 (d, J=8.0 Hz, 1H), 8.69 (d, J=4.5 Hz, 1H), 8.03 (d, J=8.5 Hz, 1H), 7.86 (d, J=8.1 Hz, 1H), 7.73 (t, J=7.3 Hz, 1H), 7.43 (t, J=6.5 Hz, 2H), 6.92 (d, J=7.8 Hz, 2H), 7.77 (d, J=7.7 Hz, 2H), 5.92 (s, 2H), 4.90 (d, J=5.4 Hz, 2H)
Compound No. 243: (3-Fluorobenzyl)-(2-pyridino-3-ylquinazolino-4-yl)-amine

¹H NMR (CDCl₃, 300 MHz) δ 9.69 (s, 1H), 8.81 (d, J=8.0 Hz, 1H), 8.68 (d, J=3.8 Hz, 1H), 8.00 (d, J=8.3 Hz, 1H), 7.87 (d, J=8.1 Hz, 1H), 7.75 (m, 1H), 7.44 (m, 2H), 7.29 (m, 2H), 7.14 (m, 1H), 6.96 (m, 1H), 6.72 (br, 1H), 5.00 (d, J=5.6 Hz, 2H)
Compound No. 244: [2-(3-Fluorophenyl)-ethyl]-(2-pyridino-3-ylquinazolino-4-yl)-amine

¹H NMR (CDCl₃, 300 MHz) δ 9.75 (s, 1H), 8.86 (d, J=8.0 Hz, 1H), 8.69 (d, J=4. Hz, 1H), 7.97 (d, J=8.4 Hz, 1H), 7.70 (m, 2H), 7.43 (m, 2H), 7.27 (m, 2H), 6.99 (m, 3H), 6.28 (br, 1H), 4.05 (m, 2H), 3.11 (t, J=6.9H, 2 Hz)
Compound No. 245: 2-[2-(2-Pyridino-3-ylquinazolino-4-ylamino)-ethoxy]-ethanol

¹H NMR (CDCl₃, 300 MHz) δ 9.66 (s, 1H), 8.73 (d, J=7.2 Hz, 1H), 8.63 (d, J=2.8 Hz, 1H), 7.86 (d, J=8.3H, 1Hz), 7.79 (d, J=8.2 Hz, 1H), 7.65 (m, 1H), 7.35 (m, 2H), 6.81 (br, 1H), 3.97 (m, 2H), 3.83 (m, 4H), 3.67 (m, 2H)
Compound No. 246: 2-(2-Pyridino-3-ylquinazolino-4-ylamino)-ethanol

¹H NMR (CDCl₃, 300 MHz) δ 9.67 (s, 1H), 8.76 (d, J=8.03 Hz, 1H), 8.66 (d, J=3.4 Hz, 1H), 7.94 (t, J=7.7 Hz, 2H), 7.72 (t, J=7.6 Hz, 1H), 7.42 (m, 2H), 6.94 (br, 1H), 4.00 (m, 4H)
Compound No. 247: Benzyl-(2-pyridino-4-ylquinazolino-4-yl)-amine

¹H NMR (CDCl₃, 300 MHz) δ 8.74 (m, 2H), 8.43 (d, J=5.5 Hz, 2H), 7.98 (d, J=8.3 Hz, 1H), 7.75 (t, J=4.4 Hz, 2H), 7.48 (m, 3H), 7.36 (m, 3H), 6.21 (br, 1H), 5.01 (d, J=5.4 Hz, 2H)
Compound No. 248: (3-Methoxybenzyl)-(2-pyridino-4-ylquinazolino-4-yl)-amine

¹H NMR (CDCl₃, 300 MHz) δ 8.73 (d, J=5.0 Hz, 2H), 8.43 (d, J=5.5 Hz, 2H), 9.98 (d, J=8.2 Hz, 1H), 7.77 (t, J=7.7 Hz, 2H), 7.49 (d, J=7.2 Hz, 1H), 7.26 (m, 2H), 6.84 (m, 1H), 6.35 (br, 1H), 4.98 (d, J=5.3 Hz, 2H)
Compound No. 249: (4-Methoxybenzyl)-(2-pyridino-4-ylquinazolino-4-yl)-amine

¹H NMR (CDCl₃, 300 MHz) δ 8.74 (d, J=5.5 Hz, 2H), 8.46 (d, J=5.5 Hz, 2H), 7.98 (d, J=8.2 Hz, 1H), 7.76 (t, J=7.6 Hz, 2H), 7.47 (t, J=7.6 Hz, 1H), 7.38 (d, J=8.5 Hz, 2H), 6.90 (d, J=8.5 Hz, 2H), 6.29 (br, 1H), 4.93 (d, J=5.4 Hz, 2H), 3.80 (s, 3H)
Compound No. 250: (2-Pyridino-4-ylquinazolino-4-yl)-(3-trifluoromethylbenzyl)-amine

¹H NMR (CDCl₃, 300 MHz) δ 8.72 (m, 2H), 8.40 (d, J=4.71 Hz, 2H), 8.02 (d, J=8.3 Hz, 1H), 7.84 (m, 2H), 7.75 (m, 1H), 7.65 (m, 1H), 7.52 (m, 3H), 6.57 (br, 1H), 5.02 (d, J=5.3 Hz, 2H)
Compound No. 251: Phenethyl-(2-pyridino-4-ylquinazolino-4-yl)-amine

¹H NMR (CDCl₃, 300 MHz) δ 8.74 (d, J=5.5 Hz, 2H), 8.45 (d, J=5.5 Hz, 2H), 7.96 (d, J=8.3 Hz, 1H), 7.76 (t, J=7.4 Hz, 1H), 7.67 (dH, J=8.2 Hz, 1), 7.46 (d, J=7.2 Hz, 1H), 7.33 (m, 5H), 6.09 (br, 1H), 4.06 (m, 2H), 3.11 (t, J=6.9 Hz, 2H)
Compound No. 252: (4-Phenylbutyl)-(2-pyridino-4-ylquinazolino-4-yl)-amine

¹H NMR (300 MHz, CDCl₃, 300 MHz) δ 8.76 (d, J=5.4 Hz, 2H), 8.42 (d, J=5.5 Hz, 2H), 7.97 (d, J=8.3 Hz, 1H), 7.75 (t, J=7.5 Hz, 2H), 7.47 (t, J=7.5 Hz, 1H), 7.25 (m, 6H), 6.10 (br, 1H), 3.82 (d, J=5.6 Hz, 2H), 2.73 (t, J=6.6 Hz, 2H), 1.84 (m, 4H)
Compound No. 253: Naphthaleno-1-ylmethyl-(2-pyridino-4-ylquinazolino-4-yl)-amine

¹H NMR (CDCl₃, 300 MHz) δ 8.76 (d, J=5.4 Hz, 2H), 8.46 (d, J=5.3 Hz, 2H), 8.12 (d, J=8.4 Hz, 1H), 7.95 (m, 3H), 7.77 (m, 1H), 7.62 (t, J=5.8 Hz, 2H), 7.50 (m, 4H), 5.99 (br, 1H), 5.44 (d, J=4.9 Hz, 2H)
Compound No. 254: Benzo[1,3]dioxol-5-ylmethyl-(2-pyridino-4-ylquinazolino-4-yl)-amine

¹H NMR (CDCl₃, 300 MHz) δ 8.74 (d, J=5.4 Hz, 2H), 8.40 (d, J=6.06 Hz, 2H), 7.96 (d, J=8.4 Hz, 1H), 7.76 (m, 2H), 7.48 (t, J=7.6 Hz, 1H), 6.92 (d, J=7.97 Hz, 2H), 6.80 (d, J=7.7 Hz, 1H), 6.07 (br, 1H), 5.96 (s, 2H), 4.91 (d, J=5.3 Hz, 2H)
Compound No. 255: (3-Fluorobenzyl)-(2-pyridino-4-ylquinazolino-4-yl)-amine

¹H NMR (CDCl₃, 300 MHz) δ 8.74 (d, J=5.4 Hz, 2H), 8.38 (d, J=5.1 Hz, 2H), 7.98 (d, J=8.4H, 1Hz), 7.79 (t, J=7.8 Hz, 2H), 7.51 (t, J=3.9 Hz, 1H), 7.35 (t, J=3.9 Hz, 1H), 7.25 (m, 1H), 7.17 (m, 1H), 7.01 (m, 1H), 6.24 (br, 1H), 5.01 (d, J=5.5 Hz, 2H)
Compound No. 256: [2-(3-Fluorophenyl)-ethyl]-(2-pyridino-4-ylquinazolino-4-yl)-amine

¹H NMR (CDCl₃, 300 MHz) δ 8.76 (d, J=5.5 Hz, 2H), 8.44 (d, J=5.3 Hz, 2H), 7.98 (d, J=8.4 Hz, 1H), 7.67 (d, J=8.0 Hz, 1H), 7.48 (d, J=7.3 Hz, 1H), 7.28 (m, 1H), 7.03 (m, 3H), 6.14 (br, 1H), 4.05 (m, 2H), 3.11 (t, J=6.9 Hz, 2H)
Compound No. 257: 2-[2-(2-Pyridino-4-ylquinazolino-4-ylamino)-ethoxy]-ethanol

¹H NMR (CDCl₃, 300 MHz) δ 8.72 (d, J=4.8 Hz, 2H), 8.36 (d, J=5.8 Hz, 2H), 7.92 (d, J=8.3 Hz, 1H), 7.83 (d, J=8.2 Hz, 1H), 7.73 (t, J=7.6 Hz, 1H), 7.45 (t, J=7.6 Hz, 1H), 6.64 (br, 1H), 4.02 (m, 2H), 3.81 (m, 4H), 3.69 (m, 2H)
Compound No. 258: 2-(2-Pyridino-4-ylquinazolino-4-ylamino)-ethanol

¹H NMR (CDCl₃, 300 MHz) δ 8.71 (d, J=4.8 Hz, 2H), 8.35 (d, J=5.5 Hz, 2H), 8.13 (d, J=8.4 Hz, 1H), 7.90 (d, J=8.2 Hz, 1H), 7.73 (t, J=8.3 Hz, 1H), 7.47 (t, J=7.6 Hz, 1H), 7.18 (br, 1H), 3.99 (m, 4H)
Compound No. 259: Benzyl-[2-(4-chlorophenyl)-quinazolino-4-yl]-amine

¹H NMR (CDCl₃, 300 MHz) δ 8.52 (d, J=8.6 Hz, 2H), 7.92 (d, J=8.2 Hz, 1H), 7.71 (m, 2H), 7.40 (m, 8H), 5.97 (br, 1H), 5.00 (d, J=5.5 Hz, 2H)
Compound No. 260: [2-(4-chlorophenyl)-quinazolino-4-yl]-(3-methoxybenzyl)-amine

¹H NMR (CDCl₃, 300 MHz) δ 8.52 (d, J=8.5 Hz, 2H), 7.92 (d, J=8.3 Hz, 1H), 7.72 (m, 2H), 7.42 (m, 3H), 7.28 (m, 1H), 7.03 (d, J=8.0 Hz, 2H), 6.87 (t, J=4.2 Hz, 1H), 5.98 (br, 1H), 4.98 (d, J=5.5 Hz, 2H), 3.78 (s, 3H)
Compound No. 261: [2-(4-Chlorophenyl)-quinazolino-4-yl]-(4-methoxybenzyl)-amine

¹H NMR (CDCl₃, 300 MHz) δ 8.53 (d, J=8.5 Hz, 2H), 7.96 (d, J=8.55 Hz, 1H), 7.72 (m, 2H), 7.38 (m, 5H), 6.90 (d, J=8.6 Hz, 2H), 4.92 (d, J=5.3 Hz, 2H), 3.80 (s, 3H)
Compound No. 262: [2-(4-Chlorophenyl)-quinazolino-4-yl]-(3-trifluoromethylbenzyl)-amine

¹H NMR (CDCl₃, 300 MHz) δ 8.45 (d, J=8.6 Hz, 2H), 7.95 (d, J=8.5 Hz 1H), 7.75 (m, 3H), 7.65 (d, J=7.7 Hz, 1H), 7.56 (d, J=7.7 Hz, 1H), 7.45 (m, 4H), 6.12 (br, 1H), 5.05 (d, J=5.5 Hz, 2H)
Compound No. 263: [2-(4-Chlorophenyl)-quinazolino-4-yl]-phenethylamine

¹H NMR (CDCl₃, 300 MHz) δ 8.54 (d, J=8.4 Hz, 2H), 7.94 (d, J=8.5 Hz, 1H), 7.70 (t, J=7.5 Hz, 1H), 7.60 (d, J=8.1 Hz, 1H), 7.46 (d, J=8.6 Hz, 2H), 7.35 (m, 6H), 6.04 (br, 1H), 4.05 (m, 2H), 3.10 (t, J=7.0 Hz, 2H)
Compound No. 264: [2-(4-Chlorophenyl)-quinazolino-4-yl]-(4-phenylbutyl)-amine

¹H NMR (CDCl₃, 300 MHz) δ 8.50 (d, J=8.5 Hz, 2H), 7.93 (d, J=8.6 Hz, 1H), 7.70 (m, 2H), 7.41 (m, 3H), 7.25 (m, 5H), 6.09 (br, 1H), 3.80 (d, J=5.5 Hz, 2H), 2.72 (m, 2H), 1.82 (m, 4H)
Compound No. 265: [2-(4-Chlorophenyl)-quinazolino-4-yl]-naphthaleno-1-ylmethylamine

¹H NMR (CDCl₃, 300 MHz) δ 8.58 (d, J=8.5 Hz, 2H), 8.17 (m, 1H), 7.91 (m, 3H), 7.63 (m, 1H), 7.51 (m, 7H), 7.36 (m, 1H), 5.87 (br, 1H), 5.42 (d, J=4.8 Hz, 2H)
Compound No. 266: Benzo[1,3]dioxol-5-ylmethyl-[2-(4-chlorophenyl)-quinazolino-4-yl]-amine

¹H NMR (CDCl₃, 300 MHz) δ 8.52 (d, J=8.6 Hz, 2H), 7.92 (d, J=8.3 Hz, 1H), 7.71 (m, 2H), 7.43 (m, 3H), 6.93 (d, J=11.4 Hz, 2H), 6.80 (d, J=10.2 Hz, 1H), 5.95 (s, 2H), 5.94 (br, 1H), 4.89 (d, J=4.9 Hz, 2H)
Compound No. 267: [2-(4-Chlorophenyl)-quinazolino-4-yl]-(3-fluorobenzyl)-amine

¹H NMR (CDCl₃, 300 MHz) δ 8.48 (d, J=8.5 Hz, 2H), 7.92 (d, J=8.5 Hz, 1H), 7.74 (m, 2H, 7.77 (m, 3H), 7.23 (m, 3H), 7.00 (m, 1H), 6.07 (br, 1H), 4.98 (d, J=5.5 Hz, 2H)
Compound No. 268: [2-(4-Chlorophenyl)-quinazolino-4-yl]-[2-(3-fluorophenyl)-ethyl]-amine

¹H NMR (CDCl₃, 300 MHz) δ 8.52 (d, J=8.6 Hz, 2H), 7.94 (d, J=8.6 Hz, 1H), 7.72 (d, J=7.2 Hz, 1H), 7.63 (d, J=8.1 Hz, 1H), 7.43 (m, 3H), 7.28 (m, 1H), 7.02 (m, 3H), 6.08 (br, 1H), 4.03 (m, 2H), 3.10 (t, J=7.0 Hz, 2H)
Compound No. 269: 2-{2-[2-(4-Chlorophenyl)-quinazolino-4-ylamino]-ethoxy}-ethanol

¹H NMR (CDCl₃, 300 MHz) δ 8.47 (d, J=8.4 Hz, 2H), 7.90 (d, J=8.3 Hz, 1H), 7.72 (m, 2H), 7.41 (m, 3H), 6.52 (br, 1H), 4.00 (m, 2H), 3.81 (m, 4H), 3.67 (m, 2H)
Compound No. 270: 2-[2-(4-Chlorophenyl)-quinazolino-4-ylamino]-ethanol

¹H NMR (CDCl₃, 300 MHz) δ 8.46 (d, J=8.5 Hz, 2H), 7.90 (d, J=8.4 Hz, 1H), 7.75 (m, 2H), 7.46 (m, 3H), 6.21 (br, 1H), 3.00 (m, 4H)
Compound No. 271: (2,4-Dimethoxybenzyl)-(2-isopropylquinazolino-4-yl)-amine

¹H NMR (CDCl₃, 300 MHz) δ 7.83 (d, J=8.1 Hz, 1H), 7.68-7.60 (m, 2H), 7.38-7.33 (m, 2H), 6.50-6.42 (m, 2H), 6.24 (br, 1H), 4.83 (d, J=5.7 Hz, 2H), 3.88 (s, 3H), 3.80 (s, 3H), 3.17 (m, 1H), 1.40 (d, J=6.9 Hz, 6H)
Compound No. 272: (2-Benzylquinazolino-4-yl)-(2,4-dimethoxybenzyl)-amine

¹H NMR (CDCl₃, 300 MHz) δ 7.77 (d, J=8.4 Hz, 1H), 7.64 (t, J=7.7 Hz, 1H), 7.56 (d, J=8.1 Hz, 1H), 7.48 (d, J=6.9 Hz, 2H), 7.30 (m, 4H), 7.09 (d, J=8.4 Hz, 1H), 6.45 (d, J=2.4 Hz, 1H), 6.32 (dd, J₁=8.4 Hz, J₂=2.4 Hz, 1H), 6.17 (br, 1H), 4.73 (d, J=6.0 Hz, 2H), 4.20 (s, 2H), 3.85 (s, 3H), 3.78 (s, 3H)
Compound No. 273: 4-(2,4-Dimethoxybenzyloxy)-2-phenylquinazoline

¹H NMR (CDCl₃, 300 MHz) δ 8.63 (m, 2H), 8.17 (d, 1H, J=7.8 Hz), 7.98 (d, J=8.1 Hz, 1H), 7.79 (m, 1H), 7.53 (m, 5H), 6.53 (m, 2H), 5.75 (s, 2H), 3.87 (s, 3H), 3.83 (s, 3H)
Compound No. 274: 4-(2,4-Dimethoxybenzylsulfanyl)-2-phenylquinazoline

¹H NMR (CDCl₃, 300 MHz) δ 8.68 (dd, J₁=7.5 Hz, J₂=2.1 Hz, 2H), 8.05 (d, J=8.4 Hz, 1H), 7.98 (d, J=8.4 Hz, 1H), 7.80 (t, J=7.5 Hz, 1H), 7.45 (m, 5H), 6.50 (d, J=2.4 Hz, 1H), 6.41 (dd, J₁=8.4 Hz, J₂=2.3 Hz, 1H), 4.80 (s, 2H), 3.88 (s, 3H), 3.78 (s, 3H)
Compound No. 275: (2-Methoxybenzyl)-(2-pyrrolidino-1-ylquinazolino-4-yl)-amine

¹H NMR (CDCl₃, 300 MHz) δ 7.48-7.38 (m, 4H), 7.26 (m, 1H), 6.99 (m, 1H), 6.92 (m, 2H), 6.02 (br, 1H), 4.82 (d, J=5.7 Hz, 2H), 3.91 (s, 3H), 3.70 (t, J=6.6 Hz, 4H), 1.98 (m, 4H)
Compound No. 276: (2-Methoxybenzyl)-(2-piperidino-1-ylquinazolino-4-yl)-amine

¹H NMR (CDCl₃, 300 MHz) δ 7.50-7.46 (m, 4H), 7.30-7.26 (m, 1H), 7.00 (t, J=7.5 Hz, 1H), 6.92 (m, 2H), 5.96 (br, 1H), 4.80 (d, J=5.4 Hz, 2H), 3.91 (s, 7H), 1.62 (m, 6H)
Compound No. 277: (2-Methoxybenzyl)-(2-morpholino-4-ylquinazolino-4-yl)-amine

¹H NMR (CDCl₃, 300 MHz) δ 7.53-7.44 (m, 3H), 7.36-7.26 (m, 2H), 7.06 (td, J₁=6.6 Hz, J₂=1.5 Hz, 1H), 6.93 (m, 2H), 6.04 (br, 1H), 4.80 (d, J=5.7 Hz, 2H), 3.90 (m, 7H), 3.79 (m, 4H)
Compound No. 278: (2-Methoxybenzyl)-[2-(4-methylpiperazino-1-yl)-quinazolino-4-yl]-amine

¹H NMR (CDCl₃, 300 MHz) δ 7.52-7.43 (m, 3H), 7.36 (d, J=7.5 Hz, 1H), 7.28 (m, 1H), 7.04 (t, J=8.1 Hz, 1H), 6.93 (m, 2H), 6.01 (br, 1H), 4.80 (d, J=5.4 Hz, 2H), 3.97 (t, J=4.8 Hz, 4H), 3.91 (s, 3H), 2.50 (t, J=4.8 Hz, 4H), 2.35 (s, 3H)
Compound No. 279: (3-Methoxybenzyl)-(2-pyrrolidino-1-ylquinazolino-4-yl)-amine

¹H NMR (CDCl₃, 300 MHz) δ 7.50-7.44 (m, 3H), 7.27 (m, 1H), 7.03-6.96 (m, 3H), 6.83 (dd, J₁=8.4 Hz, J₂=2.4 Hz, 1H), 5.73 (br, 1H), 4.80 (d, J=5.1 Hz, 2H), 3.80 (s, 3H), 3.67 (t, J=6.6 Hz, 4H), 1.96 (m, 4H)
Compound No. 280: (3-Methoxybenzyl)-(2-piperidino-1-ylquinazolino-4-yl)-amine

¹H NMR (CDCl₃, 300 MHz) δ 7.52-7.43 (m, 3H), 7.27 (m, 1H), 7.04-6.96 (m, 1H), 6.84 (dd, J₁=8.1 Hz, J₂=2.1 Hz, 1H), 5.69 (br, 1H), 4.77 (d, J=5.4 Hz, 2H), 3.87 (m, 4H), 3.80 (s, 3H), 1.62 (m, 6H)
Compound No. 281: (3-Methoxybenzyl)-(2-morpholino-4-ylquinazolino-4-yl)-amine

¹H NMR (CDCl₃, 300 MHz) δ 7.57-7.47 (m, 3H), 7.28 (m, 1H), 7.08 (t, J=7.8 Hz, 1H), 7.00-6.94 (m, 2H), 6.85 (dd, J₁=8.4 Hz, J₂=2.4 Hz, 1H), 5.76 (br, 1H), 4.77 (d, J=5.4 Hz, 2H), 3.89 (m, 4H), 3.80-3.75 (m, 7H)
Compound No. 282: (3-Methoxybenzyl)-[2-(4-methylpiperazino-1-yl)-quinazolino-4-yl]-amine

¹H NMR (CDCl₃, 300 MHz) δ 7.54-7.45 (m, 3H), 7.27 (m, 1H), 7.05 (m, 1H), 6.97 (m, 2H), 6.84 (dd, J₁=8.4 Hz, J₂=2.1 Hz, 1H), 5.74 (br, 1H), 4.77 (d, J=5.4 Hz, 2H), 3.93 (t, J=4.8 Hz, 4H), 3.80 (s, 3H), 2.47 (t, J=4.8 Hz, 4H), 2.34 (s, 3H)
Compound No. 283: (4-Methoxybenzyl)-(2-pyrrolidino-1-ylquinazolino-4-yl)-amine

¹H NMR (CDCl₃, 300 MHz) δ 7.49 (d, J=3.6 Hz, 2H), 7.44 (d, J=8.1 Hz, 1H), 7.33 (d, J=8.7 Hz, 2H), 6.99 (m, 1H), 6.88 (d, J=8.7 Hz, 2H), 5.68 (br, 1H), 4.75 (d, J=5.4 Hz, 2H), 3.81 (s, 3H), 3.68 (t, J=6.9 Hz, 4H), 1.96 (m, 4H)
Compound No. 284: (4-Methoxybenzyl)-(2-piperidino-1-ylquinazolino-4-yl)-amine

¹H NMR (CDCl₃, 300 MHz) δ 7.51-7.41 (m, 3H), 7.33 (d, J=8.7 Hz, 2H), 7.00 (m, 1H), 6.89 (d, J=8.4 Hz, 2H), 5.62 (br, 1H), 4.73 (d, J=5.4 Hz, 2H), 3.88 (m, 4H), 3.81 (s, 3H), 1.27 (m, 6H)
Compound No. 285: (4-Methoxybenzyl)-(2-morpholino-4-ylquinazolino-4-yl)-amine

¹H NMR (CDCl₃, 300 MHz) δ 7.53-7.44 (m, 3H), 7.32 (d, J=8.7 Hz, 2H), 7.06 (m, 1H), 6.89 (d, J=8.7 Hz, 2H), 5.69 (br, 1H), 4.72 (d, J=5.1 Hz, 2H), 3.88 (m, 4H), 3.81 (s, 3H), 3.77 (m, 4H)
Compound No. 286: (4-Methoxybenzyl)-[2-(4-methylpiperazino-1-yl)-quinazolino-4-yl]-amine

¹H NMR (CDCl₃, 300 MHz) δ 7.54-7.42 (m, 3H), 7.33 (d, J=8.7 Hz, 2H), 7.03 (m, 1H), 6.89 (d, J=8.7 Hz, 2H), 5.65 (br, 1H), 4.73 (d, J=5.4 Hz, 2H), 3.95 (t, J=4.8 Hz, 4H), 3.81 (s, 3H), 2.48 (t, J=4.8 Hz, 4H), 2.34 (s, 3H)
Compound No. 287: (2,3-Dimethoxybenzyl)-(2-pyrrolidino-1-ylquinazolino-4-yl)-amine

¹H NMR (CDCl₃, 300 MHz) δ 7.47 (m, 3H), 7.00 (m, 3H), 6.88 (dd, J₁=5.4 Hz, J₂=3.6 Hz, 1H), 6.01 (br, 1H), 4.84 (d, J=6.0 Hz, 2H), 3.92 (s, 3H), 3.88 (s, 3H), 3.69 (t, 4H), 1.97 (m, 4H)
Compound No. 288: (2,3-Dimethoxybenzyl)-(2-piperidino-1-ylquinazolino-4-yl)-amine

¹H NMR (CDCl₃, 300 MHz) δ 7.50-7.41 (m, 3H), 7.00 (m, 3H), 6.88 (dd, J₁=7.2 Hz, J₂=2.7 Hz, 1H), 5.97 (br, 1H), 4.82 (d, J=5.7 Hz, 2H), 3.92 (s, 3H), 3.88 (s, 7H), 1.65 (m, 6H)
Compound No. 289: (2,3-Dimethoxybenzyl)-(2-morpholino-4-ylquinazolino-4-yl)-amine

¹H NMR (CDCl₃, 300 MHz) δ 7.51-7.44 (m, 3H), 7.08-6.94 (m, 3H), 6.89 (dd, J₁=8.1 Hz, J₂=1.5 Hz, 1H), 6.05 (br, 1H), 4.81 (d, J=5.7 Hz, 2H), 3.93-3.89 (m, 10H), 3.79 (t, J=4.8 Hz, 4H)
Compound No. 290: (2,3-Dimethoxybenzyl)-[2-(4-methylpiperazino-1-yl)-quinazolino-4-yl]-amine

¹H NMR (CDCl₃, 300 MHz) δ 7.49-7.44 (m, 3H), 7.05-6.96 (m, 3H), 6.89 (dd, J₁=7.5 Hz, J₂=1.5 Hz, 1H), 6.00 (br, 1H), 4.82 (d, J=5.7 Hz, 2H), 3.96 (t, J=4.8 Hz, 4H), 3.92 (s, 3H), 3.89 (s, 3H), 2.50 (t, J=4.8 Hz, 4H), 2.35 (s, 3H)
Compound No. 291: (2,4-Dimethoxybenzyl)-(2-pyrrolidino-1-ylquinazolino-4-yl)-amine

¹H NMR (CDCl₃, 300 MHz) δ 7.46 (d, J=3.9 Hz, 2H), 7.42 (d, J=8.1 Hz, 1H), 7.31 (d, J=8.4 Hz, 1H), 6.98 (s, 1H), 6.49 (d, J=2.4 Hz, 1H), 6.44 (dd, J₁=8.1 Hz, J₂=2.4 Hz, 1H), 5.94 (br, 1H), 4.74 (d, J=6.0 Hz, 2H), 3.87 (s, 3H), 3.80 (s, 3H), 3.71 (t, J=6.6 Hz, 4H), 1.98 (m, 4H)
Compound No. 292: (2,4-Dimethoxybenzyl)-(2-piperidino-1-ylquinazolino-4-yl)-amine

¹H NMR (CDCl₃, 300 MHz) δ 7.49-7.39 (m, 3H), 7.27 (m, 1H), 6.99 (t, J=6.6 Hz, 1H), 6.49 (d, J=2.4 Hz, 1H), 6.44 (dd, J₁=8.1 Hz, J₂=2.1 Hz, 1H), 5.88 (br, 1H), 4.72 (d, J=5.4 Hz, 2H), 3.97 (m, 4H), 3.87 (s, 3H), 3.80 (s, 3H), 1.66 (m, 6H)
Compound No. 293: (2,4-Dimethoxybenzyl)-(2-morpholino-4-ylquinazolino-4-yl)-amine

¹H NMR (CDCl₃, 300 MHz) δ 7.53-7.43 (m, 3H), 7.26 (m, 2H), 7.05 (m, 1H), 6.50 (d, J=2.1 Hz, 1H), 6.45 (dd, J₁=8.1 Hz, J₂=2.1 Hz, 1H), 5.94 (br, 1H), 4.72 (d, J=5.7 Hz, 2H), 3.94-3.78 (m, 14H)
Compound No. 294: (2,4-Dimethoxybenzyl)-[2-(4-methylpiperazino-1-yl)-quinazolino-4-yl]-amine

¹H NMR (CDCl₃, 300 MHz) δ 7.50 (m, 2H), 7.43 (d, 1H, J=8.1 Hz), 7.26 (m, 1H), 7.06 (m, 1H), 6.50 (d, J=2.1 Hz, 1H), 6.45 (dd, J₁=8.1 Hz, J₂=2.1 Hz, 1H), 6.00 (br, 1H), 4.72 (d, J=5.4 Hz, 2H), 4.01 (m, 4H), 3.87 (s, 3H), 3.81 (s, 3H), 2.54 (t, J=4.8 Hz, 4H), 2.37 (s, 3H)
Compound No. 295: (2,5-Dimethoxybenzyl)-(2-pyrrolidino-1-ylquinazolino-4-yl)-amine

¹H NMR (CDCl₃, 300 MHz) δ 7.45 (m, 3H), 6.99 (m, 2H), 6.79 (m, 2H), 6.02 (br s, 1H), 4.79 (d, J=5.7 Hz, 2H), 3.86 (s, 3H), 3.75 (s, 3H), 3.71 (br t, J=6.8 Hz, 4H), 1.97 (br t, J=6.8 Hz, 4H)
Compound No. 296: (2,5-Dimethoxybenzyl)-(2-piperidino-1-ylquinazolino-4-yl)-amine

¹H NMR (CDCl₃, 300 MHz) δ 7.44 (m, 3H), 6.97 (m, 2H), 6.79 (m, 2H), 5.99 (br s, 1H), 4.77 (d, J=5.7 Hz, 2H), 3.91 (br s, 4H), 3.87 (s, 3H), 3.75 (s, 3H), 1.65 (br s, 6H)
Compound No. 297: (2,5-Dimethoxybenzyl)-(2-morpholino-4-ylquinazolino-4-yl)-amine

¹H NMR (CDCl₃, 300 MHz) δ 7.48 (m, 3H), 7.06 (br t, J=7.5 Hz, 1H), 6.94 (d, J=2.7 Hz, 1H), 6.80 (m, 2H), 6.05 (br s, 1H), 4.76 (d, J=5.7 Hz, 2H), 3.92 (m, 4H), 3.90 (s, 3H), 3.79 (m, 4H), 3.75 (s, 3H)
Compound No. 298: (2,5-Dimethoxybenzyl)-[2-(4-methylpiperazino-1-yl)-quinazolino-4-yl]-amine

¹H NMR (CDCl₃, 300 MHz) δ 7.46 (m, 3H), 7.06 (br t, J=7.8 Hz, 1H), 6.95 (d, J=3.0 Hz, 1H), 6.80 (m, 2H), 6.02 (br s, 1H), 4.77 (d, J=5.7 Hz, 2H), 3.97 (br t, J=5.8 Hz, 4H), 3.86 (s, 3H), 3.75 (s, 3H), 2.50 (br t, J=5.8 Hz, 4H), 2.35 (s, 3H)
Compound No. 299: (3,4-Dimethoxybenzyl)-(2-pyrrolidino-1-ylquinazolino-4-yl)-amine

¹H NMR (CDCl₃, 300 MHz) δ 7.49 (d, J=3.9 Hz, 2H), 7.46 (d, J=8.4 Hz, 1H), 7.00 (m, 3H), 6.84 (m, 1H), 5.71 (br s, 1H), 4.75 (d, J=5.4 Hz, 2H), 3.89 (s, 3H), 3.86 (s, 3H), 3.69 (br t, J=6.6 Hz, 4H), 1.97 (br t, J=6.6 Hz, 4H)
Compound No. 300: (3,4-Dimethoxybenzyl)-(2-piperidino-1-ylquinazolino-4-yl)-amine

¹H NMR (CDCl₃, 300 MHz) δ 7.45 (m, 3H), 6.99 (m, 3H), 6.85 (d, J=8.4 Hz, 1H), 5.64 (br s, 1H), 4.73 (d, J=5.7 Hz, 2H), 3.89 (m, 10H), 1.65 (m, 6H),
Compound No. 301: (3,4-Dimethoxybenzyl)-(2-morpholino-4-ylquinazolino-4-yl)-amine

¹H NMR (CDCl₃, 300 MHz) δ 7.52 (m, 3H), 7.08 (t, J=7.8 Hz, 1H), 6.94 (m, 2H), 6.85 (d, J=8.4 Hz, 1H), 5.71 (br s, 1H), 4.72 (d, J=4.8 Hz, 2H), 3.91 (m, 4H), 3.89 (s, 3H), 3.86 (s, 3H), 3.78 (m, 4H)
Compound No. 302: (3,4-Dimethoxybenzyl)-[2-(4-methylpiperazino-1-yl)-quinazolino-4-yl]-amine

¹H NMR (CDCl₃, 300 MHz) δ 7.49 (m, 3H), 7.05 (t, J=7.8 Hz, 1H), 6.95 (m, 2H), 6.84 (m, 1H), 5.66 (br s, 1H), 4.73 (d, J=5.4 Hz, 2H), 3.96 (br t, J=5.0 Hz, 4H), 3.89 (s, 3H), 3.86 (s, 3H), 2.49 (brvt, J=5.0 Hz, 4H), 2.35 (s, 3H)
Compound No. 303: (3,5-Dimethoxybenzyl)-(2-pyrrolidino-1-ylquinazolino-4-yl)-amine

¹H NMR (CDCl₃, 300 MHz) δ 7.50 (m, 3H), 7.01 (m, 1H), 6.56 (d, J=1.8 Hz, 2H), 6.39 (br t, J=2.3 Hz, 1H), 5.82 (br s, 1H), 4.75 (d, J=5.4 Hz, 2H), 3.77 (s, 6H), 3.67 (br t, J=6.6 Hz, 4H), 1.95 (br t, J=6.6 Hz, 4H)
Compound No. 304: (3,5-Dimethoxybenzyl)-(2-piperidino-1-ylquinazolino-4-yl)-amine

¹H NMR (CDCl₃, 300 MHz) δ 7.46 (m, 3H), 7.02 (t, J=8.1 Hz, 1H), 6.56 (br d, J=2.4 Hz, 2H), 6.40 (br t, J=1.8 Hz, 1H), 5.71 (br s, 1H), 4.73 (d J=5.4 Hz, 2H), 3.88 (m, 4H), 3.78 (s, 6H), 1.62 (s, 6H)
Compound No. 305: (3,5-Dimethoxybenzyl)-(2-morpholino-4-ylquinazolino-4-yl)-amine

¹H NMR (CDCl₃, 300 MHz) δ 7.52 (m, 3H), 7.09 (br t, J=7.8 Hz, 1H), 6.55 (br d, J=2.1 Hz, 2H), 6.40 (br t, J=2.1 Hz, 1H), 5.75 (br s, 1H), 4.73 (d J=5.4 Hz, 2H), 3.91 (m, 4H), 3.78 (m, 10H)
Compound No. 306: (3,5-Dimethoxybenzyl)-[2-(4-methylpiperazino-1-yl)-quinazolino-4-yl]-amine

¹H NMR (CDCl₃, 300 MHz) δ 7.50 (m, 3H), 7.06 (t, J=7.8 Hz, 1H), 6.56 (d, J=2.4 Hz, 2H), 6.40 (br t, J=2.1 Hz, 1H), 5.72 (br s, 1H), 4.73 (d J=5.4 Hz, 2H), 3.94 (br t, J=4.2 Hz, 4H), 3.78 (s, 6H), 2.48 (br t, J=5.0 Hz, 4H), 2.34 (s, 3H)
Compound No. 307: Benzo[1,3]dioxol-5-ylmethyl-(2-pyrrolidino-1-ylquinazolino-4-yl)-amine

¹H NMR (CDCl₃, 300 MHz) δ 7.48 (br d, J=3.9 Hz, 2H), 7.45 (d, J=8.1 Hz, 1H), 6.99 (quartet, J=4.1 Hz, 1H), 6.92 (s, 1H), 6.86 (d, J=7.8 Hz, 1H), 6.76 (d, J=7.8 Hz, 1H), 5.95 (s, 2H), 5.70 (br s, 1H), 4.72 (d, J=5.7 Hz, 2H), 3.68 (br t, J=6.6 Hz, 4H), 1.97 (m, 4H)
Compound No. 308: Benzo[1,3]dioxol-5-ylmethyl-(2-piperidino-1-ylquinazolino-4-yl)-amine

¹H NMR (CDCl₃, 300 MHz) δ 7.48 (m, 3H), 7.03 (m, 1H), 6.85 (m, 3H), 5.95 (s, 2H), 5.64 (br s, 1H), 4.69 (t, J=5.4 Hz, 2H), 3.89 (m, 4H), 1.64 (m, 6H)
Compound No. 309: Benzo[1,3]dioxol-5-ylmethyl-(2-morpholino-4-ylquinazolino-4-yl)-amine

¹H NMR (CDCl₃, 300 MHz) δ 7.51 (m, 3H), 7.07 (t, J=7.4 Hz, 1H), 6.89 (s, 1H), 6.85 (d, J=7.8 Hz, 1H), 6.78 (d, J=7.8 Hz, 1H), 5.96 (s, 2H), 5.69 (br s, 1H), 4.70 (d, J=5.4 Hz, 2H), 3.89 (m, 4H), 3.78 (m, 4H)
Compound No. 310: Benzo[1,3]dioxol-5-ylmethyl-[2-(4-methylpiperazino-1-yl)-quinazolino-4-yl]-amine

¹H NMR (CDCl₃, 300 MHz) δ 7.48 (m, 3H), 7.05 (t, J=8.1 Hz, 1H), 6.89 (s, 1H), 6.85 (d, J=7.8 Hz, 1H), 6.78 (d, J=7.8 Hz, 1H), 5.96 (s, 2H), 5.68 (br s, 1H), 4.70 (d, J=5.4 Hz, 2H), 3.95 (br t, J=4.8 Hz, 4H), 2.48 (br t, J=4.8 Hz, 4H), 2.35 (s, 3H)
Compound No. 311: (2-Pyrrolidino-1-ylquinazolino-4-yl)-(3,4,5-trimethoxybenzyl)-amine

¹H NMR (CDCl₃, 300 MHz) δ 7.57 (br t, J=7.8 Hz, 2H), 7.48 (t, J=7.7 Hz, 1H), 7.02 (t, J=8.0 Hz, 1H), 6.66 (s, 2H), 6.11 (br s, 1H), 4.74 (d, J=5.4 Hz, 2H), 3.84 (s, 9H), 3.71 (br t, J=6.2 Hz, 4H), 1.98 (br t, J=6.9 Hz, 4H)
Compound No. 312: (2-Piperidino-1-ylquinazolino-4-yl)-(3,4,5-trimethoxybenzyl)-amine

¹H NMR (CDCl₃, 300 MHz) δ 7.50 (m, 3H), 7.03 (br t, J=8.0 Hz, 1H), 6.65 (s, 2H), 5.71 (br s, 1H), 4.72 (d, J=5.1 Hz, 2H), 3.91 (m, 4H), 3.84 (s, 9H), 1.65 (m, 6H)
Compound No. 313: (2-Morpholino-4-ylquinazolino-4-yl)-(3,4,5-trimethoxybenzyl)-amine

¹H NMR (CDCl₃, 300 MHz) δ 7.53 (m, 3H), 7.11 (br t, J=8.4 Hz, 1H), 6.63 (s, 2H), 5.74 (br s, 1H), 4.72 (d, J=5.4 Hz, 2H), 3.85 (m, 16H)
Compound No. 314: [2-(4-Methylpiperazino-1-yl)-quinazolino-4-yl]-(3,4,5-trimethoxybenzyl)-amine

¹H NMR (CDCl₃, 300 MHz) δ 7.53 (m, 3H), 7.06 (m, 1H), 6.64 (s, 2H), 5.77 (br S, 1H), 4.72 (d, J=5.1 Hz, 2H), 3.97 (br t, J=4.5 Hz, 4H), 3.85 (s, 9H), 2.50 (br t, J=5.0 Hz, 4H), 2.36 (s, 3H)
Compound No. 315: (2-Pyrrolidino-1-ylquinazolino-4-yl)-(2,4,6-trimethoxybenzyl)-amine

¹H NMR (CDCl₃, 300 MHz) δ 7.46 (m, 2H), 7.38 (d, J=8.1 Hz, 1H), 6.98 (m, 1H), 6.17 (s, 2H), 5.84 (br s, 1H), 4.81 (d, J=5.1 Hz, 2H), 3.85 (s, 6H), 3.83 (s, 3H), 3.74 (br t, J=6.5 Hz, 4H), 1.98 (m, 4H)
Compound No. 316: (2-Piperidino-1-ylquinazolino-4-yl)-(2,4,6-trimethoxybenzyl)-amine

¹H NMR (CDCl₃, 300 MHz) δ 7.41 (m, 3H), 6.98 (m, 1H), 6.16 (s, 2H), 5.86 (br s, 1H), 4.80 (d, J=5.1 Hz, 2H), 3.97 (m, 4H), 3.85 (s, 6H), 3.83 (s, 3H), 1.65 (m, 6H)
Compound No. 317: (2-Morpholino-4-ylquinazolino-4-yl)-(2,4,6-trimethoxybenzyl)-amine

¹H NMR (CDCl₃, 300 MHz) δ 7.47 (m, 3H), 7.02 (m, 1H), 6.17 (s, 2H), 5.93 (br s, 1H), 4.79 (d, J=5.1 Hz, 2H), 3.99 (m, 4H), 3.86 (m, 13H)
Compound No. 318: [2-(4-Methylpiperazino-1-yl)-quinazolino-4-yl]-(2,4,6-trimethoxybenzyl)-amine

¹H NMR (CDCl₃, 300 MHz) δ 7.45 (m, 3H), 6.98 (m, 1H), 6.16 (s, 2H), 5.91 (br s, 1H), 4.80 (d, J=5.1 Hz, 2H), 4.01 (br t, J=4.7 Hz, 4H), 3.83 (s, 6H), 3.79 (s, 3H), 2.52 (br t, J=5.0 Hz, 4H), 2.04 (s, 3H)
Compound No. 319: (2,4-Dimethoxybenzyl)-(2-ethoxyquinazolino-4-yl)-amine

¹H NMR (CDCl₃, 300 MHz) δ 7.60 (m, 2H), 7.52 (d, J=8.1 Hz, 1H), 7.23 (m, 2H), 6.48 (m, 2H), 6.11 (br s, 1H), 4.78 (d, J=5.4 Hz, 2H), 4.51 (quartet, J=7.2 Hz, 2H), 3.88 (s, 3H), 3.81 (s, 3H), 1.47 (t, J=7.2 Hz, 3H)
Compound No. 320: Benzo[1,3]dioxol-5-ylmethyl-(2-ethoxyquinazolino-4-yl)-amine

¹H NMR (CDCl₃, 300 MHz) δ 7.63 (m, 3H), 7.24 (m, 1H), 6.82 (m, 3H), 5.96 (s, 2H), 5.79 (br s, 1H), 4.75 (d, J=5.4 Hz, 2H), 4.50 (quartet, J=7.2 Hz, 2H), 1.46 (t, J=7.2 Hz, 3H)
Compound No. 321: (2-Isopropylquinazolino-4-yl)-(2-methoxybenzyl)-amine

¹H NMR (CDCl₃, 300 MHz) δ 8.01 (d, J=8.1 Hz, 1H), 7.81 (d, J=8.1 Hz, 1H), 7.69 (t, J=7.3 Hz, 1H), 7.43 (t, J=7.4 Hz, 2H), 7.31 (m, 1H), 6.93 (t, J=7.0 Hz, 2H), 4.95 (d, J=5.8 Hz, 2H), 3.93 (s, 3H), 3.31 (m, 1H), 1.41 (d, J=6.8 Hz, 6H)
Compound No. 322: (2,3-Dimethoxybenzyl)-(2-isopropylquinazolino-4-yl)-amine

¹H NMR (CDCl₃, 300 MHz) δ 7.97 (d, J=8.2 Hz, 1H), 7.82 (d, J=8.3 Hz, 1H), 7.69 (t, J=7.3 Hz, 1H), 7.41 (t, J=7.6 Hz, 1H), 7.03 (m, 2H), 6.89 (m, 1H), 4.96 (d, J=5.8 Hz, 2H), 3.96 (s, 3H), 3.89 (3, 3H), 3.30 (t, J=7.1 Hz, 1H), 1.79 (d, J=6.8 Hz, 6H)
Compound No. 323: (3,5-Dimethoxybenzyl)-(2-isopropylquinazolino-4-yl)-amine

¹H NMR (CDCl₃, 300 MHz) δ 8.21 (d, J=6.3 Hz, 1H), 8.02 (d, J=8.4 Hz, 1H), 7.63 (t, J=7.7 Hz, 1H), 7.37 (t, J=7.5 Hz, 1H), 6.65 (d, J=2.1 Hz, 2H), 6.36 (d, J=2.0 Hz, 1H), 4.90 (d, J=5.5 Hz, 2H), 3.75 (s, 6H), 3.34 (t, J=7.0 Hz, 1H), 1.38 (d, J=6.7 Hz, 6H)
Compound No. 324: (3,4-Dimethoxybenzyl)-(2-isopropylquinazolino-4-yl)-amine

¹H NMR (CDCl₃, 300 MHz) δ 8.17 (d, J=6.2 Hz, 1H), 7.97 (d, J=8.5 Hz, 1H), 7.62 (t, J=7.7 Hz, 1H), 7.34 (t, J=7.5 Hz, 1H), 7.12 (s, 1H), 7.05 (d, J=8.1 Hz, 1H), 6.82 (d, J=8.2 Hz, 1H), 4.90 (d, J=5.5 Hz, 2H), 3.86 (s, 6H), 3.33 (t, J=7.1 Hz, 1H), 1.40 (d, J=6.7 Hz, 6H)
Compound No. 325: (2-Isopropylquinazolino-4-yl)-(3,4,5-trimethoxybenzyl)-amine

¹H NMR (CDCl₃, 300 MHz) δ 8.22 (d, J=6.2 Hz, 1H), 8.00 (d, J=8.5 Hz, 1H), 7.64 (t, J=7.7 Hz, 1H), 7.36 (t, J=7.4 Hz, 1H), 6.79 (s, 2H), 4.90 (d, J=5.5 Hz, 2H), 3.84 (s, 9H), 3.34 (t, J=7.2 Hz, 1H), 1.39 (d, J=6.7 Hz, 6H)
Compound No. 326: (2,5-Dimethoxybenzyl)-(2-isopropylquinazolino-4-yl)-amine

¹H NMR (CDCl₃, 300 MHz) δ 8.06 (d, J=8.1 Hz, 1H), 7.86 (d, J=8.0 Hz, 1H), 7.70 (t, J=7.3 Hz, 1H), 7.43 (t, J=7.4 Hz, 1H), 7.01 (s, 1H), 6.80 (m, 2H), 4.93 (d, J=5.6 Hz, 2H), 3.89 (s, 3H), 3.75 (s, 3H), 3.36 (t, J=7.4 Hz, 1H), 1.40 (d, J=6.9 Hz, 6H)
Compound No. 327: (2-Isopropylquinazolino-4-yl)-(2,4,6-trimethoxybenzyl)-amine

¹H NMR (CDCl₃, 300 MHz) δ 8.09 (d, J=8.0 Hz, 1H), 7.73 (d, J=8.1 Hz, 1H), 7.65 (t, J=7.2 Hz, 1H), 7.41 (t, J=7.3 Hz, 1H), 6.31 (br, 1H), 6.18 (s, 2H), 4.97 (d, J=5.3 Hz, 2H), 3.88 (s, 9H), 3.34 (t, J=7.3 Hz, 1H), 1.42 (d, J=6.5 Hz, 6H)
Compound No. 328: (2-Benzylquinazolino-4-yl)-(2-methoxybenzyl)-amine

¹H NMR (CDCl₃, 300 MHz) δ 7.78 (d, J=8.1 Hz, 1H), 7.65 (m, 1H), 7.58 (d, J=8.1 Hz, 1H), 7.37-7.18 (m, 6H), 6.87 (d, J=8.4 Hz, 1H), 6.83 (t, J=7.2 Hz, 1H), 6.21 (br, 1H), 4.81 (d, J=5.7 Hz, 2H), 4.20 (s, 2H), 3.88 (s, 3H)
Compound No. 329: (2-Benzylquinazolino-4-yl)-(2,3-dimethoxybenzyl)-amine

¹H NMR (CDCl₃, 300 MHz) δ 7.78 (d, J=8.1 Hz, 1H), 7.67 (dd, J₁=6.7 Hz, J₂=0.9 Hz, 1H), 7.60 (d, J=8.4 Hz, 1H), 7.46 (d, J=6.9 Hz, 2H), 7.36-7.17 (m, 4H), 6.93 (t, J=8.1 Hz, 1H), 6.83 (m, 2H), 6.22 (br, 1H), 4.83 (d, J=5.7 Hz, 2H), 4.20 (s, 2H), 3.90 (s, 3H), 3.86 (s, 3H)
Compound No. 330: (2-Benzylquinazolino-4-yl)-(3,5-dimethoxybenzyl)-amine

¹H NMR (CDCl₃, 300 MHz) δ 7.89 (t, J=9.0 Hz, 2H), 7.62 (t, J=7.7 Hz, 1H), 7.48 (d, J=7.1 Hz, 2H), 7.31 (m, 4H), 6.55 (d, J=1.9 Hz, 2H), 6.37 (s, 1H), 4.81 (d, J=5.3 Hz, 2H), 4.22 (s, 2H), 3.73 (s, 6H)
Compound No. 331: (2-Benzylquinazolino-4-yl)-(3,4-dimethoxybenzyl)-amine

¹H NMR (CDCl₃, 300 MHz) δ 8.11 (d, J=6.1 Hz, 1H), 7.91 (d, J=8.2 Hz, 1H), 7.52 (m, 3H), 7.23 (m, 4H), 7.02 (s, 1H), 6.92 (m, 1H), 6.75 (d, J=8.2 Hz, 1H), 4.83 (d, J=5.2 Hz, 2H), 4.27 (s, 2H), 3.86 (s, 3H), 3.69 (s, 3H)
Compound No. 332: (2-Benzylquinazolino-4-yl)-(3,4,5-trimethoxybenzyl)-amine

¹H NMR (CDCl₃, 300 MHz) δ 8.03 (d, J=7.6 Hz, 1H), 7.91 (d, J=8.4 Hz, 1H), 7.61 (t, J=7.7 Hz, 1H), 7.50 (d, J=6.8 Hz, 2H), 7.32 (t, J=7.6 Hz, 1H), 7.23 (m, 3H), 6.66 (s, 2H), 4.81 (d, J=5.2 Hz, 2H), 4.25 (s, 2H), 3.86 (s, 9H)
Compound No. 333: (2-Benzylquinazolino-4-yl)-(2,4-dimethoxybenzyl)-amine

¹H NMR (CDCl₃, 300 MHz) δ 7.95 (d, J=8.1 Hz, 1H), 7.68 (m, 2H), 7.54 (m, 2H), 7.40-7.24 (m, 4H), 7.10 (d, J=8.3 Hz, 1H), 6.45 (d, J=2.23 Hz, 1H), 6.35 (m, 1H), 4.79 (d, J=5.5 Hz, 2H), 4.28 (s, 2H), 3.86 (s, 3H), 3.81 (s, 3H)
Compound No. 334: (2-Benzylquinazolino-4-yl)-(2,5-dimethoxybenzyl)-amine

¹H NMRCDCl₃, 300 MHz) δ 8.26 (d, J=8.3 Hz, 1H), 8.13 (d, J=8.2 Hz, 1H), 7.61 (m, 3H), 7.40 (t, J=7.7 Hz, 1H), 7.24 (m, 4H), 7.79 (m, 3H), 4.94 (d, J=5.4 Hz, 2H), 4.38 (s, 2H), 3.85 (s, 3H), 3.68 (s, 3H)
Compound No. 335: (2-Benzylquinazolino-4-yl)-(2,4,6-trimethoxybenzyl)-amine

¹H NMR (CDCl₃, 300 MHz) δ 8.12 (d, J=8.1 Hz, 1H), 7.63 (m, 4H), 7.35 (m, 4H), 6.17 (s, 2H), 4.96 (d, J=5.4 Hz, 2H), 4.40 (s, 2H), 3.85 (s, 9H)
Compound No. 336: (2-Phenylquinazolino-4-yl)-(3,4,5-trimethoxybenzyl)-amine
Compound No. 337: (2-Phenylquinazolino-4-yl)-(2,4,6-trimethoxybenzyl)-amine

¹H NMR (CDCl₃, 300 MHz) δ 8.76 (d, J=5.4 Hz, 2H), 7.69 (m, 2H), 7.54 (m, 3H), 7.41 (t, J=7.6 Hz, 1H), 7.28 (s, 1H), 6.19 (s, 2H), 5.00 (d, 2H, J=5.1 Hz), 3.90 (s, 9H)
Compound No. 338: (2-Furano-2-ylquinazolino-4-yl)-(2-methoxybenzyl)-amine

¹H NMR (CDCl₃, 300 MHz) δ 8.01 (d, J=8.4 Hz, 1H), 7.70 (m, 3H), 7.48 (d, J=6.6 Hz, 2H), 7.39 (t, J=7.5 Hz, 1H), 7.29 (d, J=7.8 Hz, 1H), 6.95 (t, J=7.4 Hz, 2H), 6.60 (m, 1H), 6.87 (br, 1H), 4.97 (d, J=5.3 Hz, 2H), 3.93 (s, 3H)
Compound No. 339: (2-Furano-2-ylquinazolino-4-yl)-(3-methoxybenzyl)-amine

¹H NMR (CDCl₃, 300 MHz) δ 8.01 (d, J=8.4 Hz, 1H), 7.78 (d, J=8.1 Hz, 1H), 7.67 (m, 2H), 7.42 (m, 2H), 7.27 (m, 2H), 6.87 (d, J=2.0 Hz, 1H), 6.58 (m, 1H), 6.50 (br, 1H), 4.94 (d, J=5.2 Hz, 2H), 3.80 (s, 3H)
Compound No. 340: (2-Furano-2-ylquinazolino-4-yl)-(4-methoxybenzyl)-amine

¹H NMR (CDCl₃, 300 MHz) δ 8.01 (d, J=8.3 Hz, 1H), 7.77 (d, J=8.1 Hz, 1H), 7.67 (t, J=7.2 Hz, 2H), 7.41 (m, 4H), 6.90 (d, J=8.6 Hz, 2H), 6.59 (m, 1H), 6.50 (br, 1H), 4.89 (d, J=5.5 Hz, 2H), 3.82 (s, 3H)
Compound No. 341: Benzo[1,3]dioxol-5-ylmethyl-(2-furano-2-ylquinazolino-4-yl)-amine

¹H NMR (CDCl₃, 300 MHz) δ 7.99 (d, J=8.3 Hz, 1H), 7.78 (d, J=8.1 Hz, 1H), 7.65 (m, 2H), 7.41 (t, J=5.6 Hz, 2H), 6.94 (t, J=7.6 Hz, 2H), 6.78 (d, J=7.9 Hz, 1H), 6.57 (m, 1H), 6.41 (br, 1H), 5.95 (s, 2H), 4.85 (d, J=5.4 Hz, 2H)
Compound No. 342: (2,3-Dimethoxybenzyl)-(2-furano-2-ylquinazolino-4-yl)-amine

¹H NMR (CDCl₃, 300 MHz) δ 8.02 (d, J=8.2 Hz, 1H), 7.71 (m, 3H), 7.52 (d, J=2.3 Hz, 1H), 7.39 (m, 1H), 7.07 (t, J=4.9 Hz, 2H), 6.90 (m, 1H), 6.67 (br, 1H), 6.60 (m, 1H), 4.49 (d, J=5.5 Hz, 2H)
Compound No. 343: (3,5-Dimethoxybenzyl)-(2-furano-2-ylquinazolino-4-yl)-amine

¹H NMR (CDCl₃, 300 MHz) δ 8.02 (d, J=8.3 Hz, 1H), 7.84 (d, J=8.2 Hz, 1H), 7.68 (m, 2H), 7.45 (d, J=3.1 Hz, 1H), 7.38 (m, 1H), 6.65 (m, 2H), 6.64 (br, 1H), 6.57 (m, 1H), 6.40 (t, J=2.1 Hz, 1H), 4.90 (d, J=5.3 Hz, 2H), 3.77 (s, 6H)
Compound No. 344: (3,4-Dimethoxybenzyl)-(2-furano-2-ylquinazolino-4-yl)-amine

¹H NMR (CDCl₃, 300 MHz) δ 8.00 (d, J=8.3 Hz, 1H), 7.79 (d, J=8.1 Hz, 1H), 7.66 (t, J=2.3 Hz, 2H), 7.47 (d, J=3.2 Hz, 1H), 7.36 (m, 1H), 7.09 (s, 1H), 7.01 (m, 1H), 6.86 (d, J=8.2 Hz, 1H), 6.59 (m, 1H), 6.46 (br, 1H), 4.99 (d, J=5.3 Hz, 2H), 3.88 (s, 3H), 3.85 (s, 3H)
Compound No. 345: (2-Furano-2-ylquinazolino-4-yl)-(3,4,5-trimethoxybenzyl)-amine

¹H NMR (CDCl₃, 300 MHz) δ 8.01 (d, J=8.3 Hz, 1H), 7.86 (d, J=8.3 Hz, 1H), 7.65 (t, J=6.2 Hz, 2H), 7.54 (d, J=3.1 Hz, 1H), 7.34 (t, J=7.5 Hz, 1H), 6.80 (br, 1H), 6.77 (s, 2H), 6.60 (m, 1H), 4.99 (d, J=5.4 Hz, 2H), 3.87 (s, 9H)
Compound No. 346: (2,4-Dimethoxybenzyl)-(2-furano-2-ylquinazolino-4-yl)-amine

¹H NMR (CDCl₃, 300 MHz) δ 8.01 (d, J=8.2 Hz, 1H), 7.69 (m, 3H), 7.51 (d, J=4.6 Hz, 1H), 7.38 (t, J=7.4 Hz, 2H), 6.60 (m, 1H), 6.48 (m, 3H), 4.88 (d, J=5.4 Hz, 2H), 3.89 (s, 3H), 3.81 (s, 3H)
Compound No. 347: (2,5-Dimethoxybenzyl)-(2-furano-2-ylquinazolino-4-yl)-amine

¹H NMR (CDCl₃, 300 MHz) δ 8.01 (d, J=8.3 Hz, 1H), 7.70 (m, 3H), 7.50 (d, J=2.9 Hz, 1H), 7.41 (d, J=7.3 Hz, 1H), 7.10 (d, J=2.8 Hz, 1H), 6.84 (m, 2H), 6.58 (m, 1H), 4.94 (d, J=5.4 Hz, 2H), 3.89 (s, 3H), 3.74 (s, 3H)
Compound No. 348: (2-Furano-2-ylquinazolino-4-yl)-(2,4,6-trimethoxybenzyl)-amine

¹H NMR (CDCl₃, 300 MHz) δ 8.01 (d, J=8.2 Hz, 1H), 7.67 (m, 4H), 7.37 (t, J=7.5 Hz, 1H), 6.60 (m, 1H), 6.21 (br, 1H), 6.17 (s, 2H), 4.96 (d, J=5.4 Hz, 2H), 3.87 (s, 9H)
Compound No. 349: (2-Methoxybenzyl)-(2-thiopenyl-2-ylquinazolino-4-yl)-amine

¹H NMR (CDCl₃, 300 MHz) δ 8.21 (d, J=3.3 Hz, 1H), 7.91 (d, J=8.3 Hz, 1H), 7.68 (m, 2H), 7.52 (m, 2H), 7.37 (t, J=7.6 Hz, 2H), 7.29 (d, J=7.7 Hz, 1H), 7.19 (t, J=4.2 Hz, 1H), 6.95 (t, J=7.7 Hz, 2H), 6.49 (br, 1H), 4.98 (d, J=5.3 Hz, 2H), 3.95 (s, 3H)
Compound No. 350: (3-Methoxybenzyl)-(2-thiopenyl-2-ylquinazolino-4-yl)-amine

¹H NMR (CDCl₃, 300 MHz) δ 8.17 (d, J=3.2 Hz, 1H), 7.91 (d, J=8.3 Hz, 1H), 7.71 (m, 2H), 7.47 (m, 1H), 7.37 (m, 1H), 7.28 (m, 1H), 7.16 (m, 1H), 7.06 (d, J=7.4 Hz, 2H), 6.84 (m, 1H), 6.30 (br, 1H), 4.94 (d, J=5.5 Hz, 2H), 3.79 (s, 3H)
Compound No. 351: (4-Methoxybenzyl)-(2-thiopenyl-2-ylquinazolino-4-yl)-amine

¹H NMR (CDCl₃, 300 MHz) δ 8.17 (d, J=3.1 Hz, 1H), 7.90 (d, J=8.5 Hz, 1H), 7.70 (m, 2H), 7.46 (m, 3H), 7.36 (m, 1H), 7.17 (t, J=4.3 Hz, 1H), 6.91 (d, J=8.5 Hz, 2H), 6.23 (br, 1H), 4.90 (d, J=5.3 Hz, 2H), 3.82 (s, 3H)
Compound No. 352: Benzo[1,3]dioxol-5-ylmethyl-(2-thiopenyl-2-ylquinazolino-4-yl)-amine

¹H NMR (CDCl₃, 300 MHz) δ 8.10 (d, J=3.1 Hz, 1H), 7.88 (d, J=8.5 Hz, 1H), 7.69 (m, 2H), 7.46 (t, J=2.5 Hz, 1H), 7.38 (m, 1H), 7.17 (m, 1H), 6.96 (t, J=7.5 Hz, 2H), 6.80 (d, J=7.9 Hz, 1H), 6.11 (br, 1H), 5.96 (s, 2H), 4.87 (d, J=5.3 Hz, 2H)
Compound No. 353: (2,3-Dimethoxybenzyl)-(2-thiopenyl-2-ylquinazolino-4-yl)-amine

¹H NMR (CDCl₃, 300 MHz) δ 8.21 (d, J=3.2 Hz, 1H), 7.91 (d, J=8.2 Hz, 1H), 7.69 (m, 2H), 7.49 (d, J=4.8 Hz, 1H), 7.36 (m, 1H), 7.17 (m, 2H), 7.04 (m, 1H), 6.90 (d, J=7.2 Hz, 1H), 6.45 (br, 1H), 5.00 (d, J=5.4 Hz, 2H), 3.98 (s, 3H), 3.90 (s, 3H)
Compound No. 354: (3,5-Dimethoxybenzyl)-(2-thiopenyl-2-ylquinazolino-4-yl)-amine

¹H NMR (CDCl₃, 300 MHz) δ 8.20 (d, J=3.3 Hz, 1H), 7.92 (d, J=8.4 Hz, 1H), 7.76 (d, J=10.9 Hz, 1H), 7.68 (t, J=7.7 Hz, 1H), 7.48 (d, J=4.9 Hz, 1H), 7.36 (t, J=7.6 Hz, 1H), 7.16 (m, 1H), 6.66 (s, 2H), 6.41 (s, 1H), 4.90 (d, J=5.4 Hz, 2H), 3.78 (s, 3H)
Compound No. 355: (3,4-Dimethoxybenzyl)-(2-thiopenyl-2-ylquinazolino-4-yl)-amine

¹H NMR (CDCl₃, 300 MHz) δ 8.19 (d, J=3.0 Hz, 1H), 7.91 (d, J=8.5 Hz, 1H), 7.67 (m, 2H), 7.49 (t, J=2.5 Hz, 1H), 7.35 (m, 1H), 7.17 (m, 1H), 7.09 (m, 1H), 7.01 (m, 1H), 6.85 (d, J=8.1 Hz, 1H), 6.38 (br, 1H), 4.89 (d, J=5.3 Hz, 2H), 3.88 (s, 3H), 3.84 (s, 3H)
Compound No. 356: (2-Thiopenyl-2-ylquinazolino-4-yl)-(3,4,5-trimethoxybenzyl)-amine

¹H NMR (CDCl₃, 300 MHz) δ 8.19 (d, J=3.1 Hz, 1H), 7.91 (d, J=8.4 Hz, 1H), 7.79 (d, J=8.1 Hz, 1H), 7.68 (t, J=7.6 Hz, 1H), 7.48 (d, J=5.0 Hz, 1H), 7.36 (t, J=7.6 Hz, 1H), 7.17 (t, J=4.3 Hz, 1H), 6.75 (s, 2H), 6.43 (br, 1H), 4.88 (d, J=5.4 Hz, 2H), 3.85 (s, 9H)
Compound No. 357: (2,4-Dimethoxybenzyl)-(2-thiopenyl-2-ylquinazolino-4-yl)-amine

¹H NMR (CDCl₃, 300 MHz) δ 8.23 (d, J=3.0 Hz, 1H), 7.91 (d, J=8.2 Hz, 1H), 7.65 (m, 2H), 7.48 (t, J=7.6 Hz, 2H), 7.38 (d, J=7.9 Hz, 1H), 7.19 (m, 1H), 6.48 (m, 3H), 4.90 (d, J=5.4 Hz, 2H), 3.93 (s, 3H), 3.80 (s, 3H)
Compound No. 358: (2,5-Dimethoxybenzyl)-(2-thiopenyl-2-ylquinazolino-4-yl)-amine

¹H NMR (CDCl₃, 300 MHz) δ 8.22 (d, J=3.1 Hz, 1H), 7.91 (d, J=8.5 Hz, 1H), 7.69 (m, 2H), 7.49 (t, J=3.5 Hz, 1H), 7.37 (m, 1H), 7.18 (m, 2H), 6.81 (t, J=4.8 Hz, 2H), 6.52 (br, 1H), 4.95 (d, J=5.3 Hz, 2H), 3.90 (s, 3H), 3.74 (s, 3H)
Compound No. 359: (2-Thiopenyl-2-ylquinazolino-4-yl)-(2,4,6-trimethoxybenzyl)-amine

¹H NMR (CDCl₃, 300 MHz) δ 8.28 (d, J=3.2 Hz, 1H), 7.93 (d, J=8.4 Hz, 1H), 7.65 (m, 2H), 7.49 (d, J=4.8 Hz, 1H), 7.35 (t, J=7.6 Hz, 1H), 7.19 (t, J=4.3 Hz, 1H), 6.20 (br, 1H), 6.18 (s, 2H), 5.00 (d, J=5.3 Hz, 2H), 3.88 (s, 9H)
Compound No. 360: (2-Methoxybenzyl)-[2-(methoxyphenylmethyl)-quinazolino-4-yl]-amine

¹H NMR (CDCl₃, 300 MHz) δ 7.90 (d, J=8.4 Hz, 1H), 7.68-7.55 (m, 4H), 7.38-7.20 (m, 6H), 6.85 (m, 2H), 6.24 (br, 1H), 5.41 (s, 1H), 4.84 (d, J=6.0 Hz, 2H), 3.87 (s, 3H), 3.50 (s, 3H)
Compound No. 361: (3-Methoxybenzyl)-[2-(methoxyphenylmethyl)-quinazolino-4-yl]-amine

¹H NMR (CDCl₃, 300 MHz) δ 7.95 (d, J=8.3 Hz, 1H), 7.75 (d, J=8.1 Hz, 1H), 7.64 (m, 3H), 7.28 (m, 5H), 6.94 (s, 2H), 6.83 (d, J=8.1 Hz, 1H), 6.59 (br, 1H), 5.42 (s, 1H), 4.83 (d, J=4.9 Hz, 2H), 3.76 (s, 3H), 3.49 (s, 3H)
Compound No. 362: (4-Methoxybenzyl)-[2-(methoxyphenylmethyl)-quinazolino-4-yl]-amine

¹H NMR (CDCl₃, 300 MHz) δ 7.93 (d, J=8.4 Hz, 1H), 7.70-7.57 (m, 4H), 7.39-7.22 (m, 6H), 6.81 (d, J=8.4 Hz, 2H), 5.89 (br, 1H), 5.42 (s, 1H), 4.76 (d, J=5.4 Hz, 2H), 3.79 (s, 3H), 3.50 (s, 3H)
Compound No. 363: Benzo[1,3]dioxol-5-ylmethyl-[2-(methoxyphenylmethyl)-quinazolino-4-yl]-amine

¹H NMR (CDCl₃, 300 MHz) δ 7.95 (d, J=8.3 Hz, 1H), 7.81 (d, J=8.1 Hz, 1H), 7.64 (m, 2H), 7.35 (m, 5H), 6.83 (m, 2H), 6.70 (d, J=7.9 Hz, 1H), 5.93 (s, 2H), 5.46 (s, 1H), 4.76 (d, J=5.1 Hz, 2H), 3.51 (s, 3H)
Compound No. 364: (2,3-Dimethoxybenzyl)-[2-(methoxyphenylmethyl)-quinazolino-4-yl]-amine

¹H NMR (CDCl₃, 300 MHz) δ 8.00 (d, J=8.3 Hz, 1H), 7.69 (m, 3H), 7.32 (m, 5H), 6.89 (m, 3H), 5.50 (s, 1H), 4.90 (d, J=5.2 Hz, 2H), 3.93 (s, 3H), 3.88 (s, 3H), 3.51 (s, 3H)
Compound No. 365: (3,5-Dimethoxybenzyl)-[2-(methoxyphenylmethyl)-quinazolino-4-yl]-amine

¹H NMR (CDCl₃, 300 MHz) δ 7.95 (d, J=8.3 Hz, 1H), 7.77 (d, 1H, J=8.1 Hz), 7.65 (m, 3H), 7.31 (m, 5H), 6.60 (br, 1H), 6.55 (s, 2H), 6.39 (d, J=2.0 Hz, 1H), 5.44 (s, 1H), 4.81 (d, J=5.1 Hz, 2H), 3.75 (s, 6H), 3.49 (s, 3H)
Compound No. 366: (3,4-Dimethoxybenzyl)-[2-(methoxyphenylmethyl)-quinazolino-4-yl]-amine

¹H NMR (CDCl₃, 300 MHz) δ 7.94 (d, J=8.4 Hz, 1H), 7.78 (d, J=8.1 Hz, 1H), 7.68 (d, J=6.9 Hz, 2H), 7.58 (t, J=7.6 Hz, 1H), 7.33 (m, 5H), 6.96 (s, 1H), 6.91 (t, J=4.1 Hz, 1H), 5.45 (s, 1H), 4.80 (d, J=5.2 Hz, 2H), 3.88 (s, 3H), 3.80 (s, 3H), 3.50 (s, 3H)
Compound No. 367: [2-(Methoxyphenylmethyl)-quinazolino-4-yl]-(3,4,5-trimethoxybenzyl)-amine

¹H NMR (CDCl₃, 300 MHz) δ 7.95 (d, J=8.3 Hz, 1H), 7.83 (d, J=8.2 Hz, 1H), 7.63 (m, 3H), 7.30 (m, 5H), 6.75 (br, 1H), 6.64 (s, 2H), 5.45 (s, 1H), 4.80 (d, J=5.2 Hz, 2H), 3.78 (s, 9H), 3.48 (s, 3H)
Compound No. 368: (2,4-Dimethoxybenzyl)-[2-(methoxyphenylmethyl)-quinazolino-4-yl]-amine

¹H NMR (CDCl₃, 300 MHz) δ 7.97 (d, J=8.3 Hz, 1H), 7.67 (m, 4H), 7.33 (m, 5H), 7.18 (d, J=8.3 Hz, 1H), 6.48 (br, 1H), 6.45 (s, 1H), 6.35 (m, 1H), 5.50 (s, 1H), 4.79 (d, J=5.1 Hz, 2H), 8.85 (s, 3H), 3.79 (s, 3H), 3.52 (s, 3H)
Compound No. 369: (2,5-Dimethoxybenzyl)-[2-(methoxyphenylmethyl)-quinazolino-4-yl]-amine

¹H NMR (CDCl₃, 300 MHz) δ 7.96 (d, J=8.3 Hz, 1H), 7.66 (m, 4H), 7.34 (m, 5H), 6.97 (s, 1H), 6.80 (t, J=3.8 Hz, 1H), 6.54 (br, 1H), 5.47 (s, 1H), 4.86 (d, J=5.0 Hz, 2H), 3.86 (s, 3H), 3.72 (s, 3H), 3.50 (s, 3H)
Compound No. 370: [2-(Methoxyphenylmethyl)-quinazolino-4-yl]-(2,4,6-trimethoxybenzyl)-amine

¹H NMR (CDCl₃, 300 MHz) δ 7.89 (d, J=8.1 Hz, 1H), 7.75 (dd, J₁=8.4 Hz, J₂=1.2 Hz, 2H), 7.63 (td, J₁=6.9 Hz, J₂=1.2 Hz, 1H), 7.51 (d, J=7.2 Hz, 1H), 7.35-7.21 (m, 4H), 6.16 (s, 2H), 5.98 (br, 1H), 5.43 (s, 1H), 4.87 (d, J=5.4 Hz, 2H), 3.82 (s, 3H), 3.80 (s, 6H), 3.52 (s, 3H)
Compound No. 371: (2-Methoxybenzyl)-[2-(1-phenylethyl)-quinazolino-4-yl]-amine

¹H NMR (CDCl₃, 300 MHz) δ 7.85 (d, J=8.6 Hz, 1H), 7.64 (m, 2H), 7.56 (d, J=7.4 Hz, 2H), 7.30 (m, 6H), 6.90 (t, J=7.9 Hz, 2H), 6.55 (br, 1H), 4.90 (d, J=5.4 Hz, 2H), 4.40 (d, J=7.0 Hz, 1H), 3.92 (s, 3H), 1.79 (d, J=7.1 Hz, 3H)
Compound No. 372: (3-Methoxybenzyl)-[2-(1-phenylethyl)-quinazolino-4-yl]-amine

¹H NMR (CDCl₃, 300 MHz) δ 7.85 (d, J=8.6 Hz, 1H), 7.74 (d, J=8.1 Hz, 1H), 7.63 (m, 1H), 7.51 (d, J=7.2 Hz, 2H), 7.25 (m, 5H), 6.97 (d, J=7.9 Hz, 2H), 6.86 (d, J=2.0 Hz, 1H), 4.88 (d, J=5.4 Hz, 2H), 4.36 (m, 1H), 3.77 (s, 3H), 1.75 (d, J=7.1 Hz, 3H)
Compound No. 373: (4-Methoxybenzyl)-[2-(1-phenylethyl)-quinazolino-4-yl]-amine

¹H NMR (CDCl₃, 300 MHz) δ 7.84 (d, J=8.5 Hz, 1H), 7.73 (d, J=8.1 Hz, 1H), 7.62 (t, J=7.6 Hz, 1H), 7.53 (d, J=7.6 Hz, 2H), 7.27 (m, 6H), 6.85 (d, J=7.6 Hz, 2H), 6.38 (br, 1H), 4.81 (d, J=5.5 Hz, 2H), 4.40 (m, 1H), 3.81 (s, 3H), 1.78 (d, J=7.3 Hz, 3H)
Compound No. 374: Benzo[1,3]dioxol-5-ylmethyl-[2-(1-phenylethyl)-quinazolino-4-yl]-amine

¹H NMR (CDCl₃, 300 MHz) δ 7.85 (d, J=8.4 Hz, 1H), 7.75 (d, J=8.5 Hz, 1H), 7.63 (m, 1H), 5.52 (d, J=7.5 Hz, 2H), 7.28 (m, 4H), 6.85 (t, J=6.7 Hz, 2H), 6.73 (d, J=7.8 Hz, 1H), 6.49 (br, 1H), 5.94 (s, 2H), 4.78 (d, J=5.4 Hz, 2H), 4.40 (m, 1H), 1.78 (d, J=7.2 Hz, 3H)
Compound No. 375: (2,3-Dimethoxybenzyl)-[2-(1-phenylethyl)-quinazolino-4-yl]-amine

¹H NMR (CDCl₃, 300 MHz) δ 7.85 (d, J=8.0 Hz, 1H), 7.65 (m, 2H), 7.55 (d, J=7.5 Hz, 2H), 7.28 (m, 5H), 6.91 (m, 3H), 6.54 (br, 1H), 4.91 (d, J=5.4 Hz, 2H), 4.41 (m, 1H), 3.94 (s, 3H), 3.89 (s, 3H), 1.78 (d, J=7.4 Hz, 3H)
Compound No. 376: (3,5-Dimethoxybenzyl)-[2-(1-phenylethyl)-quinazolino-4-yl]-amine

¹H NMR (CDCl₃, 300 MHz) δ 7.84 (d, J=8.3 Hz, 1H), 7.74 (d, J=8.1 Hz, 1H), 7.63 (m, 1H), 7.52 (d, J=7.3 Hz, 2H), 7.24 (m, 4H), 6.56 (s, 2H), 6.41 (s, 1H), 4.48 (d, J=5.3 Hz, 2H), 4.38 (m, 1H), 3.75 (s, 6H), 1.78 (d, J=7.1 Hz, 3H)
Compound No. 377: (3,4-Dimethoxybenzyl)-[2-(1-phenylethyl)-quinazolino-4-yl]-amine

¹H NMR (CDCl₃, 300 MHz) δ 7.83 (d, J=8.3 Hz, 1H), 7.75 (d, J=8.2 Hz, 1H), 7.62 (d, J=7.4 Hz, 1H), 7.53 (d, J=7.2 Hz, 2H), 7.25 (m, 5H), 6.94 (d, J=5.6 Hz, 2H), 6.82 (d, J=8.6 Hz, 1H), 6.32 (br, 1H), 4.84 (d, J=5.4 Hz, 2H), 4.38 (m, 1H), 3.89 (s, 3H), 3.79 (s, 3H), 1.78 (d, J=7.1 Hz, 3H)
Compound No. 378: [2-(1-Phenylethyl)-quinazolino-4-yl]-(3,4,5-trimethoxybenzyl)-amine

¹H NMR (CDCl₃, 300 MHz) δ 7.82 (t, J=9.2 Hz, 2H), 7.54 (m, 2H), 7.27 (m, 5H), 6.65 (s, 2H), 4.86 (d, J=5.4 Hz, 2H), 4.40 (m, 1H), 3.79 (s, 9H), 1.78 (d, J=7.1 Hz, 3H)
Compound No. 379: (2,4-Dimethoxybenzyl)-[2-(1-phenylethyl)-quinazolino-4-yl]-amine

¹H NMR (CDCl₃, 300 MHz) δ 7.85 (d, J=8.4 Hz, 1H), 7.60 (m, 4H), 7.32 (m, 3H), 7.21 (t, J=6.9 Hz, 2H), 6.48 (d, J=2.0 Hz, 1H), 6.39 (br, 1H), 6.36 (d, J=8.1 Hz, 1H), 4.48 (d, J=5.3 Hz, 2H), 4.40 (m, 1H), 3.88 (s, 3H), 3.82 (s, 3H), 1.80 (d, J=7.1 Hz, 3H)
Compound No. 380: (2,5-Dimethoxybenzyl)-[2-(1-phenylethyl)-quinazolino-4-yl]-amine

¹H NMR (CDCl₃, 300 MHz) δ 7.83 (d, J=8.2 Hz, 1H), 7.63 (m, 2H), 7.56 (d, J=7.4 Hz, 2H), 7.35 (m, 1H), 7.28 (m, 2H), 7.19 (d, J=7.3 Hz, 1H), 6.98 (d, J=2.8 Hz, 1H), 6.82 (m, 2H), 6.28 (br, 1H), 4.88 (d, J=5.4 Hz, 2H), 4.38 (m, 2H), 3.88 (s, 3H), 3.79 (s, 3H), 1.79 (d, J=7.1 Hz, 3H)
Compound No. 381: [2-(1-Phenylethyl)-quinazolino-4-yl]-(2,4,6-trimethoxybenzyl)-amine

¹H NMR (CDCl₃, 300 MHz) δ 7.76 (d, J=8.4 Hz, 1H), 7.64-7.50 (m, 4H), 7.31-7.24 (m, 3H), 7.16 (t, J=7.2 Hz, 1H), 6.17 (s, 2H), 6.03 (br, 1H), 5.02-4.83 (m, 2H), 4.31 (q, J=7.2 Hz, 1H), 3.83 (s, 9H), 1.80 (d, J=7.2 Hz, 3H)
Compound No. 382: (2-Cyclopropylquinazolino-4-yl)-(2-methoxybenzyl)-amine

¹H NMR (CDCl₃, 300 MHz) δ 7.75 (d, J=8.0 Hz, 1H), 7.62 (m, 2H), 7.35 (m, 3H), 6.93 (m, 2H), 6.32 (br, 1H), 4.81 (d, J=5.4 Hz, 2H), 3.98 (s, 3H), 2.19 (m, 1H), 1.25 (m, 2H), 1.01 (m, 2H)
Compound No. 383: (2-Cyclopropylquinazolino-4-yl)-(3-methoxybenzyl)-amine

¹H NMR (CDCl₃, 300 MHz) δ 7.77-7.61 (m, 3H), 7.35-7.24 (m, 2H), 6.94 (m, 2H), 6.84 (dd, J₁=8.4 Hz, J₂=2.7 Hz, 1H), 5.93 (br, 1H), 4.78 (d, J=5.7 Hz, 2H), 3.79 (s, 3H), 2.17 (m, 1H), 1.17 (m, 2H), 0.95 (m, 2H)
Compound No. 384: (2-Cyclopropylquinazolino-4-yl)-(4-methoxybenzyl)-amine

¹H NMR (CDCl₃, 300 MHz) δ 7.78 (d, J=7.9 Hz, 1H), 7.70 (m, 2H), 7.35 (m, 3H), 6.90 (d, J=7.8 Hz, 2H), 5.98 (br, 1H), 4.74 (d, J=5.4 Hz, 2H), 3.82 (s, 3H), 2.20 (m, 1H), 1.21 (m, 2H), 1.01 (m, 2H)
Compound No. 385: Benzo[1,3]dioxol-5-ylmethyl-(2-cyclopropylquinazolino-4-yl)-amine

¹H NMR (CDCl₃, 300 MHz) δ 7.76 (d, J=8.0 Hz, 1H), 7.66 (m, 2H), 7.43 (m, 1H), 6.84 (m, 3H), 5.96 (s, 2H), 4.71 (d, J=5.4 Hz, 2H), 2.19 (m, 1H), 1.20 (m, 2H), 1.01 (m, 2H)
Compound No. 386: (2-Cyclopropylquinazolino-4-yl)-(2,3-dimethoxybenzyl)-amine

¹H NMR (CDCl₃, 300 MHz) δ 7.74 (d, J=7.9 Hz, 1H), 7.64 (m, 2H), 7.32 (d, J=8.4 Hz, 1H), 6.99 (m, 2H), 6.89 (m, 1H), 6.32 (br, 1H), 4.83 (d, J=5.4 Hz, 2H), 3.94 (s, 3H), 3.89 (s, 3H), 2.18 (m, 1H), 1.22 (m, 2H), 1.01 (m, 2H)
Compound No. 387: (2-Cyclopropylquinazolino-4-yl)-(3,5-dimethoxybenzyl)-amine

¹H NMR (CDCl₃, 300 MHz) δ 7.76 (d, J=7.8 Hz, 1H), 7.66 (m, 2H), 7.35 (m, 1H), 6.54 (s, 2H), 6.39 (s, 1H), 6.23 (br, 1H), 4.74 (d, J=5.4 Hz, 2H), 3.79 (s, 6H), 2.18 (m, 1H), 1.19 (m, 2H), 0.99 (m, 2H)
Compound No. 388: (2-Cyclopropylquinazolino-4-yl)-(3,4-dimethoxybenzyl)-amine

¹H NMR (CDCl₃, 300 MHz) δ 7.76 (d, J=7.8 Hz, 1H), 7.67 (t, J=7.4 Hz, 2H), 7.31 (t, J=3.8 Hz, 1H), 6.94 (d, J=5.0 Hz, 2H), 6.84 (t, J=4.3 Hz, 1H), 6.07 (br, 1H), 4.74 (d, J=5.4 Hz, 2H), 3.89 (s, 3H), 3.87 (s, 3H), 2.20 (m, 1H), 1.22 (m, 2H), 1.00 (m, 2H)
Compound No. 389: (2-Cyclopropylquinazolino-4-yl)-(3,4,5-trimethoxybenzyl)-amine

¹H NMR (CDCl₃, 300 MHz) δ 7.77-7.64 (m, 3H), 7.33 (t, J=7.2 Hz, 1H), 6.61 (s, 1H), 6.00 (br, 1H), 4.72 (d, J=5.4 Hz, 2H), 3.84 (s, 9H), 2.19 (m, 1H), 1.19 (m, 2H), 0.98 (m, 2H)
Compound No. 390: (2-Cyclopropylquinazolino-4-yl)-(2,4-dimethoxybenzyl)-amine

¹H NMR (CDCl₃, 300 MHz) δ 7.73 (d, J=8.3 Hz, 1H), 7.62 (m, 2H), 7.32 (d, J=9.3 Hz, 2H), 6.48 (m, 2H), 6.14 (br, 1H), 4.73 (d, J=5.4 Hz, 2H), 3.89 (s, 3H), 3.81 (s, 3H), 2.19 (m, 1H), 1.25 (m, 2H), 1.01 (m, 2H)
Compound No. 391: (2-Cyclopropylquinazolino-4-yl)-(2,5-dimethoxybenzyl)-amine

¹H NMR (CDCl₃, 300 MHz) δ 7.74 (d, J=8.5 Hz, 1H), 7.63 (m, 2H), 7.33 (t, J=7.5 Hz, 1H), 6.98 (d, J=2.1 Hz, 1H), 6.81 (m, 2H), 6.25 (br, 1H), 3.88 (s, 3H), 3.78 (s, 3H), 2.18 (m, 1H), 1.25 (m, 2H), 1.01 (m, 2H)
Compound No. 392: (2-Cyclopropylquinazolino-4-yl)-(2,4,6-trimethoxybenzyl)-amine

¹H NMR (CDCl₃, 300 MHz) δ 7.73 (d, J=8.3 Hz, 1H), 7.64 (m, 1H), 7.53 (d, J=6.2 Hz, 1H), 7.29 (m, 1H), 2.17 (s, 2H), 6.06 (br, 1H), 4.82 (d, J=5.3 Hz, 2H), 3.87 (s, 6H), 3.84 (s, 3H), 2.18 (m, 1H), 1.30 (m, 2H), 1.00 (m, 2H)
Compound No. 393: (2-Methoxybenzyl)-(2-propylquinazolino-4-yl)-amine

¹H NMR (CDCl₃, 300 MHz) δ 7.78 (d, J=8.2 Hz, 1H), 7.68 (m, 2H), 7.44 (d, J=7.3 Hz, 1H), 7.38 (d, J=7.0 Hz, 1H), 7.34 (m, 1H), 6.95 (t, J=7.8 Hz, 2H), 6.24 (br, 1H), 4.91 (d, J=5.7 Hz, 2H), 3.93 (s, 3H), 2.87 (t, J=7.7 Hz, 2H), 1.93 (m, 2H), 1.04 (t, J=7.4 Hz, 3H)
Compound No. 394: (3-Methoxybenzyl)-(2-propylquinazolino-4-yl)-amine

¹H NMR (CDCl₃, 300 MHz) δ 7.80 (d, J=8.4 Hz, 1H), 7.68 (m, 2H), 7.35 (m, 2H), 6.99 (m, 2H), 6.85 (m, 1H), 5.83 (br s, 1H), 4.86 (d, J=5.1 Hz, 2H), 3.80 (s, 3H), 2.86 (t, J=7.7 Hz, 2H), 1.90 (sextet, J=7.5 Hz, 2H), 1.02 (t, J=7.4 Hz, 3H)
Compound No. 395: (4-Methoxybenzyl)-(2-propylquinazolino-4-yl)-amine

¹H NMR (CDCl₃, 300 MHz) δ 7.79 (d, J=8.4 Hz, 1H), 7.65 (m, 2H), 7.38 (m, 3H), 6.88 (d, J=8.4 Hz, 2H), 5.81 (br s, 1H), 4.80 (d, J=5.1 Hz, 2H), 3.81 (s, 3H), 2.86 (t, J=7.8 Hz, 2H), 1.91 (sextet, J=7.5 Hz, 2H), 1.03 (t, J=7.5 Hz, 3H)
Compound No. 396: Benzo[1,3]dioxol-5-ylmethyl-(2-propylquinazolino-4-yl)-amine

¹H NMR (CDCl₃, 300 MHz) δ 7.80 (d, J=7.8 Hz, 1H), 7.68 (m, 2H), 7.37 (t, J=7.5 Hz, 1H), 6.89 (m, 2H), 6.78 (d, J=7.8 Hz, 1H), 5.95 (s, 2H), 5.80 (br s, 1H), 4.78 (d, J=5.4 Hz, 2H), 2.86 (t, J=7.8 Hz, 2H), 1.91 (sextet, J=7.5 Hz, 2H), 1.03 (t, J=7.5 Hz, 3H)
Compound No. 397: (2,3-Dimethoxybenzyl)-(2-propylquinazolino-4-yl)-amine

¹H NMR (CDCl₃, 300 MHz) δ 7.76 (d, J=8.4 Hz, 1H), 7.64 (m, 2H), 7.37 (t, J=7.2 Hz, 1H), 7.03 (d, J=4.8 Hz, 2H), 6.88 (t, J=4.8 Hz, 1H), 6.17 (br s, 1H), 4.90 (d, J=5.7 Hz, 2H), 3.94 (s, 3H), 3.89 (s, 3H), 2.86 (t, J=7.8 Hz, 2H), 1.91 (sextet, J=7.5 Hz, 2H), 1.03 (t, J=7.5 Hz, 3H)
Compound No. 398: (3,5-Dimethoxybenzyl)-(2-propylquinazolino-4-yl)-amine

¹H NMR (CDCl₃, 300 MHz) δ 7.80 (d, J=8.1 Hz, 1H), 7.70 (m, 2H), 7.38 (t, J=7.2 Hz, 1H), 6.57 (d, J=2.1 Hz, 2H), 6.41 (m, 1H), 5.83 (br s, 1H), 4.82 (d, J=5.4 Hz, 2H), 3.78 (s, 6H), 2.86 (t, J=7.8 Hz, 2H), 1.91 (sextet, J=7.5 Hz, 2H), 1.03 (t, J=7.5 Hz, 3H)
Compound No. 399: (3,4-Dimethoxybenzyl)-(2-propylquinazolino-4-yl)-amine

¹H NMR (CDCl₃, 300 MHz) δ 7.80 (d, J=8.1 Hz, 1H), 7.69 (m, 2H), 7.37 (t, J=7.5 Hz, 1H), 6.97 (d, J=7.2 Hz, 2H), 6.85 (d, J=8.4 Hz, 1H), 5.81 (br s, 1H), 4.81 (d, J=5.4 Hz, 2H), 3.88 (s, 3H), 3.86 (s, 3H), 2.86 (t, J=7.8 Hz, 2H), 1.91 (sextet, J=7.5 Hz, 2H), 1.03 (t, J=7.5 Hz, 3H)
Compound No. 400: (2-Propylquinazolino-4-yl)-(3,4,5-trimethoxybenzyl)-amine

¹H NMR (CDCl₃, 300 MHz) δ 7.81 (d, J=8.3 Hz, 3H), 7.71 (m, 2H), 4.40 (m, 1H), 6.68 (s, 2H), 6.07 (br, 1H), 4.82 (d, J=5.5 Hz, 2H), 3.86 (s, 9H), 2.88 (t, J=7.7 Hz, 2H), 1.94 (m, 2H), 1.03 (t, J=7.4 Hz, 2H)
Compound No. 401: (2,4-Dimethoxybenzyl)-(2-propylquinazolino-4-yl)-amine

¹H NMR (CDCl₃, 300 MHz) δ 7.78 (d, J=8.5 Hz, 1H), 7.64 (m, 2H), 7.36 (m, 2H), 7.47 (m, 2H), 6.14 (br, 1H), 4.82 (d, J=5.4 Hz, 2H), 3.90 (s, 3H), 3.82 (s, 3H), 2.87 (t, J=7.8 Hz, 2H), 1.96 (m, 2H), 1.05 (t, J=7.3 Hz, 2H)
Compound No. 402: (2,5-Dimethoxybenzyl)-(2-propylquinazolino-4-yl)-amine

¹H NMR (CDCl₃, 300 MHz) δ 7.78 (d, J=8.1 Hz, 1H), 7.66 (m, 2H), 7.37 (m, 1H), 7.05 (s, 1H), 6.82 (m, 2H), 6.29 (br, 1H), 4.87 (d, J=5.4 Hz, 2H), 3.88 (s, 3H), 3.76 (s, 3H), 2.86 (t, J=7.6 Hz, 2H), 1.95 (m, 2H), 1.05 (t, J=7.5 Hz, 2H)
Compound No. 403: (2-Propylquinazolino-4-yl)-(2,4,6-trimethoxybenzyl)-amine

¹H NMR (CDCl₃, 300 MHz) δ 7.76 (d, J=8.3 Hz, 1H), 7.60 (m, 2H), 7.35 (m, 1H), 6.18 (s, 2H), 5.98 (br, 1H), 4.88 (d, J=5.4 Hz, 2H), 3.87 (s, 6H), 3.85 (3, 3H), 2.88 (t, J=7.5 Hz, 2H), 1.98 (m, 2H), 1.07 (t, J=7.4 Hz, 2H)
Compound No. 404: (2-Methoxybenzyl)-quinazolino-4-ylamine

¹H NMR (CDCl₃, 300 MHz) δ 8.74 (s, 1H), 7.88 (d, J=8.2 Hz, 1H), 7.70 (m, 2H), 7.47 (m, 2H), 7.32 (m, 1H), 6.97 (d, J=7.8 Hz, 2H), 6.25 (br, 1H), 4.88 (d, J=5.5 Hz, 2H), 3.93 (s, 3H)
Compound No. 405: (3-Methoxybenzyl)-quinazolino-4-ylamine

¹H NMR (CDCl₃, 300 MHz) δ 8.73 (s, 1H), 7.86 (d, J=8.3 Hz, 1H), 7.73 (t, J=9.0 Hz, 2H), 7.47 (m, 1H), 7.31 (m, 1H), 6.99 (t, J=7.4 Hz, 2H), 6.87 (d, J=8.1 Hz, 1H), 6.01 (br, 1H), 4.87 (d, J=5.4 Hz, 2H), 3.82 (s, 3H)
Compound No. 406: (4-Methoxybenzyl)-quinazolino-4-ylamine

¹H NMR (CDCl₃, 300 MHz) δ 8.71 (s, 1H), 7.85 (d, J=8.1 Hz, 1H), 7.71 (m, 2H), 7.45 (t, J=7.5 Hz, 1H), 7.34 (d, J=8.7 Hz, 2H), 6.90 (d, J=8.7 Hz, 1H), 5.88 (br s, 1H), 4.80 (d, J=5.1 Hz, 2H), 3.82 (s, 3H)
Compound No. 407: Benzo[1,3]dioxol-5-ylmethylquinazolino-4-ylamine

¹H NMR (CDCl₃, 300 MHz) δ 8.72 (s, 1H), 7.88 (d, J=8.4 Hz, 1H), 7.40 (m, 2H), 7.48 (d, J=7.2 Hz, 1H), 6.90 (m, 2H), 6.81 (d, J=7.4 Hz, 1H), 5.98 (s, 2H), 5.96 (br, 1H), 4.78 (d, J=5.4 Hz, 2H)
Compound No. 408: (2,3-Dimethoxybenzyl)-quinazolino-4-ylamine

¹H NMR (CDCl₃, 300 MHz) δ 8.70 (s, 1H), 7.73 (m, 3H), 7.43 (m, 1H), 7.04 (m, 2H), 6.93 (m, 1H), 6.32 (br, 1H), 4.89 (d, J=5.4 Hz, 2H), 3.95 (s, 3H), 3.90 (s, 3H)
Compound No. 409: (3,5-Dimethoxybenzyl)-quinazolino-4-ylamine

¹H NMR (CDCl₃, 300 MHz) δ 8.71 (s, 1H), 7.87 (d, J=8.4 Hz, 1H), 7.73 (m, 2H), 7.47 (t, J=7.2 Hz, 1H), 6.40 (d, J=2.4 Hz, 2H), 6.41 (t, J=2.1 Hz, 1H), 5.93 (br s, 1H), 4.81 (d, J=5.1 Hz, 2H), 3.79 (s, 6H)
Compound No. 410: (3,4-Dimethoxybenzyl)-quinazolino-4-ylamine

¹H NMR (CDCl₃, 300 MHz) δ 8.73 (s, 1H), 7.87 (d, J=8.2 Hz, 1H), 7.75 (t, J=7.4 Hz, 2H), 7.47 (t, J=7.6 Hz, 1H), 6.97 (d, J=7.8 Hz, 2H), 6.86 (d, J=8.0 Hz, 1H), 6.08 (br, 1H), 4.81 (d, J=5.2 Hz, 2H), 3.89 (s, 3H), 3.87 (s, 3H)
Compound No. 411: Quinazolino-4-yl-(3,4,5-trimethoxybenzyl)-amine

¹H NMR (CDCl₃, 300 MHz) δ 8.73 (s, 1H), 7.84 (m, 1H), 7.78 (m, 1H), 7.50 (m, 1H), 6.63 (d, J=7.8 Hz, 2H), 6.23 (br, 1H), 4.80 (d, J=5.3 Hz, 2H), 3.85 (s, 9H)
Compound No. 412: (2,4-Dimethoxybenzyl)-quinazolino-4-ylamine

¹H NMR (CDCl₃, 300 MHz) δ 8.70 (s, 1H), 7.83 (d, J=8.3 Hz, 1H), 7.72 (m, 2H), 7.44 (t, J=7.6 Hz, 1H), 7.32 (d, J=8.2 Hz, 1H), 6.48 (m, 2H), 6.26 (br, 1H), 4.80 (d, J=5.4 Hz, 2H), 3.89 (s, 3H), 3.82 (s, 3H)
Compound No. 413: (2,5-Dimethoxybenzyl)-quinazolino-4-ylamine

¹H NMR (CDCl₃, 300 MHz) δ 8.69 (s, 1H), 7.84 (d, J=8.4 Hz, 1H), 7.69 (m, 2H), 7.44 (t, J=7.2 Hz, 1H), 6.99 (d, J=3.0 Hz, 1H), 6.83 (m, 2H), 6.29 (br s, 1H), 4.85 (d, J=5.4 Hz, 2H), 3.88 (s, 3H), 3.76 (s, 3H)
Compound No. 414: Quinazolino-4-yl-(2,4,6-trimethoxybenzyl)-amine

¹H NMR (CDCl₃, 300 MHz) δ 8.72 (s, 1H), 7.81 (d, J=8.1 Hz, 1H), 7.69 (t, J=7.5 Hz, 1H), 7.60 (d, J=8.1 Hz, 1H), 7.40 (t, J=7.5 Hz, 1H), 6.19 (s, 2H), 6.03 (br s, 1H), 4.83 (d, J=5.1 Hz, 2H), 3.86 (s, 6H), 3.84 (s, 3H)
Compound No. 415: 4-Benzyloxy-2-isopropylquinazoline

¹H NMR (CDCl₃, 300 MHz) δ 8.16 (d, J=8.1 Hz, 1H), 7.87 (s, J=8.4 Hz, 1H), 7.77 (m, 1H), 7.54 (d, J=7.5 Hz, 2H), 7.49-7.34 (m, 4H), 5.66 (s, 2H), 3.22 (m, 1H), 1.40 (d, J=6.9 Hz, 6H)
Compound No. 416: 4-Benzylsulfanyl-2-isopropylquinazoline

¹H NMR (CDCl₃, 300 MHz) δ 8.01 (d, J=8.4 Hz, 1H), 7.89 (d, J=8.4 Hz, 1H), 7.78 (m, 1H), 7.48 (m, 3H), 7.35-7.23 (m, 3H), 4.67 (s, 2H), 3.29 (m, 1H), 1.43 (d, J=6.9 MHz, 6H)

The compound represented by the formula 1 may be prepared into a variety of preparation forms depending on the purpose. The following are non-limiting examples of some preparation forms comprising the compound represented by the formula 1 as an active ingredient.

### Preparation Example 1

### Preparation of Tablets

A tablet for oral administration was prepared by wet granulation and dry granulation.

[Composition]

Active ingredient 200 mg, hard silicic anhydride 10 mg, magnesium stearate 2 mg, mycrocrystalline cellulose 50 mg, sodium starch glycolate 25 mg, corn starch 113 mg, anhydrous ethanol proper quantity.

### Preparation Example 2

### Preparation of Ointments

An ointment with the following composition was prepared.

[Composition]

Active ingredient 5 g, cetyl palmitate 20 g, cetanol 40 g, stearyl alcohol 40 g, isopropyl myristrate 80 g, sorbitan monostearate 20 g, polysorbate 60 g, propyl p-oxybenzoate 1 g, methyl p-oxybenzoate 1 g, phosphoric acid and purified water proper quantity.

### Preparation Example 3

### Preparation of Injections

An injection with the following composition was prepared.

[Composition]

Active ingredient 100 mg, mannitol 180 mg, dibasic sodium phosphate 25 mg, water for injection 2974 mg.

### Experimental Example 1

### Analysis of Glucose Absorption

C2C12 muscle cells were cultured in DMEM (Dulbecco's modified Eagle's medium) containing 10% bovine calf serum. When the cell density increased to about 85 to 90%, the culture medium was replaced with 1% bovine calf serum and cell differentiation was induced for 6 days. Completely differentiated cells were cultured for 3 hours with Krebs-Ringer buffer (KRB) containing 5 mM glucose. The test materials were added at a concentration of 10 μM. 30 minutes later, 0.2 μCi 2-deoxyglucose was added. KRB was removed 20 minutes later and the cells were washed for 3 times with cold PBS. The cells were lysed with 0.5N NaOH and counted using a radioactive counter.

As seen in Table 2 below, the group treated with the compound represented by the formula 1 showed glucose absorption 2.8 times as large as the control group.

TABLE 2

| Compd. No. | Glucose absorption |
|---|---|
| 1 | 1.7 |
| 2 | 1.3 |
| 3 | 1.2 |
| 4 | 1.2 |
| 5 | 0.9 |
| 6 | 1.4 |
| 7 | 1.5 |
| 8 | 1.2 |
| 9 | 2.0 |
| 10 | 1.5 |
| 11 | 1.0 |
| 12 | 0.8 |
| 13 | 0.7 |
| 14 | 1.2 |
| 15 | 0.8 |
| 16 | 1.3 |
| 17 | 1.2 |
| 18 | 1.0 |
| 19 | 1.4 |
| 20 | 1.9 |
| 21 | 1.2 |
| 22 | 2.6 |
| 23 | 0.8 |
| 24 | 2.8 |
| 25 | 2.6 |
| 26 | 1.7 |
| 27 | 1.0 |
| 28 | 1.1 |
| 29 | 1.6 |
| 30 | 1.8 |
| 31 | 1.1 |
| 32 | 1.1 |
| 33 | 1.2 |
| 34 | 1.3 |
| 35 | 1.9 |
| 36 | 1.4 |
| 37 | 2.2 |
| 38 | 1.5 |
| 39 | 0.9 |
| 40 | 1.2 |
| 41 | 1.1 |
| 42 | 1.1 |
| 43 | 1.1 |
| 44 | 1.3 |
| 45 | 1.1 |
| 46 | 1.1 |
| 47 | 0.9 |
| 48 | 1.0 |
| 49 | 1.8 |
| 50 | 1.2 |
| 51 | 1.0 |
| 52 | 1.3 |
| 53 | 1.2 |
| 54 | 1.2 |
| 55 | 1.2 |
| 56 | 1.1 |
| 57 | 1.0 |
| 58 | 1.4 |
| 59 | 1.2 |
| 60 | 1.0 |
| 61 | 1.9 |
| 62 | 1.1 |
| 63 | 1.1 |
| 64 | 1.2 |
| 65 | 1.4 |
| 66 | 1.3 |
| 67 | 0.9 |
| 68 | 1.3 |
| 69 | 1.0 |
| 70 | 1.0 |
| 71 | 1.0 |
| 72 | 1.1 |
| 73 | 1.0 |
| 74 | 0.9 |
| 75 | 1.3 |
| 76 | 1.0 |
| 77 | 1.4 |
| 78 | 1.4 |
| 79 | 1.1 |
| 80 | 1.0 |
| 81 | 0.9 |
| 82 | 1.2 |
| 83 | 0.7 |
| 84 | 1.0 |
| 85 | 1.0 |
| 86 | 1.4 |
| 87 | 1.0 |
| 88 | 1.1 |
| 89 | 1.4 |
| 90 | 1.4 |
| 91 | 1.2 |
| 92 | 1.4 |
| 93 | 1.1 |
| 94 | 1.3 |
| 95 | 1.2 |
| 96 | 1.3 |
| 97 | 1.0 |
| 98 | 1.2 |
| 99 | 1.4 |
| 100 | 1.4 |
| 101 | 1.5 |
| 102 | 1.2 |
| 103 | 2.2 |
| 104 | 1.0 |
| 105 | 1.2 |
| 106 | 1.8 |
| 107 | 0.4 |
| 108 | 2.2 |
| 109 | 1.3 |
| 110 | 2.0 |
| 111 | 2.4 |
| 112 | 1.9 |
| 113 | 2.1 |
| 114 | 2.2 |
| 115 | 2.3 |
| 116 | 2.5 |
| 117 | 1.4 |
| 118 | 2.1 |
| 119 | 2.6 |
| 120 | 1.4 |
| 121 | 2.4 |
| 122 | 1.3 |
| 123 | 2.1 |
| 124 | 1.4 |
| 125 | 2.8 |
| 126 | 2.1 |
| 127 | 1.4 |
| 128 | 2.3 |
| 129 | 1.3 |
| 130 | 2.2 |
| 131 | 2.3 |
| 132 | 1.3 |
| 133 | 2.1 |
| 134 | 2.6 |
| 135 | 2.6 |
| 136 | 1.3 |
| 137 | 2.4 |
| 138 | 2.4 |
| 139 | 2.5 |
| 140 | 2.7 |
| 141 | 2.1 |
| 142 | 1.4 |
| 143 | 2.3 |
| 144 | 1.3 |
| 145 | 2.2 |
| 146 | 2.3 |
| 147 | 1.3 |
| 148 | 2.1 |
| 149 | 2.6 |
| 150 | 2.6 |
| 151 | 1.3 |
| 152 | 2.4 |
| 153 | 2.4 |
| 154 | 2.5 |
| 155 | 2.7 |
| 156 | 1.1 |
| 157 | 2.0 |
| 158 | 2.0 |
| 159 | 2.6 |
| 160 | 2.5 |
| 161 | 1.1 |
| 162 | 1.3 |
| 163 | 2.5 |
| 164 | 3.4 |
| 165 | 2.5 |
| 166 | 2.8 |
| 167 | 3.2 |
| 168 | 3.6 |
| 169 | 2.5 |
| 170 | 2.4 |
| 171 | 2.2 |
| 172 | 2.6 |
| 173 | 1.3 |
| 174 | 1.2 |
| 175 | 2.7 |
| 176 | 2.4 |
| 177 | 2.8 |
| 178 | 4.2 |
| 179 | 2.9 |
| 180 | 4.1 |
| 181 | 3.1 |
| 182 | 3.1 |
| 183 | 3.3 |
| 184 | 3.2 |
| 185 | 1.4 |
| 186 | 1.0 |
| 187 | 2.4 |
| 188 | 1.0 |
| 189 | 1.3 |
| 190 | 1.2 |
| 191 | 2.4 |
| 192 | 2.1 |
| 193 | 1.3 |
| 194 | 1.4 |
| 195 | 1.2 |
| 196 | 2.1 |
| 197 | 1.3 |
| 198 | 1.4 |
| 199 | 2.1 |
| 200 | 2.7 |
| 201 | 1.7 |
| 202 | 1.4 |
| 203 | 2.0 |
| 204 | 1.2 |
| 205 | 1.3 |
| 206 | 1.4 |
| 207 | 2.0 |
| 208 | 2.0 |
| 209 | 1.1 |
| 210 | 0.9 |
| 211 | 1.3 |
| 212 | 2.0 |
| 213 | 1.4 |
| 214 | 2.0 |
| 215 | 2.2 |
| 216 | 2.6 |
| 217 | 1.2 |
| 218 | 2.0 |
| 219 | 2.3 |
| 220 | 2.5 |
| 221 | 1.5 |
| 222 | 1.2 |
| 223 | 1.7 |
| 224 | 1.1 |
| 225 | 1.3 |
| 226 | 1.9 |
| 227 | 1.6 |
| 228 | 2.1 |
| 229 | 1.9 |
| 230 | 1.7 |
| 231 | 1.2 |
| 232 | 1.2 |
| 233 | 1.4 |
| 234 | 1.1 |
| 235 | 1.2 |
| 236 | 1.2 |
| 237 | 0.9 |
| 238 | 1.0 |
| 239 | 0.5 |
| 240 | 1.0 |
| 241 | 1.2 |
| 242 | 0.9 |
| 243 | 1.4 |
| 244 | 0.4 |
| 245 | 1.2 |
| 246 | 1.2 |
| 247 | 1.3 |
| 248 | 1.4 |
| 249 | 0.9 |
| 250 | 1.3 |
| 251 | 0.6 |
| 252 | 0.9 |
| 253 | 1.3 |
| 254 | 1.4 |
| 255 | 1.3 |
| 256 | 0.6 |
| 257 | 1.4 |
| 258 | 1.3 |
| 259 | 1.4 |
| 260 | 1.4 |
| 261 | 1.1 |
| 262 | 1.1 |
| 263 | 1.1 |
| 264 | 1.1 |
| 265 | 1.1 |
| 266 | 1.1 |
| 267 | 1.0 |
| 268 | 1.1 |
| 269 | 1.2 |
| 270 | 1.1 |
| 271 | 0.8 |
| 272 | 0.4 |
| 273 | 0.7 |
| 274 | 0.8 |
| 275 | 0.8 |
| 276 | 0.8 |
| 283 | 1.0 |
| 285 | 0.8 |
| 288 | 0.8 |
| 291 | 0.8 |
| 292 | 0.7 |
| 293 | 0.6 |
| 296 | 0.9 |
| 298 | 0.6 |
| 299 | 0.8 |
| 300 | 0.9 |
| 301 | 0.8 |
| 302 | 0.7 |
| 303 | 1.0 |
| 304 | 0.8 |
| 305 | 0.8 |
| 306 | 0.8 |
| 307 | 0.8 |
| 308 | 0.8 |
| 309 | 0.8 |
| 310 | 0.9 |
| 321 | 1.2 |
| 322 | 1.4 |
| 323 | 1.1 |
| 324 | 1.3 |
| 325 | 1.2 |
| 326 | 1.2 |
| 327 | 1.3 |
| 328 | 1.4 |
| 329 | 1.8 |
| 330 | 1.2 |
| 331 | 1.2 |
| 332 | 1.1 |
| 333 | 1.2 |
| 334 | 1.1 |
| 335 | 1.1 |
| 336 | 1.3 |
| 337 | 1.3 |
| 341 | 0.8 |
| 343 | 1.3 |
| 345 | 1.3 |
| 353 | 1.2 |
| 355 | 1.2 |
| 360 | 1.4 |
| 361 | 1.3 |
| 362 | 1.2 |
| 363 | 1.3 |
| 364 | 1.3 |
| 368 | 1.1 |
| 369 | 1.1 |
| 370 | 1.3 |
| 371 | 1.2 |
| 372 | 1.1 |
| 373 | 1.1 |
| 374 | 1.1 |
| 375 | 1.1 |
| 376 | 1.1 |
| 377 | 1.1 |
| 378 | 0.9 |
| 379 | 1.1 |
| 380 | 1.0 |
| 381 | 1.2 |
| 382 | 1.2 |
| 383 | 1.4 |
| 384 | 1.2 |
| 387 | 1.2 |
| 388 | 1.2 |
| 389 | 1.3 |
| 390 | 1.1 |
| 391 | 1.2 |
| 392 | 1.1 |
| 394 | 1.3 |
| 395 | 1.2 |
| 396 | 1.3 |
| 397 | 1.3 |
| 398 | 1.4 |
| 399 | 1.2 |
| 404 | 2.1 |
| 405 | 2.2 |
| 406 | 2.1 |
| 407 | 1.5 |
| 408 | 1.6 |
| 409 | 1.6 |
| 411 | 1.2 |
| 412 | 1.7 |
| 413 | 1.2 |
| 414 | 1.5 |

### Experimental Example 2

### Measurement of AMPK Activity by Western Blot

C2C12 cells were cultured and differentiated in the same manner as in Experimental Example 1. Treatment with the test materials was also performed in the same manner as in Experimental Example 1 (10 μM for 50 minutes). The culture medium was removed and the cells were washed for 2 times with PBS and protein extracts were obtained using a lysis buffer (50 mM tris-HCl, pH 7.4, 1% NP-40, 0.25% sodium deoxycholate, 150 mM NaCl, 1 mM EDTA, 1 mM PMSF, 1 mM sodium o-vanadate, 1 mM NaF, 1 μg/mL apronitin, 1 μg/mL leu-peptin, 1 μg/mL pepstatin). The AMPK activity was determined from Western blot analysis of the degree of phosphorylation of serine-79 by acetyl CoA carboxylase and the degree of phosphorylation of threonine-172. The amount of ERK (extracellular signal regulated protein kinase) was confirmed by Western blot in order to quantify the proteins.

The result of Western blot analysis is given in Table 3 below, wherein “strong” means that the degree of phosphorylation of threonine-172 increased by 3 times or more, “mild” means that the degree of phosphorylation of threonine-172 increased by 1 to 3 times and “weak” means that the degree of phosphorylation of threonine-172 increased by less than 1 time.

TABLE 3

| Comp. No. | AMPK activity |
|---|---|
| 1 | 6.0^{a} |
| 2 | 4.2^{a} |
| 3 | 3.3^{a} |
| 4 | 3.5^{a} |
| 5 | 4.7^{a} |
| 6 | 4.7^{a} |
| 7 | 4.4^{a} |
| 8 | 3.0^{a} |
| 9 | 5.5^{a} |
| 10 | 5.3^{a} |
| 11 | 3.2^{a} |
| 12 | 4.0^{a} |
| 13 | 4.2^{a} |
| 14 | 3.6^{a} |
| 15 | 2.3^{a} |
| 16 | 3.1^{a} |
| 17 | 2.2^{a} |
| 18 | 6.0^{a} |
| 20 | weak |
| 22 | strong |
| 24 | strong |
| 25 | strong |
| 26 | mild |
| 29 | mild |
| 30 | mild |
| 35 | strong |
| 37 | strong |
| 49 | mild |
| 61 | weak |
| 101 | weak |
| 103 | 2.6(strong) |
| 106 | 1.3(mild) |
| 108 | 2.2(strong) |
| 109 | 1.9(strong) |
| 110 | 1.6(strong) |
| 111 | 1.8(strong) |
| 112 | 1.7(strong) |
| 113 | 2.1(strong) |
| 114 | 2.0(strong) |
| 115 | 2.3(strong) |
| 116 | 2.0(strong) |
| 118 | 2.0(strong) |
| 119 | 2.3(strong) |
| 121 | 2.2(strong) |
| 123 | 2.3(strong) |
| 125 | 1.8(strong) |
| 126 | 2.0(strong) |
| 128 | 1.7(strong) |
| 130 | 1.8(strong) |
| 131 | 1.8(strong) |
| 133 | 1.3(mild) |
| 134 | 1.8(strong) |
| 135 | 2.0(strong) |
| 137 | 3.1(strong) |
| 138 | 2.9(strong) |
| 139 | 2.1(strong) |
| 140 | 3.1(strong) |
| 142 | 1.4(mild) |
| 143 | 2.4(strong) |
| 144 | 2.1(strong) |
| 145 | 2.7(strong) |
| 147 | 1.8(strong) |
| 148 | 1.7(strong) |
| 149 | 2.5(strong) |
| 151 | 1.8(strong) |
| 153 | 1.3(mild) |
| 155 | 1.3(mild) |
| 163 | 2.8(strong) |
| 164 | 1.6(mild) |
| 165 | 5.2(strong) |
| 166 | 3.8(strong) |
| 167 | 6.7(strong) |
| 168 | 8.4(strong) |
| 169 | 6.6(strong) |
| 170 | 8.8(strong) |
| 171 | 6.6(strong) |
| 172 | 4.4(strong) |
| 175 | 4.8(strong) |
| 176 | 4.4(strong) |
| 177 | 5.5(strong) |
| 178 | 5.7(strong) |
| 179 | 3.8(strong) |
| 180 | 3.9(strong) |
| 181 | 3.1(strong) |
| 182 | 2.9(strong) |
| 183 | 2.6(strong) |
| 184 | 1.9(strong) |
| 187 | 0.7(mild) |
| 191 | 5.2(strong) |
| 192 | 6.5(strong) |
| 196 | 4.8(strong) |
| 199 | 2.3(strong) |
| 200 | 1.5(strong) |
| 201 | 2.8(strong) |
| 203 | 3.9(strong) |
| 226 | 4.3(strong) |
| 227 | 4.9(strong) |
| 228 | 7.9(strong) |
| 229 | 4.7(strong) |
| 230 | 4.9(strong) |
| 271 | 2.4 |
| 272 | 3.4 |
| 273 | 1.5 |
| 274 | 1.9 |
| 275 | 2.1 |
| 276 | 1.9 |
| 277 | 1.3 |
| 278 | 1.2 |
| 279 | 1.3 |
| 280 | 1.2 |
| 281 | 0.9 |
| 282 | 0.9 |
| 283 | 1.1 |
| 284 | 1.4 |
| 285 | 1.6 |
| 286 | 1.0 |
| 287 | 1.2 |
| 288 | 1.7 |
| 289 | 1.5 |
| 290 | 1.0 |
| 291 | 1.5 |
| 292 | 1.2 |
| 293 | 0.1 |
| 294 | 0.4 |
| 295 | 0.2 |
| 296 | 1.0 |
| 297 | 0.8 |
| 298 | 0.7 |
| 299 | 0.6 |
| 300 | 1.0 |
| 301 | 0.9 |
| 302 | 1.2 |
| 303 | 1.0 |
| 304 | 1.4 |
| 305 | 2.1 |
| 306 | 1.7 |
| 307 | 2.0 |
| 308 | 2.2 |
| 309 | 1.3 |
| 310 | 1.6 |
| 311 | 0.2 |
| 312 | 0.3 |
| 313 | 0.1 |
| 314 | 0.2 |
| 315 | 0.4 |
| 316 | 1.2 |
| 317 | 0.3 |
| 318 | 0.5 |
| 319 | 1.5 |
| 320 | 0.9 |
| 321 | 0.9 |
| 322 | 1.9 |
| 323 | 1.2 |
| 324 | 2.3 |
| 325 | 2.2 |
| 326 | 2.9 |
| 327 | 4.4 |
| 328 | 3.8 |
| 329 | 4.2 |
| 330 | 1.7 |
| 342 | 1.0 |
| 343 | 0.8 |
| 344 | 0.4 |
| 345 | 1.1 |
| 346 | 0.8 |
| 347 | 1.2 |
| 348 | 1.6 |
| 349 | 2.1 |
| 350 | 0.6 |
| 351 | 0.7 |
| 352 | 0.5 |
| 353 | 0.8 |
| 354 | 0.6 |
| 355 | 1.1 |
| 356 | 1.4 |
| 357 | 0.7 |
| 358 | 0.8 |
| 359 | 0.9 |
| 360 | 2.2 |
| 361 | 2.1 |
| 362 | 2.6 |
| 363 | 1.4 |
| 364 | 1.2 |
| 365 | 1.1 |
| 366 | 1.6 |
| 367 | 2.2 |
| 368 | 5.9 |
| 369 | 4.5 |
| 370 | 7.7 |
| 371 | 2.8 |
| 372 | 6.3 |
| 373 | 2.1 |
| 374 | 1.9 |
| 375 | 5.2 |
| 376 | 1.1 |
| 377 | 2.3 |
| 378 | 4.7 |
| 379 | 6.1 |
| 380 | 1.9 |
| 381 | 4.0 |
| 382 | 2.2 |
| 383 | 2.2 |
| 384 | 2.0 |
| 385 | 2.8 |
| 386 | 1.3 |
| 387 | 1.5 |
| 388 | 2.8 |
| 389 | 2.2 |
| 390 | 3.8 |
| 391 | 7.1 |
| 392 | 2.7 |
| 393 | 1.0 |
| 394 | 4.5 |
| 395 | 7.0 |
| 396 | 7.3 |
| 397 | 6.5 |
| 398 | 7.8 |
| 399 | 4.9 |
| 400 | 0.4 |
| 401 | 1.4 |
| 402 | 0.5 |
| 403 | 1.3 |
| 404 | 1.9 |
| 405 | 2.7 |
| 406 | 1.5 |
| 407 | 1.3 |
| 408 | 3.5 |
| 409 | 3.3 |
| 410 | 1.1 |
| 411 | 1.8 |
| 412 | 8.4 |
| 413 | 1.7 |
| 414 | 2.9 |
| 415 | 1.1 |
| 416 | 0.7 |

^{a}Western blot analysis of the degree of phosphorylation of serine-79.
^{none}Western blot analysis of the degree of phosphorylation of serine-79 and threonine-172.

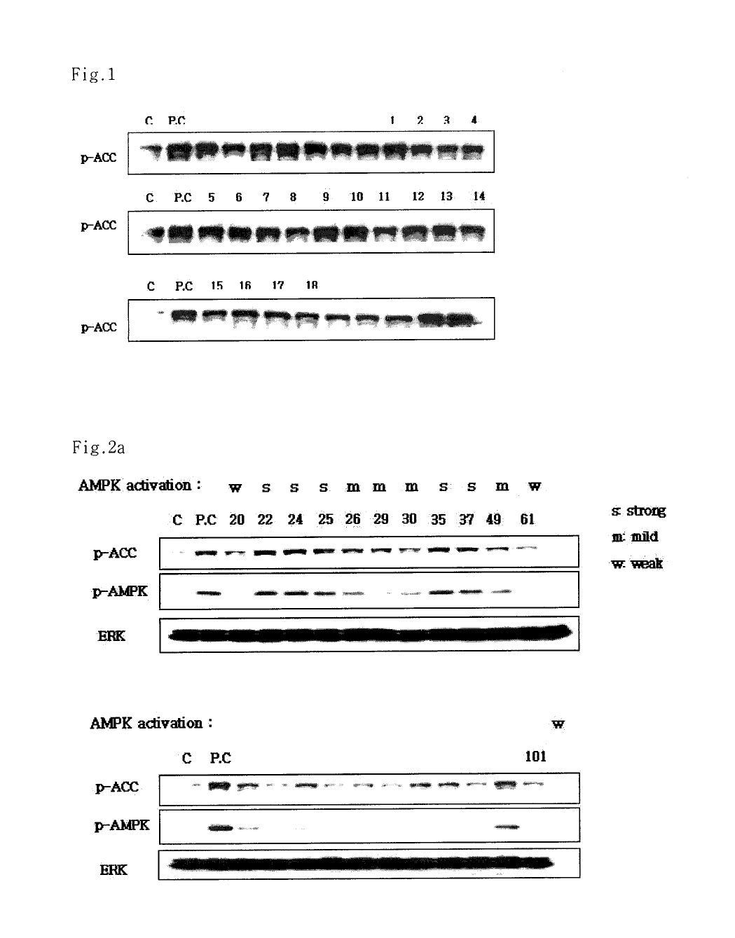

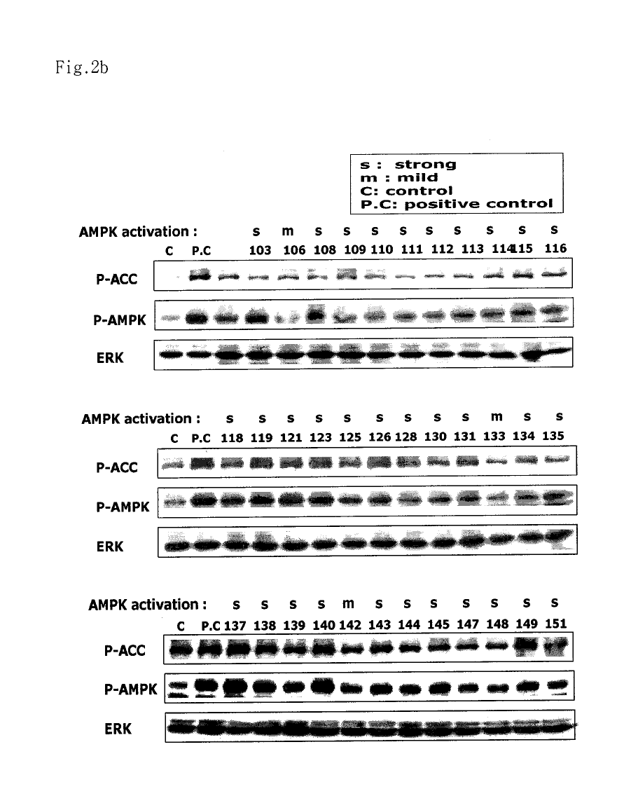

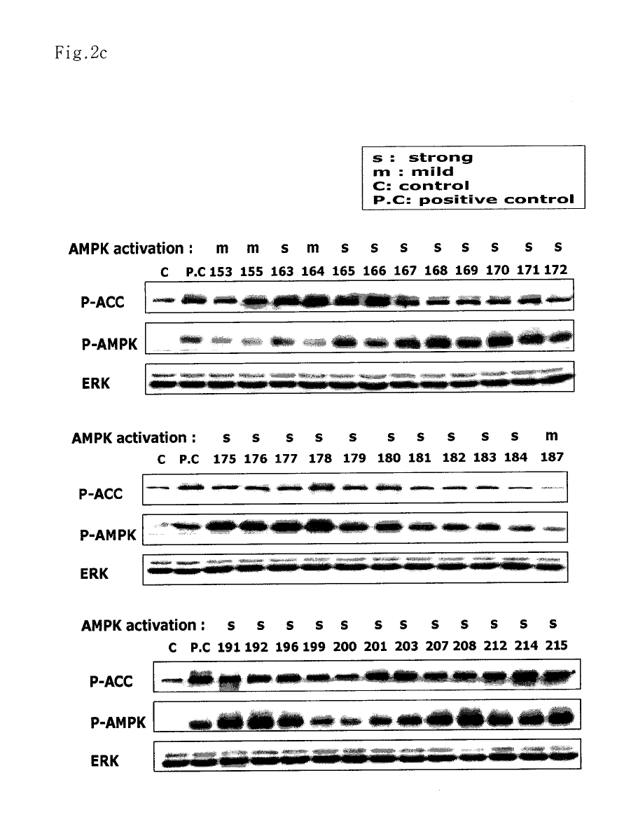

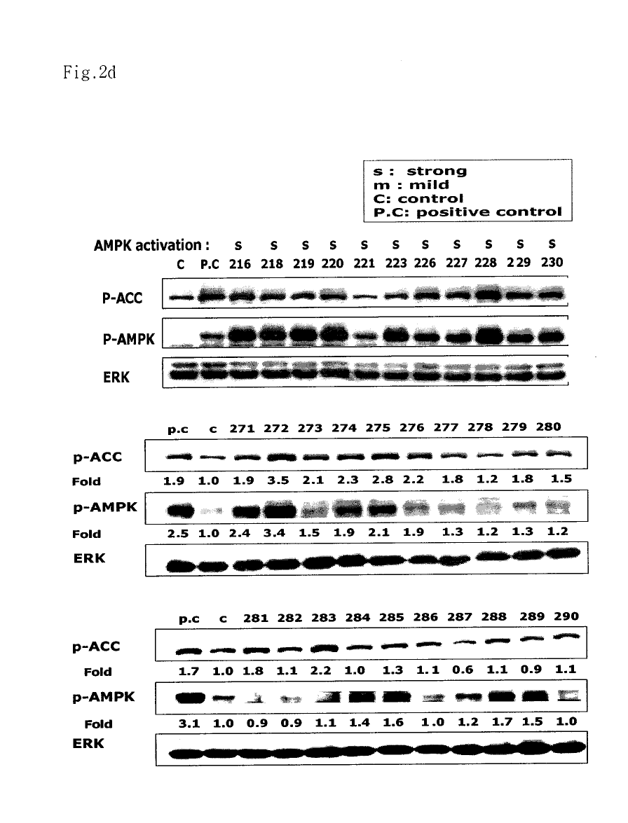

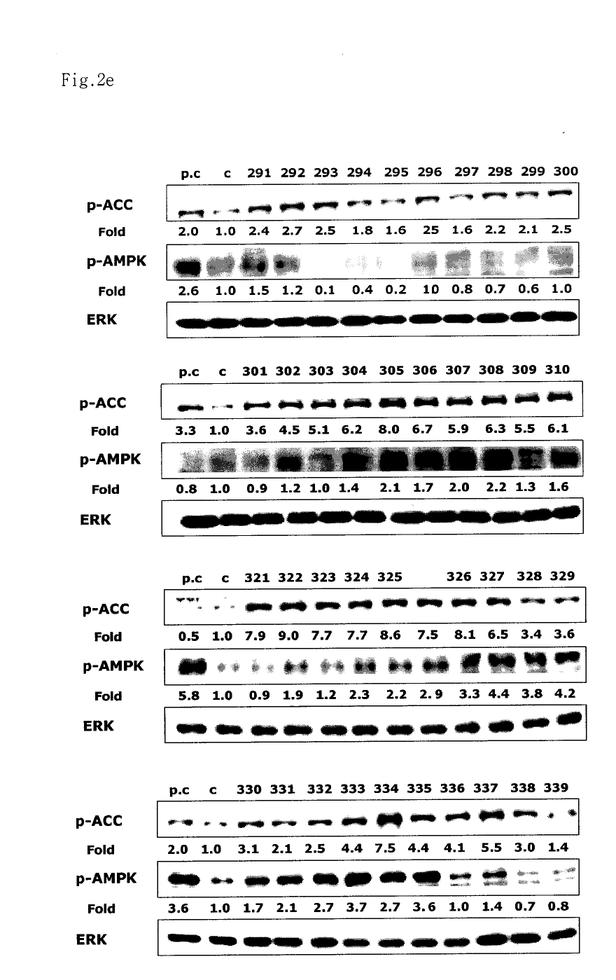

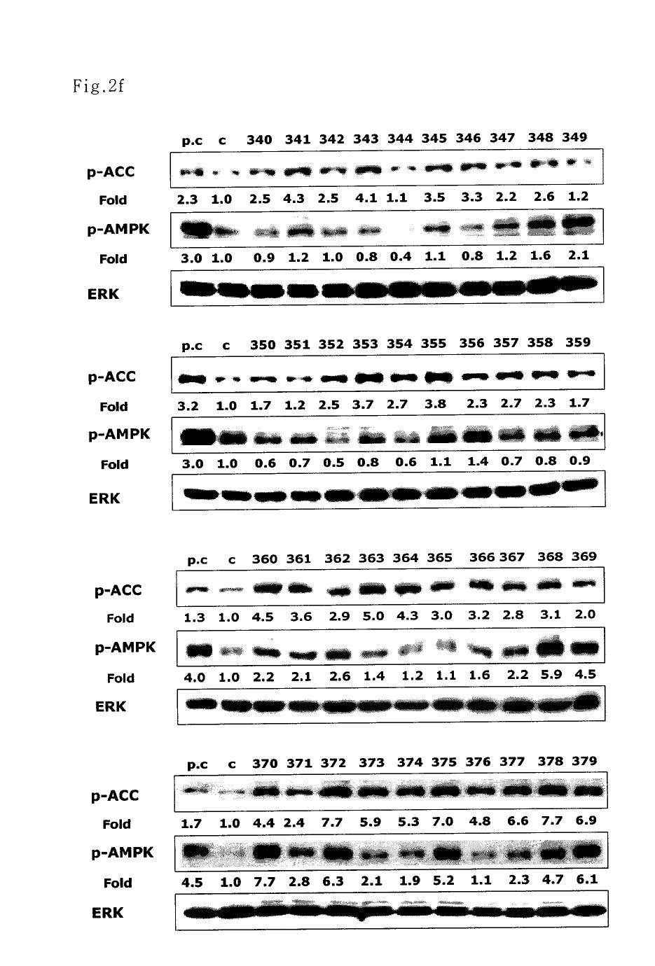

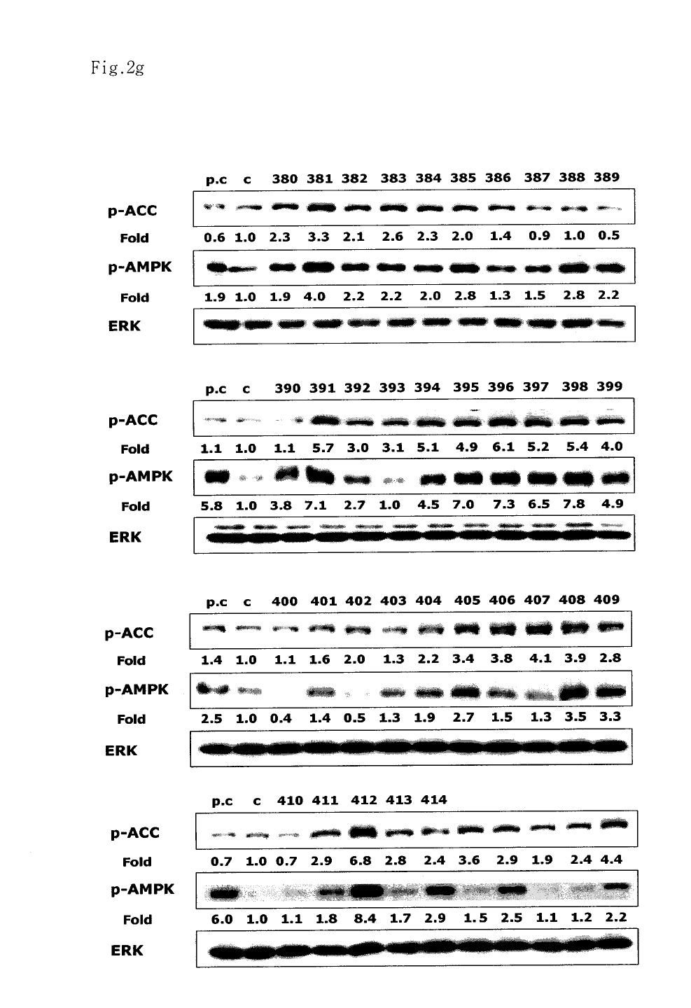

As seen in Table 3, FIG. 1 and FIG. 2a to FIG. 2g, increase of AMPK activity was observed when the muscle cells were treated with the compound represented by the formula 1.

### Experimental Example 3

### Preventive and Therapeutic Effect for Diabetes/Obesity in db/db Mice

8- to 9-week-old male db/db mice (C57BL/Ks) were tested. 0.5% carboxylmethylcellulose (CMC), rosiglitazone dissolved in 0.5% carboxylmethylcellulose (CMC) and the test material were administered orally or abdominally every afternoon to the negative control group, the positive control group and the test group, respectively. Blood glucose level measurement was made just before the administration, at an interval of 3-4 days, by incising the tail vein. Body weight and the amount of feed were measured every day just before the administration.

After 10 days of administration, blood glucose level measurement and body weight measurement, serum was separated from blood for measuring the biochemical parameters related with liver function, kidney function and lipid level.

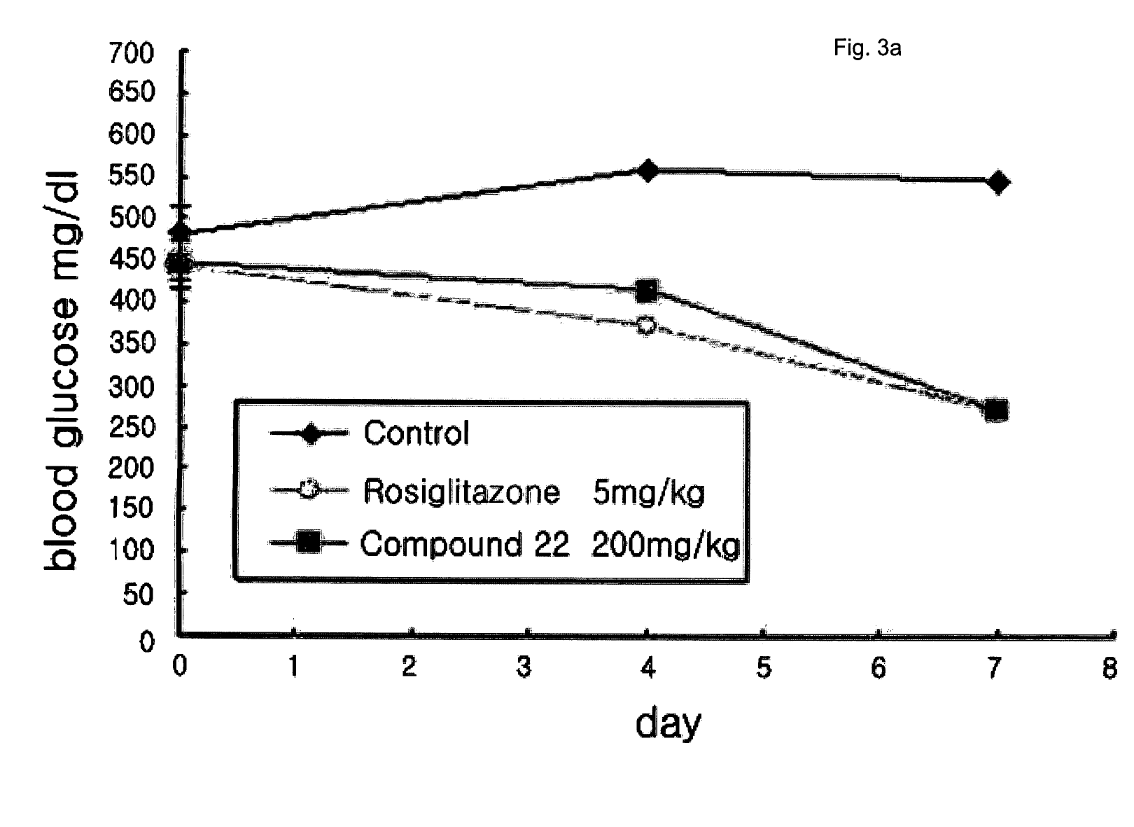

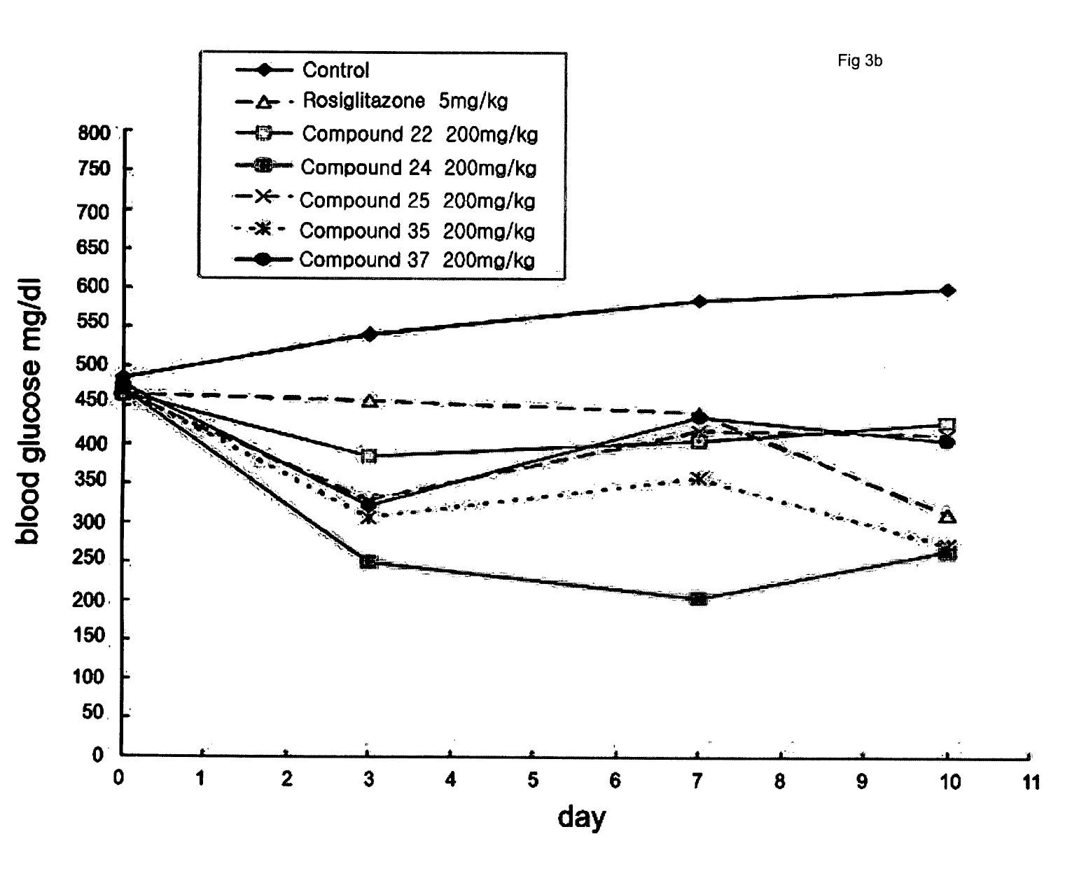

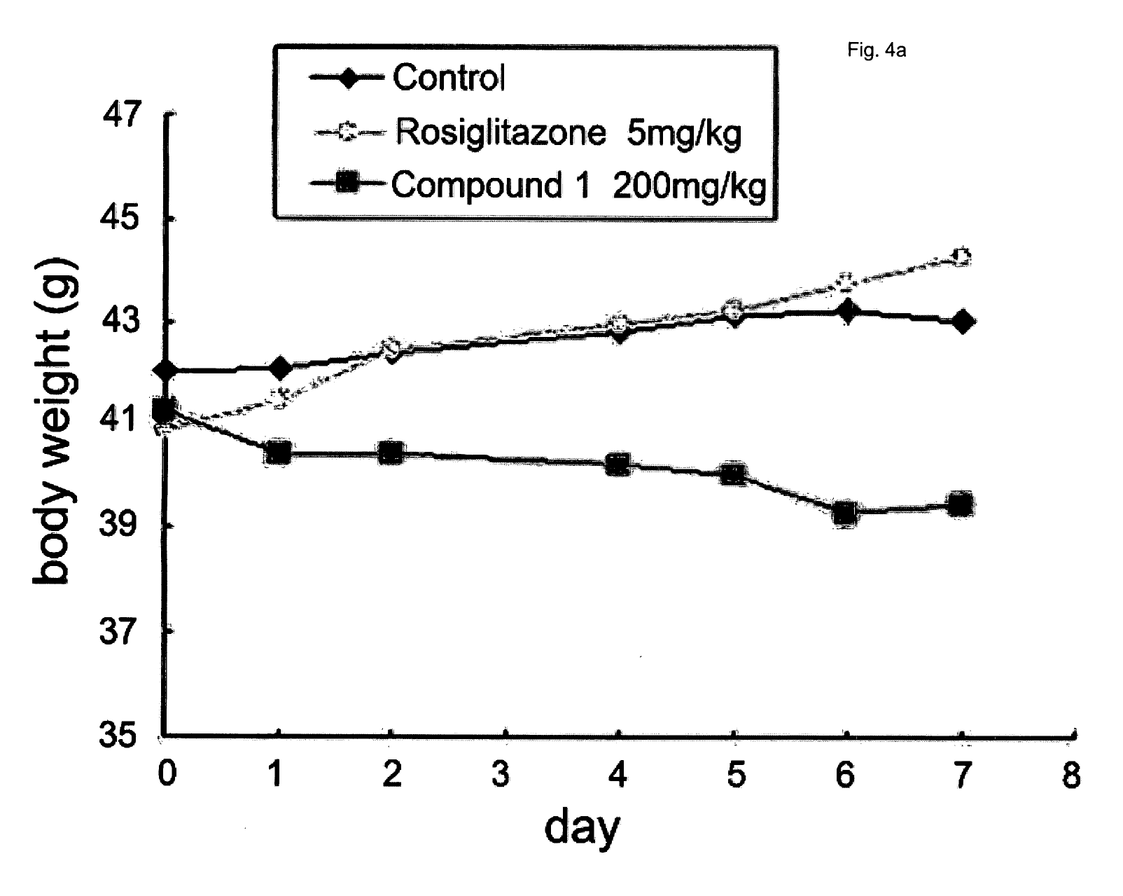

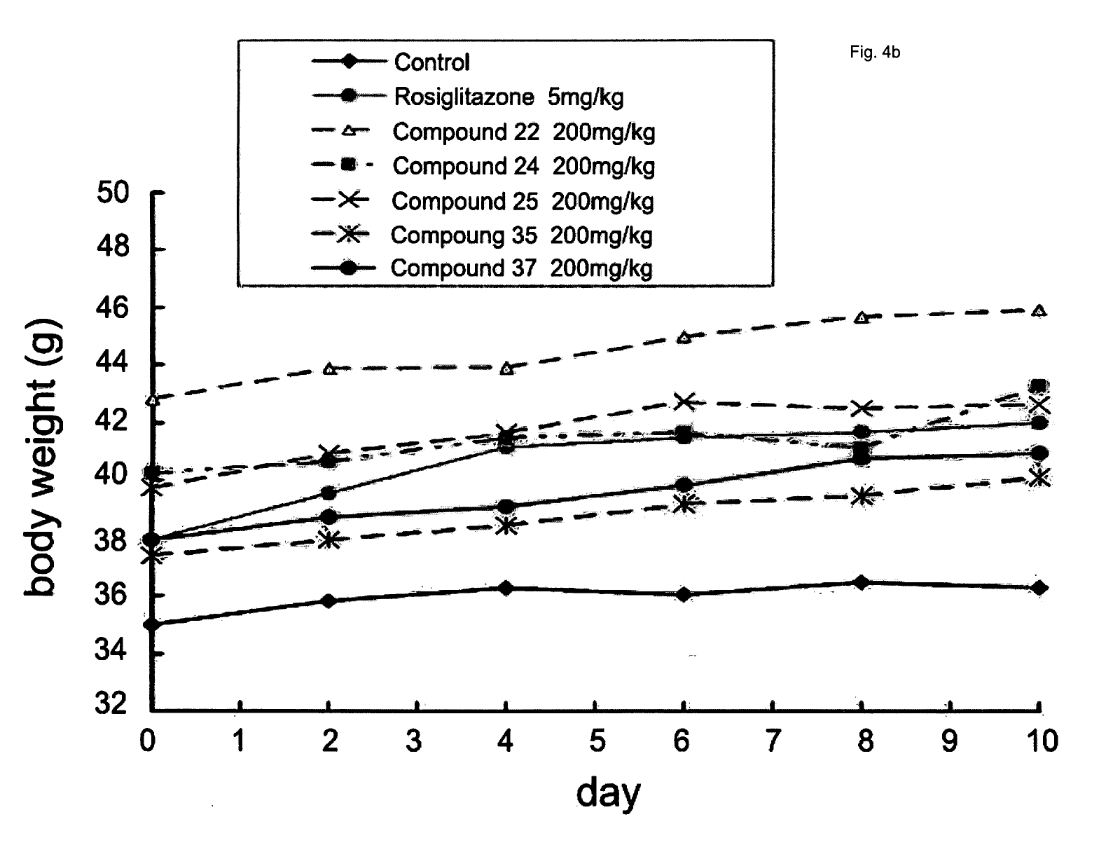

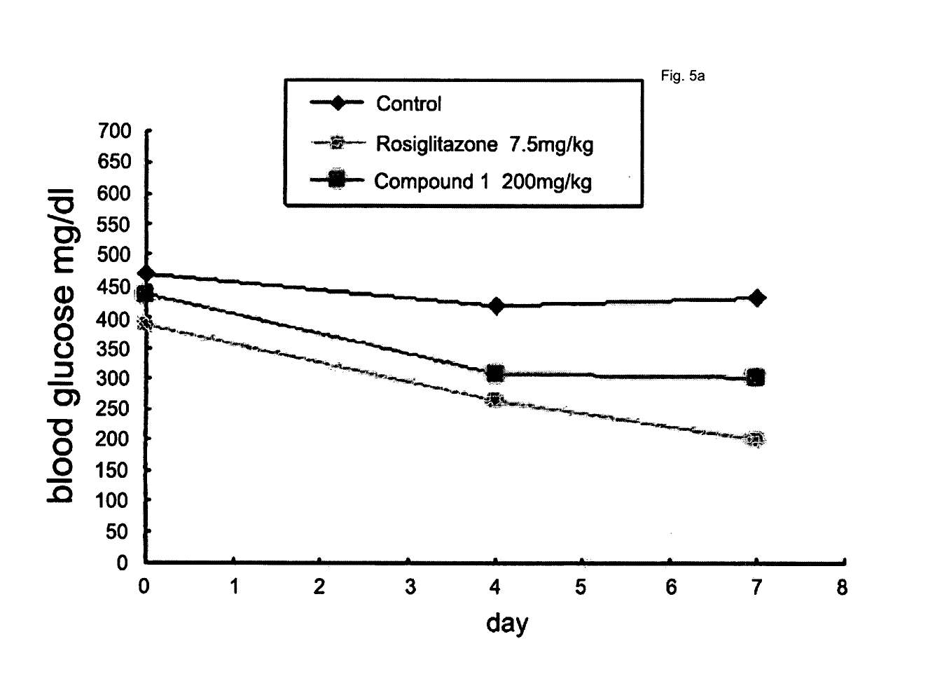

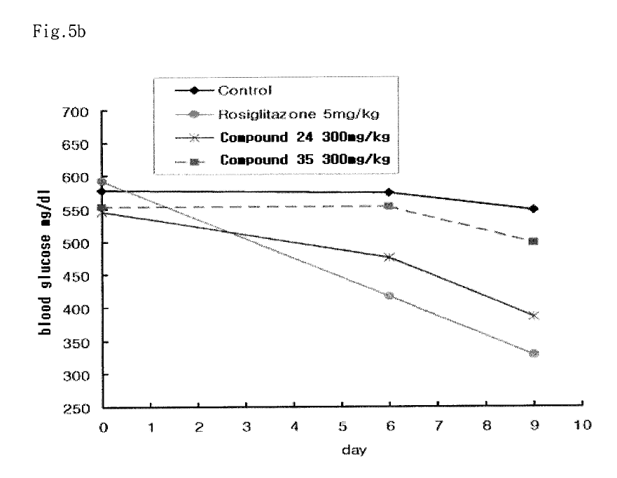

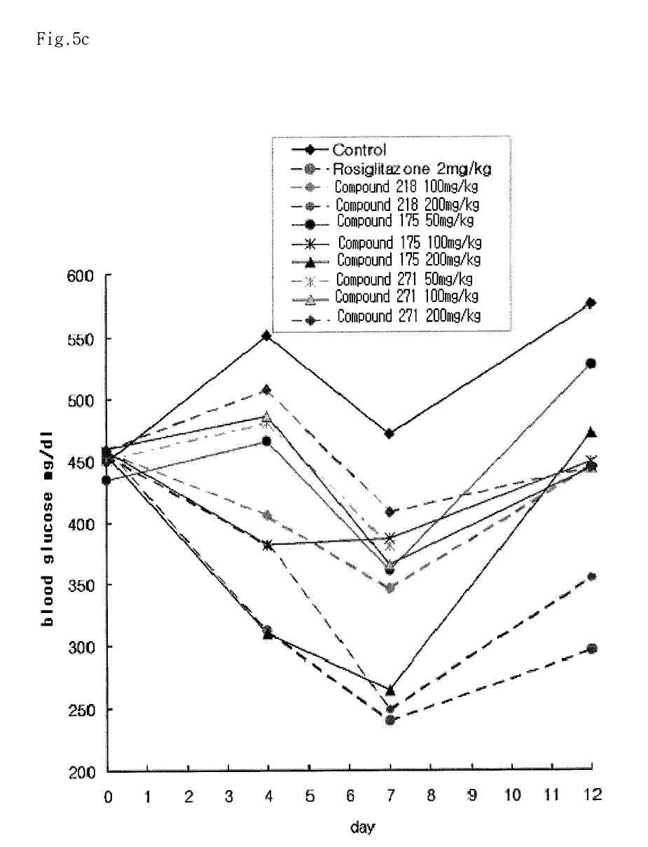

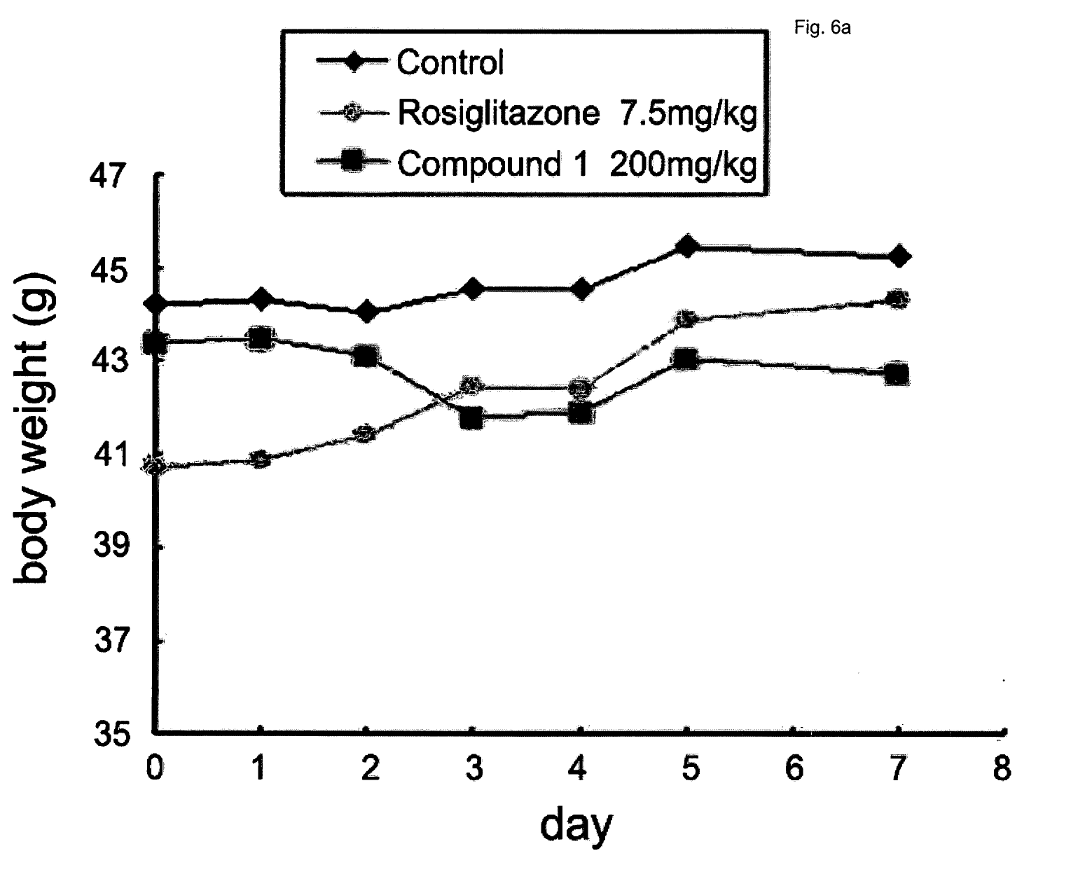

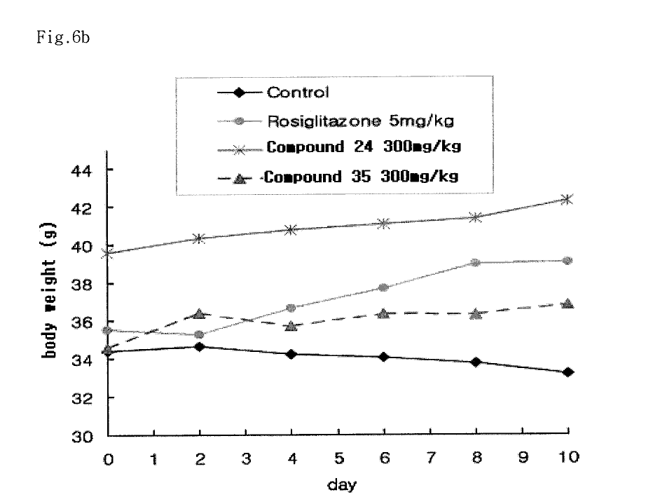

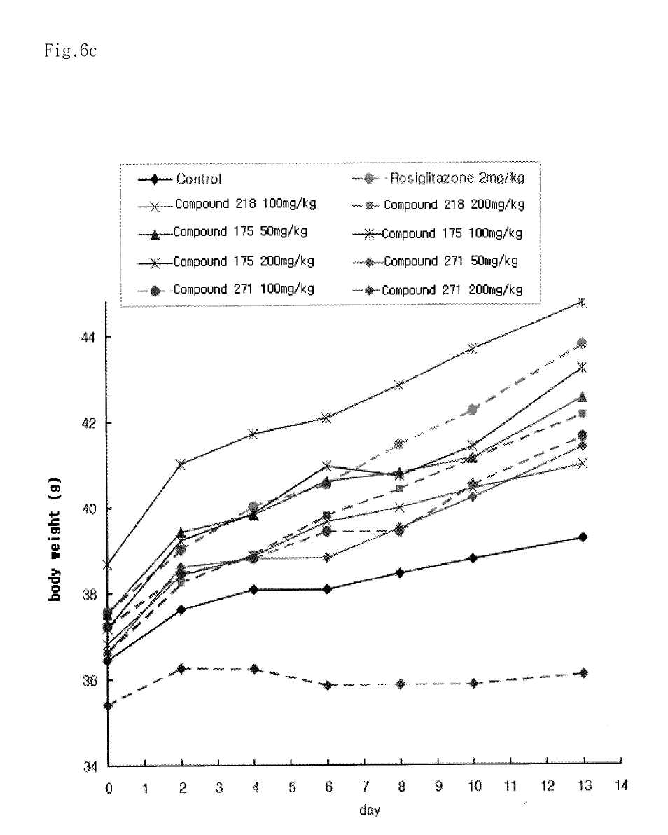

As seen in FIGS. 3a and 3b, FIGS. 4a and 4b, FIGS. 5a, 5b and 5c and FIGS. 6a, 6b and 6c, the test group to which the compound represented by the formula 1 had been administered abdominally or orally showed significant decrease in blood glucose level and body weight compared with the control group, which implies the preventive and therapeutic effect for diabetes and obesity.

### Experimental Example 4

### Preventive and Therapeutic Effect for Diabetes/Obesity in ob/ob Mice

### Experimental Example 4-1

6-week old male ob/ob mice (Harlan) were accustomed to the environment of the animal breeding facility of SK for a week. During the period, the mice were freely given feed and water. The mice were grouped based on the body weight. The mice were grouped into the control group, the positive control group (sibutramine) and the test group. The administration dosage was 15 mg/kg for the positive control group and 200 mg/kg/10 mL for the test group. Administration was given abdominally using an insulin syringe. Administration was given once a day at 5 p.m. over a 4-week period. The control group was administered with 0.5% carboxymethyl cellulose (CMC), at kg/10 mL.

Body weight was measured over the 4-week period. The difference in the body weight of the control group and the body weight of the test group (g) and the increase of the body weight (%) were calculated to evaluate the inhibitive effect against body weight increase. Also, the amount of feed intake was measured in order to evaluate the inhibitive effect against feed intake.

### Experimental Example 4-2

Oral administration test was performed for the materials that showed effectiveness in Experimental Example 1. Grouping of the mice, administration dosage, administration period, etc. were the same as in Experimental Example 3-1. Forced oral administration was performed using a bouige.

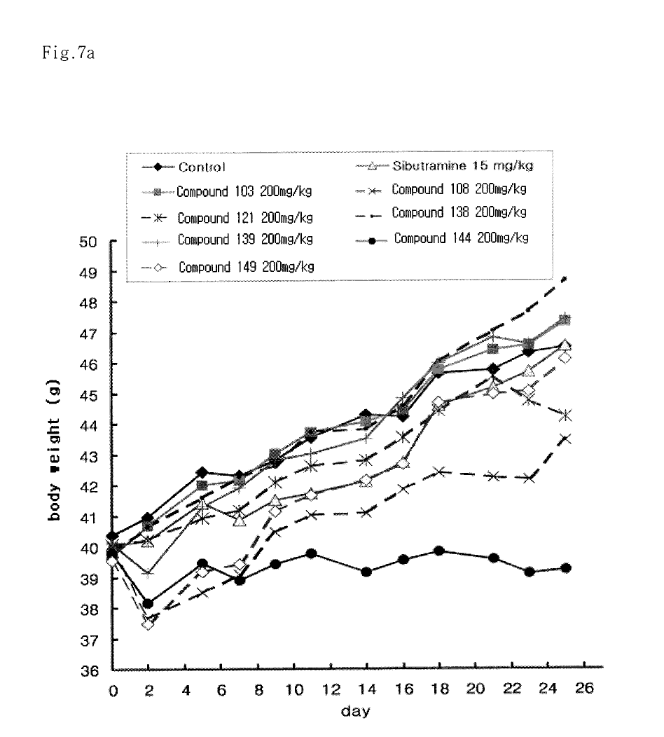

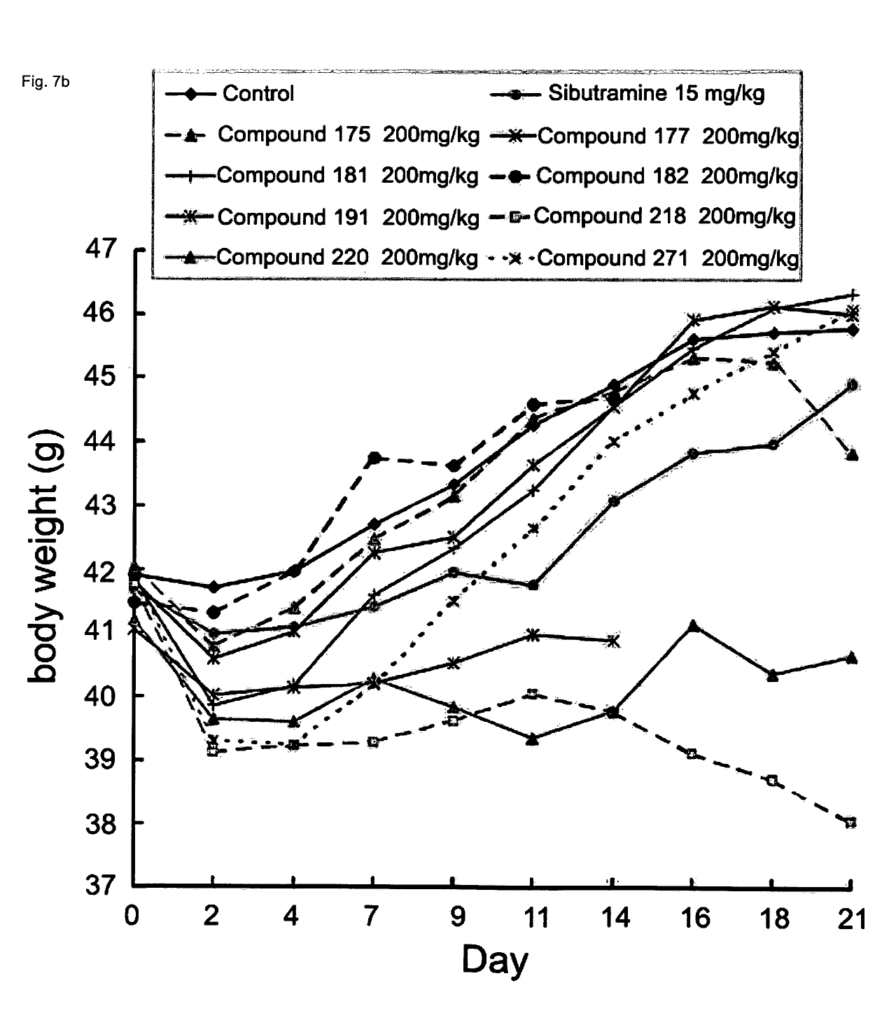

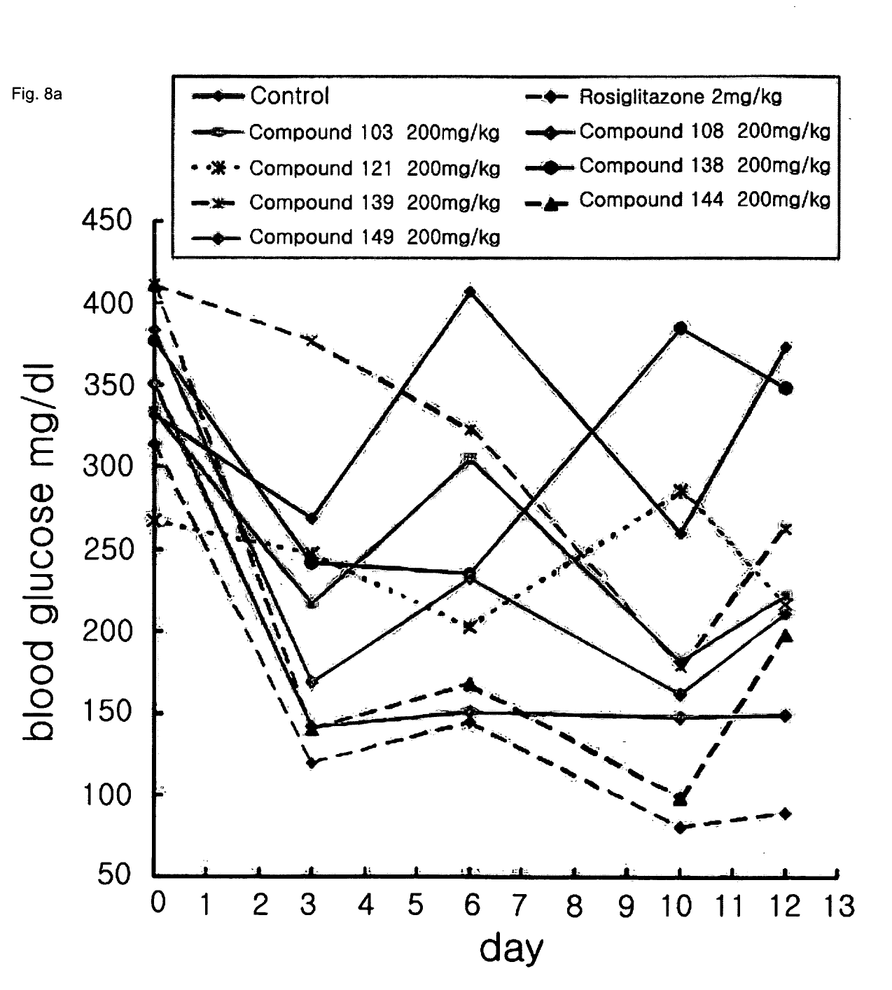

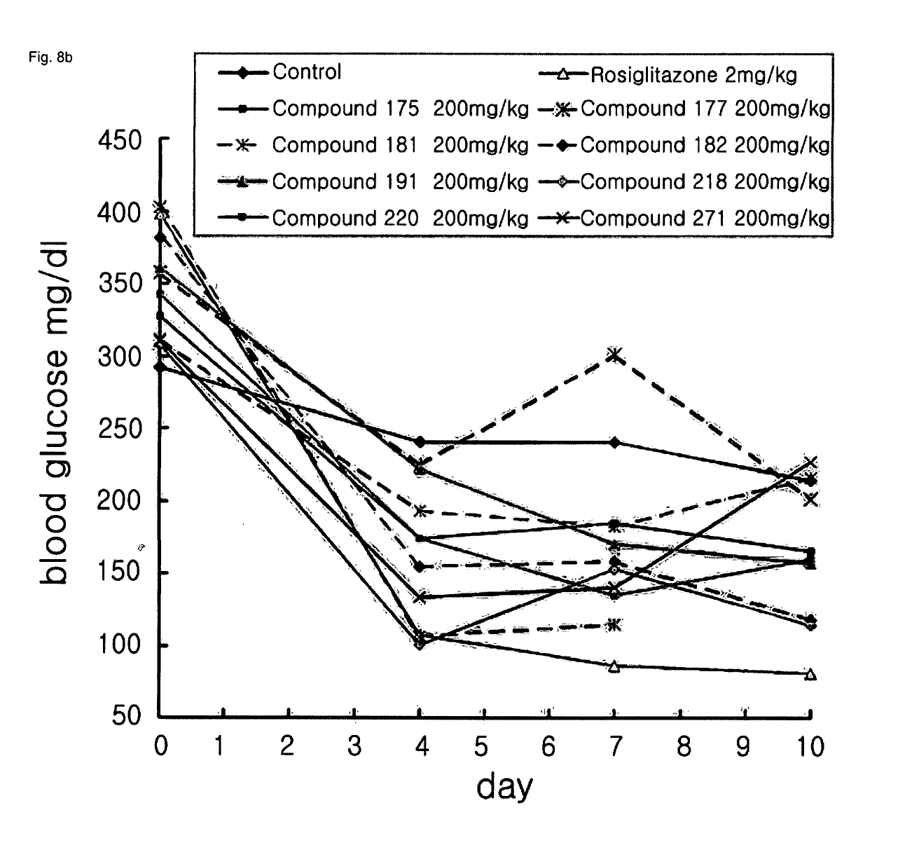

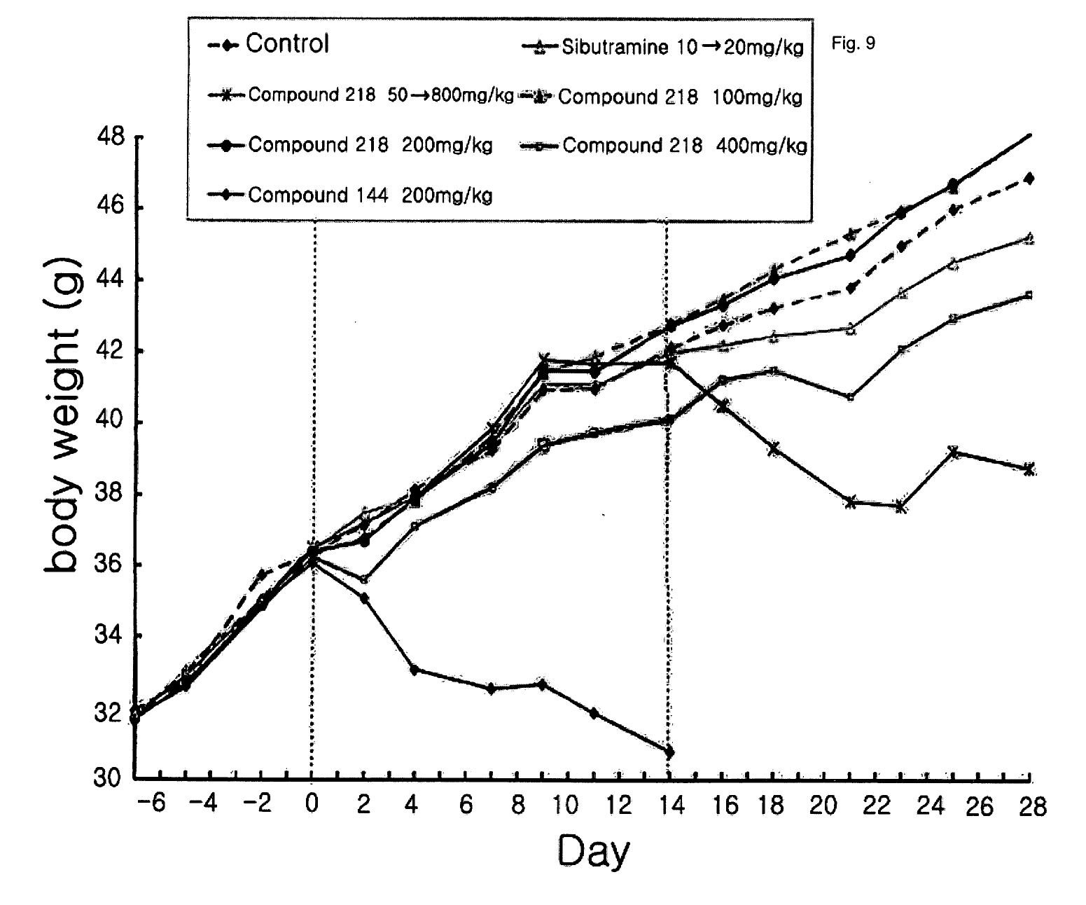

As seen in FIGS. 7a and 7b, FIGS. 8a and 8b and FIG. 9, the test group to which the compound represented by the formula 1 had been administered abdominally or orally showed significant decrease in blood glucose level and body weight compared with the control group, which implies the preventive and therapeutic effect for diabetes and obesity.

### INDUSTRIAL APPLICABILITY

As apparent from the above description, the compounds of the present invention are effective in lowering blood glucose level and body weight and can be used for treatment of diabetes and/or obesity.

While the present invention has been described in detail with reference to the preferred embodiments, those skilled in the art will appreciate that various modifications and substitutions can be made thereto without departing from the spirit and scope of the invention as set forth in the appended claims.

1. A quinazoline derivative represented by the formula 1 below or a pharmaceutically available salt thereof: 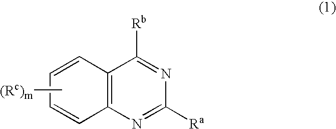
wherein
R^{a} is a substituent selected from hydrogen, (C₁-C₁₈)alkyl, (C₂-C₈)alkenyl, (C₂-C₈)alkynyl, halogeno-(C₁-C₆)alkyl, hydroxy-(C₁-C₆)alkyl, (C₁-C₆)alkoxy-(C₁-C₆)alkyl, cyano-(C₁-C₆)alkyl, amino-(C₁-C₆)alkyl, (C₁-C₆)alkylamino-(C₁-C₆)alkyl, di-[(C₁-C₆)alkyl]amino-(C₁-C₆)alkyl, (C₁-C₆)alkylthio-(C₁-C₆)alkyl, (C₁-C₆)alkylsulfinyl-(C₁-C₆)alkyl and (C₁-C₆)alkylsulfonyl-(C₁-C₆)alkyl, or —X¹-Q¹ [wherein X¹ is a direct bond or selected from O, S, SO, SO₂, N(R⁴), CO, CO₂, CH(OR⁴), CON(R⁴), N(R⁴)CO, SO₂N(R⁴), N(R⁴)SO₂, C(R⁴)₂O, C(R⁴)₂S, N(R⁴)C(R⁴)₂, N(R⁴)CON(R⁴) and N(R⁴)CSN(R⁴), wherein R⁴ is a hydrogen atom, (C₁-C₆)alkyl, aryl, aryl-(C₁-C₆)alkyl, (C₃-C₇)cycloalkyl, (C₃-C₇)cycloalkyl-(C₁-C₆)alkyl, (C₃-C₇)cycloalkenyl, (C₃-C₇)cycloalkenyl-(C₁-C₆)alkyl, heteroaryl, heteroaryl-(C₁-C₆)alkyl, heterocyclyl or heterocyclyl-(C₁-C₆)alkyl, and Q¹ and R⁴ may be identical or different],
wherein the neighboring carbon atoms of (C₂-C₆)alkylene of R^{a} may be separated by a group selected from O, S, SO, SO₂, N(R⁵), CO, CH(OR⁵), CON(R⁵), N(R⁵)CO, SO₂N(R⁵), N(R⁵)SO₂, C(R⁵)═C(R⁵) and C≡C, wherein R⁵ is a hydrogen atom or (C₁-C₆)alkyl,
any CH₂═CH— or HC≡C— of R^{a} may have a substituent, at the terminal or CH₂═ or HC≡, selected from halogeno, carboxyl, carbamoyl, (C₁-C₆)alkoxycarbonyl, N—(C₁-C₆)alkylcarbamoyl, N,N-di-[(C₁-C₆)alkyl]carbamoyl, amino-(C₁-C₆)alkyl, (C₁-C₆)alkylamino-(C₁-C₆)alkyl and di-[(C₁-C₆)alkyl]amino-(C₁-C₆)alkyl or -Q²-X² [wherein X² is a direct bond or selected from CO and N(R⁶)CO, wherein R⁶ is a hydrogen atom or (C₁-C₆)alkyl, and Q² is aryl, aryl-(C₁-C₆)alkyl, heteroaryl, heteroaryl-(C₁-C₆)alkyl, heterocyclyl or heterocyclyl-(C₁-C₆)alkyl],
any CH₂ or CH₃ of R^{a} may have at least one substituent selected from halogeno, (C₁-C₆)alkyl, hydroxy, cyano, amino, carboxyl, carbamoyl, (C₁-C₆)alkoxy, (C₁-C₆)alkylthio, (C₁-C₆)alkylsulfinyl, (C₁-C₆)alkylsulfonyl, (C₁-C₆)alkylamino, di-[(C₁-C₆)alkyl]amino, (C₁-C₆)alkoxycarbonyl, N—(C₁-C₆)alkylcarbamoyl, N,N-di-[(C₁-C₆)alkyl]carbamoyl, (C₂-C₆)alkanoyl, (C₂-C₆)alkanoyloxy, (C₂-C₆)alkanoylamino, N—(C₁-C₆)alkyl-(C₂-C₆)alkanoylamino, N—(C₁-C₆)alkylsulfamoyl, N,N-di-[(C₁-C₆)alkyl]sulfamoyl, (C₁-C₆)alkanesulfonylamino and N—(C₁-C₆)alkyl-(C₁-C₆)alkanesulfonylamino, or —X³-Q³ [wherein X³ is a direct bond or selected from O, S, SO, SO₂, N(R⁷), Co, CH(OR⁷), CON(R⁷), N(R⁷)CO, SO₂N(R⁷), N(R⁷)SO₂, C(R⁷)₂O, C(R⁷)₂S, N(R⁷)C(R⁷)₂, N(R⁷)CON(R⁷) and N(R⁷)CSN(R⁷), wherein R⁷ is a hydrogen atom, (C₁-C₆)alkyl, aryl, aryl-(C₁-C₆)alkyl, (C₃-C₇)cycloalkyl, (C₃-C₇)cycloalkyl-(C₁-C₆)alkyl, (C₃-C₇)cycloalkenyl, (C₃-C₇)cycloalkenyl-(C₁-C₆)alkyl, heteroaryl, heteroaryl-(C₁-C₆)alkyl, heterocyclyl or heterocyclyl-(C₁-C₆)alkyl, and Q³ and R⁷ may be identical or different],
any aryl, heteroaryl or heterocyclyl of R^{a} may have 0 to 3 identical or different substituents selected from halogeno, trifluoromethyl, cyano, nitro, hydroxy, amino, carboxyl, carbamoyl, (C₁-C₆)alkyl, (C₂-C₈)alkenyl, (C₂-C₈)alkynyl, (C₁-C₆)alkoxy, (C₂-C₆)alkenyloxy, (C₂-C₆)alkynyloxy, (C₁-C₆)alkylthio, (C₁-C₆)alkylsulfinyl, (C₁-C₆)alkylsulfonyl, (C₁-C₆)alkylamino, di-[(C₁-C₆)alkyl]amino, (C₁-C₆)alkoxycarbonyl, N—(C₁-C₆)alkylcarbamoyl, N,N-di-[(C₁-C₆)alkyl]carbamoyl, (C₂-C₆)alkanoyl, (C₂-C₆)alkanoyloxy, (C₂-C₆)alkanoylamino, N—(C₁-C₆)alkyl-(C₂-C₆)alkanoylamino, N—(C₁-C₆)alkylsulfamoyl, N,N-di-[(C₁-C₆)alkyl]sulfamoyl, (C₁-C₆)alkanesulfonylamino and N—(C₁-C₆)alkyl-(C₁-C₆)alkanesulfonylamino, or —X⁴—R⁸ [wherein X⁴ is a direct bond or selected from O and N(R⁹), wherein R⁹ is a hydrogen atom or (C₁-C₆)alkyl, and R⁸ is halogeno-(C₁-C₆)alkyl, hydroxy-(C₁-C₆)alkyl, (C₁-C₆)alkoxy-(C₁-C₆)alkyl, cyano-(C₁-C₆)alkyl, amino-(C₁-C₆)alkyl, (C₁-C₆)alkylamino-(C₁-C₆)alkyl, di-[(C₁-C₆)alkyl]amino-(C₁-C₆)alkyl, (C₂-C₆)alkanoylamino-(C₁-C₆)alkyl or (C₁-C₆)alkoxycarbonylamino-(C₁-C₆)alkyl], or —X⁵-Q⁴ [wherein X⁵ is a direct bond or selected from O, CO and N(R¹⁰), wherein R¹⁰ is a hydrogen atom or (C₁-C₆)alkyl, and Q⁴ is aryl, aryl-(C₁-C₆)alkyl, heteroaryl, heteroaryl-(C₁-C₆)alkyl, heterocyclyl or heterocyclyl-(C₁-C₆)alkyl, which may have 1 or 2 identical or different substituents selected from halogeno, (C₁-C₆)alkyl and (C₁-C₆)alkoxy] and
heterocyclyl of R^{a} may have 1 or 2 oxo or thioxo substituents;
R^{b} is a substituent selected from hydrogen, (C₁-C₁₈)alkyl, (C₂-C₈)alkenyl, (C₂-C₈)alkynyl, halogeno-(C₁-C₆)alkyl, hydroxy-(C₁-C₆)alkyl, (C₁-C₆)alkoxy-(C₁-C₆)alkyl, cyano-(C₁-C₆)alkyl, amino-(C₁-C₆)alkyl, (C₁-C₆)alkylamino-(C₁-C₆)alkyl, di-[(C₁-C₆)alkyl]amino-(C₁-C₆)alkyl, (C₁-C₆)alkylthio-(C₁-C₆)alkyl, (C₁-C₆)alkylsulfinyl-(C₁-C₆)alkyl and (C₁-C₆)alkylsulfonyl-(C₁-C₆)alkyl, or —X¹-Q¹ [wherein X¹ is a direct bond or selected from O, S, SO, SO₂, N(R⁴), CO, CO₂(R⁴), CH(OR⁴), CON(R⁴), N(R⁴)CO, SO₂N(R⁴), N(R⁴)SO₂, C(R⁴)₂O, C(R⁴)₂S, N(R⁴)C(R⁴)₂, N(R⁴)CON(R⁴) and N(R⁴)CSN(R⁴), wherein R⁴ is a hydrogen atom, (C₁-C₆)alkyl, aryl, aryl-(C₁-C₆)alkyl, (C₃-C₇)cycloalkyl, (C₃-C₇)cycloalkyl-(C₁-C₆)alkyl, (C₃-C₇)cycloalkenyl, (C₃-C₇)cycloalkenyl-(C₁-C₆)alkyl, heteroaryl, heteroaryl-(C₁-C₆)alkyl, heterocyclyl or heterocyclyl-(C₁-C₆)alkyl, and Q¹ and R⁴ may be identical or different],
wherein the neighboring carbon atoms of (C₂-C₆)alkylene of R^{b} may be separated by a group selected from O, S, SO, SO₂, N(R⁵), CO, CH(OR⁵), CON(R⁵), N(R⁵)CO, SO₂N(R⁵), N(R⁵)SO₂, CH═CH and C≡C, wherein R⁵ is a hydrogen atom or (C₁-C₆)alkyl,
any CH₂═CH— or HC≡C— of R^{b} may have a substituent, at the terminal or at CH₂═ or HC≡, selected from halogeno, carboxyl, carbamoyl, (C₁-C₆)alkoxycarbonyl, N—(C₁-C₆)alkylcarbamoyl, N,N-di-[(C₁-C₆)alkyl]carbamoyl, amino-(C₁-C₆)alkyl, (C₁-C₆)alkylamino-(C₁-C₆)alkyl and di-[(C₁-C₆)alkyl]amino-(C₁-C₆)alkyl, or -Q²-X² [wherein X² is a direct bond or selected from CO and N(R⁶)CO, wherein R⁶ is a hydrogen atom or (C₁-C₆)alkyl, and Q² is aryl, aryl-(C₁-C₆)alkyl, heteroaryl, heteroaryl-(C₁-C₆)alkyl, heterocyclyl or heterocyclyl-(C₁-C₆)alkyl],
any CH₂ or CH₃ of R^{b} may have at least one substituent selected from halogeno, (C₁-C₆)alkyl, hydroxy, cyano, amino, carboxyl, carbamoyl, (C₁-C₆)alkoxy, (C₁-C₆)alkylthio, (C₁-C₆)alkylsulfinyl, (C₁-C₆)alkylsulfonyl, (C₁-C₆)alkylamino, di-[(C₁-C₆)alkyl]amino, (C₁-C₆)alkoxycarbonyl, N—(C₁-C₆)alkylcarbamoyl, N,N-di-[(C₁-C₆)alkyl]carbamoyl, (C₂-C₆)alkanoyl, (C₂-C₆)alkanoyloxy, (C₂-C₆)alkanoylamino, N—(C₁-C₆)alkyl-(C₂-C₆)alkanoylamino, N—(C₁-C₆)alkylsulfamoyl, N,N-di-[(C₁-C₆)alkyl]sulfamoyl, (C₁-C₆)alkanesulfonylamino and N—(C₁-C₆)alkyl-(C₁-C₆)alkanesulfonylamino or —X³-Q³ [wherein X³ is a direct bond or selected from O, S, SO, SO₂, N(R⁷), CO, CH(OR⁷), CON(R⁷), N(R⁷)CO, SO₂N(R⁷), N(R⁷)SO₂, C(R⁷)₂O, C(R⁷)₂S, N(R⁷)C(R⁷)₂, N(R⁷)CON(R⁷) and N(R⁷)CSN(R⁷), wherein R⁷ is a hydrogen atom, (C₁-C₆)alkyl, aryl, aryl-(C₁-C₆)alkyl, (C₃-C₇)cycloalkyl, (C₃-C₇)cycloalkyl-(C₁-C₆)alkyl, (C₃-C₇)cycloalkenyl, (C₃-C₇)cycloalkenyl-(C₁-C₆)alkyl, heteroaryl, heteroaryl-(C₁-C₆)alkyl, heterocyclyl or heterocyclyl-(C₁-C₆)alkyl, and Q³ and R⁷ may be identical or different],
any aryl, heteroaryl or heterocyclyl of R^{b} may have 1, 2 or 3 identical or different substituents selected from hydrogen, halogeno, trifluoromethyl, cyano, nitro, hydroxy, amino, carboxyl, carbamoyl, (C₁-C₆)alkyl, (C₂-C₈)alkenyl, (C₂-C₈)alkynyl, (C₁-C₆)alkoxy, (C₂-C₆)alkenyloxy, (C₂-C₆)alkynyloxy, (C₁-C₆)alkylthio, (C₁-C₆)alkylsulfinyl, (C₁-C₆)alkylsulfonyl, (C₁-C₆)alkylamino, di-[(C₁-C₆)alkyl]amino, (C₁-C₆)alkoxycarbonyl, N—(C₁-C₆)alkylcarbamoyl, N,N-di-[(C₁-C₆)alkyl]carbamoyl, (C₂-C₆)alkanoyl, (C₂-C₆)alkanoyloxy, (C₂-C₆)alkanoylamino, N—(C₁-C₆)alkyl-(C₂-C₆)alkanoylamino, (C₃-C₆)alkenoylamino, N—(C₁-C₆)alkyl-(C₃-C₆)alkenoylamino, (C₃-C₆)alkynoylamino, N—(C₁-C₆)alkyl-(C₃-C₆)alkynoylamino, N—(C₁-C₆)alkylsulfamoyl, N,N-di-[(C₁-C₆)alkyl]sulfamoyl, (C₁-C₆)alkanesulfonylamino and N—(C₁-C₆)alkyl-(C₁-C₆)alkanesulfonylamino, or —X⁴—R⁸ [wherein X⁴ is a direct bond or selected from O and N(R⁹), wherein R⁹ is a hydrogen atom or (C₁-C₆)alkyl, and R⁸ is halogeno-(C₁-C₆)alkyl, hydroxy-(C₁-C₆)alkyl, (C₁-C₆)alkoxy-(C₁-C₆)alkyl, cyano-(C₁-C₆)alkyl, amino-(C₁-C₆)alkyl, (C₁-C₆)alkylamino-(C₁-C₆)alkyl or di-[(C₁-C₆)alkyl]amino-(C₁-C₆)alkyl], or —X⁵-Q⁴ [wherein X⁵ is a direct bond or selected from O, CO and N(R¹⁰), wherein R¹⁰ is a hydrogen atom or (C₁-C₆)alkyl, and Q⁴ is aryl, aryl-(C₁-C₆)alkyl, heteroaryl, heteroaryl-(C₁-C₆)alkyl, heterocyclyl or heterocyclyl-(C₁-C₆)alkyl, which may have 1 or 2 identical or different substituents selected from halogeno, (C₁-C₆)alkyl and (C₁-C₆)alkoxy],
heterocyclyl of R^{b} may have 1 or 2 oxo or thioxo substituents and
any identical or different substituents of aryl, heteroaryl or heterocyclyl of R^{b} may bond with each other to form —CH═CH—CH═CH—, —N═CH—CH═CH—, —CH═N—CH═CH—, —CH═CH—N═CH—, CH═CH—CH═N—, —N═CH—N═CH—, —CH═N—CH═N—, —N═CH—CH═N—, —N═N—CH═CH—, —CH═CH—N═N—, —CH═CH—O—, —O—CH═CH—, —CH═CH—S—, —S—CH═CH—, —C(R¹¹)₂—C(R¹¹)₂—O—, —O—C(R¹¹)₂—C(R¹¹)₂—, —C(R¹¹)₂—C(R¹¹)₂—S—, —S—C(R¹¹)₂—C(R¹¹)₂—, —O—C(R¹¹)₂—O—, —O—C(R¹¹)₂—C(R¹¹)₂—O—, —S—C(R¹¹)₂—S—, —S—C(R¹¹)₂—C(R¹¹)₂—S—, —CH═CH—NH—, —NH—CH═CH—, —C(R¹¹)₂—C(R¹¹)₂—NH—, —NH—C(R¹¹)₂—C(R¹¹)₂—, —N═CH—NH—, —NH—CH═N—, —NH—C(R¹¹)₂—NH—, —O—CH═N—, —N═CH—O—, —S—CH═N—, —N═CH—S—, —O—C(R¹¹)₂—NH—, —NH—C(R¹¹)₂—O—, —S—C(R¹¹)₂—NH—, —NH—C(R¹¹)₂—S—, —O—N═CH—, —CH═N—O—, —S—N═CH—, —CH═N—S—, —O—NH—C(R¹¹)₂—, —C(R¹¹)₂—NH—O—, —S—NH—C(R¹¹)₂—, —C(R¹¹)₂—NH—S—, —NH—N═CH—, —CH═N—NH—, —NH—NH—C(R¹¹)₂—, —C(R¹¹)₂—NH—NH, —N═N—NH— or —NH—N═N— [wherein R¹¹ is a hydrogen atom or (C₁-C₆)alkyl];
m is 0, 1, 2, 3 or 4; and
each R^{c} may be identical or different and is selected from hydrogen, halogeno, trifluoromethyl, cyano, isocyano, nitro, hydroxy, mercapto, amino, formyl, carboxyl, carbamoyl, (C₁-C₆)alkyl, (C₂-C₈)alkenyl, (C₂-C₈)alkynyl, (C₁-C₆)alkoxy, (C₂-C₆)alkenyloxy, (C₂-C₆)alkynyloxy, (C₁-C₆)alkylthio, (C₁-C₆)alkylsulfinyl, (C₁-C₆)alkylsulfonyl, (C₁-C₆)alkylamino, di-[(C₁-C₆)alkyl]amino, (C₁-C₆)alkoxycarbonyl, N—(C₁-C₆)alkylcarbamoyl, N,N-di-[(C₁-C₆)alkyl]carbamoyl, (C₂-C₆)alkanoyl, (C₂-C₆)alkanoyloxy, (C₂-C₆)alkanoylamino, N—(C₁-C₆)alkyl-(C₂-C₆)alkanoylamino, (C₃-C₆)alkenoylamino, N—(C₁-C₆)alkyl-(C₃-C₆)alkenoylamino, (C₃-C₆)alkynoylamino, N—(C₁-C₆)alkyl-(C₃-C₆)alkynoylamino, N—(C₁-C₆)alkylsulfamoyl, N,N-di-[(C₁-C₆)alkyl]sulfamoyl, (C₁-C₆)alkanesulfonylamino, N—(C₁-C₆)alkyl-(C₁-C₆)alkanesulfonylamino, aryl, aryl-(C₁-C₆)alkyl, (C₃-C₇)cycloalkyl, (C₃-C₇)cycloalkyl-(C₁-C₆)alkyl, (C₃-C₇)cycloalkenyl, (C₃-C₇)cycloalkenyl-(C₁-C₆)alkyl, heteroaryl, heteroaryl-(C₁-C₆)alkyl, heterocyclyl and heterocyclyl-(C₁-C₆)alkyl,
wherein the neighboring carbon atoms of any (C₂-C₆)alkylene of R^{c} may be separated by a group selected from O, S, SO, SO₂, N(R¹²), CO, CH(OR¹²), CON(R¹²), N(R¹²)CO, SO₂N(R¹²), N(R¹²)SO₂, CH═CH and C≡C, wherein R¹² is a hydrogen atom or (C₁-C₆)alkyl,
any CH₂═CH— or HC≡C— of R^{c} may have a substituent, at the terminal or at CH₂═ or HC≡, selected from halogeno, carboxyl, carbamoyl, (C₁-C₆)alkoxycarbonyl, N—(C₁-C₆)alkylcarbamoyl, N,N-di-[(C₁-C₆)alkyl]carbamoyl, amino-(C₁-C₆)alkyl, (C₁-C₆)alkylamino-(C₁-C₆)alkyl and di-[(C₁-C₆)alkyl]amino-(C₁-C₆)alkyl, or -Q⁵-X⁶ [wherein X⁶ is a direct bond or selected from CO and N(R¹³)CO, wherein R¹³ is a hydrogen atom or (C₁-C₆)alkyl, and Q⁵ is aryl, aryl-(C₁-C₆)alkyl, heteroaryl, heteroaryl-(C₁-C₆)alkyl, heterocyclyl or heterocyclyl-(C₁-C₆)alkyl],
any CH₂ or CH₃ of R^{c} may have at least one substituent selected from halogeno, (C₁-C₆)alkyl, hydroxy, cyano, amino, carboxyl, carbamoyl, (C₁-C₆)alkoxy, (C₁-C₆)alkylthio, (C₁-C₆)alkylsulfinyl, (C₁-C₆)alkylsulfonyl, (C₁-C₆)alkylamino, di-[(C₁-C₆)alkyl]amino, (C₁-C₆)alkoxycarbonyl, N—(C₁-C₆)alkylcarbamoyl, N,N-di-[(C₁-C₆)alkyl]carbamoyl, (C₂-C₆)alkanoyl, (C₂-C₆)alkanoyloxy, (C₂-C₆)alkanoylamino, N—(C₁-C₆)alkyl-(C₂-C₆)alkanoylamino, N—(C₁-C₆)alkylsulfamoyl, N,N-di-[(C₁-C₆)alkyl]sulfamoyl, (C₁-C₆)alkanesulfonylamino and N—(C₁-C₆)alkyl-(C₁-C₆)alkanesulfonylamino, or —X⁷-Q⁶ [wherein X⁷ is a direct bond or selected from O, S, SO, SO₂, N(R¹⁴), CO, CH(OR¹⁴), CON(R¹⁴), N(R¹⁴)CO, SO₂N(R¹⁴), N(R¹⁴)SO₂, C(R¹⁴)₂O, C(R¹⁴)₂S and N(R¹⁴)C(R¹⁴)₂, wherein R¹⁴ is a hydrogen atom or (C₁-C₆)alkyl, and Q⁶ is aryl, aryl-(C₁-C₆)alkyl, (C₃-C₇)cycloalkyl, (C₃-C₇)cycloalkyl-(C₁-C₆)alkyl, (C₃-C₇)cycloalkenyl, (C₃-C₇)cycloalkenyl-(C₁-C₆)alkyl, heteroaryl, heteroaryl-(C₁-C₆)alkyl, heterocyclyl or heterocyclyl-(C₁-C₆)alkyl],
any aryl, heteroaryl or heterocyclyl of R^{c} may have 1, 2 or 3 identical or different substituents selected from hydrogen, halogeno, trifluoromethyl, cyano, nitro, hydroxy, amino, carboxyl, carbamoyl, (C₁-C₆)alkyl, (C₂-C₈)alkenyl, (C₂-C₈)alkynyl, (C₁-C₆)alkoxy, (C₂-C₆)alkenyloxy, (C₂-C₆)alkynyloxy, (C₁-C₆)alkylthio, (C₁-C₆)alkylsulfinyl, (C₁-C₆)alkylsulfonyl, (C₁-C₆)alkylamino, di-[(C₁-C₆)alkyl]amino, (C₁-C₆)alkoxycarbonyl, N—(C₁-C₆)alkylcarbamoyl, N,N-di-[(C₁-C₆)alkyl]carbamoyl, (C₂-C₆)alkanoyl, (C₂-C₆)alkanoyloxy, (C₂-C₆)alkanoylamino, N—(C₁-C₆)alkyl-(C₂-C₆)alkanoylamino, (C₃-C₆)alkenoylamino, N—(C₁-C₆)alkyl-(C₃-C₆)alkenoylamino, (C₃-C₆)alkynoylamino, N—(C₁-C₆)alkyl-(C₃-C₆)alkynoylamino, N—(C₁-C₆)alkylsulfamoyl, N,N-di-[(C₁-C₆)alkyl]sulfamoyl, (C₁-C₆)alkanesulfonylamino and N—(C₁-C₆)alkyl-(C₁-C₆)alkanesulfonylamino, or —X⁸—R¹⁵ [wherein X⁸ is a direct bond or selected from O and N(R¹⁶), wherein R¹⁶ is a hydrogen atom or (C₁-C₆)alkyl, and R¹⁵ is halogeno-(C₁-C₆)alkyl, hydroxy-(C₁-C₆)alkyl, (C₁-C₆)alkoxy-(C₁-C₆)alkyl, cyano-(C₁-C₆)alkyl, amino-(C₁-C₆)alkyl, (C₁-C₆)alkylamino-(C₁-C₆)alkyl or di-[(C₁-C₆)alkyl]amino-(C₁-C₆)alkyl], or —X⁹-Q⁷ [wherein X⁹ is a direct bond or selected from O, CO and N(R¹⁷), wherein R¹⁷ is a hydrogen atom or (C₁-C₆)alkyl, Q⁷ is aryl, aryl-(C₁-C₆)alkyl, heteroaryl, heteroaryl-(C₁-C₆)alkyl, heterocyclyl or heterocyclyl-(C₁-C₆)alkyl, which may have 1 or 2 identical or different substituents selected from halogeno, (C₁-C₆)alkyl and (C₁-C₆)alkoxy, and any heterocyclyl of Q⁷ may have 1 or 2 oxo or thioxo substituents],
heterocyclyl of R^{c} may have 1 or 2 oxo or thioxo substituents and
any identical or different substituents of aryl, heteroaryl or heterocyclyl of R^{c} may bond with each other to form —CH═CH—CH═CH—, —N═CH—CH═CH—, —CH═N—CH═CH—, —CH═CH—N═CH—, CH═CH—CH═N—, —N═CH—N═CH—, —CH═N—CH═N—, —N═CH—CH═N—, —N═N—CH═CH—, —CH═CH—N═N—, —CH═CH—O—, —O—CH═CH—, —CH═CH—S—, —S—CH═CH—, —C(R¹⁸)₂—C(R¹⁸)₂—O—, —O—C(R¹⁸)₂—C(R¹⁸)₂—, —C(R¹⁸)₂—C(R¹⁸)₂—S—, —S—C(R¹⁸)₂—C(R¹⁸)₂—, —O—C(R¹⁸)₂—O—, —O—C(R¹⁸)₂—C(R¹⁸)₂—O—, —S—C(R¹⁸)₂—S—, —S—C(R¹⁸)₂—C(R¹⁸)₂—S—, —CH═CH—NH—, —NH—CH═CH—, —C(R¹⁸)₂—C(R¹⁸)₂—NH—, —NH—C(R¹⁸)₂—C(R¹⁸)₂—, —N═CH—NH—, —NH—CH═N—, —NH—C(R¹⁸)₂—NH—, —O—CH═N—, —N═CH—O—, —S—CH═N—, —N═CH—S—, —O—C(R¹⁸)₂—NH—, —NH—C(R¹⁸)₂—O—, —S—C(R¹⁸)₂—NH—, —NH—C(R¹⁸)₂—S—, —O—N═CH—, —CH═N—O—, —S—N═CH—, —CH═N—S—, —O—NH—C(R¹⁸)₂—, —C(R¹⁸)₂—NH—O—, —S—NH—C(R¹⁸)₂—, —C(R¹⁸)₂—NH—S—, —NH—N═CH—, —CH═N—NH—, —NH—NH—C(R¹⁸)₂—, —C(R¹⁸)₂—NH—NH, —N═N—NH— or —NH—N═N— [wherein R¹⁸ is a hydrogen atom or (C₁-C₆)alkyl].

2. The compound of claim 1, which is represented by the formula 1-1 below: 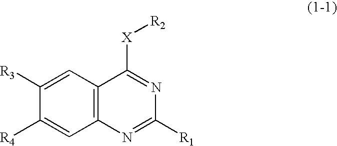
wherein
X is a direct bond or selected from O, S, SO, SO₂ and NR⁵, wherein R⁵ is a hydrogen atom, (C₁-C₆)alkyl, aryl, aryl-(C₁-C₆)alkyl, (C₃-C₇)cycloalkyl, (C₃-C₇)cycloalkyl-(C₁-C₆)alkyl, heteroaryl, heteroaryl-(C₁-C₆)alkyl, heterocyclyl or heterocyclyl-(C₁-C₆)alkyl;
R₁ is a substituent selected from hydrogen, (C₁-C₈)alkyl, halogeno-(C₁-C₆)alkyl, hydroxy-(C₁-C₆)alkyl, (C₁-C₆)alkoxy-(C₁-C₆)alkyl, amino-(C₁-C₆)alkyl, (C₁-C₆)alkylamino-(C₁-C₆)alkyl, di-[(C₁-C₆)alkyl]amino-(C₁-C₆)alkyl and (C₁-C₆)alkylsulfinyl, or —X¹-Q¹ [wherein X¹ is a direct bond or selected from O, S and NR⁵, and Q¹ and R⁵ may be identical or different],
wherein the neighboring carbon atoms of (C₂-C₆)alkylene of R₁ may be separated by a group selected from O, S and N(R⁵),
any CH₂ or CH₃ of R₁ may have at least one substituent selected from halogeno, (C₁-C₆)alkyl, hydroxy, amino, (C₁-C₆)alkoxy (C₁-C₆)alkylthio, (C₁-C₆)alkylsulfinyl, (C₁-C₆)alkylamino and di-[(C₁-C₆)alkyl]amino,
any aryl, heteroaryl or heterocyclyl of R₁ may have 0 to 3 identical or different substituents selected from halogeno, hydroxy, amino, (C₁-C₆)alkyl, aryl, aryl-(C₁-C₆)alkyl, heteroaryl, heteroaryl-(C₁-C₆)alkyl, heterocyclyl, heterocyclyl-(C₁-C₆)alkyl, (C₁-C₆)alkoxy, (C₁-C₆)alkylthio and (C₁-C₆)alkylsulfinyl and
heterocyclyl of R₁ may have 1 or 2 oxo or thioxo substituents;
R₂ is a substituent selected from hydrogen, (C₁-C₁₈)alkyl, (C₂-C₈)alkenyl, halogeno-(C₁-C₈)alkyl, hydroxy-(C₁-C₈)alkyl, (C₁-C₈)alkoxy-(C₁-C₈)alkyl, amino-(C₁-C₈)alkyl, (C₁-C₈)alkylamino-(C₁-C₈)alkyl, di-[(C₁-C₈)alkyl]amino-(C₁-C₈)alkyl, (C₁-C₈)alkylsulfinyl, aryl, aryl-(C₁-C₈)alkyl, (C₃-C₇)cycloalkyl, (C₃-C₇)cycloalkyl-(C₁-C₈)alkyl, heteroaryl, heteroaryl-(C₁-C₈)alkyl, heterocyclyl and heterocyclyl-(C₁-C₈)alkyl,
wherein the neighboring carbon atoms of (C₂-C₆)alkylene of R₂ may be separated by a group selected from O, S, N(R⁵), CO, CH(OR⁵), CON(R⁵), N(R⁵)CO, SO₂N(R⁵), N(R⁵)SO₂ and CH═CH,
any CH₂ or CH₃ of R₂ may have at least one substituent selected from halogeno, (C₁-C₆)alkyl substituent, hydroxy, cyano, amino, carboxyl, carbamoyl, (C₁-C₆)alkoxy (C₁-C₆)alkylthio, (C₁-C₆)alkylsulfinyl, (C₁-C₆)alkylamino, di-[(C₁-C₆)alkyl]amino, (C₁-C₆)alkoxycarbonyl, N—(C₁-C₆)alkylcarbamoyl, N,N-di-[(C₁-C₆)alkyl]carbamoyl, aryl, aryl-(C₁-C₆)alkyl, (C₃-C₇)cycloalkyl, (C₃-C₇)cycloalkyl-(C₁-C₆)alkyl, heteroaryl, heteroaryl-(C₁-C₆)alkyl, heterocyclyl and heterocyclyl-(C₁-C₆)alkyl,
any aryl, heteroaryl or heterocyclyl of R₂ may have 0 to 3 identical or different substituents selected from halogeno, trifluoromethyl, cyano, nitro, hydroxy, amino, carboxyl, carbamoyl, (C₁-C₆)alkyl, aryl, aryl-(C₁-C₆)alkyl, heteroaryl, heteroaryl-(C₁-C₆)alkyl, heterocyclyl, heterocyclyl-(C₁-C₆)alkyl, (C₂-C₈)alkenyl, (C₂-C₈)alkynyl, (C₁-C₆)alkoxy, (C₁-C₆)alkylthio, (C₁-C₆)alkylsulfinyl, (C₁-C₆)alkylamino, di-[(C₁-C₆)alkyl]amino, (C₁-C₆)alkoxycarbonyl, N—(C₁-C₆)alkylcarbamoyl and N,N-di-[(C₁-C₆)alkyl]carbamoyl,
heterocyclyl of R₂ may have 1 or 2 oxo or thioxo substituents and
any identical or different substituents of aryl, heteroaryl or heterocyclyl of R₂ may bond with each other to form —CH═CH—CH═CH—, —N═CH—CH═CH—, —CH═N—CH═CH—, —CH═CH—N═CH—, CH═CH—CH═N—, —N═CH—N═CH—, —CH═N—CH═N—, —N═CH—CH═N—, —CH═CH—O—, —O—CH═CH—, —CH═CH—S—, —S—CH═CH—, —C(R⁶)₂—C(R⁶)₂—O—, —O—C(R⁶)₂—C(R⁶)₂—, —C(R⁶)₂—C(R⁶)₂—S—, —S—C(R⁶)₂—C(R⁶)₂—, —O—C(R⁶)₂—O—, —O—C(R⁶)₂—C(R⁶)₂—O—, —S—C(R⁶)₂—S—, —S—C(R⁶)₂—C(R⁶)₂—S—, —CH═CH—NH—, —NH—CH═CH—, —C(R⁶)₂—C(R⁶)₂—NH—, —NH—C(R⁶)₂—C(R⁶)₂—, —N═CH—NH—, —NH—CH═N—, —NH—C(R⁶)₂—NH—, —O—CH═N—, —N═CH—O—, —S—CH═N—, —N═CH—S—, —O—C(R⁶)₂—NH—, —NH—C(R⁶)₂—O—, —S—C(R⁶)₂—NH—, —NH—C(R⁶)₂—S—, —N═N—NH— or —NH—N═N— [wherein R⁶ is a hydrogen atom or (C₁-C₃)alkyl]; and
each of R₃ and R₄ may be identical or different, and each may be independently a substituent selected from hydrogen, halogeno, trifluoromethyl, cyano, nitro, hydroxy, mercapto, amino, formyl, carboxyl, carbamoyl,(C₁-C₆)alkyl, (C₂-C₈)alkenyl, (C₂-C₆)alkoxy, (C₁-C₆)alkylthio, (C₁-C₆)alkylsulfinyl, (C₁-C₆)alkylamino, di-[(C₁-C₆)alkyl]amino, (C₁-C₆)alkoxycarbonyl, N—(C₁-C₆)alkylcarbamoyl, N,N-di-[(C₁-C₆)alkyl]carbamoyl, aryl, aryl-(C₁-C₆)alkyl, (C₃-C₇)cycloalkyl, (C₃-C₇)cycloalkyl-(C₁-C₆)alkyl, heteroaryl, heteroaryl-(C₁-C₆)alkyl, heterocyclyl and heterocyclyl-(C₁-C₆)alkyl,
any CH₂ or CH₃ of R₃ and R₄ may have at least one substituent selected from halogeno, hydroxy, amino, carboxyl, carbamoyl, (C₁-C₆)alkoxy, (C₁-C₆)alkylthio, (C₁-C₆)alkylsulfinyl, (C₁-C₆)alkylamino, di-[(C₁-C₆)alkyl]amino, (C₁-C₆)alkoxycarbonyl, N—(C₁-C₆)alkylcarbamoyl, N,N-di-[(C₁-C₆)alkyl]carbamoyl, aryl, aryl-(C₁-C₆)alkyl, (C₃-C₇)cycloalkyl, (C₃-C₇)cycloalkyl-(C₁-C₆)alkyl, heteroaryl, heteroaryl-(C₁-C₆)alkyl, heterocyclyl and heterocyclyl-(C₁-C₆)alkyl,
any aryl, heteroaryl or heterocyclyl of R₃ and R₄ may have 1, 2 or 3 identical or different substituents selected from halogeno, trifluoromethyl, nitro, hydroxy, amino, (C₁-C₆)alkoxy, (C₁-C₆)alkylthio, (C₁-C₆)alkylamino, di-[(C₁-C₆)alkyl]amino, (C₁-C₆)alkoxycarbonyl, N—(C₁-C₆)alkylcarbamoyl and N,N-di-[(C₁-C₆)alkyl]carbamoyl and
heterocyclyl of R₃ and R₄ may have 1 or 2 oxo or thioxo substituents.

3. The compound of claim 2, wherein
X is O, S or NR₅;
R₁ is a hydrogen atom, C₁-C₆ alkyl, C₁-C₄ haloalkyl, C₁-C₆ alkylhydroxy, C₁-C₆ alkylsulfanyl, phenyl, phenyl C₁-C₈ alkyl, pyridino, furano, thiopheno, C₃-C₇ cycloalkyl, pyrrolidino, piperidino, morpholino or N—(C₁-C₆)alkylpiperazino;
R₂ is C₁-C₁₈ alkyl, C₂-C₈ haloalkyl, C₃-C₇ cycloalkyl, (C₃-C₇)cycloalkyl C₁-C₈ alkyl, C₂-C₈ alkenyl, (C₃-C₇)cycloalkenyl C₁-C₈ alkyl, (C₁-C₈)alkyl C₁-C₈ alkoxyalkyl, C₁-C₈ hydroxyalkyl, hydroxy-(C₁-C₈)alkyl-(C₁-C₈)alkoxy alkyl, phenyl, phenyl C₁-C₈ alkyl, di-phenyl C₁-C₈ alkyl, phenyl C₁-C₈ alkylhydroxy, phenyl C₁-C₈ alkenyl, piperidine C₁-C₈ alkyl, N-[phenyl-(C₁-C₆)alkyl]piperidino, N—(C₁-C₆)alkylpiperidine C₁-C₈ alkyl, pyrrolidinone N—(C₁-C₈)alkyl, naphthalene C₁-C₈ alkyl, morpholine C₁-C₈ alkyl, piperazine C₁-C₈ alkyl, N—(C₁-C₆)alkylpiperazine C₁-C₈ alkyl, pyridine C₁-C₈ alkyl, pyrrolidine C₁-C₈ alkyl, N—(C₁-C₆)alkylpyrrolidine C₁-C₈ alkyl, thiopene C₁-C₈ alkyl or benzodioxol C₁-C₈ alkyl;
each of R₃ and R₄ is a hydrogen atom, nitro or C₁-C₈ alkoxy; and
R₅ is a hydrogen atom, C₁-C₄ alkyl or C₁-C₈ hydroxyalkyl,
wherein the aforementioned cyclic substituent selected from C₃-C₇ cycloalkyl, phenyl, piperidine, naphthalene, morpholine, pyridine, pyrrolidine, pyrrolidinone, piperazine, thiopene, and benzodioxol may have 1 to 3 substituents selected from C₁-C₈ alkyl, halogeno, hydroxy, C₁-C₈ alkoxy, nitro and trifluoromethyl.

4. The compound of claim 1, which is selected from the following compounds:
Compound No. 1: 2-[2-(2-phenylquinazolino-4-ylamino)-ethoxy]-ethanol
Compound No. 2: isopropyl-(2-trichloromethylquinazolino-4-yl)-amine
Compound No. 3: cyclopropyl-(2-trichloromethylquinazolino-4-yl)-amine
Compound No. 4: (2-piperidino-1-ylethyl)-(2-trichloromethylquinazolino-4-yl)-amine
Compound No. 5: (1-phenylethyl)-(2-trichloromethylquinazolino-4-yl)-amine
Compound No. 6: (4-phenylbutyl)-(2-trichloromethylquinazolino-4-yl)-amine
Compound No. 7: propyl-(2-trichloromethylquinazolino-4-yl)-amine
Compound No. 8: (2-ethylsulfanylquinazolino-4-yl)-isopropylamine
Compound No. 9: (2-ethylsulfanylquinazolino-4-yl)-(3-fluorobenzyl)-amine
Compound No. 10: (2-ethylsulfanylquinazolino-4-yl)-[2-(3-fluorophenyl)-ethyl]-amine
Compound No. 11: (2-ethylsulfanylquinazolino-4-yl)-(2-morpholino-4-ylethyl)-amine
Compound No. 12: (2-ethylsulfanylquinazolino-4-yl)-[2-(3-trifluoromethyl-phenyl)ethyl]-amine
Compound No. 13: (6-nitro-quinazolino-4-yl)-phenylamine
Compound No. 14: (6,7-diethoxyquinazolino-4-yl)-naphthaleno-1-ylmethylamine
Compound No. 15: (6,7-diethoxyquinazolino-4-yl)-(3-morpholino-4-ylpropyl)-amine
Compound No. 16: allyl-(6,7-diethoxyquinazolino-4-yl)-amine
Compound No. 17: (6,7-diethoxyquinazolino-4-yl)-(2-pyridino-2-ylethyl)-amine
Compound No. 18: (4-chlorobenzyl)-(6,7-diethoxyquinazolino-4-yl)-amine
Compound No. 19: butyl-(2-ethylsulfanylquinazolino-4-yl)-amine
Compound No. 20: sec-butyl-(2-ethylsulfanylquinazolino-4-yl)-amine
Compound No. 21: (2-ethylsulfanylquinazolino-4-yl)-isobutylamine
Compound No. 22: benzyl-(2-ethylsulfanylquinazolino-4-yl)-amine
Compound No. 23: (2-ethylsulfanylquinazolino-4-yl)-(1-phenylethyl)-amine
Compound No. 24: (2-ethylsulfanylquinazolino-4-yl)-(3-methoxybenzyl)-amine
Compound No. 25: (2-ethylsulfanylquinazolino-4-yl)-(4-methoxybenzyl)-amine
Compound No. 26: (2-ethylsulfanylquinazolino-4-yl)-(3-trifluoromethylbenzyl)-amine
Compound No. 27: cyclohexyl-(2-ethylsulfanylquinazolino-4-yl)-amine
Compound No. 28: cyclopentyl-(2-ethylsulfanylquinazolino-4-yl)-amine
Compound No. 29: (2-ethylsulfanylquinazolino-4-yl)-phenethylamine
Compound No. 30: (2-ethylsulfanylquinazolino-4-yl)-(4-phenylbutyl)-amine
Compound No. 31: (2-ethylsulfanylquinazolino-4-yl)-propylamine
Compound No. 32: (2-ethylsulfanylquinazoline-4-yl)-(3-phenylpropyl)-amine
Compound No. 33: (2-ethylsulfanylquinazolino-4-yl)-(3-isopropoxypropyl)-amine
Compound No. 34: (2-ethylsulfanylquinazolino-4-yl)-(2-methoxy-ethyl)-amine
Compound No. 35: (2-ethylsulfanylquinazolino-4-yl)-naphthaleno-1-ylmethylamine
Compound No. 36: (2-ethylsulfanylquinazolino-4-yl)-(3-morpholino-4-ylpropyl)-amine
Compound No. 37: benzo[1,3]dioxol-5-ylmethyl-(2-ethylsulfanylquinazolino-4-yl)-amine
Compound No. 38: (3,5-dimethoxybenzyl)-(2-ethylsulfanylquinazolino-4-yl)-amine
Compound No. 39: (2-ethylsulfanylquinazolino-4-yl)-(2-fluorobenzyl)-amine
Compound No. 40: (2-ethylsulfanylquinazolino-4-yl)-(1-methoxymethylpropyl)-amine
Compound No. 41: (1-benzyl-piperidino-4-yl)-(2-ethylsulfanylquinazolino-4-yl)-amine
Compound No. 42: allyl-(2-ethylsulfanylquinazolino-4-yl)-amine
Compound No. 43: (2-ethylsulfanylquinazolino-4-yl)-hexadecylamine
Compound No. 44: (2-ethylsulfanylquinazolino-4-yl)-(2-piperidino-1-ylethyl)-amine
Compound No. 45: (2-ethylsulfanylquinazolino-4-yl)-(2-pyrrolidino-1-ylethyl)-amine
Compound No. 46: (2-ethylsulfanylquinazolino-4-yl)-(2-pyridino-2-ylethyl)-amine
Compound No. 47: 2-(2-ethylsulfanylquinazolino-4-ylamino)-ethanol
Compound No. 48: (2-ethylsulfanylquinazolino-4-yl)-[3-(2-methyl-piperidino-1-yl)-propyl]-amine
Compound No. 49: (2-chloro-6-fluorobenzyl)-(2-ethylsulfanylquinazolino-4-yl)-amine
Compound No. 50: 2-[2-(2-ethylsulfanylquinazolino-4-ylamino)-ethoxy]-ethanol
Compound No. 51: cyclohexyl-(2-phenylquinazolino-4-yl)-amine
Compound No. 52: (4-phenylbutyl)-(2-phenylquinazolino-4-yl)-amine
Compound No. 53: (5-phenyl-pentyl)-(2-phenylquinazolino-4-yl)-amine
Compound No. 54: (4-methylbenzyl)-(2-phenylquinazolino-4-yl)-amine
Compound No. 55: isobutyl-(2-phenylquinazolino-4-yl)-amine
Compound No. 56: butyl-(2-phenylquinazolino-4-yl)-amine
Compound No. 57: naphthaleno-1-ylmethyl-(2-phenylquinazolino-4-yl)-amine
Compound No. 58: (2-phenylquinazolino-4-yl)-pyridino-2-ylmethylamine
Compound No. 59: (1-phenylquinazolino-4-yl)-thiopenyl-2-ylmethylamine
Compound No. 60: isopropyl-(2-phenylquinazolino-4-yl)-amine
Compound No. 61: (2-phenylquinazolino-4-yl)-propylamine
Compound No. 62: (3-isopropoxypropyl)-(2-phenylquinazolino-4-yl)-amine
Compound No. 63: (3-fluorobenzyl)-(2-phenylquinazolino-4-yl)-amine
Compound No. 64: (2-phenylquinazolino-4-yl)-[2-(3-trifluoromethyl-phenyl)-ethyl]-amine
Compound No. 65: benzo[1,3]dioxol-5-ylmethyl-(2-phenylquinazolino-4-yl)-amine
Compound No. 66: (2-phenylquinazolino-4-yl)-(3-trifluoromethylbenzyl)-amine
Compound No. 67: (2-methoxy-ethyl)-(2-phenylquinazolino-4-yl)-amine
Compound No. 68: phenethyl-(2-phenylquinazolino-4-yl)-amine
Compound No. 69: phenyl-(2-phenylquinazolino-4-yl)-amine
Compound No. 70: (4-methoxyphenyl)-(2-phenylquinazolino-4-yl)-amine
Compound No. 71: (2-phenylquinazolino-4-yl)-o-tollylamine
Compound No. 72: (4-isopropyl-phenyl)-(2-phenylquinazolino-4-yl)-amine
Compound No. 73: (2,4-dimethyl-phenyl)-(2-phenylquinazolino-4-yl)-amine
Compound No. 74: (2-phenylquinazolino-4-yl)-(4-undecyl-phenyl)-amine
Compound No. 75: (4-ethyl-phenyl)-(2-phenylquinazolino-4-yl)-amine
Compound No. 76: (4-fluorophenyl)-(2-phenylquinazolino-4-yl)-amine
Compound No. 77: (2-methoxyphenyl)-(2-phenylquinazolino-4-yl)-amine
Compound No. 78: (2-phenylquinazolino-4-yl)-(2,4,6-trimethyl-phenyl)-amine
Compound No. 79: (4-butyl-phenyl)-(2-phenylquinazolino-4-yl)-amine
Compound No. 80: (2-phenoxy-phenyl)-(2-phenylquinazolino-4-yl)-amine
Compound No. 81: (3-phenoxy-phenyl)-(2-phenylquinazolino-4-yl)-amine
Compound No. 82: (4-phenoxy-phenyl)-(2-phenylquinazolino-4-yl)-amine
Compound No. 83: (3-bromophenyl)-(2-phenylquinazolino-4-yl)-amine
Compound No. 84: 2-methoxy-5-(2-phenylquinazolino-4-ylamino)phenol
Compound No. 85: 5-(2-phenylquinazolino-4-ylamino)-pentane-1-ol
Compound No. 86: benzyl-(2-phenylquinazolino-4-yl)-amine
Compound No. 87: (1-phenylethyl)-(2-phenylquinazolino-4-yl)-amine
Compound No. 88: (4-chlorobenzyl)-(2-phenylquinazolino-4-yl)-amine
Compound No. 89: (3,4-dichlorobenzyl)-(2-phenylquinazolino-4-yl)-amine
Compound No. 90: (3-methoxybenzyl)-(2-phenylquinazolino-4-yl)-amine
Compound No. 91: (2-methoxybenzyl)-(2-phenylquinazolino-4-yl)-amine
Compound No. 92: (3,5-dimethoxybenzyl)-(2-phenylquinazolino-4-yl)-amine
Compound No. 93: (2,3-dimethoxybenzyl)-(2-phenylquinazolino-4-yl)-amine
Compound No. 94: (2-fluorobenzyl)-(2-phenylquinazolino-4-yl)-amine
Compound No. 95: cyclopentyl-(2-phenylquinazolino-4-yl)-amine
Compound No. 96: sec-butyl-(2-phenylquinazolino-4-yl)-amine
Compound No. 97: allyl-(2-phenylquinazolino-4-yl)-amine
Compound No. 98: (2-phenylquinazolino-4-yl)-(2-trifluoromethylbenzyl)-amine
Compound No. 99: hexadecyl-(2-phenylquinazolino-4-yl)-amine
Compound No. 100: (2-phenylquinazolino-4-yl)-(2-pyrrolidino-1-ylethyl)-amine
Compound No. 101: (2-phenylquinazolino-4-yl)-(2-piperidino-1-ylethyl)-amine
Compound No. 102: (2-phenylquinazolino-4-yl)-(2-pyridino-2-ylethyl)-amine
Compound No. 103: [2-(3-fluorophenyl)-ethyl]-(2-phenylquinazolino-4-yl)-amine
Compound No. 104: (1-methoxymethylpropyl)-(2-phenylquinazolino-4-yl)-amine
Compound No. 105: (1-benzyl-piperidino-4-yl)-(2-phenylquinazolino-4-yl)-amine
Compound No. 106: (2-morpholino-4-ylethyl)-(2-phenylquinazolino-4-yl)-amine
Compound No. 107: [2-(3,4-dimethoxyphenyl)-ethyl]-(2-phenylquinazolino-4-yl)-amine
Compound No. 108: (2,4-dichlorobenzyl)-(2-phenylquinazolino-4-yl)-amine
Compound No. 109: (2-chlorobenzyl)-(2-phenylquinazolino-4-yl)-amine
Compound No. 110: cyclopropyl-(2-phenylquinazolino-4-yl)-amine
Compound No. 111: (2-phenylquinazolino-4-yl)-pyridino-3-ylmethylamine
Compound No. 112: 2-(2-phenylquinazolino-4-ylamino)-propane-1-ol
Compound No. 113: 2-methoxy-4-[(2-phenylquinazolino-4-ylamino)-methyl]-phenol
Compound No. 114: (2-fluoro-ethyl)-(2-phenylquinazolino-4-yl)-amine
Compound No. 115: (2,3-dichlorobenzyl)-(2-phenylquinazolino-4-yl)-amine
Compound No. 116: (3-nitrobenzyl)-(2-phenylquinazolino-4-yl)-amine
Compound No. 117: (1-ethyl-pyrrolidino-2-ylmethyl)-(2-phenylquinazolino-4-yl)-amine
Compound No. 118: (3,4-dimethoxybenzyl)-(2-phenylquinazolino-4-yl)-amine
Compound No. 119: (2-phenylquinazolino-4-yl)-(2-p-tollylethyl)-amine
Compound No. 120: [2-(3-methoxyphenyl)-ethyl]-(2-phenylquinazolino-4-yl)-amine
Compound No. 121: (1-ethylpropyl)-(2-phenylquinazolino-4-yl)-amine
Compound No. 122: [2-(2,5-dimethoxyphenyl)-ethyl]-(2-phenylquinazolino-4-yl)-amine
Compound No. 123: [2-(4-chlorophenyl)-ethyl]-(2-phenylquinazolino-4-yl)-amine
Compound No. 124: [2-(4-methoxyphenyl)-ethyl]-(2-phenylquinazolino-4-yl)-amine
Compound No. 125: (1,3-dimethylbutyl)-(2-phenylquinazolino-4-yl)-amine
Compound No. 126: [2-(3-chlorophenyl)-ethyl]-(2-phenylquinazolino-4-yl)-amine
Compound No. 127: [2-(2,4-dichlorophenyl)-ethyl]-(2-phenylquinazolino-4-yl)-amine
Compound No. 128: [2-(3,4-difluorophenyl)-ethyl]-(2-phenylquinazolino-4-yl)-amine
Compound No. 129: (2-phenylquinazolino-4-yl)-(3,4,5-trimethoxybenzyl)-amine
Compound No. 130: (3-methylbutyl)-(2-phenylquinazolino-4-yl)-amine
Compound No. 131: (2-cyclohex-1-enylethyl)-(2-phenylquinazolino-4-yl)-amine
Compound No. 132: cyclohexylmethyl-(2-phenylquinazolino-4-yl)-amine
Compound No. 133: (2,2-diphenylethyl)-(2-phenylquinazolino-4-yl)-amine
Compound No. 134: (2,4-difluorobenzyl)-(2-phenylquinazolino-4-yl)-amine
Compound No. 135: (2,5-difluorobenzyl)-(2-phenylquinazolino-4-yl)-amine
Compound No. 136: (3,3-diphenylpropyl)-(2-phenylquinazolino-4-yl)-amine
Compound No. 137: [2-(4-bromophenyl)-ethyl]-(2-phenylquinazolino-4-yl)-amine
Compound No. 138: butylmethyl-(2-phenylquinazolino-4-yl)-amine
Compound No. 139: (2-phenylquinazolino-4-yl)-(4-trifluoromethylbenzyl)-amine
Compound No. 140: [2-(2-fluorophenyl)-ethyl]-(2-phenylquinazolino-4-yl)-amine
Compound No. 141: (2-phenylquinazolino-4-yl)-pyridino-4-ylmethylamine
Compound No. 142: (2-phenylquinazolino-4-yl)-pyridino-2-ylmethylamine
Compound No. 143: (2,3-dimethylcyclohexyl)-(2-phenylquinazolino-4-yl)-amine
Compound No. 144: (2,4-dimethoxybenzyl)-(2-phenylquinazolino-4-yl)-amine
Compound No. 145: (3-chlorobenzyl)-(2-phenylquinazolino-4-yl)-amine
Compound No. 146: 1-phenyl-2-(2-phenylquinazolino-4-ylamino)-ethanol
Compound No. 147: (2,5-dimethoxybenzyl)-(2-phenylquinazolino-4-yl)-amine
Compound No. 148: (4-bromobenzyl)-(2-phenylquinazolino-4-yl)-amine
Compound No. 149: [2-(3,4-dichlorophenyl)-ethyl]-(2-phenylquinazolino-4-yl)-amine
Compound No. 150: 1-[3-(2-phenylquinazolino-4-ylamino)-propyl]-pyrrolidino-2-one
Compound No. 151: [2-(2-chlorophenyl)-ethyl]-(2-phenylquinazolino-4-yl)-amine
Compound No. 152: 2-[2-(2-phenylquinazolino-4-ylamino)-ethyl]-phenol
Compound No. 153: (3-bromobenzyl)-(2-phenylquinazolino-4-yl)-amine
Compound No. 154: (2,6-dimethoxybenzyl)-(2-phenylquinazolino-4-yl)-amine
Compound No. 155: (2-bromobenzyl)-(2-phenylquinazolino-4-yl)-amine
Compound No. 156: (3-morpholino-4-ylpropyl)-(2-phenylquinazolino-4-yl)-amine
Compound No. 157: 2-[butyl-(2-phenylquinazolino-4-yl)-amino]-ethanol
Compound No. 158: [2-(4-methylpiperazino-1-yl)-ethyl]-(2-phenylquinazolino-4-yl)-amine
Compound No. 159: cyclopentylmethyl-(2-phenylquinazolino-4-yl)-amine
Compound No. 160: (2-phenylquinazolino-4-yl)-(2-piperazino-1-ylethyl)-amine
Compound No. 161: O-(4-nitrobenzyl)-N-(2-phenylquinazolino-4-yl)-hydroxylamine
Compound No. 162: (4-chlorophenyl)-(2-phenylquinazolino-4-yl)-amine
Compound No. 163: benzyl-(2-methylquinazolino-4-yl)-amine
Compound No. 164: (3-methoxybenzyl)-(2-methylquinazolino-4-yl)-amine
Compound No. 165: (4-methoxybenzyl)-(2-methylquinazolino-4-yl)-amine
Compound No. 166: (2-methylquinazolino-4-yl)-(3-trifluoromethylbenzyl)-amine
Compound No. 167: (2-methylquinazolino-4-yl)-phenethylamine
Compound No. 168: (2-methylquinazolino-4-yl)-(4-phenylbutyl)-amine
Compound No. 169: (2-methylquinazolino-4-yl)-naphthaleno-1-ylmethylamine
Compound No. 170: benzo[1,3]dioxol-5-ylmethyl-(2-methylquinazolino-4-yl)-amine
Compound No. 171: (3-fluorobenzyl)-(2-methylquinazolino-4-yl)-amine
Compound No. 172: [2-(3-fluorophenyl)-ethyl]-(2-methylquinazolino-4-yl)-amine
Compound No. 173: 2-[2-(2-methylquinazolino-4-ylamino)-ethoxy]-ethanol
Compound No. 174: 2-(2-methylquinazolino-4-ylamino)-ethanol
Compound No. 175: benzyl-(2-isopropylquinazolino-4-yl)-amine
Compound No. 176: (2-isopropylquinazolino-4-yl)-(3-methoxybenzyl)-amine
Compound No. 177: (2-isopropylquinazolino-4-yl)-(4-methoxybenzyl)-amine
Compound No. 178: (2-isopropylquinazolino-4-yl)-(3-trifluoromethylbenzyl)-amine
Compound No. 179: (2-isopropylquinazolino-4-yl)-phenethylamine
Compound No. 180: (2-isopropylquinazolino-4-yl)-(4-phenylbutyl)-amine
Compound No. 181: (2-isopropylquinazolino-4-yl)-naphthaleno-1-ylmethylamine
Compound No. 182: benzo[1,3]dioxol-5-ylmethyl-(2-isopropylquinazolino-4-yl)-amine
Compound No. 183: (3-fluorobenzyl)-(2-isopropylquinazolino-4-yl)-amine
Compound No. 184: [2-(3-fluorophenyl)-ethyl]-(2-isopropylquinazolino-4-yl)-amine
Compound No. 185: 2-[2-(2-isopropylquinazolino-4-ylamino)-ethoxy]-ethanol
Compound No. 186: 2-(2-isopropylquinazolino-4-ylamino)-ethanol
Compound No. 187: benzyl-(2-tert-butylquinazolino-4-yl)-amine
Compound No. 188: (2-tert-butylquinazolino-4-yl)-(3-methoxybenzyl)-amine
Compound No. 189: (2-tert-butylquinazolino-4-yl)-(4-methoxybenzyl)-amine
Compound No. 189: (2-tert-butylquinazolino-4-yl)-(4-methoxybenzyl)-amine
Compound No. 190: (2-tert-butylquinazolino-4-yl)-(3-trifluoromethylbenzyl)-amine
Compound No. 191: (2-tert-butylquinazolino-4-yl)-phenethylamine
Compound No. 192: (2-tert-butylquinazolino-4-yl)-(4-phenylbutyl)-amine
Compound No. 193: (2-tert-butylquinazolino-4-yl)-naphthaleno-1-ylmethylamine
Compound No. 194: benzo[1,3]dioxol-5-ylmethyl-(2-tert-butylquinazolino-4-yl)-amine
Compound No. 195: (2-tert-butylquinazolino-4-yl)-(3-fluorobenzyl)-amine
Compound No. 196: (2-tert-butylquinazolino-4-yl)-[2-(3-fluorophenyl)-ethyl]-amine
Compound No. 197: 2-[2-(2-tert-butylquinazolino-4-ylamino)-ethoxy]-ethanol
Compound No. 198: 2-(2-tert-butylquinazolino-4-ylamino)-ethanol
Compound No. 199: benzyl-(2-cyclohexylquinazolino-4-yl)-amine
Compound No. 200: (2-cyclohexylquinazolino-4-yl)-(3-methoxybenzyl)-amine
Compound No. 201: (2-cyclohexylquinazolino-4-yl)-(4-methoxybenzyl)-amine
Compound No. 202: (2-cyclohexylquinazolino-4-yl)-(3-trifluoromethylbenzyl)-amine
Compound No. 203: (2-cyclohexylquinazolino-4-yl)-phenethylamine
Compound No. 204: (2-cyclohexylquinazolino-4-yl)-(4-phenylbutyl)-amine
Compound No. 205: (2-cyclohexylquinazolino-4-yl)-naphthaleno-1-ylmethylamine
Compound No. 206: benzo[1,3]dioxol-5-ylmethyl-(2-cyclohexylquinazolino-4-yl)-amine
Compound No. 207: (2-cyclohexylquinazolino-4-yl)-(3-fluorobenzyl)-amine
Compound No. 208: (2-cyclohexylquinazolino-4-yl)-[2-(3-fluorophenyl)-ethyl]-amine
Compound No. 209: 2-[2-(2-cyclohexylquinazolino-4-ylamino)-ethoxy]-ethanol
Compound No. 210: 2-(2-cyclohexylquinazolino-4-ylamino)-ethanol
Compound No. 211: benzyl-(2-benzylquinazolino-4-yl)-amine
Compound No. 212: (2-benzylquinazolino-4-yl)-(3-methoxybenzyl)-amine
Compound No. 213: (2-benzylquinazolino-4-yl)-(4-methoxybenzyl)-amine
Compound No. 214: (2-benzylquinazolino-4-yl)-(3-trifluoromethylbenzyl)-amine
Compound No. 215: (2-benzylquinazolino-4-yl)-phenethylamine
Compound No. 216: (2-benzylquinazolino-4-yl)-(4-phenylbutyl)-amine
Compound No. 217: (2-benzylquinazolino-4-yl)-naphthaleno-1-ylmethylamine
Compound No. 218: benzo[1,3]dioxol-5-ylmethyl-(2-benzylquinazolino-4-yl)-amine
Compound No. 219: (2-benzylquinazolino-4-yl)-(3-fluorobenzyl)-amine
Compound No. 220: (2-benzylquinazolino-4-yl)-[2-(3-fluorophenyl)-ethyl]-amine
Compound No. 221: 2-[2-(2-benzylquinazolino-4-ylamino)-ethoxy]-ethanol
Compound No. 222: 2-(2-benzylquinazolino-4-ylamino)-ethanol
Compound No. 223: benzyl-(2-pyridino-2-ylquinazolino-4-yl)-amine
Compound No. 224: (3-methoxybenzyl)-(2-pyridino-2-ylquinazolino-4-yl)-amine
Compound No. 225: (4-methoxybenzyl)-(2-pyridino-2-ylquinazolino-4-yl)-amine
Compound No. 226: (2-pyridino-2-ylquinazolino-4-yl)-(3-trifluoromethylbenzyl)-amine
Compound No. 227: phenethyl-(2-pyridino-2-ylquinazolino-4-yl)-amine
Compound No. 228: (4-phenylbutyl)-(2-pyridino-2-ylquinazolino-4-yl)-amine
Compound No. 229: naphthaleno-1-ylmethyl-(2-pyridino-2-ylquinazolino-4-yl)-amine
Compound No. 230: benzo[1,3]dioxol-5-ylmethyl-(2-pyridino-2-ylquinazolino-4-yl)-amine
Compound No. 231: (3-fluorobenzyl)-(2-pyridino-2-ylquinazolino-4-yl)-amine
Compound No. 232: [2-(3-fluorophenyl)-ethyl]-(2-pyridino-2-ylquinazolino-4-yl)-amine
Compound No. 233: 2-[2-(2-pyridino-2-ylquinazolino-4-ylamino)-ethoxy]-ethanol
Compound No. 234: 2-(2-pyridino-2-ylquinazolino-4-ylamino)-ethanol
Compound No. 235: benzyl-(2-pyridino-3-ylquinazolino-4-yl)-amine
Compound No. 236: (3-methoxybenzyl)-(2-pyridino-3-ylquinazolino-4-yl)-amine
Compound No. 237: (4-methoxybenzyl)-(2-pyridino-3-ylquinazolino-4-yl)-amine
Compound No. 238: (2-pyridino-3-ylquinazolino-4-yl)-(3-trifluoromethylbenzyl)-amine
Compound No. 239: phenethyl-(2-pyridino-3-ylquinazolino-4-yl)-amine
Compound No. 240: (4-phenylbutyl)-(2-pyridino-3-ylquinazolino-4-yl)-amine
Compound No. 241: naphthaleno-1-ylmethyl-(2-pyridino-3-ylquinazolino-4-yl)-amine
Compound No. 242: benzo[1,3]dioxol-5-ylmethyl-(2-pyridino-3-ylquinazolino-4-yl)-amine
Compound No. 243: (3-fluorobenzyl)-(2-pyridino-3-ylquinazolino-4-yl)-amine
Compound No. 244: [2-(3-fluorophenyl)-ethyl]-(2-pyridino-3-ylquinazolino-4-yl)-amine
Compound No. 245: 2-[2-(2-pyridino-3-ylquinazolino-4-ylamino)-ethoxy]-ethanol
Compound No. 246: 2-(2-pyridino-3-ylquinazolino-4-ylamino)-ethanol
Compound No. 247: benzyl-(2-pyridino-4-ylquinazolino-4-yl)-amine
Compound No. 248: (3-methoxybenzyl)-(2-pyridino-4-ylquinazolino-4-yl)-amine
Compound No. 249: (4-methoxybenzyl)-(2-pyridino-4-ylquinazolino-4-yl)-amine
Compound No. 250: (2-pyridino-4-ylquinazolino-4-yl)-(3-trifluoromethylbenzyl)-amine
Compound No. 251: phenethyl-(2-pyridino-4-ylquinazolino-4-yl)-amine
Compound No. 252: (4-phenylbutyl)-(2-pyridino-4-ylquinazolino-4-yl)-amine
Compound No. 253: naphthaleno-1-ylmethyl-(2-pyridino-4-ylquinazolino-4-yl)-amine
Compound No. 254: benzo[1,3]dioxol-5-ylmethyl-(2-pyridino-4-ylquinazolino-4-yl)-amine
Compound No. 255: (3-fluorobenzyl)-(2-pyridino-4-ylquinazolino-4-yl)-amine
Compound No. 256: [2-(3-fluorophenyl)-ethyl]-(2-pyridino-4-ylquinazolino-4-yl)-amine
Compound No. 257: 2-[2-(2-pyridino-4-ylquinazolino-4-ylamino)-ethoxy]-ethanol
Compound No. 258: 2-(2-pyridino-4-ylquinazolino-4-ylamino)-ethanol
Compound No. 259: benzyl-[2-(4-chlorophenyl)-quinazolino-4-yl]-amine
Compound No. 260: [2-(4-chlorophenyl)-quinazolino-4-yl]-(3-methoxybenzyl)-amine
Compound No. 261: [2-(4-chlorophenyl)-quinazolino-4-yl]-(4-methoxybenzyl)-amine
Compound No. 262: [2-(4-chlorophenyl)-quinazolino-4-yl]-(3-trifluoromethylbenzyl)-amine
Compound No. 263: [2-(4-chlorophenyl)-quinazolino-4-yl]-phenethylamine
Compound No. 264: [2-(4-chlorophenyl)-quinazolino-4-yl]-(4-phenylbutyl)-amine
Compound No. 265: [2-(4-chlorophenyl)-quinazolino-4-yl]-naphthaleno-1-ylmethylamine
Compound No. 266: benzo[1,3]dioxol-5-ylmethyl-[2-(4-chlorophenyl)-quinazolino-4-yl]-amine
Compound No. 267: [2-(4-chlorophenyl)-quinazolino-4-yl]-(3-fluorobenzyl)-amine
Compound No. 268: [2-(4-chlorophenyl)-quinazolino-4-yl]-[2-(3-fluorophenyl)-ethyl]-amine
Compound No. 269: 2-{2-[2-(4-chlorophenyl)-quinazolino-4-ylamino]-ethoxy}-ethanol
Compound No. 270: 2-[2-(4-chlorophenyl)-quinazolino-4-ylamino]-ethanol
Compound No. 271: (2,4-dimethoxybenzyl)-(2-isopropylquinazolino-4-yl)-amine
Compound No. 272: (2-benzylquinazolino-4-yl)-(2,4-dimethoxybenzyl)-amine
Compound No. 273: 4-(2,4-dimethoxybenzyloxy)-2-phenylquinazoline
Compound No. 274: 4-(2,4-dimethoxybenzylsulfanyl)-2-phenylquinazoline
Compound No. 275: (2-methoxybenzyl)-(2-pyrrolidino-1-ylquinazolino-4-yl)-amine
Compound No. 276: (2-methoxybenzyl)-(2-piperidino-1-ylquinazolino-4-yl)-amine
Compound No. 277: (2-methoxybenzyl)-(2-morpholino-4-ylquinazolino-4-yl)-amine
Compound No. 278: (2-methoxybenzyl)-[2-(4-methylpiperazino-1-yl)-quinazolino-4-yl]-amine
Compound No. 279: (3-methoxybenzyl)-(2-pyrrolidino-1-ylquinazolino-4-yl)-amine
Compound No. 280: (3-methoxybenzyl)-(2-piperidino-1-ylquinazolino-4-yl)-amine
Compound No. 281: (3-methoxybenzyl)-(2-morpholino-4-ylquinazolino-4-yl)-amine
Compound No. 282: (3-methoxybenzyl)-[2-(4-methylpiperazino-1-yl)-quinazolino-4-yl]-amine
Compound No. 283: (4-methoxybenzyl)-(2-pyrrolidino-1-ylquinazolino-4-yl)-amine
Compound No. 284: (4-methoxybenzyl)-(2-piperidino-1-ylquinazolino-4-yl)-amine
Compound No. 285: (4-methoxybenzyl)-(2-morpholino-4-ylquinazolino-4-yl)-amine
Compound No. 286: (4-methoxybenzyl)-[2-(4-methylpiperazino-1-yl)-quinazolino-4-yl]-amine
Compound No. 287: (2,3-dimethoxybenzyl)-(2-pyrrolidino-1-ylquinazolino-4-yl)-amine
Compound No. 288: (2,3-dimethoxybenzyl)-(2-piperidino-1-ylquinazolino-4-yl)-amine
Compound No. 289: (2,3-dimethoxybenzyl)-(2-morpholino-4-ylquinazolino-4-yl)-amine
Compound No. 290: (2,3-dimethoxybenzyl)-[2-(4-methylpiperazino-1-yl)-quinazolino-4-yl]-amine
Compound No. 291: (2,4-dimethoxybenzyl)-(2-pyrrolidino-1-ylquinazolino-4-yl)-amine
Compound No. 292: (2,4-dimethoxybenzyl)-(2-piperidino-1-ylquinazolino-4-yl)-amine
Compound No. 293: (2,4-dimethoxybenzyl)-(2-morpholino-4-ylquinazolino-4-yl)-amine
Compound No. 294: (2,4-dimethoxybenzyl)-[2-(4-methylpiperazino-1-yl)-quinazolino-4-yl]-amine
Compound No. 295: (2,5-dimethoxybenzyl)-(2-pyrrolidino-1-ylquinazolino-4-yl)-amine
Compound No. 296: (2,5-dimethoxybenzyl)-(2-piperidino-1-ylquinazolino-4-yl)-amine
Compound No. 297: (2,5-dimethoxybenzyl)-(2-morpholino-4-ylquinazolino-4-yl)-amine
Compound No. 298: (2,5-dimethoxybenzyl)-[2-(4-methylpiperazino-1-yl)-quinazolino-4-yl]-amine
Compound No. 299: (3,4-dimethoxybenzyl)-(2-pyrrolidino-1-ylquinazolino-4-yl)-amine
Compound No. 300: (3,4-dimethoxybenzyl)-(2-piperidino-1-ylquinazolino-4-yl)-amine
Compound No. 301: (3,4-dimethoxybenzyl)-(2-morpholino-4-ylquinazolino-4-yl)-amine
Compound No. 302: (3,4-dimethoxybenzyl)-[2-(4-methylpiperazino-1-yl)-quinazolino-4-yl]-amine
Compound No. 303: (3,5-dimethoxybenzyl)-(2-pyrrolidino-1-ylquinazolino-4-yl)-amine
Compound No. 304: (3,5-dimethoxybenzyl)-(2-piperidino-1-ylquinazolino-4-yl)-amine
Compound No. 305: (3,5-dimethoxybenzyl)-(2-morpholino-4-ylquinazolino-4-yl)-amine
Compound No. 306: (3,5-dimethoxybenzyl)-[2-(4-methylpiperazino-1-yl)-quinazolino-4-yl]-amine
Compound No. 307: benzo[1,3]dioxol-5-ylmethyl-(2-pyrrolidino-1-ylquinazolino-4-yl)-amine
Compound No. 308: benzo[1,3]dioxol-5-ylmethyl-(2-piperidino-1-ylquinazolino-4-yl)-amine
Compound No. 309: benzo[1,3]dioxol-5-ylmethyl-(2-morpholino-4-ylquinazolino-4-yl)-amine
Compound No. 310: benzo[1,3]dioxol-5-ylmethyl-[2-(4-methylpiperazino-1-yl)-quinazolino-4-yl]-amine
Compound No. 311: (2-pyrrolidino-1-ylquinazolino-4-yl)-(3,4,5-trimethoxybenzyl)-amine
Compound No. 312: (2-piperidino-1-ylquinazolino-4-yl)-(3,4,5-trimethoxybenzyl)-amine
Compound No. 313: (2-morpholino-4-ylquinazolino-4-yl)-(3,4,5-trimethoxybenzyl)-amine
Compound No. 314: [2-(4-methylpiperazino-1-yl)-quinazolino-4-yl]-(3,4,5-trimethoxybenzyl)-amine
Compound No. 315: (2-pyrrolidino-1-ylquinazolino-4-yl)-(2,4,6-trimethoxybenzyl)-amine
Compound No. 316: (2-piperidino-1-ylquinazolino-4-yl)-(2,4,6-trimethoxybenzyl)-amine
Compound No. 317: (2-morpholino-4-ylquinazolino-4-yl)-(2,4,6-trimethoxybenzyl)-amine
Compound No. 318: [2-(4-methylpiperazino-1-yl)-quinazolino-4-yl]-(2,4,6-trimethoxybenzyl)-amine
Compound No. 319: (2,4-dimethoxybenzyl)-(2-ethoxyquinazolino-4-yl)-amine
Compound No. 320: benzo[1,3]dioxol-5-ylmethyl-(2-ethoxyquinazolino-4-yl)-amine
Compound No. 321: (2-isopropylquinazolino-4-yl)-(2-methoxybenzyl)-amine
Compound No. 322: (2,3-dimethoxybenzyl)-(2-isopropylquinazolino-4-yl)-amine
Compound No. 323: (3,5-dimethoxybenzyl)-(2-isopropylquinazolino-4-yl)-amine
Compound No. 324: (3,4-dimethoxybenzyl)-(2-isopropylquinazolino-4-yl)-amine
Compound No. 325: (2-isopropylquinazolino-4-yl)-(3,4,5-trimethoxybenzyl)-amine
Compound No. 326: (2,5-dimethoxybenzyl)-(2-isopropylquinazolino-4-yl)-amine
Compound No. 327: (2-isopropylquinazolino-4-yl)-(2,4,6-trimethoxybenzyl)-amine
Compound No. 328: (2-benzylquinazolino-4-yl)-(2-methoxybenzyl)-amine
Compound No. 329: (2-benzylquinazolino-4-yl)-(2,3-dimethoxybenzyl)-amine
Compound No. 330: (2-benzylquinazolino-4-yl)-(3,5-dimethoxybenzyl)-amine
Compound No. 331: (2-benzylquinazolino-4-yl)-(3,4-dimethoxybenzyl)-amine
Compound No. 332: (2-benzylquinazolino-4-yl)-(3,4,5-trimethoxybenzyl)-amine
Compound No. 333: (2-benzylquinazolino-4-yl)-(2,4-dimethoxybenzyl)-amine
Compound No. 334: (2-benzylquinazolino-4-yl)-(2,5-dimethoxybenzyl)-amine
Compound No. 335: (2-benzylquinazolino-4-yl)-(2,4,6-trimethoxybenzyl)-amine
Compound No. 336: (2-phenylquinazolino-4-yl)-(3,4,5-trimethoxybenzyl)-amine
Compound No. 337: (2-phenylquinazolino-4-yl)-(2,4,6-trimethoxybenzyl)-amine
Compound No. 338: (2-furano-2-ylquinazolino-4-yl)-(2-methoxybenzyl)-amine
Compound No. 339: (2-furano-2-ylquinazolino-4-yl)-(3-methoxybenzyl)-amine
Compound No. 340: (2-furano-2-ylquinazolino-4-yl)-(4-methoxybenzyl)-amine
Compound No. 341: benzo[1,3]dioxol-5-ylmethyl-(2-furano-2-ylquinazolino-4-yl)-amine
Compound No. 342: (2,3-dimethoxybenzyl)-(2-furano-2-ylquinazolino-4-yl)-amine
Compound No. 343: (3,5-dimethoxybenzyl)-(2-furano-2-ylquinazolino-4-yl)-amine
Compound No. 344: (3,4-dimethoxybenzyl)-(2-furano-2-ylquinazolino-4-yl)-amine
Compound No. 345: (2-furano-2-ylquinazolino-4-yl)-(3,4,5-trimethoxybenzyl)-amine
Compound No. 346: (2,4-dimethoxybenzyl)-(2-furano-2-ylquinazolino-4-yl)-amine
Compound No. 347: (2,5-dimethoxybenzyl)-(2-furano-2-ylquinazolino-4-yl)-amine
Compound No. 348: (2-furano-2-ylquinazolino-4-yl)-(2,4,6-trimethoxybenzyl)-amine
Compound No. 349: (2-methoxybenzyl)-(2-thiopenyl-2-ylquinazolino-4-yl)-amine
Compound No. 350: (3-methoxybenzyl)-(2-thiopenyl-2-ylquinazolino-4-yl)-amine
Compound No. 351: (4-methoxybenzyl)-(2-thiopenyl-2-ylquinazolino-4-yl)-amine
Compound No. 352: benzo[1,3]dioxol-5-ylmethyl-(2-thiopenyl-2-ylquinazolino-4-yl)-amine
Compound No. 353: (2,3-dimethoxybenzyl)-(2-thiopenyl-2-ylquinazolino-4-yl)-amine
Compound No. 354: (3,5-dimethoxybenzyl)-(2-thiopenyl-2-ylquinazolino-4-yl)-amine
Compound No. 355: (3,4-dimethoxybenzyl)-(2-thiopenyl-2-ylquinazolino-4-yl)-amine
Compound No. 356: (2-thiopenyl-2-ylquinazolino-4-yl)-(3,4,5-trimethoxybenzyl)-amine
Compound No. 357: (2,4-dimethoxybenzyl)-(2-thiopenyl-2-ylquinazolino-4-yl)-amine
Compound No. 358: (2,5-dimethoxybenzyl)-(2-thiopenyl-2-ylquinazolino-4-yl)-amine
Compound No. 359: (2-thiopenyl-2-ylquinazolino-4-yl)-(2,4,6-trimethoxybenzyl)-amine
Compound No. 360: (2-methoxybenzyl)-[2-(methoxyphenylmethyl)-quinazolino-4-yl]-amine
Compound No. 361: (3-methoxybenzyl)-[2-(methoxyphenylmethyl)-quinazolino-4-yl]-amine
Compound No. 362: (4-methoxybenzyl)-[2-(methoxyphenylmethyl)-quinazolino-4-yl]-amine
Compound No. 363: benzo[1,3]dioxol-5-ylmethyl-[2-(methoxyphenylmethyl)-quinazolino-4-yl]-a mine
Compound No. 364: (2,3-dimethoxybenzyl)-[2-(methoxyphenylmethyl)-quinazolino-4-yl]-amine
Compound No. 365: (3,5-dimethoxybenzyl)-[2-(methoxyphenylmethyl)-quinazolino-4-yl]-amine
Compound No. 366: (3,4-dimethoxybenzyl)-[2-(methoxyphenylmethyl)-quinazolino-4-yl]-amine
Compound No. 367: [2-(methoxyphenylmethyl)-quinazolino-4-yl]-(3,4,5-trimethoxybenzyl)-amine
Compound No. 368: (2,4-dimethoxybenzyl)-[2-(methoxyphenylmethyl)-quinazolino-4-yl]-amine
Compound No. 369: (2,5-dimethoxybenzyl)-2-(methoxyphenylmethyl)-quinazolino-4-yl]-amine
Compound No. 370: [2-(methoxyphenylmethyl)-quinazolino-4-yl]-(2,4,6-trimethoxybenzyl)-amine
Compound No. 371: (2-methoxybenzyl)-[2-(1-phenylethyl)-quinazolino-4-yl]-amine
Compound No. 372: (3-methoxybenzyl)-[2-(-phenylethyl)-quinazolino-4-yl]-amine
Compound No. 373: (4-methoxybenzyl)-[2-(1-phenylethyl)-quinazolino-4-yl]-amine
Compound No. 374: benzo[1,3]dioxol-5-ylmethyl-[2-(1-phenylethyl)-quinazolino-4-yl]-amine
Compound No. 375: (2,3-dimethoxybenzyl)-[2-(1-phenylethyl)-quinazolino-4-yl]-amine
Compound No. 376: (3,5-dimethoxybenzyl)-[2-(-phenylethyl)-quinazolino-4-yl]-amine
Compound No. 377: (3,4-dimethoxybenzyl)-[2-(1-phenylethyl)-quinazolino-4-yl]-amine
Compound No. 378: [2-(1-phenylethyl)-quinazolino-4-yl]-(3,4,5-trimethoxybenzyl)-amine
Compound No. 379: (2,4-dimethoxybenzyl)-[2-(1-phenylethyl)-quinazolino-4-yl]-amine
Compound No. 380: (2,5-dimethoxybenzyl)-[2-(1-phenylethyl)-quinazolino-4-yl]-amine
Compound No. 381: [2-(1-phenylethyl)-quinazolino-4-yl]-(2,4,6-trimethoxybenzyl)-amine
Compound No. 382: (2-cyclopropylquinazolino-4-yl)-(2-methoxybenzyl)-amine
Compound No. 383: (2-cyclopropylquinazolino-4-yl)-(3-methoxybenzyl)-amine
Compound No. 384: (2-cyclopropylquinazolino-4-yl)-(4-methoxybenzyl)-amine
Compound No. 385: benzo[1,3]dioxol-5-ylmethyl-(2-cyclopropylquinazolino-4-yl)-amine
Compound No. 386: (2-cyclopropylquinazolino-4-yl)-(2,3-dimethoxybenzyl)-amine
Compound No. 387: (2-cyclopropylquinazolino-4-yl)-(3,5-dimethoxybenzyl)-amine
Compound No. 388: (2-cyclopropylquinazolino-4-yl)-(3,4-dimethoxybenzyl)-amine
Compound No. 389: (2-cyclopropylquinazolino-4-yl)-(3,4,5-trimethoxybenzyl)-amine
Compound No. 390: (2-cyclopropylquinazolino-4-yl)-(2,4-dimethoxybenzyl)-amine
Compound No. 391: (2-cyclopropylquinazolino-4-yl)-(2,5-dimethoxybenzyl)-amine
Compound No. 392: (2-cyclopropylquinazolino-4-yl)-(2,4,6-trimethoxybenzyl)-amine
Compound No. 393: (2-methoxybenzyl)-(2-propylquinazolino-4-yl)-amine
Compound No. 394: (3-methoxybenzyl)-(2-propylquinazolino-4-yl)-amine
Compound No. 395: (4-methoxybenzyl)-(2-propylquinazolino-4-yl)-amine
Compound No. 396: benzo[1,3]dioxol-5-ylmethyl-(2-propylquinazolino-4-yl)-amine
Compound No. 397: (2,3-dimethoxybenzyl)-(2-propylquinazolino-4-yl)-amine
Compound No. 398: (3,5-dimethoxybenzyl)-(2-propylquinazolino-4-yl)-amine
Compound No. 399: (3,4-dimethoxybenzyl)-(2-propylquinazolino-4-yl)-amine
Compound No. 400: (2-propylquinazolino-4-yl)-(3,4,5-trimethoxybenzyl)-amine
Compound No. 401: (2,4-dimethoxybenzyl)-(2-propylquinazolino-4-yl)-amine
Compound No. 402: (2,5-dimethoxybenzyl)-(2-propylquinazolino-4-yl)-amine
Compound No. 403: (2-propylquinazolino-4-yl)-(2,4,6-trimethoxybenzyl)-amine
Compound No. 404: (2-methoxybenzyl)-quinazolino-4-ylamine
Compound No. 405: (3-methoxybenzyl)-quinazolino-4-ylamine
Compound No. 406: (4-methoxybenzyl)-quinazolino-4-ylamine
Compound No. 407: benzo[1,3]dioxol-5-ylmethylquinazolino-4-ylamine
Compound No. 408: (2,3-dimethoxybenzyl)-quinazolino-4-ylamine
Compound No. 409: (3,5-dimethoxybenzyl)-quinazolino-4-ylamine
Compound No. 410: (3,4-dimethoxybenzyl)-quinazolino-4-ylamine
Compound No. 411. quinazolino-4-yl-(3,4,5-trimethoxybenzyl)-amine
Compound No. 412: (2,4-dimethoxybenzyl)-quinazolino-4-ylamine
Compound No. 413: (2,5-dimethoxybenzyl)-quinazolino-4-ylamine
Compound No. 414: quinazolino-4-yl-(2,4,6-trimethoxybenzyl)-amine
Compound No. 415: 4-benzyloxy-2-isopropylquinazoline
Compound No. 416: 4-benzylsulfanyl-2-isopropylquinazoline.

5. A pharmaceutical composition comprising the quinazoline derivatives represented by the formula 1 below or a pharmaceutically available salt or prodrug thereof: 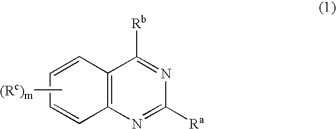
wherein R^{a}, R^{b}, R^{c} and m are the same as defined in claim 1.

6. A medicine for treatment and prevention of diabetes comprising the quinazoline derivatives represented by the formula 1 below or a pharmaceutically available salt or prodrug thereof: 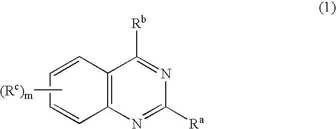
wherein R^{a}, R^{b}, R^{c} and m are the same as defined in claim 1.

7. A medicine for treatment and prevention of obesity comprising the quinazoline derivatives represented by the formula 1 below or a pharmaceutically available salt or prodrug thereof: 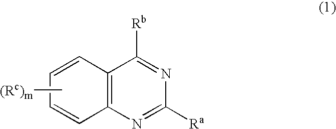
wherein R^{a}, R^{b}, R^{c} and m are the same as defined in claim 1.
